(19) United States
(12) Patent Application Publication
(10) Pub. No.: US 2026/0098007 A1
(43) Pub. Date: Apr. 9, 2026

(54) Compounds and Their Use in Filtering Ultraviolet and/or High-Energy Visible Light

(71) Applicant: Soliome Inc., San Francisco, CA (US)

(72) Inventors: Micah Nelp, San Francisco, CA (US); Anthony Young, San Francisco, CA (US)

(21) Appl. No.: 19/110,764

(22) PCT Filed: Sep. 13, 2023
(86) PCT No.: PCT/US2023/032611

§ 371 Date: Mar. 11, 2025

Related U.S. Application Data
(60) Provisional application No. 63/506,758, filed on Jun. 7, 2023.

(30) Foreign Application Priority Data
Sep. 13, 2022 (EP) 22195447.2

(51) Int. Cl. C07C 237/20; A61K 8/41; A61K 8/42; A61K 8/49; A61Q 17/04; C07C 229/34; C07C 229/38; C07D 207/40; C07D 303/46; C07D 303/48

(52) U.S. Cl. CPC C07C 237/20; A61K 8/41; A61K 8/42; A61K 8/4913; A61K 8/4973; A61Q 17/04; C07C 229/34; C07C 229/38; C07D 207/40; C07D 303/46; C07D 303/48; A61K 2800/52

(57) ABSTRACT

This invention provides compounds of formula (II) or tautomers, stereoisomers, hydrates, solvates, salts, or polymorphs thereof.

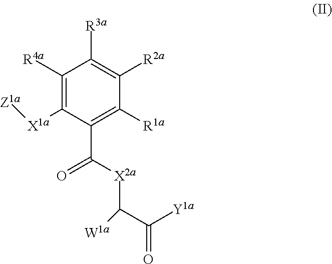

wherein Z^{1a}, X^{1a}, Ar¹, X^{2a}, W^{1a}, Y^{1a}, R^{1a}, R^{2a}, R^{3a}, and R^{4a} have the same meaning as that defined in the description and claims. The invention also provides these compounds for use in filtering ultraviolet (UV) and high-energy visible light. The invention also relates to compositions comprising said compounds. The present invention also relates to consumer products and packaging material comprising said compounds.

### CROSS REFERENCE TO RELATED APPLICATIONS

This application is a national phase entry under 35 U.S.C. § 371 of International Patent Application PCT/US2023/032611, filed Sep. 13, 2023, designating the United States of America and published in English as International Patent Publication WO2024/059119 on Mar. 21, 2024, which claims the benefit under Article 8 of the Patent Cooperation Treaty to U.S. Provisional Application Ser. No. 63/506,758, filed Jun. 7, 2023, and European Patent Application Serial No. 22195447.2, filed Sep. 13, 2022, the entireties of which are hereby incorporated by reference.

### FIELD OF THE INVENTION

The present invention relates to novel compounds and to their use as ultraviolet (UV) and/or high-energy visible (HEV) light absorbers and/or filters. The present invention also relates to compositions comprising said compounds. The present invention also relates to consumer products and packaging material comprising said compounds.

### BACKGROUND OF THE INVENTION

High-energy light from the sun and manmade light sources such as UV light and high-energy visible light, is known to be responsible for cellular damage. This light ranges mainly from 290-320 nm (UVB), 320-400 nm (UVA), and 400-450 nm (HEV). Photons in this energy range can be absorbed by many biological, pharmaceutical, and cosmetic molecules causing direct photo-degradation or transfer of that energy to oxygen leading to a cascade of oxidative reactions. In living tissue, UV and high-energy visible light can damage critical cellular components like DNA and lipids leading to mutations and cellular death that may manifest as cancer, erythema, and accelerated skin aging. The absorption of UV and high-energy visible light can also degrade critical pharmaceutical components like the sunscreen active ingredient avobenzone that typically requires photo-stabilizers to maintain its UVA-protective effect. Food items are also susceptible to UV and high-energy visible light damage, particularly unsaturated fatty acids like olive oil and the essential amino acid tryptophan in proteins that is readily oxidized by photo-excited molecules and oxygen.

Molecules that absorb UV and high-energy visible light and dissipate that energy as heat are thus desirable. Such filters are particularly useful if they are photo-stable so that they can be used more sparingly, have low fluorescence so they dissipate most of the energy as heat, are entirely biodegradable so that they do not persist in the environment or in our tissues, and do not readily absorb through skin so that they can remain at the surface to provide protective effects and to prevent significant flux through biodegradation pathways in our tissues. Many UV filters currently approved as sunscreen active ingredients in the United States fail at one or more of these tests, persisting in the environment either unchanged or as stable xenobiotic degradation products that also readily absorb through skin and accumulate in serum. These sunscreen active ingredients also show distressing evidence that they effectively mimic hormones in our tissue and that they cause environmental damage in sensitive marine ecosystems.

There is thus a significant need for UV and/or high-energy visible light filtering compounds that are preferably biodegradable, even by human cells, that can match the performance of existing UV filters.

### SUMMARY OF THE INVENTION

The present invention is directed to a compound of formula (I):

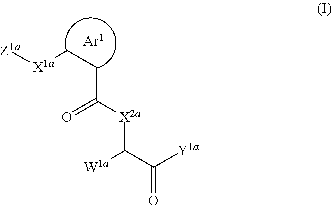

or a tautomer, a stereoisomer, a hydrate, a solvate, a salt, or a polymorph thereof, wherein
Ar¹ is aryl or heteroaryl; wherein each of said aryl or heteroaryl can be unsubstituted or substituted with one or more R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene; wherein each of said alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene, can be unsubstituted or substituted with one or more R^{za1}; wherein each R^{za1} is independently selected from the group comprising —COO—R^{h},

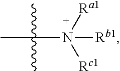

—NR^{a1}R^{b1}, halo, alkyl, haloalkyl, alkylcarbonyl, alkenyl, and alkynyl;
R^{h} is selected from the group comprising hydrogen, alkyl, alkenyl, and alkynyl,
R^{a1} is selected from the group comprising hydrogen, alkyl, alkenyl, and alkynyl;
R^{b1} is selected from the group comprising hydrogen, alkyl, alkenyl, and alkynyl;
R^{c1} is selected from the group comprising hydrogen, alkyl, alkenyl, and alkynyl;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is selected from —NR^{b}—, —O—, —S—, SO₂—O—, or —SO—O—;
Z^{1a} is selected from the group comprising hydrogen, —CO—R^{a}, —CO—R^{e}—CO—O—R^{b}, —CO—R^{f}-R^{g}, —R^{f}—R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; wherein n is an integer selected from 1, 2 or 3;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more O or S;
R^{g} is selected from N-maleimidyl, N-maleimidyloxy, or N-maleimidyloxycarbonyl;
W^{1a} is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is selected from —OR^{a}, —R^{a}, or —NR^{a}R^{b}, or Y^{1a} is a moiety of formula (A);

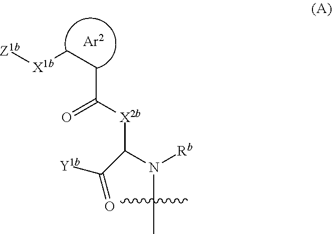

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a}; X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}—R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; and
Y^{1b} is selected from —OR^{a}, —NR^{b}R^{a}, or —R^{a};
with the proviso that

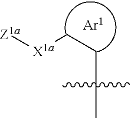

is not

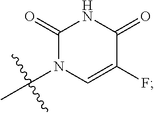 [SMILES: *n1cc(F)c(=O)[nH]c1=O]

and
with the proviso that said compound is not:
2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(2-amino-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid);
2-amino-4-oxo-4-(2-(((2S,3R,4R,5S,6R)-3,4,5-trihydroxy-6-(hydroxymethyl)tetrahydro-2H-pyran-2-yl)amino)phenyl)butanoic acid;
methyl 2-amino-4-(2-aminophenyl)-4-oxobutanoate;
4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
(2R,3R,4S,5R)-2-(acetoxymethyl)-6-(2-amino-3-(4-methoxy-4-oxobutanoyl)phenoxy)tetrahydro-2H-pyran-3,4,5-triyl triacetate;
2-amino-4-(2-amino-5-hydroxyphenyl)-4-oxobutanoic acid.

In some embodiment, the present invention is directed to a compound of formula (I):

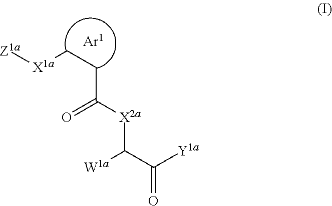

or a tautomer, a stereoisomer, a hydrate, a solvate, a salt, or a polymorph thereof, wherein
Ar¹ is aryl or heteroaryl; wherein each of said aryl or heteroaryl can be unsubstituted or substituted with one or more R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is selected from —NR^{b}—, —O—, —S—, SO₂—O—, or —SO—O—;
Z^{1a} is selected from the group comprising hydrogen, —CO—R^{a}, —CO—R^{e}—CO—O—R^{b}, —CO—R^{f}-R^{g}, —R^{f}—R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; n is an integer selected from 1, 2 or 3;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more as 0 or S;
R^{g} is selected from N-maleimidyl, N-maleimidyloxy, or N-maleimidyloxycarbonyl;
W^{1a} is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is a moiety of formula (A) or Y^{1a} is selected from —OR^{a}, —R^{a}, or —NR^{a}R^{b};

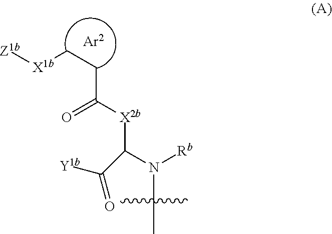

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}—R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; m is an integer selected from 1, 2 or 3; and
Y^{1b} is selected from —OR^{a}, —NR^{b}R^{a}, or —R^{a};
with the proviso that said compound is not:
2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
2-amino-4-oxo-4-(2-(((2S,3R,4R,5S,6R)-3,4,5-trihydroxy-6-(hydroxymethyl)tetrahydro-2H-pyran-2-yl)amino)phenyl)butanoic acid, and
methyl 2-amino-4-(2-aminophenyl)-4-oxobutanoate.

In some embodiment, the present invention is directed to a compound of formula (II):

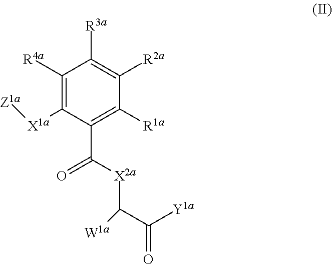

or a tautomer, a stereoisomer, a hydrate, a solvate, a salt, or a polymorph thereof, wherein
R^{1a}, R^{2a}, R^{3a}, and R^{4a} are each independently R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, and —R^{f1}-R^{g1}; wherein each of said alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene, can be unsubstituted or substituted with one or more R^{za1}; wherein each R^{za1} is independently selected from the group comprising —COO—R^{h},

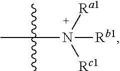

—NR^{a1}R^{b1}, —R^{f1}-R^{g1}, halo, alkyl, haloalkyl, alkylcarbonyl, alkenyl, and alkynyl; wherein R^{h} is selected from the group comprising hydrogen, alkyl, alkenyl, and alkynyl; R^{a1} is selected from the group comprising hydrogen, alkyl, alkenyl, and alkynyl; R^{b1} is selected from the group comprising hydrogen, alkyl, alkenyl, and alkynyl; R^{c1} is selected from the group comprising hydrogen, alkyl, alkenyl, and alkynyl;
R^{f1} is alkylene, wherein said alkylene can be interrupted by one or more O or S;
R^{g1} is hydrogen, or alkyl;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}—R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; wherein n is an integer selected from 1, 2 or 3;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more O or S;
R^{g} is selected from hydrogen, alkyl, N-maleimidyl, N-maleimidyloxy, or N-maleimidyloxycarbonyl;
W^{1a} is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is selected from —OR^{a}, —R^{a}, or —NR^{a}R^{b}, or Y^{1a} is a moiety of formula (A);

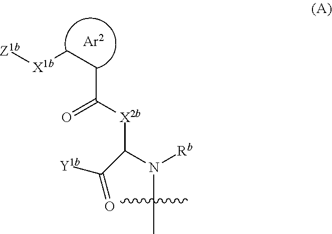

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}—R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; and
Y^{1b} is selected from —OR^{a}, —NR^{b}R^{a}, or —R^{a};
with the proviso that said compound is not:
2-amino-4-(2-aminophenyl)-4-oxobutanoic acid;
(R)-2-amino-4-(2-aminophenyl)-4-oxobutanoic acid;
(S)-2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
2-amino-4-oxo-4-(2-(((3R,4R,5S,6R)-3,4,5-trihydroxy-6-(hydroxymethyl)tetrahydro-2H-pyran-2-yl)amino)phenyl)butanoic acid;
(2S)-2-amino-4-oxo-4-(2-(((3R,4R,5S,6R)-3,4,5-trihydroxy-6-(hydroxymethyl)tetrahydro-2H-pyran-2-yl)amino)phenyl)butanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
methyl 2-amino-4-(2-aminophenyl)-4-oxobutanoate;
4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
(S)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(R)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(2R,3R,4S,5R)-2-(acetoxymethyl)-6-(2-amino-3-(4-methoxy-4-oxobutanoyl)phenoxy)tetrahydro-2H-pyran-3,4,5-triyl triacetate;
2-amino-4-(2-amino-5-hydroxyphenyl)-4-oxobutanoic acid;
(S)-2-amino-4-(2-amino-5-hydroxyphenyl)-4-oxobutanoic acid;
(R)-2-amino-4-(2-amino-5-hydroxyphenyl)-4-oxobutanoic acid;
5-(2-aminophenyl)-5-oxopentanoic acid;
4-(2-aminophenyl)-4-oxobutanoic acid;
methyl 6-(2-aminophenyl)-6-oxohexanoate;
methyl 4-(2-aminophenyl)-2-((tert-butoxycarbonyl)amino)-4-oxobutanoate;
butyl 2-amino-4-(2-aminophenyl)-4-oxobutanoate;
ethyl (S)-2-amino-4-(2-aminophenyl)-4-oxobutanoate;
(S)-2-amino-4-(2-aminophenyl)-4-oxobutanoic acid diacetate;
(R)-2-amino-4-(2-formamidophenyl)-4-oxobutanoic acid;
(S)-2-amino-4-(2-formamidophenyl)-4-oxobutanoic acid;
2-amino-4-(2-formamidophenyl)-4-oxobutanoic acid;
methyl 2-acetamido-4-(2-acetamidophenyl)-4-oxobutanoate;
4-(2-amino-3-(((2R,3S,4R,5R,6S)-3,4,5-trihydroxy-6-((((2S,3S,4R,5R,6S)-3,4,5-trihydroxy-6-(hydroxymethyl)tetrahydro-2H-pyran-2-yl)oxy)methyl)tetrahydro-2H-pyran-2-yl)oxy)phenyl)-4-oxobutanoic acid;
4-(2-amino-3-hydroxyphenyl)-4-oxobutanoic acid O-beta-D-diglucoside); 4-(2-amino-3-(((2R,3S,4R,5R,6S)-3,4-5-trihydroxy-6-(hydroxymethyl)tetrahydro-2H-pyran-2-yl)oxy)phenyl)-4-oxobutanoic acid;
4-(2-amino-3-hydroxyphenyl)-4-oxobutanoic acid O-beta-d-glucoside;
2-amino-4-(2-formamido-5-hydroxyphenyl)-4-oxobutanoic acid;
2-amino-4-(2-amino-3-chlorophenyl)-4-oxobutanoic acid;
(R)-2-amino-4-(2-amino-3-chlorophenyl)-4-oxobutanoic acid;
(S)-2-amino-4-(2-amino-3-chlorophenyl)-4-oxobutanoic acid;
2-amino-4-(2-amino-4-chlorophenyl)-4-oxobutanoic acid;
(R)-2-amino-4-(2-amino-4-chlorophenyl)-4-oxobutanoic acid;
(S)-2-amino-4-(2-amino-4-chlorophenyl)-4-oxobutanoic acid;
2-amino-4-(2-amino-5-fluorophenyl)-4-oxobutanoic acid;
(R)-2-amino-4-(2-amino-5-fluorophenyl)-4-oxobutanoic acid;
(S)-2-amino-4-(2-amino-5-fluorophenyl)-4-oxobutanoic acid;
2-amino-4-(2-amino-5-bromophenyl)-4-oxobutanoic acid;
(R)-2-amino-4-(2-amino-5-bromophenyl)-4-oxobutanoic acid;
(S)-2-amino-4-(2-amino-5-bromophenyl)-4-oxobutanoic acid;
2-amino-4-(2-amino-5-methylphenyl)-4-oxobutanoic acid;
(R)-2-amino-4-(2-amino-5-methylphenyl)-4-oxobutanoic acid;
(S)-2-amino-4-(2-amino-5-methylphenyl)-4-oxobutanoic acid;
2-amino-4-(2-amino-5-ethylphenyl)-4-oxobutanoic acid;
(R)-2-amino-4-(2-amino-5-ethylphenyl)-4-oxobutanoic acid;
(S)-2-amino-4-(2-amino-5-ethylphenyl)-4-oxobutanoic acid;
2-amino-4-(2-amino-5-propylphenyl)-4-oxobutanoic acid;
(R)-2-amino-4-(2-amino-5-propylphenyl)-4-oxobutanoic acid;
(S)-2-amino-4-(2-amino-5-propylphenyl)-4-oxobutanoic acid;
2-amino-4-(2-amino-5-isopropylphenyl)-4-oxobutanoic acid;
(R)-2-amino-4-(2-amino-5-isopropylphenyl)-4-oxobutanoic acid;
(S)-2-amino-4-(2-amino-5-isopropylphenyl)-4-oxobutanoic acid;
2-amino-4-(2-amino-5-cyclohexylphenyl)-4-oxobutanoic acid;
(R)-2-amino-4-(2-amino-5-cyclohexylphenyl)-4-oxobutanoic acid;
(S)-2-amino-4-(2-amino-5-cyclohexylphenyl)-4-oxobutanoic acid;
2-amino-4-(2-(methylamino)phenyl)-4-oxobutanoic acid;
(S)-2-amino-4-(2-(methylamino)phenyl)-4-oxobutanoic acid;
(R)-2-amino-4-(2-(methylamino)phenyl)-4-oxobutanoic acid;
2-amino-4-(2-(dimethylamino)phenyl)-4-oxobutanoic acid;
(S)-2-amino-4-(2-(dimethylamino)phenyl)-4-oxobutanoic acid;
(R)-2-amino-4-(2-(dimethylamino)phenyl)-4-oxobutanoic acid;
2-amino-4-(2-amino-5-chlorophenyl)-4-oxobutanoic acid;
(R)-2-amino-4-(2-amino-5-chlorophenyl)-4-oxobutanoic acid;
(S)-2-amino-4-(2-amino-5-chlorophenyl)-4-oxobutanoic acid;
2-amino-4-(2-amino-6-chlorophenyl)-4-oxobutanoic acid;
(R)-2-amino-4-(2-amino-6-chlorophenyl)-4-oxobutanoic acid;
(S)-2-amino-4-(2-amino-6-chlorophenyl)-4-oxobutanoic acid;
2-acetamido-4-(2-amino-5-chlorophenyl)-4-oxobutanoic acid;
(R)-2-acetamido-4-(2-amino-5-chlorophenyl)-4-oxobutanoic acid;
(S)-2-acetamido-4-(2-amino-5-chlorophenyl)-4-oxobutanoic acid;
2-amino-4-(2-amino-3-hydroxyphenyl)-4-oxobutanoic acid;
(S-)2-amino-4-(2-amino-3-hydroxyphenyl)-4-oxobutanoic acid;
(R-)2-amino-4-(2-amino-3-hydroxyphenyl)-4-oxobutanoic acid;
methyl (S-)2-amino-4-(2-aminophenyl)-4-oxobutanoate;
2-amino-4-(2-aminophenyl)-4-oxobutanamide;
(R)-2-amino-4-(2-aminophenyl)-4-oxobutanamide;
(S)-2-amino-4-(2-aminophenyl)-4-oxobutanamide;
(S)-2-acetamido-4-(2-acetamidophenyl)-4-oxobutanoic acid;
(S)-2-formamido-4-(2-formamidophenyl)-4-oxobutanoic acid;
4-(2-aminophenyl)-4-oxo-2-phenylbutanamide.

The present invention also relates to a cosmetic or pharmaceutical composition, comprising at least one compound according to the invention, and optionally at least one dermatologically or pharmaceutically acceptable carrier.

The present invention is based on the unexpected finding that the herein described compounds are compounds that absorb UV and/or high-energy visible light. These compounds can be particularly useful in filtering UV and/or high-energy visible light. The compounds can be particularly useful for dissipation of the aforementioned energy as heat, in particular for the absorption of UV light from solar irradiance on the skin that would otherwise penetrate into living tissue and cause cellular damage leading to among other things cancer, erythema, and accelerated aging. They can be also useful for the protection of body parts from UV damage. These compound can therefore be used for preventing cancer, erythema, and/or accelerated aging.

The compounds can also be used for photo-protection of hair, food, plants, and in the manufacture of photo-protective materials such as plastics for food packaging. They can be also useful in food packaging to protect food items from UV and/or high-energy visible radiations damage.

They can absorb UV and/or high-energy visible light, and can be used to prevent the photodegradation of ingredients, such as pharmaceutical and cosmetic ingredients. They can also be used for the prevention of coloration that results from absorption of high-energy visible light.

In some embodiments, the compounds have the further advantage of being biodegradable. They also have high-extinction coefficient, high photo-stability, low fluorescence, low singlet oxygen yield. They also have the advantage of having a low likelihood of readily absorbing through skin.

In some embodiments, the present compounds can act as antioxidant, and can also effectively prevent damage from hydroxyl radicals that can form from pollution and UV radiation.

The present invention therefore also relates to the use of at least one compound according to the invention as UV absorbing filter and/or high-energy visible light filters.

The present invention also relates to the use of at least one compound according to the invention for protecting from UV and/or high-energy visible radiations.

The compounds can be used for the photo-protection of pharmaceutical and cosmetic formulations, such as cosmetic skin formulations.

The present invention also relates to the use of at least one compound according to the invention as a light stabilizer to protect ingredients against photolytic degradation.

The present invention also relates to the use of at least one compound according to the invention as a UV and/or high-energy visible light absorbing filter in a consumer product, or in a packaging.

The present invention also encompasses a consumer product comprising at least one compound according to the invention.

The present invention also encompasses a packaging material comprising at least one compound according to the invention.

The above and other characteristics, features, and advantages of the present invention will become apparent from the following detailed description, which illustrate, by way of example, the principles of the invention.

### BRIEF DESCRIPTION OF THE DRAWINGS

FIG. 1 is a line graph showing the absorbance spectrum of compound 1. It shows absorbance up to 410 nm, thereby useful in providing high-energy visible light protection.

FIG. 2 is a line graph showing the absorbance spectrum of compound 2. It shows strong absorbance in the UVB region.

FIG. 3 is a line graph showing the combined absorbance spectrum of compounds 1 and 2. The absorbance spectrum of both compounds can be combined to give broad spectrum UVB and UVA protection.

FIG. 4 is a line graph showing the absorbance spectrum of compound 4.

FIG. 5 is a line graph showing the absorbance spectrum of compound 5.

FIG. 6 is a line graph showing the hydroxyl radical antioxidant capacity (HORAC) testing of Compound 4.

FIG. 7 is a line graph showing the absorbance spectrum of compound 42.

FIG. 8 is a line graph showing the absorbance spectrum of compound 43.

FIG. 9 is a line graph showing the absorbance spectrum of compound 44.

FIG. 10 is a line graph showing the absorbance spectrum of compound 45.

FIG. 11 is a line graph showing the absorbance spectrum of compound 46.

FIG. 12 is a line graph showing the absorbance spectrum of compound 6.

FIG. 13 is a line graph showing the absorbance spectrum of compound 47.

FIG. 14 is a line graph showing the absorbance spectrum of compound 48.

FIG. 15 is a line graph showing the absorbance spectrum of compound 49.

FIG. 16 is a line graph showing the absorbance spectrum of compound 50.

FIG. 17 is a photograph showing the protective effect of compound 42 against damage to skin by UV.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. When describing the compounds, processes, method and uses of the invention, the terms used are to be construed in accordance with the following definitions, unless the context dictates otherwise.

As used herein, the singular forms “a”, “an”, and “the” include both singular and plural referents unless the context clearly dictates otherwise. By way of example, “a compound” means one compound or more than one compound.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The terms “comprising”, “comprises” and “comprised of” as used herein are synonymous with “including”, “includes” or “containing”, “contains”, and are inclusive or open-ended and do not exclude additional, non-recited members, elements, or method steps. The terms “comprising”, “comprises” and “comprised of” also include the term “consisting of”.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g., from 1.0 to 5.0 includes both 1.0 and 5.0). Any numerical range recited herein is intended to include all sub-ranges subsumed therein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims and statements, any of the embodiments can be used in any combination.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e., a compound that is sufficiently robust to survive isolation from a reaction mixture.

The term “halo” or “halogen” as a group or part of a group is generic for fluoro, chloro, bromo, iodo.

The term “hydroxyl” or “hydroxy” as used herein refers to the group —OH.

The term “thio” or “thiol” as used herein refers to the group —SH.

The term “alkyl” as a group or part of a group, refers to a hydrocarbyl group of formula CₙH₂ₙ₊₁ wherein n is a number greater than or equal to 1, with no site of unsaturation. Alkyl groups may be linear or branched and may be substituted as indicated herein. Generally, alkyl groups of this invention comprise from 1 to 20 carbon atoms, preferably from 1 to 10 carbon atoms, more preferably from 1 to 6 carbon atoms, more preferably from 1 to 4 carbon atoms. When a subscript is used herein following a carbon atom, the subscript refers to the number of carbon atoms that the named group may contain. For example, the term “C₁₋₂₀alkyl”, as a group or part of a group, refers to a hydrocarbyl group of formula CₙH₂ₙ₊₁ wherein n is a number ranging from 1 to 20. Thus, for example, “C₁₋₆alkyl” includes all linear or branched alkyl groups with between 1 and 6 carbon atoms, and thus includes methyl, ethyl, n-propyl, i-propyl, butyl, and its isomers (e.g., n-butyl, i-butyl, and t-butyl); pentyl and its isomers, hexyl, and its isomers, etc. For example, C₁₋₄alkyl includes all linear or branched alkyl groups having 1 to 4 carbon atoms, and thus includes for example methyl, ethyl, n-propyl, i-propyl, 2-methyl-ethyl, butyl, and its isomers (e.g., n-butyl, i-butyl, and t-butyl), and the like. In particular embodiments, the term alkyl refers to C₁₋₁₂alkyl (C₁₋₁₂ hydrocarbons), yet more in particular to C₁₋₉alkyl (C₁₋₉ hydrocarbons), yet more in particular to C₁₋₆alkyl (C₁₋₆ hydrocarbons) as further defined herein above. Non-limiting examples of alkyl include methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl.

When the suffix “ene” is used in conjunction with an alkyl group, i.e., “alkylene”, this is intended to mean the alkyl group as defined herein having two single bonds as points of attachment to other groups. As used herein, the term “alkylene” also referred as “alkanediyl”, by itself or as part of another substituent, refers to alkyl groups that are divalent, i.e., having two monovalent group centers derived by the removal of two hydrogen atoms from the same or two different carbon atoms of a parent alkane, i.e., with two single bonds for attachment to two other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “hydrocarbyl” group is used herein in accordance with the definition specified by IUPAC as follows: a univalent group formed by removing a hydrogen atom from a hydrocarbon (that is, a group containing only carbon and hydrogen).

The term “alkenyl” as a group or part of a group, refers to an unsaturated hydrocarbyl group which may be linear, or branched, comprising one or more with at least one site (usually 1 to 3, preferably 1) of unsaturation, namely at least one sp² carbon-sp² carbon double bond. Generally, alkenyl groups of this invention comprise from 2 to 20 carbon atoms, preferably from 2 to 10 carbon atoms, preferably from 2 to 8 carbon atoms, more preferably 2 to 6 carbon atoms. When a subscript is used herein following a carbon atom, the subscript refers to the number of carbon atoms that the named group may contain. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like. The double bond may be in the cis or trans configuration.

When the suffix “ene” is used in conjunction with an alkenyl group, i.e., “alkenylene”, this is intended to mean the alkenyl group as defined herein having two single bonds as points of attachment to other groups. As used herein, the term “alkenylene” by itself or as part of another substituent, refers to alkenyl groups that are divalent, i.e., having two monovalent centers derived by the removal of two hydrogen atoms from the same or two different carbon atoms of a parent alkene, i.e., with two single bonds for attachment to two other groups. Alkenylene groups may be linear or branched and may be substituted as indicated herein. Non-limiting examples of alkenylene groups include —CH═CH—, —C(CH₃)═CH—, —C(CH₃)═C(CH₃)—, —CH═CH—CH₂—, —CH₂—C(CH₃)═CH—, —CH₂—CH═C(CH₃)—, —CH₂—CH₂—CH═CH—, and the like.

The term “alkynyl” as a group or part of a group, refers to a branched or straight chain hydrocarbon comprising at least one site (usually 1 to 3, preferably 1) of unsaturation, namely a sp¹ carbon-sp¹ carbon triple bond. In particular embodiments, the term alkynyl refers to C₂₋₁₂ alkynyl (C₂₋₁₂ hydrocarbons), preferably to C₂₋₉ alkynyl (C₂₋₉ hydrocarbons) yet more preferably to C₂₋₆ alkynyl (C₂₋₆ hydrocarbons) as further defined herein above with at least one site (usually 1 to 3, preferably 1) of unsaturation, namely at least one sp¹ carbon-sp¹ carbon triple bond. Examples of alkynyl include but are not limited to: ethynyl (—C≡CH), 3-ethyl-cyclohept-1-ynylene, and 1-propynyl (propargyl, —CH₂C≡CH).

When the suffix “ene” is used in conjunction with an alkynyl group, i.e., “alkynylene”, this is intended to mean the alkynyl group as defined herein having two single bonds as points of attachment to other groups. As used herein, the term “alkynylene” by itself or as part of another substituent, refers to alkynyl groups that are divalent, i.e., with two single bonds for attachment to two other groups. Alkynylene groups may be linear or branched and may be substituted as indicated herein. Non-limiting examples of alkynylene groups include —C≡C—, —CH₂—C≡C—, —C≡C—CH₂—, —CH₂—CH₂—C≡C—, and the like.

The term “cycloalkyl”, as a group or part of a group, refers to a cyclic alkyl group, that is a monovalent, saturated, hydrocarbyl group having 1 or more cyclic structure, and comprising from 3 to 20 carbon atoms, more preferably from 3 to 10 carbon atoms, more preferably from 3 to 8 carbon atoms; more preferably from 3 to 6 carbon atoms. Cycloalkyl includes all saturated hydrocarbon groups containing 1 or more rings, including monocyclic, bicyclic groups or tricyclic. For example, cycloalkyl comprises a C₃₋₁₀ monocyclic or C₇₋₁₈ polycyclic saturated hydrocarbon, such as for instance cyclopropyl, cyclobutyl, cyclopentyl, cyclopropylethylene, methylcyclopropylene, cyclohexyl, cycloheptyl, cyclooctyl, cyclooctylmethylene, norbornyl, fenchyl, trimethyltricycloheptyl, decalinyl, adamantyl and the like. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₁₀cycloalkyl”, refers to a cyclic alkyl group comprising from 3 to 10 carbon atoms. For example, the term “C₃₋₈cycloalkyl”, refers to a cyclic alkyl group comprising from 3 to 8 carbon atoms. For example, the term “C₃₋₆cycloalkyl”, refers to a cyclic alkyl group comprising from 3 to 6 carbon atoms. For the avoidance of doubt, fused systems of a cycloalkyl ring with a heterocyclic ring are considered as heterocycle irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “cycloalkylalkylene” or “cycloalkyl-alkylene”, as a group or part of a group, refers to a group of formula R^{z}R^{y} wherein R^{z} is cycloalkyl, and R^{y} is alkylene as defined herein.

The term “alkoxy” or “alkyloxy”, as a group or part of a group, refers to a group of formula —OR^{x} wherein R^{x} is alkyl as defined herein. Non-limiting examples of suitable C₁₋₆alkoxy include methoxy, ethoxy, propoxy, isopropoxy, butoxy, isobutoxy, sec-butoxy, tert-butoxy, pentyloxy, and hexyloxy.

The term “alkenyloxy”, as a group or part of a group, refers to a group of formula —OR^{w} wherein R^{w} is alkenyl as defined herein.

The term “alkynyloxy”, as a group or part of a group, refers to a group of formula —OR^{v} wherein R^{v} is alkynyl as defined herein.

The term “aryl”, as a group or part of a group, refers to a polyunsaturated, aromatic hydrocarbyl group having a single ring (i.e., phenyl) or multiple aromatic rings fused together (e.g., naphthyl), or linked covalently, typically containing 6 to 20 atoms; preferably 6 to 10, wherein at least one ring is aromatic. Typical aryl groups include, but are not limited to 1 ring, or 2 or 3 rings fused together, derived from benzene, naphthalene, anthracene, biphenyl, and the like. The aromatic ring may optionally include one to two additional rings. Fused systems of an aryl ring with a cycloalkyl ring, or a cycloalkenyl ring, or a cycloalkynyl ring, are considered as aryl irrespective of the ring that is bound to the core structure. Fused systems of an aryl ring with a heterocycle are considered as heterocycle irrespective of the ring that is bound to the core structure. Fused systems of an aryl ring with a heteroaryl are considered as heteroaryl irrespective of the ring that is bound to the core structure. Examples of suitable aryl include C₆₋₂₀aryl, preferably C₆₋₁₀aryl, more preferably C₆₋₉aryl. Non-limiting examples of aryl comprise phenyl, biphenylyl, biphenylenyl, or 1- or 2-naphthalenyl; 1-, 2-, 3-, 4-, 5- or 6-tetralinyl (also known as “1,2,3,4-tetrahydronaphtalene); 1-, 2-, 3-, 4-, 5-, 6-, 7- or 8-azulenyl, 4-, 5-, 6 or 7-indenyl; 4- or 5-indanyl; 5-, 6-, 7- or 8-tetrahydronaphthyl; 1,2,3,4-tetrahydronaphthyl; and 1,4-dihydronaphthyl; 1-, 2-, 3-, 4- or 5-pyrenyl.

The term “aryl-alkylene”, as a group or part of a group, refers to an alkyl as defined herein, wherein at least one hydrogen atom is replaced by at least one aryl as defined herein. Non-limiting examples of aryl-alkylene group include benzyl, phenethyl, dibenzylmethyl, benzyl, 2-phenylethan-1-yl, 2-phenylethen-1-yl, naphthylmethyl, 2-naphthylethyl, and the like. The term “C₆₋₁₀aryl-C₁₋₆alkylene” means that the alkylene moiety of the aryl-alkylene group can comprise 1 to 6 carbon atoms and the aryl moiety is 6 to 10 carbon atoms.

The term “aryl-alkenylene” as a group or part of a group, refers to an alkenylene in which one of the hydrogen atoms bonded to a carbon atom, is replaced with an aryl. The term “C₆₋₁₀arylC₂₋₆alkenylene” means that the alkenylene moiety of the arylalkenyl group can comprise 2 to 6 carbon atoms and the aryl moiety 6 to 10 carbon atoms.

The term “aryl-alkynylene” as a group or part of a group, refers to an alkynylene in which one of the hydrogen atoms bonded to a carbon atom, is replaced with an aryl. The term “C₆₋₁₀arylC₂₋₆alkynylene” means that the alkenylene moiety of the arylalkynyl group can comprise 2 to 6 carbon atoms and the aryl moiety 6 to 10 carbon atoms.

The term “haloalkyl”, as a group or part of a group, refers to an alkyl group having the meaning as defined herein, wherein one or more hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl and the like.

The term “glycosyl”, as used herein, refers to a chemical group which is obtained by replacing the hydroxyl group from the hemiacetal function of a mono-, di-, tri-, oligo- or a poly-saccharide. A glycosyl moiety having more than one monosaccharide may represent a linear or branched structure.

The term “monosaccharide”, as used herein, refers to a simple form of a sugar that consists of a single saccharide unit which cannot be further decomposed to smaller saccharide building blocks or moieties. Common examples of monosaccharides include glucose (dextrose), fructose, galactose, mannose, and ribose. The monosaccharide can be linear or cyclic (i.e., can be referred to as furanoses, pyranoses). Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. The above monosaccharides encompass both D- and L-monosaccharides.

Alternatively, the monosaccharide can be a monosaccharide derivative, in which the saccharide unit comprises one or more substituents other than hydroxyls. Such derivatives can be, but are not limited to, ethers, esters, amides, acids, phosphates and amines. Amine derivatives include, for example, glucosamine, galactosamine, fructosamine and mannosamine. Amide derivatives include, for example, N-acetylated amine derivatives of saccharides (e.g., N-acetylglucosamine, N-acetylgalactosamine).

The term “disaccharide”, as used herein, refers to a molecular moiety containing two monosaccharides covalently bound to each other. Disaccharides include maltose [glucose-glucose], lactose [galactose-glucose]and sucrose [fructose-glucose].

The term “trisaccharide”, as used herein, refers to a molecular moiety containing three monosaccharides covalently bound to each other.

The term “oligosaccharide”, as used herein, refers to a molecular moiety containing at least 4 monosaccharides covalently bound to each other. Oligosaccharides include tetrasaccharides, pentasaccharides, hexasaccharides, heptasaccharides, octasaccharides, and the like. Covalent linkages for linking sugars generally consist of glycosidic linkages (i.e., C—O—C bonds) formed from the hydroxyl groups of adjacent sugars. Linkages can occur between the 1-carbon (the anomeric carbon) and the 4-carbon of adjacent sugars (i.e., a 1-4 linkage), the 1-carbon (the anomeric carbon) and the 3-carbon of adjacent sugars (i.e., a 1-3 linkage), the 1-carbon (the anomeric carbon) and the 6-carbon of adjacent sugars (i.e., a 1-6 linkage), or the 1-carbon (the anomeric carbon) and the 2-carbon of adjacent sugars (i.e., a 1-2 linkage). A sugar can be linked within an oligosaccharide such that the anomeric carbon is in the a- or β-configuration. The oligosaccharides prepared according to the methods of the invention can also include linkages between carbon atoms other than the 1-, 2-, 3-, 4-, and 6-carbons.

The term “polysaccharide”, as used herein, refers to a molecular moiety containing at least 9 monosaccharides covalently bound to each other. Polysaccharides include starch, hyaluronic acid, amylose, amylopectin, dextran, cyclodextrin, cellulose, agar, chitin, gum arabic and glycogen.

The skilled person will understand that the term “O-glycosyl moiety” is a glycosyl moiety that is linked to another molecular moiety via an O-glycosidic linkage. Similarly, an “S-glycosyl moiety” is a glycosyl moiety that is linked to another molecular moiety via an S-glycosidic linkage, an “N-glycosyl moiety” is a glycosyl moiety that is linked to another molecular moiety via an N-glycosidic linkage and a “C-glycosyl moiety” is a glycosyl moiety that is linked to another molecular moiety via a C-glycosidic linkage.

In some embodiments, the preferred glycosyl can be selected from the group comprising glucosyl, allosyl, altrosyl, galactosyl, gulosyl, idosyl, mannosyl, talosyl, fructosyl, psicosyl, sorbosyl tagatosyl, sucrosyl, maltosyl, lactosyl, cellobiosyl, mannotriosyl, raffinosyl, melezitosyl; amylopectinyl, Syalyl Lewis X (SiaLex), preferably glucosyl.

The term “carbonyl” as a group or part of a group, refers to the group —C(═O)—, also written as —CO—.

The term “alkylcarbonyl”, as a group or part of a group, refers to a group of formula —C(═O)R^{x}, wherein R^{x} is alkyl as defined herein.

The term “heterocyclyl” as used herein refer to non-aromatic, fully saturated or partially unsaturated ring system comprising from 3 to 18 atoms including at least one N, O, S, or P, preferably 3 to 14 atoms (3-14 membered heterocyclyl), for example, 3 to 7 member monocyclic, 7 to 14 member bicyclic, preferably comprising a total of 3 to 10 ring atoms (3-10 membered heterocyclyl), more preferably 3 to 8 atoms (4-6 membered heterocyclyl), yet more preferably 3 to 5 atoms (3-5 membered heterocyclyl). Each ring of the heterocycle or heterocyclyl may have 1, 2, 3 or 4 heteroatoms selected from N, O, P and/or S, where the N and S heteroatoms may optionally be oxidized, and the N heteroatoms may optionally be quaternized; and wherein at least one carbon atom of heterocyclyl can be oxidized to form at least one C═O. The heterocyclyl may be attached at any heteroatom or carbon atom of the ring or ring system, where valence allows. The rings of multi-ring heterocyclyls or heterocycles may be fused, bridged and/or joined through one or more spiro atoms. Fused systems of a heterocycle or heterocyclyl with an aryl ring are considered as heterocycle or heterocyclyl irrespective of the ring that is bound to the core structure. Fused systems of a heterocycle or heterocyclyl with a heteroaryl ring are considered as heteroaryl irrespective of the ring that is bound to the core structure.

Non limiting exemplary heterocycles or heterocyclic groups include piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, imidazolinyl, pyrazolidinyl imidazolidinyl, oxazolinyl, isoxazolinyl, oxazolidinyl, isoxazolidinyl, thiazolidinyl, isothiazolidinyl, succinimidyl, indolinyl, isoindolinyl, chromanyl (also known as 3,4-dihydrobenzo[b]pyranyl), 2H-pyrrolyl, pyrrolinyl (such as 1-pyrrolinyl, 2-pyrrolinyl, 3-pyrrolinyl), 4H-quinolizinyl, 2-oxopiperazinyl, pyrazolinyl (such as 2-pyrazolinyl, 3-pyrazolinyl), tetrahydro-2H-pyranyl, 2H-pyranyl, 4H-pyranyl, dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formyl-piperazinyl, thiomorpholinyl, dihydrofuranyl, dihydrothienyl, tetrahydrothienyl, dihydropyrazolyl, dihydroimidazolyl, isothiazolinyl, thiazolinyl, triazolinyl, triazolidinyl, oxadiazolinyl, oxadiazolidinyl, thiadiazolinyl, thiadiazolidinyl, tetrazolinyl, tetrazolidinyl, dihydro-pyridinyl, tetrahydro-pyridinyl, 1,2,3,6-tetrahydropyridinyl, hexahydro-pyridinyl, dihydro-pyrimidinyl, tetrahydro-pyrimidinyl, 1,4,5,6-tetrahydropyrimidinyl, dihydro-pyrazinyl, tetrahydro-pyrazinyl, dihydro-pyridazinyl, tetrahydro-pyridazinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzooxazolyl, 2,3-dihydrobenzo[d]oxazolyl, tetrahydro-benzooxazolyl, dihydro-benzooxazinyl, 3,4-dihydro-2H-benzo[b][1,4]oxazinyl, tetrahydro-benzooxazinyl, benzo[1,3]dioxolyl, benzo[1,4]dioxanyl, dihydro-purinyl, tetrahydro-purinyl, dihydro-quinolinyl, 1,2,3,4-tetrahydroquinolinyl, dihydro-isoquinolinyl, 3,4-dihydroisoquinolin-(1H)-yl, tetrahydro-isoquinolinyl, 1,2,3,4-tetrahydroisoquinolinyl, dihydro-quinazolinyl, tetrahydro-quinazolinyl, dihydro-quinoxalinyl, tetrahydro-quinoxalinyl, 1,2,3,4-tetrahydroquinoxalinyl, 2,5-dihydro-1H-pyrrolyl, 4,5-dihydro-1H-imidazolyl, hexahydropyrrolo[3,4-b][1,4]oxazin-(2H)-yl, 3,4-dihydro-2H-pyrido[3,2-b][1,4]oxazinyl, (cis)-octahydrocyclopenta[c]pyrrolyl, hexahydropyrrolo[3,4-b]pyrrol-(1H)-yl, 5H-pyrrolo[3,4-b]pyridin-(7H)-yl, 5,7-dihydro-6H-pyrrolo[3,4-b]pyridinyl, tetrahydro-1H-pyrrolo[3,4-b]pyridin-(2H,7H,7aH)-yl, hexahydro-1H-pyrrolo[3,4-b]pyridin-(2H)-yl, (octahydro-6H-pyrrolo[3,4-b]pyridinyl, hexahydropyrrolo[1,2-a]pyrazin-(1H)-yl, 3,4,6,7,8,8a-hexahydro-1H-pyrrolo[1,2-a]pyrazinyl, 2,3,4,9-tetrahydro-1H-carbazolyl, 1,2,3,4-tetrahydropyrazino[1,2-a]indolyl, 2,3-dihydro-1H-pyrrolo[1,2-a]indolyl, 1,3-dihydro-2H-isoindolyl, octahydro-2H-isoindolyl, 2,5-diazabicyclo[2.2.1]heptanyl, 2-azabicyclo[2.2.1]heptenyl, 3-azabicyclo[3.1.0]hexanyl, 3,6-diazabicyclo[3.1.0]hexanyl, 5-azaspiro[2.4]heptanyl, 4,7-diazaspiro[2.5]octanyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 2,7-diazaspiro[4.4]nonanyl, 2-azaspiro[4.5]decanyl, 2,8-diazaspiro[4.5]decanyl, 3,6-diazabicyclo[3.2.1]octyl, 1,4-dihydroindeno[1,2-c]pyrazolyl, dihydropyranyl, dihydropyridinyl, dihydroquinolinyl, 8H-indeno[1,2-d]thiazolyl, tetrahydroimidazo[1,2-a]pyridinyl, pyridin-2(1H)-one, 8-azabicyclo[3.2.1]oct-2-enyl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxan-5-yl, 1,2,4-trioxan-3-yl, 1,2,4-trioxan-5-yl, 1,2,4-trioxan-6-yl and 1,3,4-trioxan-2-yl. The term “azepanyl” as used herein includes azepan-1-yl, azepan-2-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “chromanyl” as used herein includes chroman-2-yl, chroman-3-yl, chroman-4-yl, chroman-5-yl, chroman-6-yl, chroman-7-yl and chroman-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heterocyclylalkylene” or “heterocyclyl-alkylene”, as a group or part of a group, refers to an alkylene as defined herein, wherein at least one hydrogen atom is replaced by at least one heterocyclyl as defined herein, and can be represented by a group of formula R^{y}R^{r} wherein R^{y} is alkylene and R^{r} is heterocyclyl as defined herein. The term “3 to 10 membered heterocyclyl-C₁₋₆alkylene” refers to a heterocyclyl-alkylene wherein the alkylene moiety comprises from 1 to 6 carbon atoms and the heterocyclyl moiety is non-aromatic, fully saturated or partially unsaturated ring system of 3 to 10 atoms including at least one N, O, S, or P.

The term “heterocyclylalkenylene” or “heterocyclyl-alkenylene”, as a group or part of a group, refers to an alkenylene as defined herein, wherein at least one hydrogen atom is replaced by at least one heterocyclyl as defined herein, and can be represented by a group of formula R^{t}R^{r} wherein R^{t} is alkenylene and R^{r} is heterocyclyl as defined herein. The term “3 to 10 membered heterocyclyl-C₂₋₆alkenylene” refers to a heterocyclyl-alkenylene wherein the alkenylene moiety comprises from 2 to 6 carbon atoms and the heterocyclyl moiety is non-aromatic, fully saturated or partially unsaturated ring system of 3 to 10 atoms including at least one N, O, S, or P.

The term “heterocyclylalkynylene” or “heterocyclyl-alkynylene”, as a group or part of a group, refers to an alkynylene as defined herein, wherein at least one hydrogen atom is replaced by at least one heterocyclyl as defined herein, and can be represented by a group of formula R^{s}R^{r} wherein R^{s} is alkynylene and R^{r} is heterocyclyl as defined herein. The term “3 to 10 membered heterocyclyl-C₂₋₆alkynylene” refers to a heterocyclyl-alkynylene wherein the alkynylene moiety comprises from 2 to 6 carbon atoms and the heterocyclyl moiety is non-aromatic, fully saturated or partially unsaturated ring system of 3 to 10 atoms including at least one N, O, S, or P.

The term “heteroaryl” refers to an aromatic ring system comprising from 5 to 18 atoms including at least one N, O, S, or P, containing 1 or 2 rings which can be fused together or linked covalently, preferably 5 to 14 atoms (5-14 membered heteroaryl), yet more preferably 5 to 10 atoms (5-10 membered heteroaryl), each ring typically containing 5 to 6 atoms; at least one of said rings is aromatic, where the N and S heteroatoms may optionally be oxidized and the N heteroatoms may optionally be quaternized, and wherein at least one carbon atom of said heteroaryl can be oxidized to form at least one C═O. Fused systems of a heteroaryl ring with a cycloalkyl ring, or a cycloalkenyl ring, or a cycloalkynyl ring, are considered as heteroaryl irrespective of the ring that is bound to the core structure. Fused systems of a heteroaryl ring with a heterocycle are considered as heteroaryl irrespective of the ring that is bound to the core structure. Fused systems of a hetero aryl ring with an aryl ring are considered as heteroaryl irrespective of the ring that is bound to the core structure. Non-limiting examples of such heteroaryl, include: pyridinyl, pyrrolyl, thiophenyl (also referred as thienyl), furanyl, thiazolyl, isothiazolyl, thiadiazolyl, triazol-2-yl, 1H-pyrazol-5-yl, pyrazolyl, imidazolyl, oxazolyl, isoxazolyl, triazolyl, oxadiazolyl, tetrazolyl, oxatriazolyl, thiatriazolyl, pyrimidinyl, pyrazinyl, pyridazinyl, oxazinyl, dioxinyl, thiazinyl, triazinyl, pyranyl, thiopyranyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, benzooxazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, benzo[c][1,2,5]oxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, 9H-purinyl, imidazo[1,2-a]pyridinyl, imidazo[1,2-a]pyrazinyl, imidazo[5,1-a]isoquinolinyl, imidazo[1,5-a]pyridinyl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; acridinyl, phthalazinyl, 1,4-dihydroindeno[1,2-c]-1H-pyrazolyl, 2,3-dihydro-1H-inden-1-one, 2,3-dihydro-1H-indenyl, 3,4-dihydroquinolin-2(1H)-one, 5,6-dihydroimidazo[5,1-a]isoquinolinyl, 8H-indeno[1,2-d]thiazolyl, benzo[d]oxazol-2(3H)-one, quinolin-2(1H)-one, quinazolin-4(1H)-one, quinazoline-2,4(1H,3H)-dione, benzo-[d]oxazolyl, and pyrazolo[1,5-a]pyridinyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl or 1H-pyrazol-5-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes triazol-2-yl, 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl, indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

Heteroaryl and heterocycle or heterocyclyl as used herein includes by way of example and not limitation these groups described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

The term “heteroarylalkylene” or “heteroaryl-alkylene”, as a group or part of a group, refers to an alkylene as defined herein, wherein at least one hydrogen atom is replaced by at least one heteroaryl as defined herein, and can be represented by a group of formula R^{y}R^{u} wherein R^{y} is alkylene and R^{u} is heteroaryl as defined herein. The term “5 to 10 membered heteroaryl-C₁₋₆alkylene” refers to a heteroaryl-alkylene wherein the alkylene moiety comprises from 1 to 6 carbon atoms and the heteroaryl moiety is an aromatic ring system comprising from 5 to 10 atoms including at least one N, O, S, or P.

The term “heteroarylalkenylene” or “heteroaryl-alkenylene”, as a group or part of a group, refers to an alkenylene as defined herein, wherein at least one hydrogen atom is replaced by at least one heteroaryl as defined herein, and can be represented by a group of formula R^{t}R^{u} wherein R^{t} is alkenylene and R^{u} is heteroaryl as defined herein. The term “5 to 10 membered heteroaryl-C₂₋₆alkenylene” refers to a heteroaryl-alkenyl wherein the alkenylene moiety comprises from 2 to 6 carbon atoms and the heteroaryl moiety is an aromatic ring system comprising from 5 to 10 atoms including at least one N, O, S, or P.

The term “heteroarylalkynylene” or “heteroaryl-alkynylene”, as a group or part of a group, refers to an alkynylene as defined herein, wherein at least one hydrogen atom is replaced by at least one heteroaryl as defined herein, and can be represented by a group of formula R^{s}R^{u} wherein R^{s} is alkynylene and R^{u} is heteroaryl as defined herein. The term “5 to 10 membered heteroaryl-C₂₋₆alkynylene” refers to a heteroaryl-alkynylene wherein the alkynylene moiety comprises from 2 to 6 carbon atoms and the heteroaryl moiety is an aromatic ring system comprising from 5 to 10 atoms including at least one N, O, S, or P.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

Any reference to a “compound according to the invention”, or “compound of formula (I)” also includes isomers such as stereoisomers and tautomers, salts such as pharmaceutically and/or physiologically acceptable salts, hydrates, solvates, polymorphs of such compounds unless expressly indicated otherwise.

The term “isomers” as used herein means all possible isomeric forms, including tautomeric and stereochemical forms, which the compounds of formulae herein may possess, but not including position isomers. Typically, the structures shown herein exemplify one tautomeric or resonance form of the compounds, but the corresponding alternative configurations are contemplated as well.

Depending on its substitution pattern, the compounds of the present invention may or may not have one or more optical stereocenters and may or may not exist as different enantiomers or diastereomers. Any such enantiomers, diastereomers or other optical isomers are encompassed by the scope of the invention. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration. The terms R- or S-configuration are used herein in accordance with Chemical Abstracts nomenclature. The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

Separation of stereoisomers is accomplished by standard methods known to those in the art. One enantiomer of a compound can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol, and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The term “salts” relates to any salts that the compounds may form. They can be cosmetically or pharmaceutically acceptable. Therefore, the compounds of this invention optionally comprise salts of the compounds herein, especially cosmetically or pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, ethanolamine, and the like. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g., hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e., ethanedioic), malonic, succinic (i.e., butanedioic acid), maleic, fumaric, malic, tartaric, citric, methanesulfonic, ethanesulfonic, benzenesulfonic, p-toluenesulfonic, cyclohexanesulfamic, salicylic (i.e., 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein can comprise compounds of the invention in their unionized, as well as zwitterionic form, and combinations with stoichiometric amounts of water as in hydrates.

Also included within the scope of this invention are the salts of the compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄₊ (wherein X is C₁-C₄ alkyl). Physiologically acceptable salts of a hydrogen atom or an amino group include salts of organic carboxylic acids such as acetic, benzoic, lactic, fumaric, tartaric, maleic, malonic, malic, isethionic, lactobionic, and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄₊ (wherein X typically is independently selected from H or a C₁-C₄ alkyl group). However, salts of acids or bases which are not physiologically acceptable may also find use, for example, in the preparation or purification of a physiologically acceptable compound.

All salts, whether or not derived from a physiologically acceptable acid or base, are within the scope of the present invention. Non-limiting examples of suitable such salts include but are not limited to acid addition salts, formed either with inorganic acids such as hydrochloric acid, hydrobromic acid, sulfuric acid, nitric acid, phosphoric acid, and the like, or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. Other salts include 2,2-dichloroacetate, adipate, alginate, ascorbate, aspartate, 2-acetamidobenzoate, caproate, caprate, camphorate, cyclamate, laurylsulfate, edisilate, esylate, isethionate, formate, galactarate, gentisate, gluceptate, glucuronate, oxoglutarate, hippurate, lactobionate, napadisilate, xinafoate, nicotinate, oleate, orotate, oxalate, palmitate, embonate, pidolate, p-aminosalicylate, sebacate, tannate, rhodanide, undecylenate, and the like; or salts formed when an acidic proton present in the parent compound is replaced, such as with ammonia, arginine, benethamine, benzathine, calcium, choline, deanol, diethanolamine, diethylamine, ethanolamine, ethylendiamine, meglumine, glycine, hydrabamine, imidazole, lysine, magnesium, hydroxyethylmorpholine, piperazine, potassium, epolamine, sodium, trolamine, tromethamine, or zinc.

The present invention includes within its scope solvates of the compounds as defined herein. The term “solvates” refers to crystals formed by an active compound and a second component (solvent) which, in isolated form, is liquid at room temperature. Such solvates may be formed with common organic solvents, e.g., hydrocarbon solvents such as benzene or toluene; chlorinated solvents such as chloroform or dichloromethane; alcoholic solvents such as methanol, ethanol, or isopropanol; ethereal solvents such as diethyl ether or tetrahydrofuran; or ester solvents such as ethyl acetate. Alternatively, the solvates of the compounds herein may be formed with water, in which case they will be hydrates.

The compounds of formula (I) may be amorphous or may exist in one or more different crystalline states (polymorphs) which may have different macroscopic properties such as stability or show different biological properties such as activities. The present invention relates to amorphous and crystalline compounds of formula (I), mixtures of different crystalline states of the respective compound of formula (I).

The term “polymorph” refers to a particular crystalline form of a chemical compound that can crystallize in different crystalline forms, these forms having different arrangements and/or conformations of the molecules in the crystal lattice. Different crystalline forms usually have different X-ray diffraction patterns, infrared spectra, melting points, density, hardness, crystal shape, optical and electrical properties, stability, and solubility. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound described herein can exhibit beneficial effects (e.g., suitability for preparation of useful formulations, improved biological performance) relative to another polymorph or a mixture of polymorphs of the same compound. Preparation and isolation of a particular polymorph of a compound can be achieved by methods known to those skilled in the art including, for example, crystallization using selected solvents and temperatures. Recrystallization solvent, rate of crystallization, storage temperature, and other factors may cause one crystal form to dominate. Various polymorphs of a compound can be prepared by crystallization under different conditions. For a comprehensive discussion of polymorphism see Rolf Hilfiker, Ed., Polymorphism in the Pharmaceutical Industry, Wiley-VCH, Weinheim, 2006.

When referring to compositions and products and the weight percent of the therein comprised ingredients it is to be understood that according to the present invention the overall amount of ingredients does not exceed 100% (±1% due to rounding).

In the present specification, the term “non-therapeutic” refers to the concept which excludes medical practice, that is, treatments to human body by a medical therapy.

Preferred statements (features) and embodiments of the methods, compositions, and uses of this invention are set herein below. Each statement and embodiment of the invention so defined may be combined with any other statement and/or embodiment, unless clearly indicated to the contrary. In particular, any feature indicated as being preferred or advantageous may be combined with any other features or statements indicated as being preferred or advantageous. Hereto, the present invention is in particular captured by any one or any combination of one or more of the below numbered statements and embodiments, with any other aspect and/or embodiment.
1. A compound of formula (I)

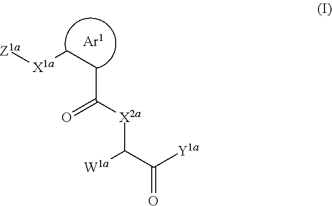

or a tautomer, a stereoisomer, a hydrate, a solvate, a salt, or a polymorph thereof, wherein
Ar¹ is aryl or heteroaryl; wherein each of said aryl or heteroaryl can be unsubstituted or substituted with one or more R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene; wherein each of said alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene, can be unsubstituted or substituted with one or more R^{za1}; wherein each R^{za1} is independently selected from the group comprising —COO—R^{h},

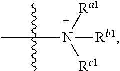

—NR^{a1}R^{b1} halo, alkyl, haloalkyl, alkylcarbonyl, alkenyl, and alkynyl;
   R^{h} is selected from the group comprising hydrogen, alkyl, alkenyl, and alkynyl,
   R^{a1} is selected from the group comprising hydrogen, alkyl, alkenyl, and alkynyl;
   R^{b1} is selected from the group comprising hydrogen, alkyl, alkenyl, and alkynyl;
   R^{c1} is selected from the group comprising hydrogen, alkyl, alkenyl, and alkynyl;
   each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
   X^{1a} is selected from —NR^{b}—, —O—, —S—, SO₂—O—, or —SO—O—;
   Z^{1a} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
   X^{2a} is —(CR^{c}R^{d})ₙ—; wherein n is an integer selected from 1, 2 or 3;
   each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
   R^{e} is selected from the group comprising alkylene, alkenylene, alkynylene, arylene, heterocyclylene, heteroarylene, and cycloalkylene;
   R^{f} is alkylene, wherein said alkylene can be interrupted by one or more O or S;
   R^{g} is selected from N-maleimidyl, N-maleimidyloxy, or N-maleimidyloxycarbonyl;
   W^{1a} is selected from —NR^{b}Z^{2a}, —R^{a}, or —OR^{a};
   Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
   Y^{1a} is selected from —OR^{a}, —R^{a}, or —NR^{a}R^{b}, or Y^{1a} is a moiety of formula (A);

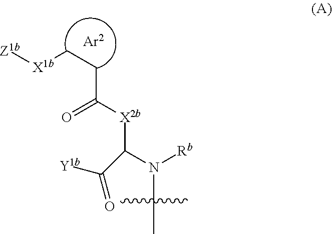

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; and Y^{1b} is selected from —OR^{a}, —NR^{b}R^{a}, or —R^{a};
with the proviso that

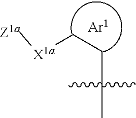

is not

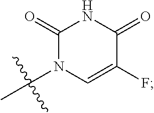 [SMILES: *n1cc(F)c(=O)[nH]c1=O]

and
with the proviso that said compound is not:
2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
2-amino-4-oxo-4-(2-(((2S,3R,4R,5S,6R)-3,4,5-trihydroxy-6-(hydroxymethyl)tetrahydro-2H-pyran-2-yl)amino)phenyl)butanoic acid;
methyl 2-amino-4-(2-aminophenyl)-4-oxobutanoate;
4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
(2R,3R,4S,5R)-2-(acetoxymethyl)-6-(2-amino-3-(4-methoxy-4-oxobutanoyl)phenoxy)tetrahydro-2H-pyran-3,4,5-triyl triacetate;
2-amino-4-(2-amino-5-hydroxyphenyl)-4-oxobutanoic acid.
   2. A compound of formula (I)

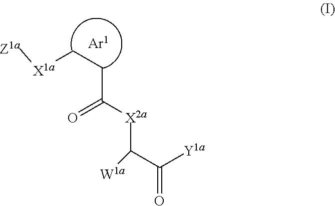

or a tautomer, a stereoisomer, a hydrate, a solvate, a salt, or a polymorph thereof, wherein
Ar¹ is aryl or heteroaryl; wherein each of said aryl or heteroaryl can be unsubstituted or substituted with one or more R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is selected from —NR^{b}—, —O—, —S—, SO₂—O—, or —SO—O—;
Z^{1a} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; wherein n is an integer selected from 1, 2 or 3; preferably 1 or 2; preferably n is 1;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more O or S;
R^{g} is selected from N-maleimidyl, N-maleimidyloxy, or N-maleimidyloxycarbonyl;
W¹a is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is a moiety of formula (A) or Y^{1a} is selected from —OR^{a}, —R^{a}, or —NR^{a}R^{b};

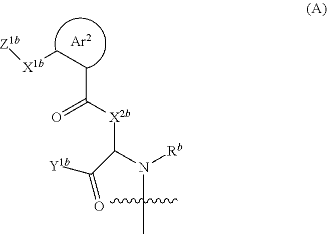

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; preferably 1 or 2; preferably
m is 1; and
Y^{1b} is selected from —OR^{a}, —NR^{b}R^{a}, or —R^{a};
with the proviso that

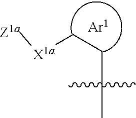

is not

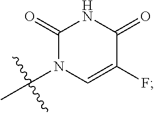 [SMILES: *n1cc(F)c(=O)[nH]c1=O]

and
with the proviso that said compound is not:
2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
2-amino-4-oxo-4-(2-(((2S,3R,4R,5S,6R)-3,4,5-trihydroxy-6-(hydroxymethyl)tetrahydro-2H-pyran-2-yl)amino)phenyl)butanoic acid, and
methyl 2-amino-4-(2-aminophenyl)-4-oxobutanoate,
4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
(2R,3R,4S,5R)-2-(acetoxymethyl)-6-(2-amino-3-(4-methoxy-4-oxobutanoyl)phenoxy)tetrahydro-2H-pyran-3,4,5-triyl triacetate;
2-amino-4-(2-amino-5-hydroxyphenyl)-4-oxobutanoic acid.
   3. A compound of formula (II):

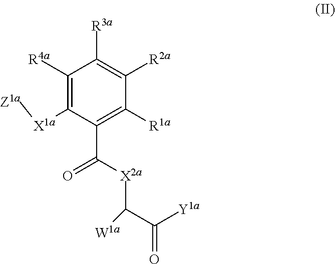

or a tautomer, a stereoisomer, a hydrate, a solvate, a salt, or a polymorph thereof, wherein R^{1a}, R^{2a}, R^{3a}, and R^{4a} are each independently R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, and —R^{f1}-R^{g1}; wherein each of said alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene, can be unsubstituted or substituted with one or more R^{za1}; wherein each R^{za1} is independently selected
from the group comprising —COO—R^{h},

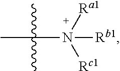

—NR^{a1}R^{b1}, —R^{f1}-R^{g1}, halo, alkyl, haloalkyl, alkylcarbonyl, alkenyl, and alkynyl; wherein R^{h} is selected from the group comprising hydrogen, alkyl, alkenyl, and alkynyl; R^{a1} is selected from the group comprising hydrogen, alkyl, alkenyl, and alkynyl; R^{b1} is selected from the group comprising hydrogen, alkyl, alkenyl, and alkynyl; R^{c1} is selected from the group comprising hydrogen, alkyl, alkenyl, and alkynyl;
R^{f1} is alkylene, wherein said alkylene can be interrupted by one or more O or S;
R^{g1} is hydrogen, or alkyl;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; wherein n is an integer selected from 1, 2 or 3;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more O or S;
R^{g} is selected from hydrogen, alkyl, N-maleimidyl, N-maleimidyloxy, or N-maleimidyloxycarbonyl;
W¹a is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is selected from —OR^{a}, —R^{a}, or —NR^{a}R^{b}, or Y^{1a} is a moiety of formula (A);

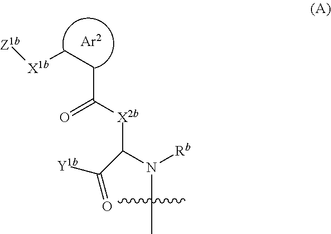

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; and
Y^{1b} is selected from —OR^{a}, —NR^{b}R^{a}, or —R^{a}.
4. A compound of formula (II):

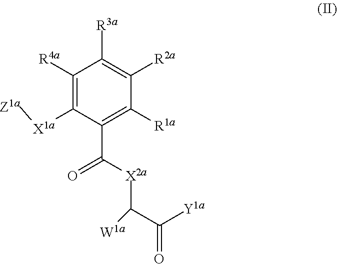

or a tautomer, a stereoisomer, a hydrate, a solvate, a salt, or a polymorph thereof, wherein R^{1a}, R^{2a}, R^{3a}, and R^{4a} are each independently R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, and —R^{f1}-R^{g1}; wherein each of said alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene, can be unsubstituted or substituted with one or more R^{za1}; wherein each R^{za1} is independently selected from the group comprising —COO—R^{h},

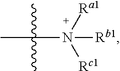

NR^{a1}R^{b1}, —R^{f1}-R^{g1}, halo, alkyl, haloalkyl, alkylcarbonyl, alkenyl, and alkynyl; wherein R^{h} is selected from the group comprising hydrogen, alkyl, alkenyl, and alkynyl; R^{a1} is selected from the group comprising hydrogen, alkyl, alkenyl, and alkynyl; R^{b1} is selected from the group comprising hydrogen, alkyl, alkenyl, and alkynyl; R¹ is selected from the group comprising hydrogen, alkyl, alkenyl, and alkynyl;
R^{f1} is alkylene, wherein said alkylene can be interrupted by one or more O or S;
R^{g1} is hydrogen, or alkyl;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; wherein n is an integer selected from 1, 2 or 3;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more O or S;
R^{g} is selected from hydrogen, alkyl, N-maleimidyl, N-maleimidyloxy, or N-maleimidyloxycarbonyl;
W^{1a} is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is selected from —OR^{a}, —R^{a}, or —NR^{a}R^{b}, or Y^{1a} is a moiety of formula (A);

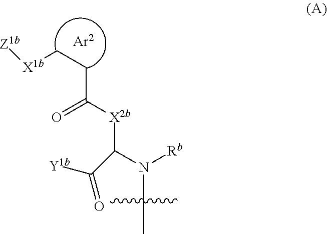

wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; and
Y^{1b} is selected from —OR^{a}, —NR^{b}R^{a}, or —R^{a};
with the proviso that said compound is not:
2-amino-4-(2-aminophenyl)-4-oxobutanoic acid;
(R)-2-amino-4-(2-aminophenyl)-4-oxobutanoic acid;
(S)-2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
2-amino-4-oxo-4-(2-(((3R,4R,5S,6R)-3,4,5-trihydroxy-6-(hydroxymethyl)tetrahydro-2H-pyran-2-yl)amino)phenyl)butanoic acid;
(2S)-2-amino-4-oxo-4-(2-(((3R,4R,5S,6R)-3,4,5-trihydroxy-6-(hydroxymethyl)tetrahydro-2H-pyran-2-yl)amino)phenyl)butanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
methyl 2-amino-4-(2-aminophenyl)-4-oxobutanoate;
4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
(S)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(R)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(2R,3R,4S,5R)-2-(acetoxymethyl)-6-(2-amino-3-(4-methoxy-4-oxobutanoyl)phenoxy)tetrahydro-2H-pyran-3,4,5-triyl triacetate;
2-amino-4-(2-amino-5-hydroxyphenyl)-4-oxobutanoic acid;
(S)-2-amino-4-(2-amino-5-hydroxyphenyl)-4-oxobutanoic acid;
(R)-2-amino-4-(2-amino-5-hydroxyphenyl)-4-oxobutanoic acid;
5-(2-aminophenyl)-5-oxopentanoic acid;
4-(2-aminophenyl)-4-oxobutanoic acid;
methyl 6-(2-aminophenyl)-6-oxohexanoate;
methyl 4-(2-aminophenyl)-2-((tert-butoxycarbonyl)amino)-4-oxobutanoate;
butyl 2-amino-4-(2-aminophenyl)-4-oxobutanoate;
ethyl (S)-2-amino-4-(2-aminophenyl)-4-oxobutanoate;
(S)-2-amino-4-(2-aminophenyl)-4-oxobutanoic acid diacetate;
(R)-2-amino-4-(2-formamidophenyl)-4-oxobutanoic acid;
(S)-2-amino-4-(2-formamidophenyl)-4-oxobutanoic acid;
2-amino-4-(2-formamidophenyl)-4-oxobutanoic acid;
methyl 2-acetamido-4-(2-acetamidophenyl)-4-oxobutanoate;
4-(2-amino-3-(((2R,3S,4R,5R,6S)-3,4,5-trihydroxy-6-((((2S,3S,4R,5R,6S)-3,4,5-trihydroxy-6-(hydroxymethyl)tetrahydro-2H-pyran-2-yl)oxy)methyl)tetrahydro-2H-pyran-2-yl)oxy)phenyl)-4-oxobutanoic acid;
4-(2-amino-3-hydroxyphenyl)-4-oxobutanoic acid O-beta-D-diglucoside); 4-(2-amino-3-(((2R,3S,4R,5R,6S)-3,4-5-trihydroxy-6-(hydroxymethyl)tetrahydro-2H-pyran-2-yl)oxy)phenyl)-4-oxobutanoic acid;
4-(2-amino-3-hydroxyphenyl)-4-oxobutanoic acid O-beta-d-glucoside;
2-amino-4-(2-formamido-5-hydroxyphenyl)-4-oxobutanoic acid;
2-amino-4-(2-amino-3-chlorophenyl)-4-oxobutanoic acid;
(R)-2-amino-4-(2-amino-3-chlorophenyl)-4-oxobutanoic acid;
(S)-2-amino-4-(2-amino-3-chlorophenyl)-4-oxobutanoic acid;
2-amino-4-(2-amino-4-chlorophenyl)-4-oxobutanoic acid;
(R)-2-amino-4-(2-amino-4-chlorophenyl)-4-oxobutanoic acid;
(S)-2-amino-4-(2-amino-4-chlorophenyl)-4-oxobutanoic acid;
2-amino-4-(2-amino-5-fluorophenyl)-4-oxobutanoic acid;
(R)-2-amino-4-(2-amino-5-fluorophenyl)-4-oxobutanoic acid;
(S)-2-amino-4-(2-amino-5-fluorophenyl)-4-oxobutanoic acid;
2-amino-4-(2-amino-5-bromophenyl)-4-oxobutanoic acid;
(R)-2-amino-4-(2-amino-5-bromophenyl)-4-oxobutanoic acid;
(S)-2-amino-4-(2-amino-5-bromophenyl)-4-oxobutanoic acid;
2-amino-4-(2-amino-5-methylphenyl)-4-oxobutanoic acid;
(R)-2-amino-4-(2-amino-5-methylphenyl)-4-oxobutanoic acid;
(S)-2-amino-4-(2-amino-5-methylphenyl)-4-oxobutanoic acid;
2-amino-4-(2-amino-5-ethylphenyl)-4-oxobutanoic acid;
(R)-2-amino-4-(2-amino-5-ethylphenyl)-4-oxobutanoic acid;
(S)-2-amino-4-(2-amino-5-ethylphenyl)-4-oxobutanoic acid;
2-amino-4-(2-amino-5-propylphenyl)-4-oxobutanoic acid;
(R)-2-amino-4-(2-amino-5-propylphenyl)-4-oxobutanoic acid;
(S)-2-amino-4-(2-amino-5-propylphenyl)-4-oxobutanoic acid;
2-amino-4-(2-amino-5-isopropylphenyl)-4-oxobutanoic acid;
(R)-2-amino-4-(2-amino-5-isopropylphenyl)-4-oxobutanoic acid;
(S)-2-amino-4-(2-amino-5-isopropylphenyl)-4-oxobutanoic acid;
2-amino-4-(2-amino-5-cyclohexylphenyl)-4-oxobutanoic acid;
(R)-2-amino-4-(2-amino-5-cyclohexylphenyl)-4-oxobutanoic acid;
(S)-2-amino-4-(2-amino-5-cyclohexylphenyl)-4-oxobutanoic acid;
2-amino-4-(2-(methylamino)phenyl)-4-oxobutanoic acid;
(S)-2-amino-4-(2-(methylamino)phenyl)-4-oxobutanoic acid;
(R)-2-amino-4-(2-(methylamino)phenyl)-4-oxobutanoic acid;
2-amino-4-(2-(dimethylamino)phenyl)-4-oxobutanoic acid;
(S)-2-amino-4-(2-(dimethylamino)phenyl)-4-oxobutanoic acid;
(R)-2-amino-4-(2-(dimethylamino)phenyl)-4-oxobutanoic acid;
2-amino-4-(2-amino-5-chlorophenyl)-4-oxobutanoic acid;
(R)-2-amino-4-(2-amino-5-chlorophenyl)-4-oxobutanoic acid;
(S)-2-amino-4-(2-amino-5-chlorophenyl)-4-oxobutanoic acid;
2-amino-4-(2-amino-6-chlorophenyl)-4-oxobutanoic acid;
(R)-2-amino-4-(2-amino-6-chlorophenyl)-4-oxobutanoic acid;
(S)-2-amino-4-(2-amino-6-chlorophenyl)-4-oxobutanoic acid;
2-acetamido-4-(2-amino-5-chlorophenyl)-4-oxobutanoic acid;
(R)-2-acetamido-4-(2-amino-5-chlorophenyl)-4-oxobutanoic acid;
(S)-2-acetamido-4-(2-amino-5-chlorophenyl)-4-oxobutanoic acid;
2-amino-4-(2-amino-3-hydroxyphenyl)-4-oxobutanoic acid;
(S-)2-amino-4-(2-amino-3-hydroxyphenyl)-4-oxobutanoic acid;
(R-)2-amino-4-(2-amino-3-hydroxyphenyl)-4-oxobutanoic acid;
methyl (S-)2-amino-4-(2-aminophenyl)-4-oxobutanoate;
2-amino-4-(2-aminophenyl)-4-oxobutanamide;
(R)-2-amino-4-(2-aminophenyl)-4-oxobutanamide;
(S)-2-amino-4-(2-aminophenyl)-4-oxobutanamide;
(S)-2-acetamido-4-(2-acetamidophenyl)-4-oxobutanoic acid;
(S)-2-formamido-4-(2-formamidophenyl)-4-oxobutanoic acid; and
4-(2-aminophenyl)-4-oxo-2-phenylbutanamide.
   5. The compound according to any one of statements 1 or 2, wherein
      Ar¹ is C₆₋₁₀aryl or a 5-10 membered heteroaryl; wherein each of said aryl or heteroaryl can be unsubstituted or substituted with one or more R^{za}; preferably Ar¹ is C₆₋₁₀aryl or a 5-6 membered heteroaryl, preferably containing at least one N; wherein each of said aryl or heteroaryl can be unsubstituted or substituted with one or more R^{za}; preferably Ar¹ is C₆₋₁₀aryl or a 6 membered heteroaryl containing at least one N; wherein each of said aryl or heteroaryl can be unsubstituted or substituted with one or more R^{za}; preferably Ar¹ is phenyl; wherein said phenyl can be unsubstituted or substituted with one or more R^{za}; preferably Ar¹ is phenyl; each R^{za} is independently selected from the group comprising hydrogen, halo, C₁₋₆alkyl, haloC₁₋₆alkyl, C₁₋₆alkylcarbonyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, C₃₋₆cycloalkyl-C₂₋₆alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glucosyl, non-acylated or acylated —O-glucosyl, non-acylated or acylated —NR^{b}-glucosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, and —OR^{a}; preferably each R^{za} is independently selected from the group comprising hydrogen, halo, C₁₋₆alkyl, haloC₁₋₆alkyl, and haloC₁₋₆alkyloxy; preferably each R^{za} is independently selected from the group comprising hydrogen, halo, C₁₋₄alkyl, haloC₁₋₄alkyl, and haloC₁₋₄alkyloxy; preferably each R^{za} is independently selected from the group comprising hydrogen, halo, and C₁₋₄alkyl; preferably each R^{za} is independently selected hydrogen, halo, or C₁₋₆alkyl; preferably each R^{za} is hydrogen,
      each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene, can be unsubstituted or substituted with one or more R^{za1}; wherein each R^{za1} is independently selected from the group comprising —COO—R^{h},

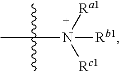

—NR^{a1}R^{b1}, halo, C₁₋₆alkyl, haloC₁₋₆alkyl, C₁₋₆alkylcarbonyl, C₂₋₆alkenyl, and C₂₋₆alkynyl; R^{h} is selected from the group comprising hydrogen, C₁₋₆alkyl, C₂₋₆alkenyl, and C₂₋₆alkynyl; R^{a1} is selected from the group comprising hydrogen, C₁₋₆alkyl, C₂₋₆alkenyl, and C₂₋₆alkynyl; R^{b1} is selected from the group comprising hydrogen, C₁₋₆alkyl, C₂₋₆alkenyl, and C₂₋₆alkynyl; R^{c1} is selected from the group comprising hydrogen, C₁₋₆alkyl, C₂₋₆alkenyl, and C₂₋₆alkynyl; preferably each R^{a} is independently selected from the group comprising hydrogen, halo, C₁₋₁₈alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; wherein each of said C₁₋₁₈alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

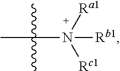

—NR^{a1}R^{b1}, halo, C₁₋₆alkyl, haloC₁₋₆alkyl, and C₁₋₆alkylcarbonyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₅alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₃₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; wherein each of said C₁₋₁₆alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

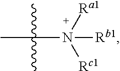

—NR^{a1}R^{b1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; preferably each R^{a} is independently selected from the group comprising hydrogen, halo, C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl; wherein each of said C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

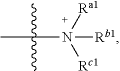

—NR^{a1}R^{b1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl; wherein each of said C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

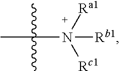

—NR^{a1}R^{b1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-4 membered heterocyclyl containing at least one O, and phenyl; wherein each of said C₁₋₁₅alkyl, C₂₋₆alkenyl, haloC₁₋₆alkyl, C₁₋₈alkylcarbonyl, 3-4 membered heterocyclyl containing at least one O, and phenyl, can be unsubstituted or substituted with one or more R^{za1}; wherein each R^{za1} is independently selected from the group comprising —COO—R^{h},

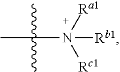

—NR^{a1}R^{b1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; preferably each R^{a} is independently selected from the group comprising hydrogen, halo, C₁₋₁₅alkyl, C₂₋₆alkenyl, oxiranyl, and C₁₋₆alkylcarbonyl; wherein each of said C₁₋₁₅alkyl, C₂₋₆alkenyl, oxiranyl, and C₁₋₆alkylcarbonyl, can be unsubstituted or substituted with one or more R^{za1}; wherein each R^{za1} is independently selected from the group comprising —COO—R^{h},

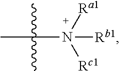

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; preferably each R^{a} is independently selected from the group comprising hydrogen, halo, C₁₋₁₅alkyl, C₂₋₄alkenyl, and oxiranyl; wherein each of said C₁₋₁₅alkyl, C₂₋₆alkenyl, oxiranyl, and C₁₋₆alkylcarbonyl, can be unsubstituted or substituted with one or more R^{za1}; wherein each R^{za1} is independently selected from the group comprising —COO—R^{h},

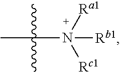

—NR^{a1}R^{b1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R¹ is selected from hydrogen, or C₁₋₄alkyl; for example, each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably each R^{a} is independently selected from the group comprising hydrogen, halo, C₁₋₁₈alkyl, haloC₁₋₆alkyl, C₁₋₁₂alkylcarbonyl, C₂₋₁₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{a} is independently selected from the group comprising hydrogen, halo, C₁₋₁₆alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl; preferably each R^{a} is independently selected from the group comprising hydrogen, halo, C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl; preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-4 membered heterocyclyl containing at least one 0, and phenyl; preferably each R^{a} is independently selected from the group comprising hydrogen, halo, C₁₋₁₅alkyl, C₂₋₆alkenyl, oxiranyl, and C₁₋₈ alkylcarbonyl; preferably each R^{a} is independently selected from the group comprising hydrogen, halo, C₁₋₁₅alkyl, C₂₋₄alkenyl, and oxiranyl;
each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 5-10 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl containing at least one N, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, and C₆₋₁₀aryl; preferably each R^{b} is independently selected from the group comprising hydrogen, C₁₋₆alkyl, and haloC₁₋₆alkyl; preferably each R^{b} is independently selected from hydrogen, or C₁₋₆alkyl; preferably each R^{b} is hydrogen;
X^{1a} is selected from —NR^{b}—, —O—, —S—, SO₂—O—, or —SO—O—; preferably X^{1a} is selected from —NR^{b}—, —O—, —S—; preferably X^{1a} is selected from —NR^{b}—, or —O—; preferably X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{t}-maleimidyl, halo, C₁₋₆alkyl, haloC₁₋₆alkyl, C₁₋₆alkylcarbonyl, C₂₋₈alkenylcarbonyl, C₆₋₁₀aryl, C₃₋₆cycloalkyl, 3-6 membered heterocyclyl-C₁₋₆alkylene containing at least one N or one O, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{f}-maleimidyl, halo, C₁₋₈alkyl, haloC₁₋₆alkyl, 3-5 membered heterocyclyl-C₁₋₆alkylene containing at least one O, C₁₋₈alkylcarbonyl, and C₂₋₆alkenylcarbonyl; preferably Z^{1a} is selected from the group comprising hydrogen, —CO—R^{a}, —CO—C₂₋₄alkenylene-CO—O—C₁₋₄alkyl, —CO—C₁₋₆alkylene-maleimidyl, C₁₋₈alkyl, 3-4 membered heterocyclyl-C₁₋₂alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₂alkylene, C₁₋₈alkylcarbonyl, and C₂₋₆alkenylcarbonyl; preferably Z^{1a} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl;
X^{2a} is —(CR^{c}R^{d})ₙ—; preferably X^{2a} is —(CR^{c}H)ₙ—; preferably X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—;
each R^{c} and R^{d} is independently selected from the group comprising hydrogen, halo, C₁₋₆alkyl, haloC₁₋₆alkyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably each R^{c} and R^{d} is independently selected from the group comprising hydrogen, halo, C₁₋₆alkyl, haloC₁₋₆alkyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{c} and R^{d} is independently selected from the group comprising hydrogen, halo, C₁₋₆alkyl, haloC₁₋₆alkyl, C₆₋₁₀aryl, 3-10 membered heterocyclyl, 5-10 membered heteroaryl, and C₃₋₆cycloalkyl; preferably each R^{c} and R^{d} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{c} and R^{d} is independently selected from the group comprising hydrogen, halo, C₁₋₆alkyl, haloC₁₋₆alkyl, C₆₋₁₀aryl, 3-6 membered heterocyclyl containing at least one N or one O, 5-6 membered heteroaryl containing at least one N, and C₃₋₆cycloalkyl; preferably each R^{c} and R^{d} is independently selected from the group comprising hydrogen, halo, C₁₋₆alkyl, haloC₁₋₆alkyl, and C₆₋₁₀aryl; preferably each R^{c} and R^{d} is independently selected from hydrogen, or C₁₋₆alkyl; preferably each R^{c} and R^{d} is independently is hydrogen;
R^{e} is selected from the group comprising C₁₋₁₂alkylene, C₂₋₁₂alkenylene, C₂₋₁₂alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₂₋₆alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₈alkenylene;
R^{f} is C₁₋₂₀alkylene, wherein said alkylene can be interrupted by one or more O or S; preferably
R^{f} is C₁₋₁₆alkylene, wherein said alkylene can be interrupted by one or more O or S; preferably
R^{f} is C₁₋₁₄alkylene, wherein said alkylene can be interrupted by one or more O or S; preferably
R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from N-maleimidyl, N-maleimidyloxy, or N-maleimidyloxycarbonyl; preferably
R^{g} is N-maleimidyl, or N-maleimidyloxy; preferably R^{g} is N-maleimidyl;
W^{1a} is selected from —NR^{b}Z^{2a}, —R^{a}, or —OR^{a}; preferably W^{1a} is selected from —NR^{b}Z^{2a}, or —R^{a}; preferably W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W^{1a} is —NHCO—C₁₋₆alkyl; preferably W^{1a} is —NHCO—C₁₋₆alkyl;
Z^{2a} is selected from the group comprising hydrogen, —CO—R^{a}, halo, C₁₋₁₂alkyl, haloC₁₋₆alkyl, C₁₋₁₂alkylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{2a} is selected from the group comprising hydrogen, —CO—R^{a}, halo, C₁₋₁₂alkyl, haloC₁₋₆alkyl, C₁₋₁₂alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₆alkyl, C₁₋₆alkylcarbonyl, C₆₋₁₀aryl, 3-6 membered heterocyclyl, 5-6 membered heteroaryl, and C₃₋₆cycloalkyl; preferably Z^{2a} is selected from the group comprising hydrogen, —CO—R^{a}, C₁₋₆alkyl, and C₁₋₆alkylcarbonyl; preferably Z^{2a} is selected from the group comprising hydrogen, C₁₋₆alkyl, and C₁₋₆alkylcarbonyl; preferably Z^{2a} is C₁₋₆alkylcarbonyl;
Y^{1a} is a moiety of formula (A), or is selected from —OR^{a}, —R^{a}, or —NR^{a}R^{b};

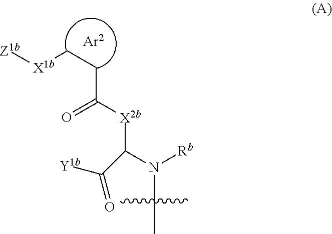

preferably Y^{1a} is a moiety of formula (A) or is —OR^{a}, or —NHR^{a}; preferably Y^{1a} is a moiety of formula (A), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is a moiety of formula (A), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is a moiety of formula (A), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (A), or is selected from the group comprising —OH, —NH₂, —O—C₁₋₁₂alkyl, and —NHC₁₋₁₂alkyl; preferably Y^{1a} is a moiety of formula (A), or is selected from the group comprising —OH, —NH₂, —O—C₁₋₁₀alkyl, and —NHC₁₋₁₀alkyl; preferably Y^{1a} is a moiety of formula (A),
Ar² is C₆₋₁₀aryl or a 5-10 membered heteroaryl; wherein each of said aryl or heteroaryl can be unsubstituted or substituted with one or more R^{zb}; preferably Ar² is C₆₋₁₀aryl or a 5-6 membered heteroaryl, preferably containing at least one N; wherein each of said aryl or heteroaryl can be unsubstituted or substituted with one or more R^{zb}; preferably Ar² is C₆₋₁₀aryl or a 6 membered heteroaryl containing at least one N; wherein each of said aryl or heteroaryl can be unsubstituted or substituted with one or more R^{zb}; preferably Ar² is phenyl; wherein said phenyl can be unsubstituted or substituted with one or more R^{zb}; preferably Ar² is phenyl;
each R^{zb} is independently selected from the group comprising hydrogen, halo, C₁₋₆alkyl, haloC₁₋₆alkyl, C₁₋₆alkylcarbonyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, C₃₋₆cycloalkyl-C₂₋₆alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glucosyl, non-acylated or acylated —O-glucosyl, non-acylated or acylated —NR^{b}-glucosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, and —OR^{a}; preferably each R^{zb} is independently selected from the group comprising hydrogen, halo, C₁₋₆alkyl, haloC₁₋₆alkyl, and haloC₁₋₆alkyloxy; preferably each R^{zb} is independently selected from the group comprising hydrogen, halo, C₁₋₄alkyl, haloC₁₋₄alkyl, and haloC₁₋₄alkyloxy; preferably each R^{zb} is independently selected from the group comprising hydrogen, halo, and C₁₋₄alkyl; preferably each R^{zb} is independently selected hydrogen, halo, or C₁₋₆alkyl; preferably each R^{zb} is hydrogen,
X^{1b} is selected from —NR^{b}—, —O—, —S—, SO₂—O—, or —SO—O—; preferably X^{1b} is selected from —NR^{b}—, —O—, —S—; preferably X^{1b} is selected from —NR^{b}—, or —O—; preferably X^{1b} is —NR^{b}—;
Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₆alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C_{2- 10}alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N or one 0, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, C₁₋₆alkylcarbonyl, C₂₋₈alkenylcarbonyl, C₆₋₁₀aryl, C₃₋₆cycloalkyl, 3-6 membered heterocyclyl-C₁₋₆alkylene containing at least one N or one O, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₈alkyl, haloC₁₋₆alkyl, 3-5 membered heterocyclyl-C₁₋₆alkylene containing at least one O, C₁₋₈alkylcarbonyl, and C₂₋₆alkenylcarbonyl; preferably Z^{1b} is selected from the group comprising hydrogen, —CO—R^{a}, —CO—C₂₋₄alkenylene-CO—O—C₁₋₄alkyl, —CO—R^{f}-maleimidyl, C₁₋₆alkyl, 3-4 membered heterocyclyl-C₁₋₂alkylene containing one O, C₁₋₆alkylcarbonyl, and C₂₋₆alkenylcarbonyl; preferably Z^{1b} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₂alkylene, C₁₋₈alkylcarbonyl, and C₂₋₆alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₈alkylcarbonyl, and C₂₋₄alkenylcarbonyl;
X^{2b} is —(CR^{c}R^{d})ₘ—; preferably X^{2b} is —(CR^{c}H)ₘ—; preferably X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—; and
Y^{1b} is selected from OR^{a}, —NR^{b}R^{a}, or R^{a}; preferably Y^{1b} is —OR^{a}, or —NHR^{a}; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl.
6. The compound according to any one of statements 1-2, 5, wherein
   Ar¹ is C₆₋₁₀aryl or a 5-6 membered heteroaryl, preferably containing at least one N; wherein each of said aryl or heteroaryl can be unsubstituted or substituted with one or more R^{za}, preferably said aryl or heteroaryl can be unsubstituted or substituted with one, two, three or four R^{za};
   Y^{1a} is a moiety of formula (A), or is selected from —OR^{a}, —R^{a}, or —NR^{a}R^{b}; Ar² is C₆₋₁₀aryl or a 5-6 membered heteroaryl, preferably containing at least one N; wherein each of said aryl or heteroaryl can be unsubstituted or substituted with one or more R^{zb}; preferably said aryl or heteroaryl can be unsubstituted or substituted with one, two, three or four R^{zb}
   wherein R^{za}, R^{zb}, R^{a}, R^{b}, have the same meaning as that defined in any one of statements 1-2, 5.
7. The compound according to any one of statements 1-2, 5-6, wherein
   Ar¹ is C₆₋₁₀aryl or a 5-6 membered heteroaryl, preferably containing at least one N; wherein each of said aryl or heteroaryl can be unsubstituted or substituted with one or more R^{za}; preferably Ar¹ is C₆₋₁₀aryl or a 6 membered heteroaryl containing at least one N; wherein each of said aryl or heteroaryl can be unsubstituted or substituted with one or more R^{za}; preferably Ar¹ is phenyl; wherein said phenyl can be unsubstituted or substituted with one or more R^{za}; preferably Ar¹ is phenyl;
   each R^{za} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, and —OR^{a}; preferably each R^{za} is independently selected from the group comprising hydrogen, halo, C₁₋₆alkyl, haloC₁₋₆alkyl, and haloC₁₋₆alkyloxy; preferably each R^{za} is independently selected from the group comprising hydrogen, halo, C₁₋₄alkyl, haloC₁₋₄alkyl, and haloC₁₋₄alkyloxy; preferably each R^{za} is independently selected from the group comprising hydrogen, halo, and C₁₋₄alkyl; preferably each R^{za} is independently selected hydrogen, halo, or C₁₋₆alkyl; preferably each R^{z}a is hydrogen,
   Y^{1a} is a moiety of formula (A), or is selected from —OR^{a}, —R^{a}, or —NR^{a}R^{b}; preferably Y^{1a} is a moiety of formula (A);

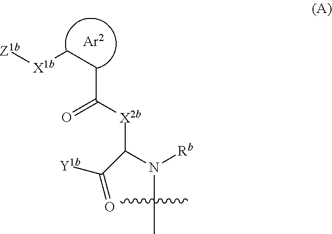

Ar² is C₆₋₁₀aryl or a 5-6 membered heteroaryl, preferably containing at least one N; wherein each of said aryl or heteroaryl can be unsubstituted or substituted with one or more R^{zb}; preferably Ar² is C₆₋₁₀aryl or a 6 membered heteroaryl containing at least one N; wherein each of said aryl or heteroaryl can be unsubstituted or substituted with one or more R^{zb}; preferably Ar² is phenyl; wherein said phenyl can be unsubstituted or substituted with one or more R^{zb}; preferably Ar² is phenyl;
each R^{zb} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, and —OR^{a}; preferably each R^{zb} is independently selected from the group comprising hydrogen, halo, C₁₋₆alkyl, haloC₁₋₆alkyl, and haloC₁₋₆alkyloxy; preferably each R^{zb} is independently selected from the group comprising hydrogen, halo, C₁₋₄alkyl, haloC₁₋₄alkyl, and haloC₁₋₄alkyloxy; preferably each R^{zb} is independently selected from the group comprising hydrogen, halo, and C₁₋₄alkyl; preferably each R^{zb} is independently selected hydrogen, halo, or C₁₋₆alkyl; preferably each R^{zb} is hydrogen.
8. The compound according to any one of statements 3 or 4, wherein
   R^{1a}, R^{2a}, R^{3a}, and R^{4a} are each independently R^{za};
   each R^{za} is independently selected from the group comprising hydrogen, halo, C₁₋₆alkyl, haloC₁₋₆alkyl, C₁₋₆alkylcarbonyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, C₃₋₆cycloalkyl-C₂₋₆alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glucosyl, non-acylated or acylated —O-glucosyl, non-acylated or acylated —NR^{b}-glucosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, and —OR^{a}; preferably each R^{za} is independently selected from the group comprising hydrogen, halo, C₁₋₆alkyl, haloC₁₋₆alkyl, and haloC₁₋₆alkyloxy; preferably each R^{za} is independently selected from the group comprising hydrogen, halo, C₁₋₄alkyl, haloC₁₋₄alkyl, and haloC₁₋₄alkyloxy; preferably each R^{za} is independently selected from the group comprising hydrogen, halo, and C₁₋₄alkyl; preferably each R^{za} is independently selected hydrogen, halo, or C₁₋₆alkyl; preferably each R^{za} is hydrogen,
   each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, C₃₋₆cycloalkyl-C₂₋₆alkynylene, and —R^{f1}-R^{g1}; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene, can be unsubstituted or substituted with one or more R^{za1}; wherein each R^{za1} is independently selected from the group comprising —COO—R^{h},

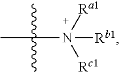

—NR^{a1}R^{b1}, —R^{f1}-R^{g1}, halo, C₁₋₆alkyl, haloC₁₋₆alkyl, C₁₋₆alkylcarbonyl, C₂₋₆alkenyl, and C₂₋₆alkynyl; R^{h} is selected from the group comprising hydrogen, C₁₋₆alkyl, C₂₋₆alkenyl, and C₂₋₆alkynyl; R^{a1} is selected from the group comprising hydrogen, C₁₋₆alkyl, C₂₋₆alkenyl, and C₂₋₆alkynyl; R^{b1} is selected from the group comprising hydrogen, C₁₋₆alkyl, C₂₋₆alkenyl, and C₂₋₆alkynyl; R^{c1} is selected from the group comprising hydrogen, C₁₋₆alkyl, C₂₋₆alkenyl, and C₂₋₆alkynyl; R^{f1} is C₁₋₂₀alkylene, wherein said C₁₋₂₀alkylene can be interrupted by one or more O or S; R^{g1} is hydrogen, or C₁₋₂₀alkyl; preferably each R^{a} is independently selected from the group comprising hydrogen, halo, C₁₋₁₈alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, C₃₋₆-cycloalkyl-C₁₋₆alkylene, and —R^{f1}-R^{g1}; wherein each of said C₁₋₁₈alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

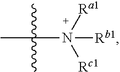

—NR^{a1}R^{b1}, —R^{f1}-R^{g1}, halo, C₁₋₆alkyl, haloC₁₋₆alkyl, and C₁₋₆alkylcarbonyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₁₀alkylene, wherein said C₁₋₁₀alkylene can be interrupted by one or more O or S; R^{g1} is hydrogen, or C₁₋₁₀alkyl; preferably each R^{a} is independently selected from the group comprising hydrogen, halo, C₁₋₅alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, C₃₋₆cycloalkyl-C₁₋₆alkylene, and —R^{f1}-R^{g1}; wherein each of said C₁₋₁₆alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

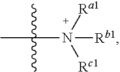

—NR^{a1}R^{b1}, —R^{f1}-R^{g1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl; preferably each R^{a} is independently selected from the group comprising hydrogen, halo, C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, C₃₋₆cycloalkyl, and —R^{f1}-R^{g1}; wherein each of said C₁₋₁₅alkyl, C₂₋₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

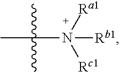

—NR^{a1}R^{b1}, —R^{f1}-R^{g1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl; preferably each R^{a} is independently selected from the group comprising hydrogen, halo, C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, C₅₋₆cycloalkyl, and —R^{f1}-R^{g1}; wherein each of said C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

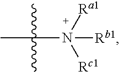

—NR^{a1}R^{b1}, —R^{f1}-R^{g1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl; preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-4 membered heterocyclyl containing at least one O, phenyl, —R^{f1}-R^{g1}; wherein each of said C₁₋₁₅alkyl, C₂₋₆alkenyl, haloC₁₋₆alkyl, C₁₋₆alkylcarbonyl, 3-4 membered heterocyclyl containing at least one O, and phenyl, can be unsubstituted or substituted with one or more R^{za1}; wherein each R^{za1} is independently selected from the group comprising —COO—R^{h},

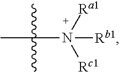

—NR^{a1}R^{b1}, —R^{f1}-R^{g1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{b1} is C₁₋₆alkylene, wherein said C₁₋₆alkylene can be interrupted by one or more O; R^{g1} is hydrogen, or C₁₋₆alkyl; preferably each R^{a} is independently selected from the group comprising hydrogen, halo, C₁₋₁₅alkyl, C₂₋₆alkenyl, oxiranyl, C₁₋₆alkylcarbonyl, and —R^{f1}-R^{g1}; wherein each of said C₁₋₁₅alkyl, C₂₋₆alkenyl, oxiranyl, and C₁₋₆alkylcarbonyl, can be unsubstituted or substituted with one or more R^{za1}; wherein each R^{za1} is independently selected from the group comprising —COO—R^{h},

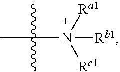

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O; R^{g1} is hydrogen, or C₁₋₆alkyl; preferably each R^{a} is independently selected from the group comprising hydrogen, halo, C₁₋₁₅alkyl, C₂₋₄alkenyl, and oxiranyl; wherein each of said C₁₋₁₅alkyl, C₂₋₆alkenyl, and oxiranyl, can be unsubstituted or substituted with one or more R^{za1}; wherein each R^{za1} is independently selected from the group comprising —COO—R^{h},

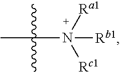

—NR^{a1}R^{b1}, —R^{f1}-R^{g1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R¹ is selected from hydrogen, or C₁₋₄alkyl; R^{f1} is C₁₋₆alkylene, wherein said C₁₋₆alkylene can be interrupted by one or more O; R^{g1} is hydrogen, or C₁₋₄alkyl; for example, each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably each R^{a} is independently selected from the group comprising hydrogen, halo, C₁₋₆alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{a} is independently selected from the group comprising hydrogen, halo, C₁₋₆alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl; preferably each R^{a} is independently selected from the group comprising hydrogen, halo, C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl; preferably each R^{a} is independently selected from the group comprising hydrogen, halo, C₁₋₁₅alkyl, C₂₋₈alkenyl, haloC₁₋₆alkyl, C₁₋₆alkylcarbonyl, 3-4 membered heterocyclyl containing at least one O, and phenyl; preferably each R^{a} is independently selected from the group comprising hydrogen, halo, C₁₋₁₅alkyl, C₂₋₆alkenyl, oxiranyl, and C₁₋₆alkylcarbonyl; preferably each R^{a} is independently selected from the group comprising hydrogen, halo, C₁₋₁₅alkyl, C₂₋₄alkenyl, and oxiranyl;
each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 5-10 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl containing at least one N, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, and C₆₋₁₀aryl; preferably each R^{b} is independently selected from the group comprising hydrogen, C₁₋₆alkyl, and haloC₁₋₆alkyl; preferably each R^{b} is independently selected from hydrogen, or C₁₋₆alkyl; preferably each R^{b} is hydrogen;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{a}-maleimidyl, halo, C₁₋₆alkyl, haloC₁₋₆alkyl, C₁₋₆alkylcarbonyl, C₂₋₈alkenylcarbonyl, C₆₋₁₀aryl, C₃₋₆cycloalkyl, 3-6 membered heterocyclyl-C₁₋₆alkylene containing at least one N or one O, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{a}-maleimidyl, halo, C₁₋₈alkyl, haloC₁₋₆alkyl, 3-5 membered heterocyclyl-C₁₋₆alkylene containing at least one O, C₁₋₈alkylcarbonyl, and C₂₋₆alkenylcarbonyl; preferably Z^{1a} is selected from the group comprising hydrogen, —CO—R^{a}, —CO—C₂₋₄alkenylene-CO—O—C₁₋₄alkyl, —CO—C₁₋₆alkylene-maleimidyl, C₁₋₈alkyl, 3-4 membered heterocyclyl-C₁₋₂alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₂alkylene, C₁₋₈alkylcarbonyl, and C₂₋₆alkenylcarbonyl; preferably Z^{1a} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl;
X^{2a} is —(CR^{c}R^{d})ₙ—; preferably X^{2a} is —(CR^{c}H)ₙ—; preferably X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—;
each R^{c} and R^{d} is independently selected from the group comprising hydrogen, halo, C₁₋₆alkyl, haloC₁₋₆alkyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably each R^{c} and R^{d} is independently selected from the group comprising hydrogen, halo, C₁₋₆alkyl, haloC₁₋₆alkyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₂₋₆alkylene; preferably each R^{c} and R^{d} is independently selected from the group comprising hydrogen, halo, C₁₋₆alkyl, haloC₁₋₆alkyl, C₆₋₁₀aryl, 3-10 membered heterocyclyl, 5-10 membered heteroaryl, and C₃₋₆cycloalkyl; preferably each R^{c} and R^{d} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{c} and R^{d} is independently selected from the group comprising hydrogen, halo, C₁₋₆alkyl, haloC₁₋₆alkyl, C₆₋₁₀aryl, 3-6 membered heterocyclyl containing at least one N or one O, 5-6 membered heteroaryl containing at least one N, and C₃₋₆cycloalkyl; preferably each R^{c} and R^{d} is independently selected from the group comprising hydrogen, halo, C₁₋₆alkyl, haloC₁₋₆alkyl, and C₆₋₁₀aryl; preferably each R^{c} and R^{d} is independently selected from hydrogen, or C₁₋₆alkyl; preferably each R^{c} and R^{d} is independently is hydrogen;
R^{e} is selected from the group comprising C₁₋₁₂alkylene, C₂₋₁₂alkenylene, C₂₋₁₂alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₂₋₆alkynylene, C₆₋₁₀arylene, 4-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₃alkylene; preferably R^{e} is C₂₋₄alkenylene; preferably R^{e} is C₂₋₈alkenylene;
R^{f} is C₁₋₂₀alkylene, wherein said alkylene can be interrupted by one or more O or S; preferably R^{f} is C₁₋₁₆alkylene, wherein said alkylene can be interrupted by one or more O or S; preferably R^{f} is C₁₋₁₄alkylene, wherein said alkylene can be interrupted by one or more O or S; preferably R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from hydrogen, C₁₋₂₀alkyl, N-maleimidyl, N-maleimidyloxy, or N-maleimidyloxycarbonyl; preferably R^{g} is hydrogen, C₁₋₁₀alkyl, N-maleimidyl, or N-maleimidyloxy; preferably R^{g} is hydrogen, C₁₋₆alkyl, N-maleimidyl;
W^{1a} is selected from —NR^{b}Z^{2a}, —R^{a}, or —OR^{a}; preferably W^{1a} is selected from —NR^{1a}Z^{2a}, or —R^{a}; preferably W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W^{1a} is —NHCO—C₁₋₈alkyl; preferably W^{1a} is —NHCO—C₁₋₆alkyl;
Z^{2a} is selected from the group comprising hydrogen, —CO—R^{a}, halo, C₁₋₁₂alkyl, haloC₁₋₆alkyl, C₁₋₁₂alkylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{2a} is selected from the group comprising hydrogen, —CO—R^{a}, halo, C₁₋₁₂alkyl, haloC₁₋₆alkyl, C₁₋₁₂alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₆alkyl, C₁₋₆alkylcarbonyl, C₆₋₁₀aryl, 3-6 membered heterocyclyl, 5-6 membered heteroaryl, and C₃₋₆cycloalkyl; preferably Z^{2a} is selected from the group comprising hydrogen, —CO—R^{a}, C₁₋₆alkyl, and C₁₋₆alkylcarbonyl; preferably Z^{2a} is selected from the group comprising hydrogen, C₁₋₆alkyl, and C₁₋₆alkylcarbonyl; preferably Z^{2a} is C₁₋₆alkylcarbonyl;
Y^{1a} is a moiety of formula (A), or is selected from —OR^{a}, —R^{a}, or —NR^{a}R^{b};

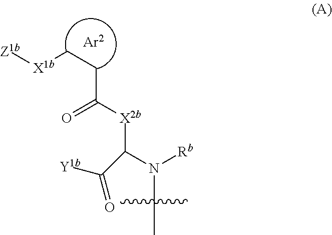

preferably Y^{1a} is a moiety of formula (A) or is —OR^{a}, or —NHR^{a}; preferably Y^{1a} is a moiety of formula (A), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is a moiety of formula (A), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is a moiety of formula (A), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (A), or is selected from the group comprising —OH, —NH₂, —O—C₁₋₁₂alkyl, and —NHC₁₋₁₂alkyl; preferably Y^{1a} is a moiety of formula (A), or is selected from the group comprising —OH, —NH₂, —O—C₁₋₁₀alkyl, and —NHC₁₋₁₀alkyl; preferably Y^{1a} is a moiety of formula (A),
Ar² is C₆₋₁₀aryl or a 5-10 membered heteroaryl; wherein each of said aryl or heteroaryl can be unsubstituted or substituted with one or more R^{zb}; preferably Ar² is C₆₋₁₀aryl or a 5-6 membered heteroaryl, preferably containing at least one N; wherein each of said aryl or heteroaryl can be unsubstituted or substituted with one or more R^{zb}; preferably Ar² is C₆₋₁₀aryl or a 6 membered heteroaryl containing at least one N; wherein each of said aryl or heteroaryl can be unsubstituted or substituted with one or more R^{zb}; preferably Ar² is phenyl; wherein said phenyl can be unsubstituted or substituted with one or more R^{zb}; preferably Ar² is phenyl;
each R^{zb} is independently selected from the group comprising hydrogen, halo, C₁₋₆alkyl, haloC₁₋₆alkyl, C₁₋₆alkylcarbonyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, C₃₋₆cycloalkyl-C₂₋₆alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glucosyl, non-acylated or acylated —O-glucosyl, non-acylated or acylated —NR^{b}-glucosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, and —OR^{a}; preferably each R^{zb} is independently selected from the group comprising hydrogen, halo, C₁₋₆alkyl, haloC₁₋₆alkyl, and haloC₁₋₆alkyloxy; preferably each R^{zb} is independently selected from the group comprising hydrogen, halo, C₁₋₄alkyl, haloC₁₋₄alkyl, and haloC₁₋₄alkyloxy; preferably each R^{zb} is independently selected from the group comprising hydrogen, halo, and C₁₋₄alkyl; preferably each R^{zb} is independently selected hydrogen, halo, or C₁₋₆alkyl; preferably each R^{zb} is hydrogen,
X^{1b} is selected from —NR^{b}—, —O—, —S—, SO₂—O—, or —SO—O—; preferably X^{1b} is selected from —NR^{b}—, —O—, —S—; preferably X^{1b} is selected from —NR^{b}—, or —O—; preferably X^{1b} is —NR^{b}—;
Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₆alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₂₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C_{2- 10}alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈ alkylcarbonyl, C₂₋₆alkenylcarbonyl, C₆₋₁₀aryl, 3-6 membered heterocyclyl containing at least one N or one 0, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, C₁₋₆alkylcarbonyl, C₂₋₈alkenylcarbonyl, C₆₋₁₀aryl, C₃₋₆cycloalkyl, 3-6 membered heterocyclyl-C₁₋₆alkylene containing at least one N or one 0, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₈alkyl, haloC₁₋₆alkyl, 3-5 membered heterocyclyl-C₁₋₆alkylene containing at least one O, C₁₋₈alkylcarbonyl, and C₂₋₆alkenylcarbonyl; preferably Z^{1b} is selected from the group comprising hydrogen, —CO—R^{a}, —CO—C₂₋₄alkenylene-CO—O—C₁₋₄alkyl, —CO—R^{f}-maleimidyl, C₁₋₆alkyl, 3-4 membered heterocyclyl-C₁₋₂alkylene containing one O, C₁₋₆alkylcarbonyl, and C₂₋₆alkenylcarbonyl; preferably Z^{1b} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₂alkylene, C₁₋₈alkylcarbonyl, and C₂₋₆alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl;
X^{2b} is —(CR^{c}R^{d})ₘ—; preferably X^{2b} is —(CR^{c}H)ₘ—; preferably X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—; and
Y^{1b} is selected from OR^{a}, —NR^{b}R^{a}, or R^{a}; preferably Y^{1b} is —OR^{a}, or —NHR^{a}; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably
Y^{1b} is —O—C₁₋₁₀alkyl, or —NH—C₁₋₁₀alkyl.
9. The compound according to any one of statements 1-8, wherein
   Y^{1a} is a moiety of formula (A), or is selected from —OR^{a}, —R^{a}, or —NR^{a}R^{b};
   Ar² is C₆₋₁₀aryl or a 5-6 membered heteroaryl, preferably containing at least one N; wherein each of said aryl or heteroaryl can be unsubstituted or substituted with one or more R^{zb}-preferably said aryl or heteroaryl can be unsubstituted or substituted with one, two, three or four R^{zb}
   wherein R^{za}, R^{zb}, R^{a}, R^{b}, have the same meaning as that defined in any one of statements 1-8.
10. The compound according to any one of statements 1-9, wherein
   each R^{za} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{za} is independently selected from the group comprising hydrogen, halo, C₁₋₆alkyl, haloC₁₋₆alkyl, and —OR^{a}; preferably each R^{za} is independently selected from the group comprising hydrogen, halo, C₁₋₆alkyl, haloC₁₋₆alkyl, and haloC₁₋₆alkyloxy; preferably each R^{za} is independently selected from the group comprising hydrogen, halo, C₁₋₄alkyl, haloC₁₋₄alkyl, and haloC₁₋₄alkyloxy; preferably each R^{za} is independently selected from the group comprising hydrogen, halo, and C₁₋₄alkyl; preferably each R^{za} is independently selected hydrogen, halo, or C₁₋₆alkyl; preferably each R^{z}a is hydrogen,
   Y^{1a} is a moiety of formula (A), or is selected from —OR^{a}, —R^{a}, or —NR^{a}R^{b};
   Ar² is C₆₋₁₀aryl or a 5-6 membered heteroaryl, preferably containing at least one N; wherein each of said aryl or heteroaryl can be unsubstituted or substituted with one or more R^{zb}; preferably Ar² is C₆₋₁₀aryl or a 6 membered heteroaryl containing at least one N; wherein each of said aryl or heteroaryl can be unsubstituted or substituted with one or more R^{zb}; preferably Ar² is phenyl; wherein said phenyl can be unsubstituted or substituted with one or more R^{zb}; preferably Ar² is phenyl;
   each R^{zb} is independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably each R^{zb} is independently selected from the group comprising hydrogen, halo, C₁₋₆alkyl, haloC₁₋₆alkyl, and —OR^{a}; preferably each R^{zb} is independently selected from the group comprising hydrogen, halo, C₁₋₆alkyl, haloC₁₋₆alkyl, and haloC₁₋₆alkyloxy; preferably each R^{zb} is independently selected from the group comprising hydrogen, halo, C₁₋₄alkyl, haloC₁₋₄alkyl, and haloC₁₋₄alkyloxy; preferably each R^{zb} is independently selected from the group comprising hydrogen, halo, and C₁₋₄alkyl; preferably each R^{zb} is independently selected hydrogen, halo, or C₁₋₆alkyl; preferably each R^{zb} is hydrogen.
11. The compound according to any one of statements 1-10, having structural formula (II);

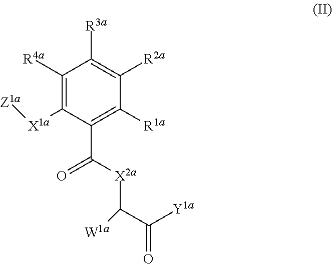

wherein Y^{1a} is a moiety of formula (B), or is selected from OR^{a}, R^{a}, NR^{a}R^{b},

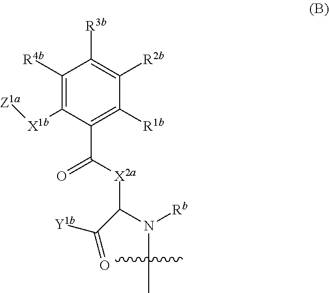

wherein R^{1a}, R^{2a}, R^{3a}, and R^{4a} are each independently R^{za}; and R^{1b}, R^{2b}, R^{3b}, and R^{4b} are each independently R^{zb}, and
wherein Z^{1a}, X^{1a}, X^{2a}, W^{1a}, Z^{1b}, X^{1b}, X^{2b}, Y^{1b}, R^{b}, R^{za}, and R^{zb}, have the same meaning as that defined in any one of statements 1-4;
preferably R^{1a}, R^{2a}, R^{3a}, R^{4a}, R^{1b}, R^{2b}, R^{3b}, and R^{4b} are each independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, —C(O)—R^{a}, —S(O)R^{a}, —NR^{a}R^{b}, and —NR^{b}C(O)R^{a}; preferably R^{1a}, R^{2a}, R^{3a}, R^{4a}, R^{1b}, R^{2b}, R^{3b}, R^{4b}, are each independently selected from the group comprising hydrogen, halo, C₁₋₆alkyl, haloCₗ₋₆alkyl, C₁₋₆alkylcarbonyl, C₂₋₆alkenyl, C₂₋₆alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, C₃₋₆cycloalkyl-C₂₋₆alkynylene, —OR^{a}, —SR^{a}, —S(O)R^{a}, —NR^{a}R^{b}, and —NR^{b}C(O)R^{a}; preferably R^{1a}, R^{2a}, R^{3a}, R^{4a}, R^{1b}, R^{2b}, R^{3b}, R^{4b}, are each independently selected from the group comprising hydrogen, halo, C₁₋₆alkyl, haloC₁₋₆alkyl, C₁₋₆alkylcarbonyl, C₆₋₁₀aryl, C₃₋₆cycloalkyl, C₆₋₁₀aryl-C₁₋₆alkylene, C₃₋₆cycloalkyl-C₁₋₆alkylene, —OR^{a}, —SR^{a}, —S(O)R^{a}, —NR^{a}R^{b}, and —NR^{b}C(O)R^{a}; preferably R^{1a}, R^{2a}, R^{3a}, R^{4a}, R^{1b}, R^{2b}, R^{3b}, R^{4b}, are each independently selected from the group comprising hydrogen, halo, C₁₋₆alkyl, haloC₁₋₆alkyl, C₆₋₁₀aryl, C₃₋₆cycloalkyl, —OR^{a}, —NR^{a}R^{b}, and —NR^{b}C(O)R^{a}; preferably R^{1a}, R^{2a}, R^{3a}, R^{4a}, R^{1b}, R^{2b}, R^{3b}, R^{4b}, are each independently selected from the group comprising hydrogen, halo, C₁₋₆alkyl, haloC₁₋₆alkyl, and —OR^{a}; preferably R^{1a}, R^{2a}, R^{3a}, R^{4a}, R^{1b}, R^{2b}, R^{3b}, R^{4b}, are each independently selected from the group comprising hydrogen, halo, C₁₋₆alkyl, haloC₁₋₆alkyl, and haloC₁₋₆alkyloxy; preferably R^{1a}, R^{2a}, R^{3a}, R^{4a}, R^{1b}, R^{2b}, R^{3b}, R^{4b}, are each independently selected from the group comprising hydrogen, halo, C₁₋₄alkyl, haloC₁₋₄alkyl, and haloC₁₋₄alkyloxy; preferably Ria, R^{2a}, R^{3a}, R^{4a}, R^{1b}, R^{2b}, R^{3b}, R^{4b}, are each independently selected from the group comprising hydrogen, halo, and C₁₋₄alkyl; preferably Ria, R^{2a}, R^{3a}, R^{4a}, R^{1b}, R^{2b}, R^{3b}, R^{4b}, are each independently selected from hydrogen, halo, or C₁₋₃alkyl; preferably Ria, R^{2a}, R^{3a}, R^{4a}, Rib, R^{2b}, R^{3b}, R^{4b}, are each independently hydrogen.
12. The compound according to any one of statements 1-2, 5-11, wherein
   each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

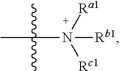

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; wherein each of said C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

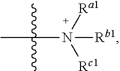

—NR^{a1}R^{b1} and C₁₋₆alkyl; R^{h} is selected from hydrogen, or C₁₋₆alkyl; R^{a1} is selected from hydrogen, or C₁₋₆alkyl;
R^{b1} is selected from hydrogen, or C₁₋₆alkyl; R¹ is selected from hydrogen, or C₁₋₆alkyl; preferably each R^{a} is independently selected from the group comprising hydrogen, halo, C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl; wherein each of said C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

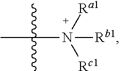

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₅alkyl, C₂₋₆alkenyl, haloC₁₋₆alkyl, C₆₋₁₀alkylcarbonyl, phenyl, 6 membered heteroaryl containing at least one N, 3-6 membered heterocyclyl containing at least one N or one O, and C₅₋₆cycloalkyl; wherein each of said C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

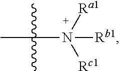

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-5 membered heterocyclyl containing at least one N or one O, and phenyl; wherein each of said C₁₋₁₅alkyl, C₂₋₆alkenyl, haloC₁₋₆alkyl, C₁₋₆alkylcarbonyl, 3-5 membered heterocyclyl containing at least one N or one O, and phenyl, can be unsubstituted or substituted with one or more R^{za1}; wherein each R^{za1} is independently selected from the group comprising —COO—R^{h},

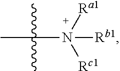

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₅alkyl, C₂₋₆alkenyl, 3-4 membered heterocyclyl containing at least one O, and C₁₋₈alkylcarbonyl; wherein each of said C₁₋₁₅alkyl, C₂₋₆alkenyl, 3-4 membered heterocyclyl containing at least one O, and C₁₋₆alkylcarbonyl, can be unsubstituted or substituted with one or more R^{za1}; wherein each R^{za1} is independently selected from the group comprising —COO—R^{h},

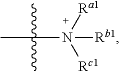

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₅alkyl, C₂₋₄alkenyl, and oxiranyl; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

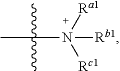

—NR^{a1}R^{b1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl;
for example each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{a} is independently selected from the group comprising hydrogen, halo, C₁₋₁₆alkyl, C₂₋₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl; preferably each R^{a} is independently selected from the group comprising hydrogen, halo, C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl; preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-5 membered heterocyclyl containing at least one N or one O, and phenyl; preferably each R^{a} is independently selected from the group comprising hydrogen, halo, C₁₋₁₅alkyl, C₂₋₆alkenyl, 3-4 membered heterocyclyl containing at least one O, and C₁₋₈alkylcarbonyl; preferably each R^{a} is independently selected from the group comprising hydrogen, halo, C₁₋₁₅alkyl, C₂₋₄alkenyl, and oxiranyl;
each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 3-6 membered heterocyclyl-C₁₋₆alkylene, 5-10 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl, 3-6 membered heterocyclyl-C₁₋₆alkylene, 5-6 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl containing at least one N, 3-6 membered heterocyclyl-C₁₋₆alkylene containing at least one 0, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, 3-5 membered heterocyclyl-C₁₋₆alkylene containing at least one O, and C₆₋₁₀aryl; preferably each R^{b} is independently selected from the group comprising hydrogen, C₁₋₆alkyl, 3-4 membered heterocyclyl-C₁₋₄alkylene containing one O, and haloC₁₋₆alkyl; preferably each R^{b} is independently selected from hydrogen, oxiranyl-C₁₋₂alkylene, or C₁₋₆alkyl; preferably each R^{b} is hydrogen;
X^{1a} is selected from —NR^{b}—, or —O—; preferably X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁-alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N, 3-6 membered heterocyclyl-C₁₋₆alkylene containing at least one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1a} is selected from the group comprising hydrogen, —CO—R^{a}, —CO—C₂₋₄alkenylene-CO—O—C₁₋₄alkyl, —CO—R^{f}-maleimidyl, C₁₋₈alkyl, 3-4 membered heterocyclyl-C₁₋₆alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene-, C₁₋₈alkylcarbonyl, and, and C₂₋₄alkenylcarbonyl; preferably Z^{1a} is selected from the group comprising hydrogen, C₁₋₆alkylcarbonyl, —CO—C₂₋₄alkenylene-CO—O—C₁₋₄alkyl, —CO—C₁₋₆alkylene-maleimidyl, oxiranyl-C₁₋₂alkylene, and C₂₋₄alkenylcarbonyl;
X^{2a} is —(CR^{c}H)ₙ—; preferably X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—;
each R^{c} is independently selected from the group comprising hydrogen, halo, C₁₋₆alkyl, haloC₁₋₆alkyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{c} is independently selected from the group comprising hydrogen, halo, C₁₋₆alkyl, haloC₁₋₆alkyl, C₆₋₁₀aryl, 3-10 membered heterocyclyl, 5-10 membered heteroaryl, and C₃₋₆cycloalkyl; preferably each R^{c} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{c} is independently selected from the group comprising hydrogen, halo, C₁₋₆alkyl, haloC₁₋₆alkyl, C₆₋₁₀aryl, 3-6 membered heterocyclyl containing at least one N or one O, 5-6 membered heteroaryl containing at least one N, and C₃₋₆cycloalkyl; preferably each R^{c} is independently selected from the group comprising hydrogen, halo, C₁₋₆alkyl, haloC₁₋₆alkyl, and C₆₋₁₀aryl; preferably each R^{c} is independently selected from hydrogen, or C₁₋₆alkyl; preferably each R^{c} is hydrogen;
R^{e} is selected from the group comprising C₁₋₆alkylene, C₂₋₆alkenylene, C₂₋₆alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₆alkenylene;
R^{f} is C₁₋₂₀alkylene, wherein said alkylene can be interrupted by one or more O or S; preferably R^{f} is C₁₋₁₄alkylene, wherein said alkylene can be interrupted by one or more O or S; preferably R^{f} is C₁₋₁₄alkylene, wherein said alkylene can be interrupted by one or more O or S; preferably R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from N-maleimidyl, N-maleimidyloxy, or N-maleimidyloxycarbonyl; preferably R^{g} is N-maleimidyl, or N-maleimidyloxy; preferably R^{g} is N-maleimidyl;
W^{1a} is selected from —NR^{1a}Z^{2a}, or —R^{a}; preferably W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W^{1a} is —NHCO—C₁₋₈alkyl; preferably W¹a is —NHCO—C₁₋₈alkyl;
Z^{2a} is selected from the group comprising hydrogen, —CO—R^{a}, halo, C₁₋₁₂alkyl, haloC₁₋₆alkyl, C₁₋₁₂alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₈alkyl, C₁₋₆alkylcarbonyl, C₆₋₁₀aryl, 3-6 membered heterocyclyl, 5-6 membered heteroaryl, and C₃₋₆cycloalkyl; preferably Z^{2a} is selected from the group comprising hydrogen, —CO—R^{a}, C₁₋₈alkyl, and C₁₋₈alkylcarbonyl; preferably Z^{2a} is selected from the group comprising hydrogen, C₁₋₈ alkyl, and C₁₋₆alkylcarbonyl; preferably Z^{2a} is C₁₋₆alkylcarbonyl;
   Y^{1a} is a moiety of formula (A) or (B) or is —OR^{a}, or —NHR^{a}; preferably Y^{1a} is a moiety of formula (A) or (B), or Y^{1a} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃-cycloalkyl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (A) or (B), or Y^{1a} is selected from the group comprising —OH, —NH₂, —O—C₁₋₁₂alkyl, and —NHC₁₋₁₂alkyl; preferably Y^{1a} is a moiety of formula (A) or (B), or is selected from the group comprising —OH, —NH₂, —O—C₁₋₁₀alkyl, and —NHC₁₋₁₀alkyl; preferably Y^{1a} is a moiety of formula (B),
   X^{1b} is selected from —NR^{b}—, or —O—; preferably X^{1b} is —NR^{b}—;
   Z^{1b} is selected from the group comprising hydrogen, —CO—R^{a}, —CO—R^{e}—CO—O—R¹, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃-cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N, 3-6 membered heterocyclyl-C₁₋₆alkylene containing at least 0, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-5 membered heterocyclyl-C₁₋₆alkylene containing at least one 0, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, 3-4 membered heterocyclyl-C₁₋₆alkylene containing at least one O, C₁₋₆alkylcarbonyl, and C₂₋₄alkenylcarbonyl; preferably Z^{1b} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₈alkylcarbonyl, and C₂₋₆alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, C₁₋₆alkyl, oxiranyl-C₁₋₄alkylene, C₁₋₆alkylcarbonyl, and C₂₋₄alkenylcarbonyl; preferably Z^{1b} is selected from the group comprising hydrogen, oxiranyl-C₁₋₂alkylene, —CO—C₂₋₄alkenylene-CO—O—C₁₋₄alkyl, —CO—C₁₋₆alkylene-maleimidyl, C₁₋₆alkylcarbonyl, and C₂₋₄alkenylcarbonyl;
   X^{2b} is —(CR^{c}H)ₘ—; preferably X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—; and
   Y^{1b} is —OR^{a}, or —NHR^{a}; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₆₋₁₀alkyl, and —NHC₁₋₁₀alkyl; preferably Y^{1b} is —O—C₁₋₁₀alkyl, or —NH—C₁₋₁₀alkyl.
13. The compound according to any one of statements 1-12, wherein
   each R^{a} is independently selected from the group comprising hydrogen, halo, C₁₋₂₀alkyl, C₂₋₂₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; wherein each of said C₁₋₂₀alkyl, C₂₋₂₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

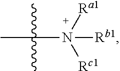

—NR^{a1}R^{b1}, and C₁₋₆alkyl; R^{h} is selected from hydrogen, or C₁₋₆alkyl; R^{a1} is selected from hydrogen, or C₁₋₆alkyl;
R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₈alkyl, C₂₋₁₈alkenyl, haloC₁₋₆alkyl, C₁₋₁₂alkylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, and C₃₋₆cycloalkyl; wherein each of said C₁₋₈alkyl, C₂₋₁₈alkenyl, haloC₁₋₆alkyl, C₁₋₁₂alkylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

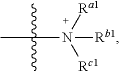

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₆alkyl, C₂₋₁₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl; wherein each of said C₁₋₁₆alkyl, C₂₋₁₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

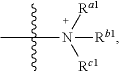

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₅alkyl, C₂₋₁₀alkenyl, haloC₁₋₆alkyl, C₁₋₆alkylcarbonyl, 3-5 membered heterocyclyl containing at least one O, and phenyl; wherein each of said C₁₋₁₅alkyl, C₂₋₁₀alkenyl, haloC₁₋₆alkyl, C₁₋₈alkylcarbonyl, 3-5 membered heterocyclyl containing at least one O, and phenyl, can be unsubstituted or substituted with one or more R^{za1}; wherein each R^{za1} is independently selected from the group comprising —COO—R^{h},

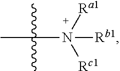

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{a1} is selected from hydrogen, or C₁₋₆alkyl;
preferably each R^{a} is independently selected from the group comprising hydrogen, halo, C₁₋₁₅alkyl, C₂₋₆alkenyl, 3-4 membered heterocyclyl containing at least one O, and C₁₋₈alkylcarbonyl; wherein each of said C₁₋₁₅alkyl, C₂₋₆alkenyl, 3-4 membered heterocyclyl containing at least one O, and C₁₋₆alkylcarbonyl, can be unsubstituted or substituted with one or more R^{za1}; wherein each R^{za1} is independently selected from the group comprising —COO—R^{h},

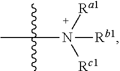

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₀alkyl, C₂₋₆alkenyl, 3-4 membered heterocyclyl containing at least one O, and C₁₋₆alkylcarbonyl; wherein each of said C₁₋₁₀alkyl, C₂₋₆alkenyl, 3-4 membered heterocyclyl containing at least one O, and C₁₋₆alkylcarbonyl, can be unsubstituted or substituted with one or more R^{za1}; wherein each R^{za1} is independently selected from the group comprising —COO—R^{h},

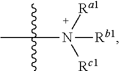

—NR^{a1}R^{b1} and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl;
preferably each R^{a} is independently selected from the group comprising hydrogen, halo, C₁₋₁₅alkyl, C₂₋₄alkenyl, and oxiranyl; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

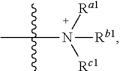

—NR^{a1}R^{b1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; for example each R^{a} is independently selected from the group comprising hydrogen, halo, C₁₋₂₀alkyl, C₂₋₂₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₁₈alkyl, C₂₋₁₈alkenyl, haloC₁₋₆alkyl, C₁₋₁₂alkylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, and C₃₋₆cycloalkyl; preferably each R^{a} is independently selected from the group comprising hydrogen, halo, C₁₋₁₆alkyl, C₂₋₁₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl; preferably each R^{a} is independently selected from the group comprising hydrogen, halo, C₁₋₁₅alkyl, C₂₋₁₀alkenyl, haloC₁₋₆alkyl, C₁₋₆alkylcarbonyl, 3-5 membered heterocyclyl containing at least one O, and phenyl; preferably each R^{a} is independently selected from the group comprising hydrogen, halo, C₁₋₁₅alkyl, C₂₋₆alkenyl, 3-4 membered heterocyclyl containing at least one O, and C₁₋₈alkylcarbonyl; preferably each R^{a} is independently selected from the group comprising hydrogen, halo, C₁₋₅alkyl, C₂₋₄alkenyl, and oxiranyl;
each R^{b} is independently selected from the group comprising hydrogen, halo, C₁₋₆alkyl, haloC₁₋₆alkyl, C₆₋₁₀aryl, 3-6 membered heterocyclyl, 3-10 membered heterocyclyl-C₁₋₆alkylene, 5-6 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl containing at least one N, 3-6 membered heterocyclyl-C₁₋₆alkylene containing at least one O, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, 3-5 membered heterocyclyl-C₁₋₆alkylene containing at least one O, and C₆₋₁₀aryl; preferably each R^{b} is independently selected from the group comprising hydrogen, C₁₋₆alkyl, 3-4 membered heterocyclyl-C₁₋₄alkylene containing one O, and haloC₁₋₆alkyl; preferably each R^{b} is independently selected from hydrogen, oxiranyl-C₁₋₂alkylene, or C₁₋₆alkyl; preferably each R^{b} is hydrogen;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1a} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₆alkylcarbonyl, and C₂₋₆alkenylcarbonyl; preferably Z^{1a} is selected from the group comprising hydrogen, C₁₋₈alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₆alkenylcarbonyl; preferably Z^{1a} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₈alkylcarbonyl, and C₂₋₄alkenylcarbonyl;
R^{e} is selected from the group comprising C₁₋₆alkylene, C₂₋₆alkenylene, C₂₋₆alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₆alkenylene;
R^{f} is C₁₋₂₀alkylene, wherein said alkylene can be interrupted by one or more O or S; preferably
R^{f} is C₁₋₁₆alkylene, wherein said alkylene can be interrupted by one or more O or S; preferably
R^{f} is C₁₋₁₄alkylene, wherein said alkylene can be interrupted by one or more O or S; preferably
R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from N-maleimidyl, N-maleimidyloxy, or N-maleimidyloxycarbonyl; preferably R^{g} is N-maleimidyl, or N-maleimidyloxy; preferably R^{g} is N-maleimidyl;
X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—;
W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W^{1a} is —NHCO—C₁₋₆alkyl; preferably W^{1a} is —NHCO—C₁₋₆alkyl;
Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₆alkyl, C₁₋₆alkylcarbonyl, C₆₋₁₀aryl, 3-6 membered heterocyclyl, 3-6 membered heterocyclyl-C₁₋₆alkylene, 5-6 membered heteroaryl, and C₃₋₆cycloalkyl; preferably Z^{2a} is selected from the group comprising hydrogen, —CO—R^{a}, C₁₋₆alkyl, and C₁₋₆alkylcarbonyl; preferably Z^{2a} is selected from the group comprising hydrogen, C₁₋₆alkyl, and C₁₋₈alkylcarbonyl; preferably Z^{2a} is C₁₋₆alkylcarbonyl;
Y^{1a} is a moiety of formula (A) or (B), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (B), or is selected from the group comprising —OH, —NH₂, —O—C₁₋₁₂alkyl, and —NHC₁₋₁₂alkyl; preferably Y^{1a} is a moiety of formula (B), or is selected from the group comprising —OH, —NH₂, —O—C₁₋₁₀alkyl, and —NHC₁₋₁₀alkyl; preferably Y^{1a} is a moiety of formula (B),
X¹b is —NR^{b}—;
Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₆alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1b} is selected from the group comprising hydrogen, —CO—R^{a}, —CO—C₂-alkenylene-CO—O—R^{b}, —CO—R^{f}-maleimidyl, halo, C₁₋₆alkyl, haloC₁₋₆alkyl, 3-5 membered heterocyclyl-C₁-alkylene containing at least one O, C₁₋₆alkylcarbonyl, and C₂₋₆alkenylcarbonyl; preferably Z^{1b} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₆alkylcarbonyl, and C₂₋₄alkenylcarbonyl; preferably Z^{1b} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₄alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₈alkylcarbonyl, and C₂₋₄alkenylcarbonyl;
X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—; and
Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁-alkylene-O—, 3-10 membered heterocyclyl-C₁-alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl.
14. The compound according to any one of statements 1-13, wherein
   each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₈cycloalkyl, C₁₋₁₀aryl-C₁-alkylene, 5-10 membered heteroaryl-C₁₋₆-alkylene, 3-10 membered heterocyclyl-C₁₋₆alkylene, C₃₋₆cycloalkyl-C₁₋₆alkylene, and —R^{f1}-R^{g1}; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

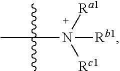

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₂₀alkylene, wherein said C₁₋₂₀alkylene can be interrupted by one or more O or S; R^{g1} is hydrogen, or C₁₋₂₀alkyl;
preferably each R^{a} is independently selected from the group comprising hydrogen, halo, C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, C₃₋₆cycloalkyl-C₁₋₆alkylene, and —R^{f1}-R^{g1}; wherein each of said C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

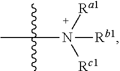

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{a1} is selected from hydrogen, or C₁₋₆alkyl; R^{f1} is C₁₋₁₅alkylene, wherein said C₁₋₁₅alkylene can be interrupted by one or more O or S; R^{g1} is hydrogen, or C₁₋₁₅alkyl;
preferably each R^{a} is independently selected from the group comprising hydrogen, halo, C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, C₃₋₆cycloalkyl, and —R^{f1}-R^{g1}; wherein each of said C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

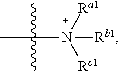

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; R^{f1} is C₁₋₁₀alkylene, wherein said C₁₋₁₀alkylene can be interrupted by one or more O or S; R^{g1} is hydrogen, or C₁₋₁₀alkyl; preferably each R^{a} is independently selected from the group comprising hydrogen, halo, C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, C₅₋₆cycloalkyl, and —R^{f1}-R^{g1}; wherein each of said C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

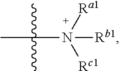

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₈alkylene, wherein said C₁₋₈alkylene can be interrupted by one or more O or S; R^{g1} is hydrogen, or C₁₋₈alkyl;
preferably each R^{a} is independently selected from the group comprising hydrogen, halo, C₁₋₁₅alkyl, C₂₋₆alkenyl, haloC₁₋₆alkyl, C₁₋₈alkylcarbonyl, 3-5 membered heterocyclyl containing at least one N or one O, phenyl, and —R^{f1}-R^{g1}; wherein each of said C₁₋₁₅alkyl, C₂₋₆alkenyl, haloC₁₋₆alkyl, C₁₋₈alkylcarbonyl, 3-5 membered heterocyclyl containing at least one N or one O, and phenyl, can be unsubstituted or substituted with one or more R^{za1}; wherein each R^{za1} is independently selected from the group comprising —COO—R^{h},

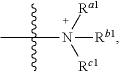

—NR^{a1}R^{b1}, —R^{f1}-R^{g1} and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl;
preferably each R^{a} is independently selected from the group comprising hydrogen, halo, C₁₋₁₆alkyl, C₂₋₆alkenyl, 3-4 membered heterocyclyl containing at least one O, C₁₋₈alkylcarbonyl, and —R^{f1}-R^{g1}; wherein each of said C₁₋₁₆alkyl, C₂₋₆alkenyl, 3-4 membered heterocyclyl containing at least one O, and C₁₋₈alkylcarbonyl, can be unsubstituted or substituted with one or more R^{za1}; wherein each R^{za1} is independently selected from the group comprising —COO—R^{h},

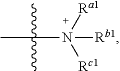

—NR^{a1}R^{b1}, —R^{f1}-R^{g1}, and C₁₋₆alkyl; R^{h} is selected from hydrogen, or C₁₋₆alkyl; R^{a}1 is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₄alkyl;
preferably each R^{a} is independently selected from the group comprising hydrogen, halo, C₁₋₁₅alkyl, C₂₋₄alkenyl, oxiranyl, and —R^{f1}-R^{g1}; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

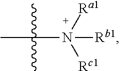

—NR^{a1}R^{b1}, —R^{f1}-R^{g1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; R^{f1} is C₁₋₆alkylene, wherein said C₁₋₆alkylene can be interrupted by one or more O or S; R^{g1} is hydrogen, or C₁₋₄alkyl;
for example each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{a} is independently selected from the group comprising hydrogen, halo, C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl; preferably each R^{a} is independently selected from the group comprising hydrogen, halo, C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl; preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-5 membered heterocyclyl containing at least one N or one O, and phenyl; preferably each R^{a} is independently selected from the group comprising hydrogen, halo, C₁₋₁₅alkyl, C₂₋₆alkenyl, 3-4 membered heterocyclyl containing at least one O, and C₁₋₈alkylcarbonyl; preferably each R^{a} is independently selected from the group comprising hydrogen, halo, C₁₋₁₅alkyl, C₂₋₄alkenyl, and oxiranyl;
each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 3-6 membered heterocyclyl-C₁₋₆alkylene, 5-10 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl, 3-6 membered heterocyclyl-C₁₋₆alkylene, 5-6 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl containing at least one N, 3-6 membered heterocyclyl-C₁₋₆alkylene containing at least one O, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, 3-5 membered heterocyclyl-C₁₋₆alkylene containing at least one O, and C₆₋₁₀aryl; preferably each R^{b} is independently selected from the group comprising hydrogen, C₁₋₆alkyl, 3-4 membered heterocyclyl-C₁₋₄alkylene containing one O, and haloC₁₋₆alkyl; preferably each R^{b} is independently selected from hydrogen, oxiranyl-C₁₋₂alkylene, or C₁₋₆alkyl; preferably each R^{b} is hydrogen;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃-cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N, 3-6 membered heterocyclyl-C₁₋₆alkylene containing at least one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1a} is selected from the group comprising hydrogen, —CO—R^{a}, —CO—C₂₋₄alkenylene-CO—O—C₁₋₄alkyl, —CO—R^{f}-maleimidyl, C₁₋₈alkyl, 3-4 membered heterocyclyl-C₁₋₆alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene-, C₁₋₈alkylcarbonyl, and, and C₂₋₄alkenylcarbonyl; preferably Z^{1a} is selected from the group comprising hydrogen, C₁₋₆alkylcarbonyl, —CO—C₂₋₄alkenylene-CO—O—C₁₋₄alkyl, —CO—C₁₋₆alkylene-maleimidyl, oxiranyl-C₁₋₂alkylene, and C₂₋₄alkenylcarbonyl;
X^{2a} is —(CR^{c}H)ₙ—; preferably X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—;
each R^{c} is independently selected from the group comprising hydrogen, halo, C₁₋₆alkyl, haloC₁₋₆alkyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{c} is independently selected from the group comprising hydrogen, halo, C₁₋₆alkyl, haloC₁₋₆alkyl, C₆₋₁₀aryl, 3-10 membered heterocyclyl, 5-10 membered heteroaryl, and C₃₋₆cycloalkyl; preferably each R^{c} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{c} is independently selected from the group comprising hydrogen, halo, C₁₋₆alkyl, haloC₁₋₆alkyl, C₆₋₁₀aryl, 3-6 membered heterocyclyl containing at least one N or one O, 5-6 membered heteroaryl containing at least one N, and C₃₋₆cycloalkyl; preferably each R^{c} is independently selected from the group comprising hydrogen, halo, C₁₋₆alkyl, haloC₁₋₆alkyl, and C₆₋₁₀aryl; preferably each R^{c} is independently selected from hydrogen, or C₁₋₆alkyl; preferably each R^{c} is hydrogen;
R^{e} is selected from the group comprising C₁₋₆alkylene, C₂₋₆alkenylene, C₂₋₆alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₆alkenylene;
R^{f} is C₁₋₂₀alkylene, wherein said alkylene can be interrupted by one or more O or S; preferably R^{f} is C₁₋₁₆alkylene, wherein said alkylene can be interrupted by one or more O or S; preferably R^{f} is C₁₋₁₄alkylene, wherein said alkylene can be interrupted by one or more O or S; preferably R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from hydrogen, C₁₋₂₀alkyl, N-maleimidyl, N-maleimidyloxy, or hydrogen, C₁₋₁₀alkyl, N-maleimidyloxycarbonyl; preferably R^{g} is hydrogen, C₁₋₆alkyl, N-maleimidyl, or N-maleimidyloxy; preferably R^{g} is hydrogen, C₁₋₄alkyl, N-maleimidyl;
W^{1a} is selected from —NR^{b}Z^{2a}, or —R^{a}; preferably W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W¹a is —NHCO—C₁₋₆alkyl; preferably W¹a is —NHCO—C₁₋₆alkyl;
Z^{2a} is selected from the group comprising hydrogen, —CO—R^{a}, halo, C₁₋₁₂alkyl, haloC₁₋₆alkyl, C₁₋₁₂alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₈alkyl, C₁₋₆alkylcarbonyl, C₆₋₁₀aryl, 3-6 membered heterocyclyl, 5-6 membered heteroaryl, and C₃₋₆cycloalkyl; preferably Z^{2a} is selected from the group comprising hydrogen, —CO—R^{a}, C₁₋₈alkyl, and C₁₋₈alkylcarbonyl; preferably Z^{2a} is selected from the group comprising hydrogen, C₁₋₈alkyl, and C₁₋₆alkylcarbonyl; preferably Z^{2a} is C₁₋₆alkylcarbonyl;
Y^{1a} is a moiety of formula (A) or (B) or is —OR^{a}, or —NHR^{a}; preferably Y^{1a} is a moiety of formula (A) or (B), or Y^{1a} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (A) or (B), or Y^{1a} is selected from the group comprising —OH, —NH₂, —O—C₁₋₁₂alkyl, and —NHC₁₋₁₂alkyl; preferably Y^{1a} is a moiety of formula (A) or (B), or is selected from the group comprising —OH, —NH₂, —O—C₁₋₁₀alkyl, and —NHC₁₋₁₀alkyl; preferably Y^{1a} is a moiety of formula (B),
X^{1b} is selected from —NR^{b}—, or —O—; preferably X^{1b} is —NR^{b}—;
Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₈cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 3-6 membered heterocyclyl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₈alkylcarbonyl, C₂₋₆alkenylcarbonyl, C₆₋₁₀aryl, 3-6 membered heterocyclyl containing at least one N, 3-6 membered heterocyclyl-C₁₋₆alkylene containing at least O, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-5 membered heterocyclyl-C₁₋₆alkylene containing at least one O, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, 3-4 membered heterocyclyl-C₁₋₆alkylene containing at least one O, C₁₋₆alkylcarbonyl, and C₂₋₄alkenylcarbonyl; preferably Z^{1b} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₈alkylcarbonyl, and C₂₋₆alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, C₁₋₆alkyl, oxiranyl-C₁₋₄alkylene, C₁₋₆alkylcarbonyl, and C₂₋₄alkenylcarbonyl; preferably Z^{1b} is selected from the group comprising hydrogen, oxiranyl-C₁₋₂alkylene, —CO—C₂₋₄alkenylene-CO—O—C₁₋₄alkyl, —CO—C₁₋₆alkylene-maleimidyl, C₁₋₆alkylcarbonyl, and C₂₋₄alkenylcarbonyl;
X^{2b} is —(CR^{c}H)ₘ—; preferably X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—; and
Y^{1b} is —OR^{a}, or —NHR^{a}; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl.
15. The compound according to any one of statements 3-14, wherein
   each R^{a} is independently selected from the group comprising hydrogen, halo, C₁₋₂₀alkyl, C₂₋₂₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, C₃₋₆cycloalkyl-C₁₋₆alkylene, and —R^{f1}-R^{g1}; wherein each of said C₁₋₂₀alkyl, C₂₋₂₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

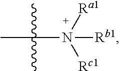

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₁₆alkylene, wherein said C₁₋₁₆alkylene can be interrupted by one or more O or S; R^{g1} is hydrogen, or C₁₋₁₆alkyl;
preferably each R^{a} is independently selected from the group comprising hydrogen, halo, C₁₋₁₈alkyl, C₂₋₁₈alkenyl, haloC₁₋₆alkyl, C₁₋₂alkylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and —R^{f1}-R^{g1}; wherein each of said C₁₋₁₈alkyl, C₂₋₁₈alkenyl, haloC₁₋₆alkyl, C₁₋₁₂alkylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

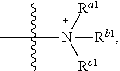

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₁₀alkylene, wherein said C₁₋₁₀alkylene can be interrupted by one or more O or S; R^{g1} is hydrogen, or C₆₋₁₀alkyl;
preferably each R^{a} is independently selected from the group comprising hydrogen, halo, C₁₋₁₆alkyl, C₂₋₁₆alkenyl, haloC₁₋₆alkyl, C₁₋₁₀alkylcarbonyl, phenyl, 6 membered heteroaryl containing at least one N, 3-6 membered heterocyclyl containing at least one N or one O, C₅₋₆cycloalkyl, and —R^{f1}-R^{g1}; wherein each of said C₁₋₁₆alkyl, C₂₋₁₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

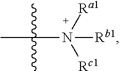

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl;
preferably each R^{a} is independently selected from the group comprising hydrogen, halo, C₁₋₁₅alkyl, C₂₋₁₀alkenyl, haloC₁₋₆alkyl, C₁₋₆alkylcarbonyl, 3-5 membered heterocyclyl containing at least one O, phenyl, and —R^{f1}-R^{g1}; wherein each of said C₁₋₁₅alkyl, C₂₋₁₀alkenyl, haloC₁₋₆alkyl, C₁₋₈alkylcarbonyl, 3-5 membered heterocyclyl containing at least one O, and phenyl, can be unsubstituted or substituted with one or more R^{za1}; wherein each R^{za1} is independently selected from the group comprising —COO—R^{h},

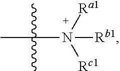

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl;
preferably each R^{a} is independently selected from the group comprising hydrogen, halo, C₁₋₁₅alkyl, C₂₋₆alkenyl, 3-4 membered heterocyclyl containing at least one O, C₁₋₈alkylcarbonyl, and —R^{f1}-R^{g1}; wherein each of said C₁₋₁₅alkyl, C₂₋₆alkenyl, 3-4 membered heterocyclyl containing at least one O, and C₁₋₆alkylcarbonyl, can be unsubstituted or substituted with one or more R^{za1}; wherein each R^{za1} is independently selected from the group comprising —COO—R^{h},

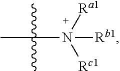

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl; preferably can be unsubstituted or substituted with one or more R^{za1}; wherein each R^{za1} is independently selected from the group comprising —COO—R^{h},

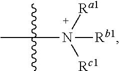

—NR^{a1}R^{b1}, —NR^{f1}-R^{g1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; R^{f1} is C₁₋₄alkylene, wherein said C₁₋₄alkylene can be interrupted by one or more O or S; R^{g1} is hydrogen, or C₁₋₄alkyl;
preferably each R^{a} is independently selected from the group comprising hydrogen, halo, C₁₋₁₅alkyl, C₂₋₄alkenyl, oxiranyl, and —R^{f1}-R^{g1}; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

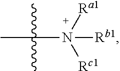

—NR^{a1}R^{b1}, —R^{f1}-R^{g1}, and C₁-alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; R^{f1} is C₁₋₄alkylene, wherein said C₁₋₄alkylene can be interrupted by one or more O or S; R^{g1} is hydrogen, or C₁₋₄alkyl; for example each R^{a} is independently selected from the group comprising hydrogen, halo, C₁₋₂₀alkyl, C₂₋₂₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₆alkyl, C₂₋₁₈alkenyl, haloC₁₋₆alkyl, C₁₋₁₂alkylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, and C₃₋₆cycloalkyl; preferably each R^{a} is independently selected from the group comprising hydrogen, halo, C₁₋₁₈alkyl, C₂₋₁₈alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl; preferably each R^{a} is independently selected from the group comprising hydrogen, halo, C₁₋₁₅alkyl, C₂₋₁₀alkenyl, haloC₁₋₆alkyl, C₁₋₈alkylcarbonyl, 3-5 membered heterocyclyl containing at least one O, and phenyl; preferably each R^{a} is independently selected from the group comprising hydrogen, halo, C₁₋₁₅alkyl, C₂₋₆alkenyl, 3-4 membered heterocyclyl containing at least one O, and C₁₋₈alkylcarbonyl; preferably each R^{a} is independently selected from the group comprising hydrogen, halo, C₁₋₁₅alkyl, C₂₋₄alkenyl, and oxiranyl;
each R^{b} is independently selected from the group comprising hydrogen, halo, C₁₋₆alkyl, haloC₁₋₆alkyl, C₆₋₁₀aryl, 3-6 membered heterocyclyl containing at least one N, 3-6 membered heterocyclyl-C₁₋₆alkylene containing at least one O, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, 3-5 membered heterocyclyl-C₁₋₆alkylene containing at least one O, and C₆₋₁₀aryl; preferably each R^{b} is independently selected from the group comprising hydrogen, C₁₋₆alkyl, 3-4 membered heterocyclyl-C₁₋₄alkylene containing one O, and haloC₁₋₆alkyl; preferably each R^{b} is independently selected from hydrogen, oxiranyl-C₁₋₂alkylene, or C₁₋₆alkyl; preferably each R^{b} is hydrogen;
X^{1a} is —NR^{b}—;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₈cycloalkyl, C₁₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₈cycloalkyl, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1a} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₆alkenylcarbonyl; preferably Z^{1a} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₈alkylcarbonyl, and C₂₋₄alkenylcarbonyl;
R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₆alkenylene;
R^{f} is C₁₋₂₀alkylene, wherein said alkylene can be interrupted by one or more O or S; preferably R^{f} is C₁₋₁₆alkylene, wherein said alkylene can be interrupted by one or more O or S; preferably R^{f} is C₁₋₁₄alkylene, wherein said alkylene can be interrupted by one or more O or S; preferably R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from hydrogen, C₁₋₂₀alkyl, N-maleimidyl, N-maleimidyloxy, or N-maleimidyloxycarbonyl; preferably R^{g} is hydrogen, C₁₋₁₀alkyl, N-maleimidyl, or N-maleimidyloxy; preferably R^{g} is hydrogen, C₁₋₆alkyl, N-maleimidyl;
X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—;
W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W^{1a} is —NHCO—C₁₋₆alkyl; preferably W^{1a} is —NHCO—C₁₋₆alkyl;
Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₆alkyl, C₁₋₆alkylcarbonyl, C₆₋₁₀aryl, 3-6 membered heterocyclyl, 3-6 membered heterocyclyl-C₁₋₆alkylene, 5-6 membered heteroaryl, and C₃₋₆cycloalkyl; preferably Z^{2a} is selected from the group comprising hydrogen, —CO—R^{a}, C₁₋₈alkyl, and C₁₋₈alkylcarbonyl; preferably Z^{2a} is selected from the group comprising hydrogen, C₁₋₈alkyl, and C₁₋₈alkylcarbonyl; preferably Z^{2a} is C₁₋₆alkylcarbonyl;
Y^{1a} is a moiety of formula (A) or (B), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is a moiety of formula (A) or (B), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (B), or is selected from the group comprising —OH, —NH₂, —O—C₁₋₁₂alkyl, and —NHC₁₋₁₂alkyl; preferably Y^{1a} is a moiety of formula (B), or is selected from the group comprising —OH, —NH₂, —O—C₁₋₁₀alkyl, and —NHC₁₋₁₀alkyl; preferably Y^{1a} is a moiety of formula (B),
X^{1b} is —NR^{b}—;
Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₂₋₁₀alkenyl, C₂₋₁₀alkynyl, C₆₋₁₀aryl, 3-6 membered heterocyclyl, 5-6 membered heteroaryl, C₃₋₈cycloalkyl, C₁₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₈cycloalkyl, C₁₋₁₀aryl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₈cycloalkyl, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1b} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₆alkylcarbonyl, and C₂₋₄alkenylcarbonyl; preferably Z^{1b} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₄alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl;
X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—; and
Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl.
16. The compound according to any one of statements 1 to 15, having structural formula (IIB1) or (IIA1a)

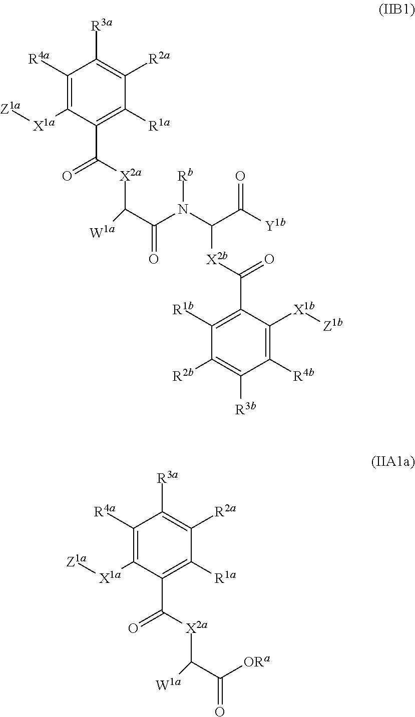

wherein Z^{1a}, X^{1a}, X^{2a}, W^{1a}, Z^{1b}, X^{1b}, X^{2b}, Y^{1a}, Y^{1b}, R^{b}, R^{1a}, R^{2a}, R^{3a}, R^{4a}, R^{1b}, R^{2b}, R^{3b}, and R^{4b} have the same meaning as that defined in any one of statements 1-15.
17. The compound according to any one of statements 1-16, having structural formula (IIA12), (IIA13), (IIA14), (IIA15), (IIA16), (IIA17), (IIA18), (IIB2), (IIB3), (IIB4), (IIB5), (IIB6), (IIB7), or (IIB8),

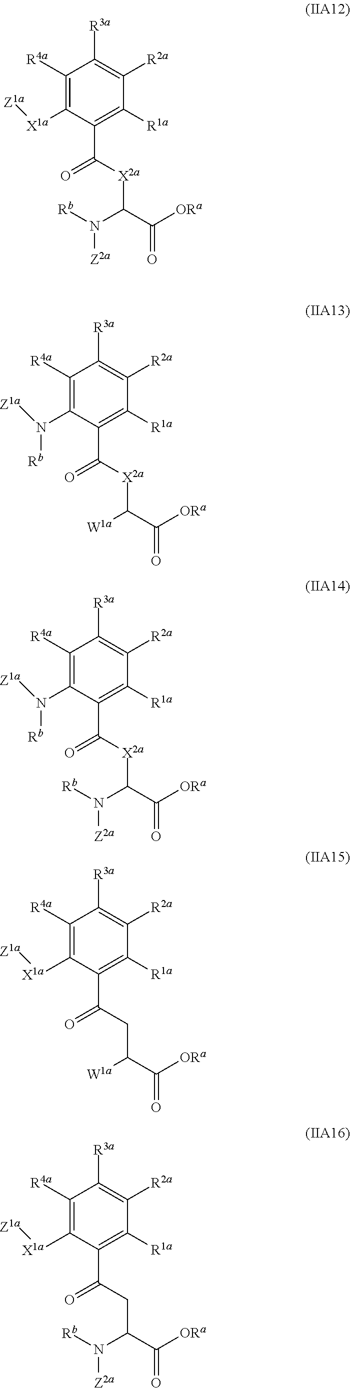

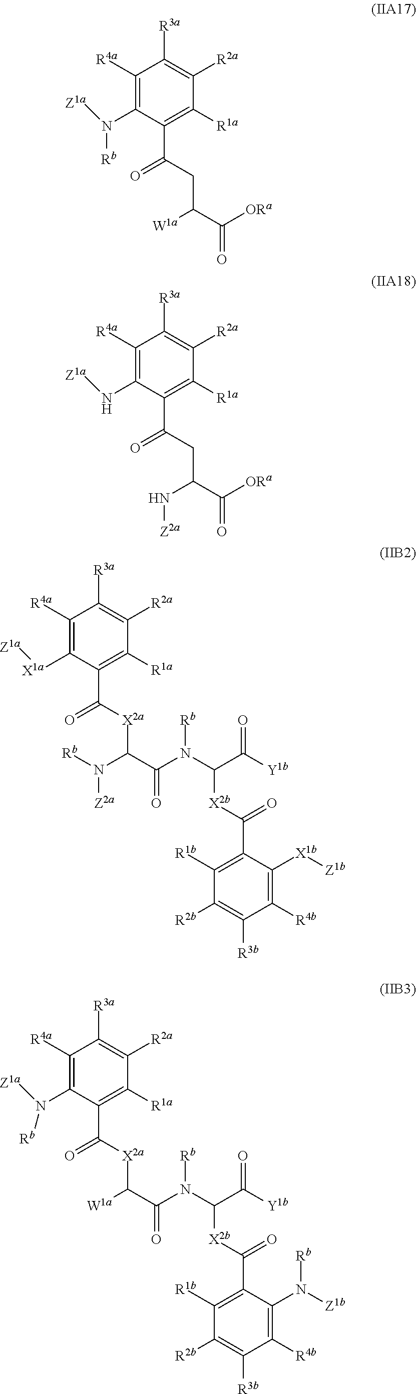

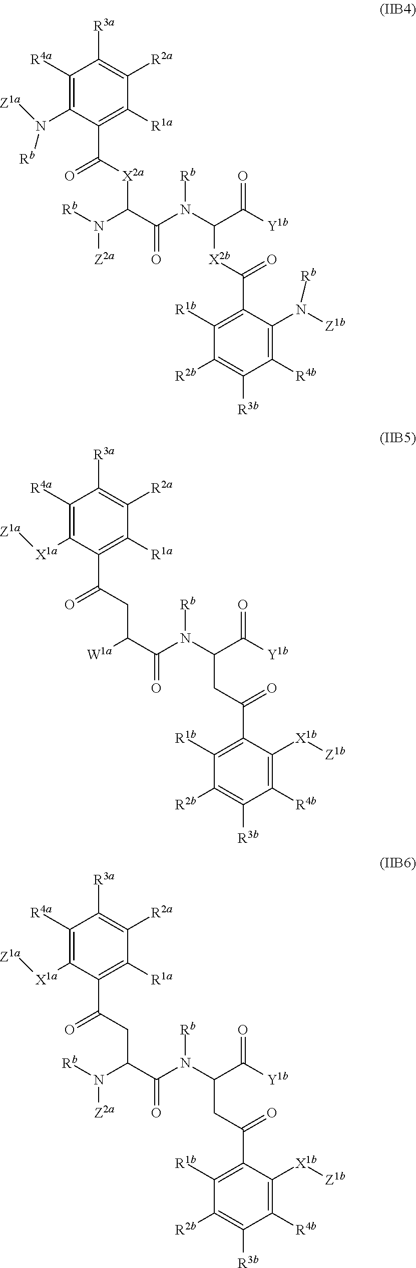

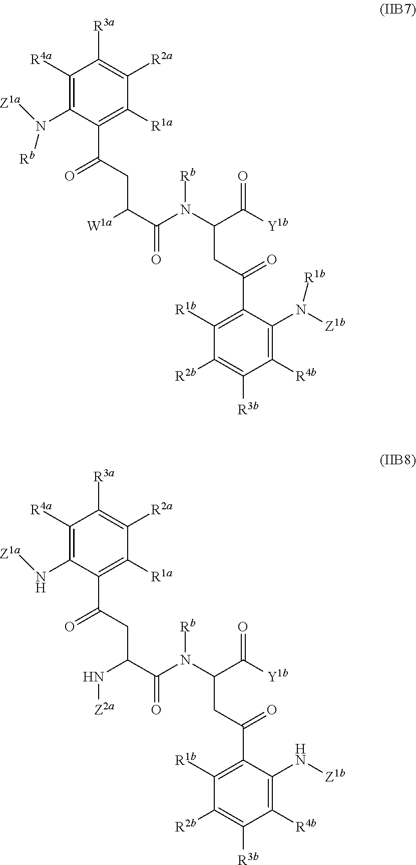

wherein Z^{1a}, X^{1a}, X^{2a}, Z^{2a}, W^{1a}, Z^{1b}, X^{1b}, X^{2b}, Y^{1a}, Y^{1b}, R^{a}, R^{b}, R^{1a}, R^{2a}, R^{3a}, R^{4a}, R^{1b}, R^{2b}, R^{3b}, and R^{4b} have the same meaning as that defined in any one of statements 1-16.
18. The compound according to any one of statements 1-17, having structural formula (IIA19) or (IIB9)

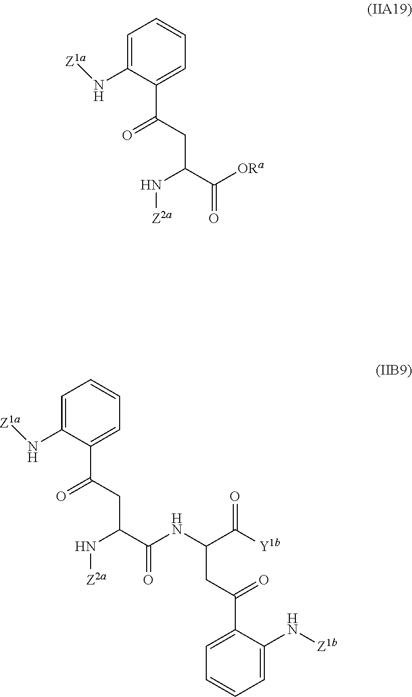

wherein Z^{1a}, Z^{2a}, R^{a}, Y^{1b}, and Z^{1b}, have the same meaning as that defined in any one of statements 1-17.
19. The compound according to any one of statements 1-18, wherein each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, C₃₋₆cycloalkyl-C₁₋₆alkylene, and —R^{f1}-R^{g1}; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

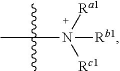

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; R^{f1} is C₁₋₂₀alkylene, wherein said C₁₋₂₀alkylene can be interrupted by one or more O or S; R^{g1} is hydrogen, or C₁₋₂₀alkyl;
preferably each R^{a} is independently selected from the group comprising hydrogen, halo, C₁₋₂₀alkyl, C₂₋₂₀alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl; wherein each of said C₁₋₂₀alkyl, C₂₋₂₀alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

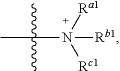

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₈alkyl, C₂₋₁₆alkenyl, haloC₁₋₆alkyl, C₁₋₁₀alkylcarbonyl, phenyl, 6-membered heteroaryl containing at least one N, 3-6 membered heterocyclyl containing at least one N or one O, and C₅-cycloalkyl; wherein each of said C₁₋₆alkyl, C₂₋₁₆alkenyl, haloC₁₋₆alkyl, C₁₋₁₀alkylcarbonyl, phenyl, 6-membered heteroaryl containing at least one N, 3-6 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

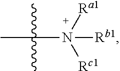

—NR^{a1}R^{b1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
preferably each R^{a} is independently selected from the group comprising hydrogen, halo, C₁₋₁₅alkyl, C₂₋₁₀alkenyl, haloC₁₋₆alkyl, C₁₋₆alkylcarbonyl, 3-5 membered heterocyclyl containing at least one O, and phenyl; wherein each of said C₁₋₁₅alkyl, C₂₋₁₀alkenyl, haloC₁₋₆alkyl, C₁₋₈alkylcarbonyl, 3-5 membered heterocyclyl containing at least one O, and phenyl, can be unsubstituted or substituted with one or more R^{za1}; wherein each R^{za1} is independently selected from the group comprising —COO—R^{h},

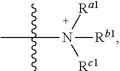

—NR^{a1}R^{b1} and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl;
preferably each R^{a} is independently selected from the group comprising hydrogen, halo, C₁₋₁₅alkyl, C₂₋₄alkenyl, 3-4 membered heterocyclyl containing at least one O, and C₁₋₈alkylcarbonyl; wherein each of said C₁₋₁₅alkyl, C₂₋₄alkenyl, 3-4 membered heterocyclyl containing at least one O, and C₁₋₆alkylcarbonyl, can be unsubstituted or substituted with one or more R^{za1}; wherein each R^{za1} is independently selected from the group comprising —COO—R^{h},

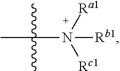

—NR^{a1}R^{b1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R¹ is selected from hydrogen, or C₁₋₄alkyl;
preferably each R^{a} is independently selected from the group comprising hydrogen, halo, C₁₋₁₅alkyl, C₂₋₄alkenyl, and oxiranyl; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

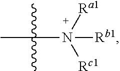

—NR^{a1}R^{b1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R¹ is selected from hydrogen, or C₁₋₄alkyl;
for example each R^{a} is independently selected from the group comprising hydrogen, halo, C₁₋₂₀alkyl, C₂₋₂₀alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl; preferably each R^{a} is independently selected from the group comprising hydrogen, halo, C₁₋₁₈alkyl, C₂₋₁₆alkenyl, haloC₁₋₆alkyl, C₁₋₁₀alkylcarbonyl, phenyl, 6-membered heteroaryl containing at least one N, 3-6 membered heterocyclyl containing at least one N or one O, and C₅₋₆cycloalkyl; preferably each R^{a} is independently selected from the group comprising hydrogen, halo, C₁₋₁₅alkyl, C₂₋₁₀alkenyl, haloC₁₋₆alkyl, C₁₋₆alkylcarbonyl, 3-5 membered heterocyclyl containing at least one O, and phenyl; preferably each R^{a} is independently selected from the group comprising hydrogen, halo, C₁₋₁₅alkyl, C₂₋₄alkenyl, 3-4 membered heterocyclyl containing at least one O, and C₁₋₆alkylcarbonyl; preferably each R^{a} is independently selected from the group comprising hydrogen, halo, C₁₋₁₅alkyl, C₂₋₄alkenyl, and oxiranyl;
Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{f}-maleimidyl, halo, C₁₋₈alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one N or one O, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1a} is selected from the group comprising hydrogen, —CO—R^{a}, —CO—C₂-alkenylene-CO—O—R^{b}, —CO—R^{f}-maleimidyl, C₁₋₆alkyl, 3-4 membered heterocyclyl-C₁₋₆alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₄alkenylcarbonyl; preferably Z^{1a} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl;
Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-6 membered heterocyclyl-C₁₋₆alkylene containing at least one N or one O, C₁₋₆alkylcarbonyl, C₂-alkenylcarbonyl, C₆₋₁₀aryl, C₃-cycloalkyl, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₆alkyl, 3-5 membered heterocyclyl-C₁₋₆alkylene containing at least one O, haloC₁₋₆alkyl, and C₁₋₆alkylcarbonyl, and C₂₋₆alkenylcarbonyl; preferably Z^{1b} is selected from the group comprising hydrogen, —CO—R^{a}, —CO—C₂₋₆alkenylene-CO—O—R^{b}, —CO—C₁₋₆alkylene-maleimidyl, C₁₋₆alkyl, 3-4 membered heterocyclyl-C₁₋₆alkylene containing one O, C₁₋₆alkylcarbonyl, and C₂₋₆alkenylcarbonyl; preferably Z^{1b} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₄alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl;
R^{e} is selected from the group comprising C₁₋₆alkylene, C₂₋₆alkenylene, C₂₋₆alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₆alkenylene;
R^{f} is C₁₋₂₀alkylene, wherein said alkylene can be interrupted by one or more O or S; preferably R^{f} is C₁₋₁₆alkylene, wherein said alkylene can be interrupted by one or more O or S; preferably R^{f} is C₁₋₁₄alkylene, wherein said alkylene can be interrupted by one or more O or S; preferably R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene;
R^{g} is selected from hydrogen, C₁₋₁₀alkyl, N-maleimidyl, N-maleimidyloxy, or N-maleimidyloxycarbonyl; preferably R^{g} is hydrogen, C₁₋₆alkyl, N-maleimidyl, or N-maleimidyloxy; preferably R^{g} is hydrogen, C₁₋₆alkyl, N-maleimidyl;
Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₈cycloalkyl, Ca-10aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₈cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl.
20. The compound according to any one of statements 1-19, having structural formula (IIA10), (IIB10) or (IIB11)

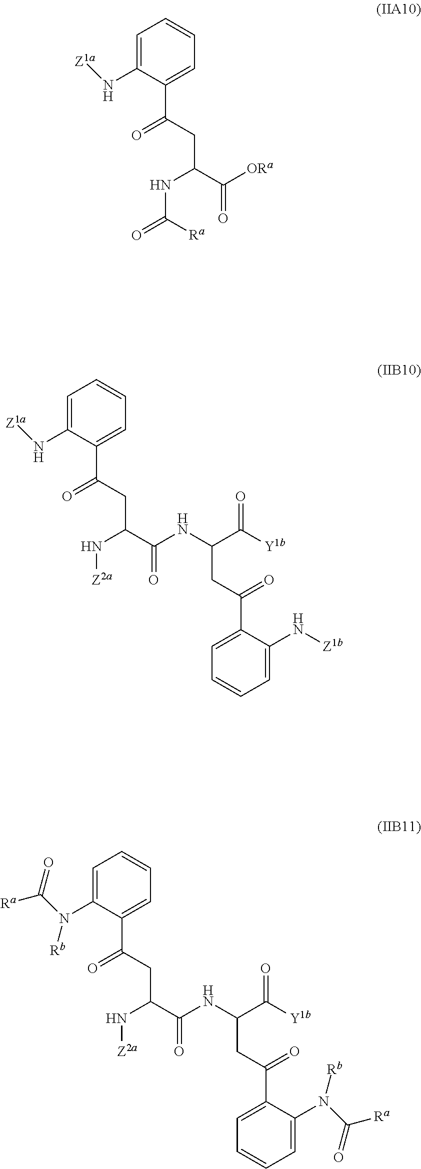

wherein R^{a}, Z^{2a}, Z^{b}, Y^{1b}, R^{b}, have the same meaning as that defined in any one of statements 1-19.
21. The compound according to any one of statements 1-20, wherein Z^{2a} is selected from the group comprising hydrogen, —CO—R^{a}, C₁₋₆alkyl, C₁₋₆alkylcarbonyl, C₆₋₁₀aryl, 3-6 membered heterocyclyl, 5-6 membered heteroaryl, and C₃₋₆cycloalkyl; preferably Z^{2a} is selected from the group comprising hydrogen, —CO—R^{a}, C₁₋₆alkyl, and C₁₋₆alkylcarbonyl; preferably Z^{2a} is selected from the group comprising hydrogen, C₁₋₆alkyl, and C₁₋₆alkylcarbonyl; preferably Z^{2a} is C₁₋₆alkylcarbonyl;
22. The compound according to any one of statements 1-21, wherein
   each R^{a} is independently selected from the group comprising hydrogen, halo, C₁₋₁₈alkyl, C₂₋₆alkenyl, haloC₁₋₆alkyl, C₁₋₆alkylcarbonyl, 3-5 membered heterocyclyl containing at least one N or one O, phenyl, and —R^{f1}-R^{g1}; wherein each of said C₁₋₁₈alkyl, C₂₋₆alkenyl, haloC₁₋₆alkyl, C₁₋₆alkylcarbonyl, 3-5 membered heterocyclyl containing at least one N or one O, and phenyl, can be unsubstituted or substituted with one or more R^{za1}; wherein each R^{za1} is independently selected from the group comprising —COO—R^{h},

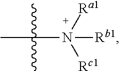

NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl;
preferably each R^{a} is independently selected from the group comprising hydrogen, halo, C₁₋₁₅alkyl, C₂₋₆alkenyl, 3-4 membered heterocyclyl containing at least one O, C₁₋₆alkylcarbonyl, and —R^{f1}-R^{g1}; wherein each of said C₁₋₁₅alkyl, C₂₋₆alkenyl, 3-4 membered heterocyclyl containing at least one O, and C₁₋₆alkylcarbonyl, can be unsubstituted or substituted with one or more R^{za1}; wherein each R^{za1} is independently selected from the group comprising —COO—R^{h},

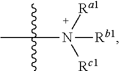

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₄alkyl;
preferably each R^{a} is independently selected from the group comprising hydrogen, halo, C₁₋₁₈alkyl, C₂₋₆alkenyl, haloC₁₋₆alkyl, C₁₋₁₀alkylcarbonyl, phenyl, 6 membered heteroaryl containing at least one N, 3-4 membered heterocyclyl containing one O, and C₅₋₆cycloalkyl; wherein each of said C₁₋₁₈alkyl, C₂₋₆alkenyl, haloC₁₋₆alkyl, C₁₋₁₀alkylcarbonyl, phenyl, 6 membered heteroaryl containing at least one N, 3-4 membered heterocyclyl containing one O, and C₅₋₆cycloalkyl, can be unsubstituted or substituted with one or more R^{za1}; wherein each
R^{za1} is independently selected from the group comprising —COO—R^{h},

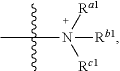

—NR^{a1}R^{b1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl;
for example each R^{a} is independently selected from the group comprising hydrogen, halo, C₁₋₁₈alkyl, C₂₋₆alkenyl, haloC₁₋₆alkyl, C₁₋₁₀alkylcarbonyl, phenyl, 6 membered heteroaryl containing at least one N, 3-4 membered heterocyclyl containing one O, and C₅₋₆cycloalkyl; each R^{b} is independently selected from the group comprising hydrogen, halo, C₁₋₆alkyl, haloC₁₋₆alkyl, C₆₋₁₀aryl, 3-6 membered heterocyclyl containing at least one N, 3-6 membered heterocyclyl-C₁₋₆alkylene containing at least one O, 5-6 membered heteroaryl containing at least one N, and C₃₋₆cycloalkyl;
Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl.
23. The compound according to any one of statements 1-22, wherein
   each R^{a} is independently selected from the group comprising hydrogen, halo, C₁₋₁₆alkyl, C₂₋₆alkenyl, 3-4 membered heterocyclyl containing at least one O, C₁₋₆alkylcarbonyl, and —R^{f1}-R^{g1}; wherein each of said C₁₋₁₆alkyl, C₂₋₆alkenyl, 3-4 membered heterocyclyl containing at least one O, and C₁₋₈alkylcarbonyl, can be unsubstituted or substituted with one or more R^{za1};
   wherein each R^{za1} is independently selected from the group comprising —COO—R^{h},

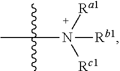

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₄alkyl;
preferably each R^{a} is independently selected from the group comprising hydrogen, halo, C₁₋₁₆alkyl, C₂₋₆alkenyl, haloC₁₋₆alkyl, C₁₋₆alkylcarbonyl, oxiranyl, and phenyl; wherein each of said C₁₋₁₆alkyl, C₂₋₆alkenyl, haloC₁₋₆alkyl, C₁₋₆alkylcarbonyl, oxiranyl, and phenyl, can be unsubstituted or substituted with one or more R^{za1}; wherein each R^{za1} is independently selected from the group comprising —COO—R^{h},

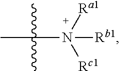

—NR^{a1}R^{b1} halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; for example each R^{a} is independently selected from the group comprising hydrogen, halo, C₁₋₁₆alkyl, C₂₋₆alkenyl, haloC₁₋₆alkyl, C₁₋₆alkylcarbonyl, oxiranyl, and phenyl;
each R^{b} is independently selected from the group comprising hydrogen, halo, C₁₋₆alkyl, haloC₁₋₆alkyl, 3-5 membered heterocyclyl-C₁₋₆alkylene containing at least one O, and C₆₋₁₀aryl; Y^{1b} is selected from the group comprising —OH, —NH₂, —O—C₁₋₁₂alkyl, and —NHC₁₋₁₂alkyl.
24. The compound according to any one of statements 1-23, wherein
   each R^{a} is independently selected from the group comprising hydrogen, halo, C₁₋₁₅alkyl, C₂₋₄alkenyl, oxiranyl, and —R^{f1}-R^{g1}; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

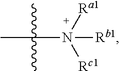

—NR^{a1}R^{b1}, —R^{f1}-R^{g1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; R^{f1} is C₁₋₆alkylene, wherein said C₁₋₆alkylene can be interrupted by one or more O or S; R^{g1} is hydrogen, or C₁₋₄alkyl;
preferably each R^{a} is independently selected from the group comprising hydrogen, halo, C₁₋₁₈alkyl, C₂₋₆alkenyl, oxiranyl, and C₁₋₆alkylcarbonyl; wherein each of said C₁₋₁₈alkyl, C₂₋₆alkenyl, oxiranyl, and C₁₋₆alkylcarbonyl, can be unsubstituted or substituted with one or more R^{za1};
wherein each R^{za1} is independently selected from the group comprising —COO—R^{h},

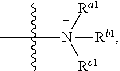

—NR^{a1}R^{b1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R¹ is selected from hydrogen, or C₁₋₄alkyl;
preferably each R^{a} is independently selected from the group comprising hydrogen, halo, C₁₋₁₆alkyl, C₂₋₄alkenyl, and oxiranyl; wherein each of said C₁₋₅alkyl, C₂₋₄alkenyl, and oxiranyl, can be unsubstituted or substituted with one or more R^{za1}; wherein each R^{za1} is independently selected from the group comprising —COO—R^{h},

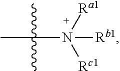

—NR^{a1}R^{b1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R¹ is selected from hydrogen, or C₁₋₄alkyl;
for example each R^{a} is independently selected from the group comprising hydrogen, halo, C₁₋₁₈alkyl, C₂₋₆alkenyl, oxiranyl, and C₁₋₆alkylcarbonyl; preferably each R^{a} is independently selected from the group comprising hydrogen, halo, C₁₋₁₆alkyl, C₂₋₄alkenyl, and oxiranyl; each R^{b} is independently selected from the group comprising hydrogen, C₁₋₆alkyl, 3-4 membered heterocyclyl-C₁₋₄alkylene containing one O, and haloC₁₋₆alkyl; preferably each R^{b} is independently selected from hydrogen, oxiranyl-C₁₋₂alkylene, or C₁₋₆alkyl; preferably each R^{b} is hydrogen;
Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl; preferably Y^{1b} is —O—C₁₋₁₀alkyl.
25. The compound according to any one of statements 1-24, wherein
   each R^{a} is independently selected from the group comprising hydrogen, halo, C₁₋₁₅alkyl, C₂₋₄alkenyl, oxiranyl, and —R^{f1}-R^{g1}; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

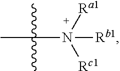

—NR^{a1}R^{b1}, —R^{f1}-R^{g1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; R^{f1} is C₁₋₆alkylene, wherein said C₁₋₆alkylene can be interrupted by one or more O or S; R^{g1} is hydrogen, or C₁₋₄alkyl;
preferably each R^{a} is independently selected from the group comprising hydrogen, halo, C₁₋₁₆alkyl, C₂₋₄alkenyl, and oxiranyl; wherein each of said C₁₋₁₆alkyl, C₂₋₄alkenyl, and oxiranyl, can be unsubstituted or substituted with one or more R^{za1}; wherein each R^{za1} is independently selected from the group comprising —COO—R^{h},

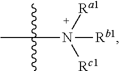

—NR^{a1}R^{b1}, and C₁₋₄alkyl; R^{h} is selected from hydrogen, or C₁₋₂alkyl; R^{a1} is selected from hydrogen, or C₁₋₂alkyl; R^{b1} is selected from hydrogen, or C₁₋₂alkyl; R^{c1} is selected from hydrogen, or C₁₋₂alkyl;
for example each R^{a} is independently selected from the group comprising hydrogen, halo, C₁₋₁₆alkyl, C₂₋₄alkenyl, and oxiranyl;
each R^{b} is independently selected from hydrogen, or C₁₋₆alkyl; preferably each R^{b} is hydrogen; Y^{1b} is —O—C₁₋₁₀alkyl, or —NH—C₁₋₁₀alkyl, preferably Y^{1b} is —O—C₁₋₁₀alkyl.
26. A composition comprising at least one compound according to any one of statements 1-25.
27. A cosmetic or pharmaceutical composition, comprising at least one compound according to any one of statements 1-25, and optionally at least one dermatologically or pharmaceutically acceptable carrier.
28. The composition according to any one of statements 26-27, further comprising at least one additional UV filter, different to the compound of formula (I), preferably wherein the at least one additional UV filter is a natural and/or biodegradable UV filter.
29. The composition according to any one of statements 26-28, further comprising one or more thickening agents, one or more film forming agents, one or more surfactants, one or more skin conditioning agents, or a combination thereof.
30. Use of at least one compound according to any one of statements 1-25, or at least one composition according to any one of statement 26-29 as UV absorbing filter and/or high-energy visible light filters.
31. Use of at least one compound according to any one of statements 1-25, or at least one composition according to any one of statements 26-29, for protecting from UV and/or high-energy visible radiations.
32. The use according to any one of statements 30-31, wherein compound of formula (I) provides protection centered at from about 290 nm to 410 nm, for example from about 300 nm to about 410 nm, for example from about 310 nm to about 410 nm, for example from about 320 nm to about 370 nm, for example from about 290 nm to about 320 nm, for example from about 320 nm to about 400 nm, for example from about 330 nm to about 410 nm, for example from about 330 nm to about 350 nm, for example from about 340 nm to about 360 nm, for example from about 350 nm to about 380 nm, for example from about 340 nm to about 390 nm, for example from about 330 nm to about 400 nm, or for example from about 390 nm to about 410 nm.
33. A compound according to any one of statements 1-25 or a cosmetic or pharmaceutical composition according to any one of statements 26-29 for use to protect skin against UV and/or high-energy visible radiations.
34. Use of at least one compound according to any one of statements 1-25 in a consumer product as a light stabilizer to protect ingredients against photolytic degradation.
35. Use according to statement 34, wherein the consumer product is selected from the group comprising cosmetic (body care product), foodstuff, pharmaceutical, textile, household chemical and lawn and gardening product.
36. Use of at least one compound according to any one of statements 1-25, or at least one composition according to any one of statements 26-29, as an UV and/or high-energy visible light absorbing filter in a consumer product, preferably in a cosmetic product.
37. Consumer product comprising at least one compound according to any one of statements 1-25.
38. The consumer product according to statement 37, wherein said consumer product is a cosmetic product.
39. Cosmetic product comprising at least one compound according to any one of statements 1-25, or at least cosmetic composition according to any one of statements 27-29.
40. The cosmetic product according to any one of statements 38-39, wherein the cosmetic product is selected from the group comprising a sunscreen, a moisturizer, makeup, lip protection, a shampoo, and a conditioner, preferably the product is a sunscreen characterized by photostability and nonradiative relaxation that provides UV-A and/or UV-B protection.
41. The cosmetic product according to any one of statement 38-40, wherein the product is characterized by photostability and nonradiative relaxation that provides UV-A and/or UV-B protection.
42. The product according to any one of statement 38-41, wherein the product is selected from the group comprising a cream, a lotion, a light ointment, an oil, a gel, an emulsion, a suspension, a mousse, an aerosol, a powder, a pack, a sheet, a patch, a stick, and a cake.
43. The product according to any one of statement 38-42, further comprising a product vehicle.
44. The product according to any one of statement 38-43, further comprising a product vehicle, wherein the vehicle is selected from the group comprising water, a solvent or diluent, an oil, and a combination thereof.
45. The product according to any one of statement 38-44, further comprising one or more of an oil component, a moisturizing agent, a surfactant, and a carrier.
46. The product according to any one of statement 38-45, wherein the product comprises from about 0.001% to about 50.0% by weight of the compound of formula (I), relative to the total weight of the product, for example from about 0.01% to about 40.0% by weight, for example from about 0.01% to about 30.0% by weight, for example from about 0.1% to about 25.0% by weight, for example from about 0.1% to about 20.0% by weight, for example from about 0.1% to about 15.0% by weight, for example from about 0.1% to about 10.0% by weight, for example from about 0.1% to about 5.0% by weight, for example from about 0.1% to about 2.5% by weight, relative to the total weight of the product.
47. The product according to any one of statement 38-46, further comprising one or more thickening agents, film forming agents, surfactants, skin conditioning agents, or a combination thereof.
48. The product according to any one of statement 38-47, wherein the product has a pH of from about 2.0 to about 11.0, for example from about 3.0 to about 10.0, for example from about 4.0 to about 9.0, for example from about 5.0 to about 9.0, for example from about 5.0 to about 8.5, for example from about 5.5 to about 8.5, for example from about 6.0 to about 8.5, or for example from about 6.5 to about 8.5.
49. Use of at least one compound according to any one of statements 1-25, as a UV and/or high-energy visible light absorbing filter in packaging.
50. A packaging material comprising at least one compound according to any one of statements 1-25.
51. The packaging material according to statement 50, wherein the at least one compound is in a coating applied to the surface of the packaging material.
52. The packaging material according to any one of statement 50-51, wherein the at least one compound is dispersed throughout the packaging material.
53. The packaging material according to any one of statement 50-52, wherein the packaging material is selected from the group comprising thermoplastic polymer, polyvinyl chloride (PVC), polyethylene, polyethylene terephthalate (PET), low density polyethylene (LDPE), high density polyethylene (HDPE), polystyrene (PS), plastic wrap, aluminum, aluminum foil, laminates and metallized films, tin-free steel, tin-coated steel, ceramics, protein derived plastics (such as protein derived thermoplastics for example from sources such as wheat, rice, corn, soy, and casein), and glass.
54. The packaging material according to any one of statement 50-53, wherein the packaging material is manufactured in the form of a container, a bottle, a cap, or a flexible outer film.
55. The packaging material according to any one of statement 50-54, wherein the packaging material comprises from about 0.1% to about 25.0% by weight of the compound, relative to the total weight of the packaging material, for example from about 0.1% to about 20.0% by weight, for example from about 0.1% to about 15.0% by weight, for example from about 0.1% to about 10.0% by weight, for example from about 0.1% to about 7.5% by weight, for example from about 0.1% to about 5.0% by weight, for example from about 0.1% to about 2.5% by weight, for example from about 0.1% to about 2.0% by weight, for example from about 0.1% to about 1.0% by weight.
56. The packaging material according to any one of statements 50-55, wherein the packaging material is transparent or translucent.
57. The packaging material according to any one of statements 50-56, wherein the packaging material is used for food packaging, cosmetics, or medical products.
58. The packaging material according to any one of statements 50-57, wherein the packaging material is a food packaging material.
59. A packaging system, comprising: at least one thermoplastic polymer; and at least one compound according to any one of statements 1-25, preferably dispersed within the thermoplastic polymer.
60. A food system, comprising: a food or food condiment; and an additive in contact with the food or food condiment, the additive being a compound according to any one of statements 1-25.
61. The food system according to statement 60, wherein the food is a food for humans, pets, or livestock.
62. The compound as listed in Table 1.
63. Use of at least one compound according to any one of statements 1-25, 62, as antioxidant.
64. Use of at least one compound according to any one of statements 1-25, 62, as antioxidant against reactive oxygen species, preferably hydroxyl radical species.
65. A method to protect a composition or consumer product from oxidation or to impart antioxidant properties to a composition, comprising adding at least one compound according to any one of statements 1-25, 62, to said composition or consumer product.
66. Use of at least one compound according to any one of statements 1-25, 62, in a consumer product as antioxidant.
67. A compound according to any one of statements 1-25, 62, or a cosmetic or pharmaceutical composition according to any one of statements 27-29 for use to protect a substrate against reactive oxygen species, preferably hydroxyl radical species.
68. The compound, the cosmetic or pharmaceutical composition according to statement 67, wherein the substrate is skin.
69. Use of a compound according to any one of statements 1-25, 62, or a cosmetic or pharmaceutical composition according to any one of statements 27-29 as anti-aging agent.
70. Use of a compound according to any one of statements 1-25, 62, or a cosmetic or pharmaceutical composition according to any one of statements 27-29 for protecting the skin from reactive oxygen and nitrogen species.

As stated before, the present invention provides a compound of formula (I) as defined herein (including all embodiments thereof as described herein):

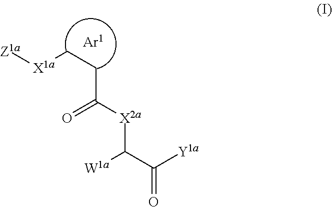

or a tautomer, a stereoisomer, a hydrate, a solvate, a salt, or a polymorph thereof.

In some embodiments, said compound is not a compound selected from the group consisting of 2-amino-4-(2-aminophenyl)-4-oxobutanoic acid; (R)-2-amino-4-(2-aminophenyl)-4-oxobutanoic acid; (S)-2-amino-4-(2-aminophenyl)-4-oxobutanoic acid; 4-(2-acetamidophenyl)-2-amino-4-oxobutanoic acid; 2-acetamido-4-(2-aminophenyl)-4-oxobutanoic acid; (S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid; 2-amino-4-oxo-4-(2-(((3R,4R,5S,6R)-3,4,5-trihydroxy-6-(hydroxymethyl)tetrahydro-2H-pyran-2-yl)amino)phenyl)butanoic acid; (2S)-2-amino-4-oxo-4-(2-(((3R,4R,5S,6R)-3,4,5-trihydroxy-6-(hydroxymethyl)tetrahydro-2H-pyran-2-yl)amino)phenyl)butanoic acid; (S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid; methyl 2-amino-4-(2-aminophenyl)-4-oxobutanoate; 4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid; 2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid; (S)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid; (R)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid; (2R,3R,4S,5R)-2-(acetoxymethyl)-6-(2-amino-3-(4-methoxy-4-oxobutanoyl)phenoxy)tetrahydro-2H-pyran-3,4,5-triyl triacetate; 2-amino-4-(2-amino-5-hydroxyphenyl)-4-oxobutanoic acid; (S)-2-amino-4-(2-amino-5-hydroxyphenyl)-4-oxobutanoic acid; (R)-2-amino-4-(2-amino-5-hydroxyphenyl)-4-oxobutanoic acid; 5-(2-aminophenyl)-5-oxopentanoic acid; 4-(2-aminophenyl)-4-oxobutanoic acid; methyl 6-(2-aminophenyl)-6-oxohexanoate; methyl 4-(2-aminophenyl)-2-((tert-butoxycarbonyl)amino)-4-oxobutanoate; butyl 2-amino-4-(2-aminophenyl)-4-oxobutanoate; ethyl (S)-2-amino-4-(2-aminophenyl)-4-oxobutanoate; (S)-2-amino-4-(2-aminophenyl)-4-oxobutanoic acid diacetate; (R)-2-amino-4-(2-formamidophenyl)-4-oxobutanoic acid; (S)-2-amino-4-(2-formamidophenyl)-4-oxobutanoic acid;
2-amino-4-(2-formamidophenyl)-4-oxobutanoic acid; methyl 2-acetamido-4-(2-acetamidophenyl)-4-oxobutanoate;4-(2-amino-3-(((2R,3S,4R,5R,6S)-3,4,5-trihydroxy-6-((((2S,3S,4R,5R,6S)-3,4,5-trihydroxy-6-(hydroxymethyl)tetrahydro-2H-pyran-2-yl)oxy)methyl)tetrahydro-2H-pyran-2-yl)oxy)phenyl)-4-oxobutanoic acid; 4-(2-amino-3-hydroxyphenyl)-4-oxobutanoic acid O-beta-D-diglucoside); 4-(2-amino-3-(((2R,3S,4R,5R,6S)-3,4-5-trihydroxy-6-(hydroxymethyl)tetrahydro-2H-pyran-2-yl)oxy)phenyl)-4-oxobutanoic acid; 4-(2-amino-3-hydroxyphenyl)-4-oxobutanoic acid O-beta-d-glucoside; 2-amino-4-(2-formamido-5-hydroxyphenyl)-4-oxobutanoic acid; 2-amino-4-(2-amino-3-chlorophenyl)-4-oxobutanoic acid; (R)-2-amino-4-(2-amino-3-chlorophenyl)-4-oxobutanoic acid; (S)-2-amino-4-(2-amino-3-chlorophenyl)-4-oxobutanoic acid; 2-amino-4-(2-amino-4-chlorophenyl)-4-oxobutanoic acid; (R)-2-amino-4-(2-amino-4-chlorophenyl)-4-oxobutanoic acid; (S)-2-amino-4-(2-amino-4-chlorophenyl)-4-oxobutanoic acid; -2-amino-4-(2-amino-5-fluorophenyl)-4-oxobutanoic acid; (R)-2-amino-4-(2-amino-5-fluorophenyl)-4-oxobutanoic acid; (S)-2-amino-4-(2-amino-5-fluorophenyl)-4-oxobutanoic acid; 2-amino-4-(2-amino-5-bromophenyl)-4-oxobutanoic acid; (R)-2-amino-4-(2-amino-5-bromophenyl)-4-oxobutanoic acid; (S)-2-amino-4-(2-amino-5-bromophenyl)-4-oxobutanoic acid; 2-amino-4-(2-amino-5-methylphenyl)-4-oxobutanoic acid; (R)-2-amino-4-(2-amino-5-methylphenyl)-4-oxobutanoic acid; (S)-2-amino-4-(2-amino-5-methylphenyl)-4-oxobutanoic acid; 2-amino-4-(2-amino-5-ethylphenyl)-4-oxobutanoic acid; (R)-2-amino-4-(2-amino-5-ethylphenyl)-4-oxobutanoic acid; (S)-2-amino-4-(2-amino-5-ethylphenyl)-4-oxobutanoic acid; 2-amino-4-(2-amino-5-propylphenyl)-4-oxobutanoic acid; (R)-2-amino-4-(2-amino-5-propylphenyl)-4-oxobutanoic acid; (S)-2-amino-4-(2-amino-5-propylphenyl)-4-oxobutanoic acid; 2-amino-4-(2-amino-5-isopropylphenyl)-4-oxobutanoic acid; (R)-2-amino-4-(2-amino-5-isopropylphenyl)-4-oxobutanoic acid; (S)-2-amino-4-(2-amino-5-isopropylphenyl)-4-oxobutanoic acid; 2-amino-4-(2-amino-5-cyclohexylphenyl)-4-oxobutanoic acid; (R)-2-amino-4-(2-amino-5-cyclohexylphenyl)-4-oxobutanoic acid; (S)-2-amino-4-(2-amino-5-cyclohexylphenyl)-4-oxobutanoic acid; 2-amino-4-(2-(methylamino)phenyl)-4-oxobutanoic acid; (S)-2-amino-4-(2-(methylamino)phenyl)-4-oxobutanoic acid; (R)-2-amino-4-(2-(methylamino)phenyl)-4-oxobutanoic acid;2-amino-4-(2-(dimethylamino)phenyl)-4-oxobutanoic acid; (S)-2-amino-4-(2-(dimethylamino)phenyl)-4-oxobutanoic acid; (R)-2-amino-4-(2-(dimethylamino)phenyl)-4-oxobutanoic acid; 2-amino-4-(2-amino-5-chlorophenyl)-4-oxobutanoic acid; (R)-2-amino-4-(2-amino-5-chlorophenyl)-4-oxobutanoic acid; (S)-2-amino-4-(2-amino-5-chlorophenyl)-4-oxobutanoic acid; 2-amino-4-(2-amino-6-chlorophenyl)-4-oxobutanoic acid; (R)-2-amino-4-(2-amino-6-chlorophenyl)-4-oxobutanoic acid; (S)-2-amino-4-(2-amino-6-chlorophenyl)-4-oxobutanoic acid; 2-acetamido-4-(2-amino-5-chlorophenyl)-4-oxobutanoic acid; (R)-2-acetamido-4-(2-amino-5-chlorophenyl)-4-oxobutanoic acid; (S)-2-acetamido-4-(2-amino-5-chlorophenyl)-4-oxobutanoic acid; 2-amino-4-(2-amino-3-hydroxyphenyl)-4-oxobutanoic acid; (S-)2-amino-4-(2-amino-3-hydroxyphenyl)-4-oxobutanoic acid; (R-)2-amino-4-(2-amino-3-hydroxyphenyl)-4-oxobutanoic acid; methyl (S-)2-amino-4-(2-aminophenyl)-4-oxobutanoate; 2-amino-4-(2-aminophenyl)-4-oxobutanamide; (R)-2-amino-4-(2-aminophenyl)-4-oxobutanamide; (S)-2-amino-4-(2-aminophenyl)-4-oxobutanamide; (S)-2-acetamido-4-(2-acetamidophenyl)-4-oxobutanoic acid; (S)-2-formamido-4-(2-formamidophenyl)-4-oxobutanoic acid; 4-(2-aminophenyl)-4-oxo-2-phenylbutanamide.

In some embodiments, the invention provides compound of formula (I), wherein Ar¹ is preferably phenyl or pyridyl; wherein said phenyl or pyridyl can be unsubstituted or substituted with one, two, three or four R^{za}; preferably Ar¹ is phenyl which can be unsubstituted or substituted with one, two, three or four R^{za}.

In some embodiments, each R^{za} is independently selected from the group comprising hydrogen, halo, C₁₋₆alkyl, haloC₁₋₆alkyl, and —OR^{a}; preferably each R^{za} is independently selected from the group comprising hydrogen, halo, C₁₋₆alkyl, haloC₁₋₆alkyl, and haloC₁₋₆alkyloxy; preferably each R^{za} is independently selected from the group comprising hydrogen, halo, C₁₋₄alkyl, haloC₁₋₄alkyl, and haloC₁₋₄alkyloxy; preferably each R^{za} is independently selected from the group comprising hydrogen, halo, and C₁₋₄alkyl; preferably each R^{za} is independently selected hydrogen, halo, or C₁₋₆alkyl; preferably each R^{za} is hydrogen.

In some embodiments, each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, C₃₋₆cycloalkyl-C₁₋₆alkylene, and —R^{f1}-R^{g1}; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

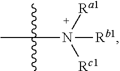

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₂₀alkylene, wherein said C₁₋₂₀alkylene can be interrupted by one or more O or S; R^{g1} is hydrogen, or C₁₋₂₀alkyl; preferably each R^{a} is independently selected from the group comprising hydrogen, halo, C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, C₃₋₆cycloalkyl-C₁₋₆alkylene, and —R^{f1}-R^{g1}; wherein each of said C₁₋₁₈alkyl, C₂₋₁₈alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

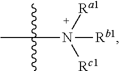

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₁₅alkylene, wherein said C₁₋₁₅alkylene can be interrupted by one or more O or S; R^{g1} is hydrogen, or C₁₋₁₅alkyl; preferably each R^{a} is independently selected from the group comprising hydrogen, halo, C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, C₃₋₆cycloalkyl, and —R^{f1}-R^{g1}; wherein each of said C₁₋₁₆alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

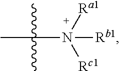

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{f1} is C₁₋₁₀alkylene, wherein said C₁₋₁₀alkylene can be interrupted by one or more O or S; R^{g1} is hydrogen, or C₁₋₁₀alkyl; preferably each R^{a} is independently selected from the group comprising hydrogen, halo, C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, C₅₋₆cycloalkyl, and —R^{f1}-R^{g1}; wherein each of said C₁₋₁₅alkyl, C₂₋₆alkenyl, haloC₁₋₆alkyl, C₁₋₁₀alkylcarbonyl, phenyl, 6 membered heteroaryl containing at least one N, 3-6 membered heterocyclyl containing at least one N or one O, and C₅₋₆cycloalkyl, can be unsubstituted or substituted with one or more R^{za1}; wherein each R^{za1} is independently selected from the group comprising —COO—R^{h},

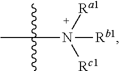

—NR^{a1}R^{b1}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; R^{f1} is C₂₋₆alkylene, wherein said C₁-alkylene can be interrupted by one or more O or S; R^{g1} is hydrogen, or C₁₋₈alkyl; preferably each R^{a} is independently selected from the group comprising hydrogen, halo, C₁₋₁₅alkyl, C₂₋₆alkenyl, haloC₁₋₆alkyl, C₁₋₈alkylcarbonyl, 3-5 membered heterocyclyl containing at least one N or one O, phenyl, and —R^{f1}-R^{g1}; wherein each of said C₁₋₁₅alkyl, C₂₋₆alkenyl, haloC₁₋₆alkyl, C₁₋₆alkylcarbonyl, 3-5 membered heterocyclyl containing at least one N or one O, and phenyl, can be unsubstituted or substituted with one or more R^{za1}; wherein each R^{za1} is independently selected from the group comprising —COO—R^{h},

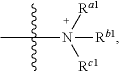

—NR^{a1}R^{b}, —R^{f1}-R^{g1}, and C₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; R^{f1} is C₁₋₆alkylene, wherein said C₁₋₆alkylene can be interrupted by one or more O or S; R^{g1} is hydrogen, or C₁₋₆alkyl; preferably each R^{a} is independently selected from the group comprising hydrogen, halo, C₁₋₁₆alkyl, C₂₋₆alkenyl, 3-4 membered heterocyclyl containing at least one O, C₁₋₈alkylcarbonyl, and —R^{f1}-R^{g1}; wherein each of said C₁₋₁₆alkyl, C₂₋₆alkenyl, 3-4 membered heterocyclyl containing at least one O, and C₁₋₈alkylcarbonyl, can be unsubstituted or substituted with one or more R^{za1}; wherein each R^{za1} is independently selected from the group comprising —COO—R^{h},

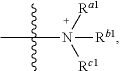

—NR^{a1}R^{b1}, —R^{f1}-R^{g1}, and C₁₋₆alkyl; R^{h} is selected from hydrogen, or C₂₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; R^{b1} is C₁₋₆alkylene, wherein said C₁₋₆alkylene can be interrupted by one or more O or S; R^{g1} is hydrogen, or C₁₋₄alkyl; preferably each R^{a} is independently selected from the group comprising hydrogen, halo, C₁₋₁₅alkyl, C₂₋₄alkenyl, oxiranyl, and —R^{f1}-R^{g1}; wherein each of said C₁₋₁₅alkyl, C₂₋₄alkenyl, and oxiranyl, can be unsubstituted or substituted with one or more R^{za1}; wherein each R^{za1} is independently selected from the group comprising —COO—R^{h},

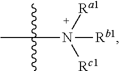

NR^{a1}R^{b1}, —R^{f1}-R^{g1}, and C₁₋₆alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl; R^{f1} is C₁₋₆alkylene, wherein said C₁₋₆alkylene can be interrupted by one or more O or S; R^{g1} is hydrogen, or C₁₋₄alkyl.

In some embodiments, each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene, can be unsubstituted or substituted with one or more R^{za1}; wherein each R^{za1} is independently selected from the group comprising —COO—R^{h},

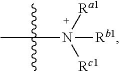

—NR^{a1}R^{b1}, halo, C₁₋₆alkyl, haloC₁₋₈alkyl, C₁₋₆alkylcarbonyl, C₂₋₆alkenyl, and C₂₋₆alkynyl; R^{h} is selected from the group comprising hydrogen, C₁₋₆alkyl, C₂₋₆alkenyl, and C₂₋₆alkynyl; R^{a1} is selected from the group comprising hydrogen, C₁₋₆alkyl, C₂₋₆alkenyl, and C₂₋₆alkynyl; R^{b1} is selected from the group comprising hydrogen, C₁₋₆alkyl, C₂₋₆alkenyl, and C₂₋₆alkynyl; R^{c1} is selected from the group comprising hydrogen, C₁₋₆alkyl, C₂₋₆alkenyl, and C₂₋₆alkynyl; preferably each R^{a} is independently selected from the group comprising hydrogen, halo, C₁₋₁₈alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; wherein each of said C₁₋₁₈alkyl, haloC₁₋₆alkyl, C₁₋₁₂alkylcarbonyl, C₂₋₆alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

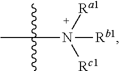

—NR^{a1}R^{b1}, halo, C₁₋₆alkyl, haloC₁₋₆alkyl, and C₁₋₆alkylcarbonyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₆alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; wherein each of said C₁₋₁₆alkyl, C₂₋₁₆alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h},

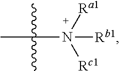

—NR^{a1}R^{b1}, halo, C₂₋₆alkyl, and haloC₂₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R¹ is selected from hydrogen, or C₁₋₆alkyl; preferably each R^{a} is independently selected from the group comprising hydrogen, halo, C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl; wherein each of said C₁₋₁₅alkyl, C₂₋₁₅alkenyl, haloC₁₋₆alkyl, C₁₋₁₂alkylcarbonyl, C₆₋₁₀aryl, 5-6 membered heterocyclyl containing at least one N or O, 5-6 membered heteroaryl containing at least one N, and C₃₋₆cycloalkyl, can be unsubstituted or substituted with one or more R^{za1}; wherein each R^{za1} is independently selected from the group comprising —COO—R^{h},

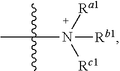

—NR^{a1}R^{b1}, halo, C₁₋₈alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl; wherein each of said C₁₋₁₅alkyl, C₂₋₁₀alkenyl, haloC₁₋₆alkyl, C₁₋₁₀alkylcarbonyl, phenyl, 6 membered heteroaryl containing at least one N, 3-5 membered heterocyclyl containing at least one N or one O, and C₅₋₆cycloalkyl, can be unsubstituted or substituted with one or more R^{za1};
wherein each R^{za1} is independently selected from the group comprising —COO—R^{h},

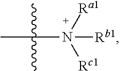

—NR^{a1}R^{b1}, halo, C₁₋₆alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₅alkyl, C₂₋₆alkenyl, haloC₁₋₆alkyl, C₁₋₆alkylcarbonyl, 3-4 membered heterocyclyl containing at least one O, and phenyl; wherein each of said C₁₋₁₅alkyl, C₂₋₆alkenyl, haloC₁₋₆alkyl, C₁₋₆alkylcarbonyl, 3-4 membered heterocyclyl containing at least one O, and phenyl, can be unsubstituted or substituted with one or more R^{za1}; wherein each R^{za1} is independently selected from the group comprising —COO—R^{h},

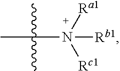

—NR^{a1}R^{b1}, halo, C₁₋₈alkyl, and haloC₁₋₆alkyl; R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₁₅alkyl, C₂₋₆alkenyl, oxiranyl, and C₁₋₆alkylcarbonyl; wherein each of said C₁₋₁₅alkyl, C₂₋₆alkenyl, oxiranyl, and C₁₋₈alkylcarbonyl, can be unsubstituted or substituted with one or more R^{za1}; wherein each R^{za1} is independently selected from the group comprising —COO—R^{h},

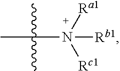

—NR^{a1}R^{b1}, and C₁₋₈alkyl;
R^{h} is selected from hydrogen, or C₁₋₆alkyl; R^{a1} is selected from hydrogen, or C₁₋₆alkyl; R^{b1} is selected from hydrogen, or C₁₋₆alkyl; R^{c1} is selected from hydrogen, or C₁₋₆alkyl; preferably each R^{a} is independently selected from the group comprising hydrogen, halo, C₁₋₅alkyl, C₂₋₄alkenyl, and oxiranyl; wherein each of said C₁₋₁₅alkyl, C₂₋₆alkenyl, oxiranyl, and C₁₋₆alkylcarbonyl, can be unsubstituted or substituted with one or more R^{za1}; wherein each R^{za1} is independently selected from the group comprising —COO—R^{h},

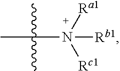

—NR^{a1}R^{b1}, and C₁₋₄alkyl; R^{h} is selected from hydrogen, or C₁₋₄alkyl; R^{a1} is selected from hydrogen, or C₁₋₄alkyl; R^{b1} is selected from hydrogen, or C₁₋₄alkyl; R^{c1} is selected from hydrogen, or C₁₋₄alkyl.

In some embodiments, each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃-cycloalkyl-C₁₋₆alkylene, C₃-cycloalkyl-C₂₋₆alkenylene, and C₃₋₆cycloalkyl-C₂₋₆alkynylene; preferably each R^{a} is independently selected from the group comprising hydrogen, halo, C₁₋₂₀alkyl, haloC₁₋₆alkyl, C₁₋₁₂alkylcarbonyl, C₂₋₁₈alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{a} is independently selected from the group comprising hydrogen, halo, C₁₋₁₈alkyl, C₂₋₁₀alkenyl, haloC₁₋₆alkyl, C₁₋₁₂alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably each R^{a} is independently selected from the group comprising hydrogen, halo, C₁₋₁₈alkyl, C₂₋₆alkenyl, haloC₁₋₆alkyl, C₁₋₁₂alkylcarbonyl, C₆₋₁₀aryl, 3-6 membered heterocyclyl containing at least one N or one O, 5-6 membered heteroaryl containing at least one N, and C₃₋₆cycloalkyl; preferably each R^{a} is independently selected from the group comprising hydrogen, halo, C₁₋₁₆alkyl, C₂₋₆alkenyl, haloC₁₋₆alkyl, C₁₋₁₀alkylcarbonyl, phenyl, 6 membered heteroaryl containing at least one N, 3-5 membered heterocyclyl containing at least one O, and C₅₋₆cycloalkyl; preferably each R^{a} is independently selected from the group comprising hydrogen, halo, C₁₋₁₆alkyl, C₂₋₆alkenyl, haloC₁₋₆alkyl, 3-5 membered heterocyclyl containing at least one O, C₁₋₆alkylcarbonyl, and phenyl; preferably each R^{a} is independently selected from the group comprising hydrogen, halo, C₁₋₁₅alkyl, C₂₋₆alkenyl, 3-4 membered heterocyclyl containing one O, and C₁₋₆alkylcarbonyl; preferably each R^{a} is independently selected from the group comprising hydrogen, halo, and C₁₋₁₅alkyl, C₂₋₄alkenyl, and oxiranyl.

In some embodiments, each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 3-10 membered heterocyclyl-C₁₋₆alkylene, 5-10 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl, 3-6 membered heterocyclyl-C₁₋₆alkylene, 5-6 membered heteroaryl, and C₃₋₆cycloalkyl; preferably each R^{b} is independently selected from the group comprising hydrogen, halo, C₁₋₆alkyl, haloC₁₋₆alkyl, C₆₋₁₀aryl, 3-6 membered heterocyclyl containing at least one N, 3-6 membered heterocyclyl-C₁₋₆alkylene containing at least one 0, 5-6 membered heteroaryl containing at least one N, and C₃₋₆cycloalkyl; preferably each R^{b} is independently selected from the group comprising hydrogen, halo, C₁₋₆alkyl, haloC₁₋₆alkyl, 3-5 membered heterocyclyl-C₁₋₆alkylene containing at least one O, and C₆₋₁₀aryl; preferably each R^{b} is independently selected from the group comprising hydrogen, C₁₋₆alkyl, 3-4 membered heterocyclyl-C₁₋₄alkylene containing one O, and haloC₁₋₆alkyl; preferably each R^{b} is independently selected from hydrogen, oxiranyl-C₁₋₂alkylene, or C₁₋₆alkyl; preferably each R^{b} is hydrogen.

In some embodiments, X^{1a} is selected from —NR^{b}—, —O—, or —S—; preferably X^{1a} is selected from —NR^{b}—, or —O—; preferably X^{1a} is —NR^{b}—, preferably —NH—.

In some embodiments, Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₆₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1a} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1a} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1a} is selected from the group comprising hydrogen, —CO—R^{a}, —CO—C₂₋₄alkenylene-CO—O—C₁₋₄alkyl, —CO—C₁₋₆alkylene-maleimidyl, C₁₋₆alkyl, 3-4 membered heterocyclyl-C₁₋₆alkylene containing one O, C₁₋₆alkylcarbonyl, and C₂₋₆alkenylcarbonyl; preferably Z^{1a} is selected from the group comprising hydrogen, C₁₋₆alkyl, —CO—C₂₋₄alkenylene-CO—O—C₁₋₄alkyl, —CO—C₁₋₆alkylene-maleimidyl, oxiranyl-C₁₋₄alkylene, C₁₋₆alkylcarbonyl, and C₂₋₄alkenylcarbonyl; preferably Z^{1a} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, C₁₋₆alkylcarbonyl, and C₂₋₄alkenylcarbonyl.

In some embodiments, X^{2a} is —(CR^{c}R^{d})ₙ—; preferably X^{2a} is —(CR^{c}H)ₙ—; preferably X^{2a} is —(CH₂)ₙ—; preferably X^{2a} is —(CH₂)ₙ—; wherein n is an integer selected from 1, 2 or 3; preferably 1 or 2; preferably n is 1; preferably X^{2a} is —CH₂—.

In some embodiments, each R^{c} and R^{d} is independently selected from the group comprising hydrogen, halo, C₁₋₆alkyl, haloC₁₋₆alkyl, C₂₋₆alkenyl, C₂₋₆alkynyl, C₆₋₁₀aryl, 3-10 membered heterocyclyl, 5-10 membered heteroaryl, C₃₋₆cycloalkyl, C₁₋₁₀aryl-C₁₋₆alkylene, C₁₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably each R^{c} and R^{d} is independently selected from the group comprising hydrogen, halo, C₁₋₆alkyl, haloC₁₋₆alkyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably each R^{c} and R^{d} is independently selected from the group comprising hydrogen, halo, C₁₋₆alkyl, haloC₁₋₆alkyl, C₆₋₁₀aryl, 3-10 membered heterocyclyl, 5-10 membered heteroaryl, and C₃₋₆cycloalkyl; preferably each R^{c} and R^{d} is independently selected from the group comprising hydrogen, halo, C₁₋₆alkyl, haloC₁₋₆alkyl, C₆₋₁₀aryl, 3-6 membered heterocyclyl, 5-6 membered heteroaryl, and C₃₋₆cycloalkyl; preferably each R^{c} and R^{d} is independently selected from the group comprising hydrogen, halo, C₁₋₆alkyl, haloC₁₋₆alkyl, C₆₋₁₀aryl, 3-6 membered heterocyclyl containing at least one N, 5-6 membered heteroaryl containing at least one N, and C₃₋₆cycloalkyl; preferably each R^{c} and R^{d} is independently selected from the group comprising hydrogen, halo, C₁₋₆alkyl, haloC₁₋₆alkyl, and C₆₋₁₀aryl; preferably each R^{b} is independently selected from the group comprising hydrogen, C₁₋₆alkyl, and haloC₁₋₆alkyl; preferably each R^{c} and R^{d} is independently selected from hydrogen, or C₁₋₆alkyl; preferably each R^{c} and R^{d} is independently is hydrogen.

In some embodiments, R^{e} is selected from the group comprising C₁₋₁₂alkylene, C₂₋₁₂alkenylene, C₂₋₁₂alkynylene, C₆₋₁₀arylene, 3-6 membered heterocyclylene, 5-6 membered heteroarylene, and C₃-cycloalkylene; preferably R^{e} is selected from the group comprising C₁₋₆alkylene, C₂₋₆alkenylene, C₂₋₆alkynylene, C₆₋₁₀arylene, 3-6 membered heterocyclylene, 5-6 membered heteroarylene, and C₃₋₆cycloalkylene; preferably R^{e} is selected from the group comprising C₁₋₆alkylene, C₂₋₆alkenylene, C₆₋₁₀arylene, 3-5 membered heterocyclylene, and C₃₋₆cycloalkylene; preferably R^{e} is selected from the group comprising C₁₋₆alkylene, C₂₋₆alkenylene, and C₃₋₆cycloalkylene; preferably R^{e} is C₁₋₄alkylene or C₂₋₄alkenylene; preferably R^{e} is C₂₋₄alkenylene or C₁₋₆alkylene; preferably R^{e} is C₂₋₄alkenylene; preferably R^{e} is C₂₋₆alkenylene.

In some embodiments, R^{f} is C₁₋₂₀alkylene, wherein said alkylene can be interrupted by one or more O or S; preferably R^{f} is C₁₋₁₆alkylene, wherein said alkylene can be interrupted by one or more O or S; preferably R^{f} is C₁₋₁₄alkylene, wherein said alkylene can be interrupted by one or more O or S; preferably R^{f} is C₁₋₁₀alkylene, wherein said alkylene can be interrupted by one or more O; preferably R^{f} is C₁₋₆alkylene, wherein said alkylene can be interrupted by one or more O; preferably R^{f} is C₁₋₆alkylene.

In some embodiments, R^{g} is selected from hydrogen, C₁₋₂₀alkyl, N-maleimidyl, N-maleimidyloxy, or hydrogen, C₁₋₁₀alkyl, N-maleimidyloxycarbonyl; preferably R^{g} is hydrogen, C₁₋₆alkyl, N-maleimidyl, or N-maleimidyloxy; preferably R^{g} is hydrogen, C₁₋₄alkyl, N-maleimidyl.

In some embodiments, R^{g} is selected from N-maleimidyl, N-maleimidyloxy, or N-maleimidyloxycarbonyl; preferably R^{g} is N-maleimidyl, or N-maleimidyloxy; preferably R^{g} is N-maleimidyl.

In some embodiments, W^{1a} is selected from —NR^{b}Z^{2a}, —R^{a}, or —OR^{a}; preferably W^{1a} is selected from —NR^{b}Z^{2a}, or —R^{a}; preferably W^{1a} is selected from the group comprising —NHZ^{2a}, hydrogen, halo, C₁₋₁₂alkyl, haloC₁₋₆alkyl, C₁₋₁₂alkylcarbonyl, C₂₋₁₂alkenyl, C₂₋₁₂alkynyl, and C₆₋₁₀aryl; preferably W^{1a} is selected from the group comprising —NHCO—R^{a}, hydrogen, halo, C₁₋₁₂alkyl, haloC₁₋₆alkyl, C₁₋₁₂alkylcarbonyl, and C₆₋₁₀aryl; preferably W^{1a} is selected from the group comprising NHCO—R^{a}, hydrogen, C₁₋₆alkyl, C₁₋₁₂alkylcarbonyl, and C₆₋₁₀aryl; preferably W^{1a} is —NHCO—R^{a}, C₁₋₆alkyl, C₁₋₆alkylcarbonyl, and C₆₋₁₀aryl; preferably W^{1a} is —NHCO—R^{a}; preferably W^{1a} is —NHCO—C₁₋₁₂alkyl; preferably W^{1a} is —NHCO—C₁₋₁₀alkyl; preferably W^{1a} is —NHCO—C₁₋₆alkyl; preferably W^{1a} is —NHCO—C₁₋₆alkyl.

In some embodiments, Z^{2a} is selected from the group comprising hydrogen, —CO—R^{a}, halo, C₁₋₁₂alkyl, haloC₁₋₆alkyl, C₁₋₁₂alkylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{2a} is selected from the group comprising hydrogen, —CO—R^{a}, halo, C₁₋₁₂alkyl, haloC₁₋₆alkyl, C₁₋₁₂alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₁₀alkyl, C₁₋₁₀alkylcarbonyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{2a} is selected from the group comprising hydrogen, —CO—R^{a}, C₁₋₆alkyl, C₁₋₆alkylcarbonyl, C₆₋₁₀aryl, 3-6 membered heterocyclyl, 5-6 membered heteroaryl, and C₃₋₆cycloalkyl; preferably Z^{2a} is selected from the group comprising hydrogen, —CO—R^{a}, C₁₋₈alkyl, and C₁₋₆alkylcarbonyl; preferably Z^{2a} is selected from the group comprising hydrogen, C₁₋₈alkyl, and C₁₋₆alkylcarbonyl; preferably Z^{2a} is C₁₋₆alkylcarbonyl.

In some embodiments, Y^{1a} is a moiety of formula (A), or is selected from —OR^{a}, —R^{a}, or —NR^{a}R^{b};

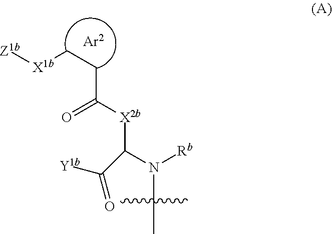

preferably Y^{1a} is a moiety of formula (A) or is —OR^{a}, or —NHR^{a}; preferably Y^{1a} is a moiety of formula (A), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is a moiety of formula (A), or is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀ aryl, —O—C₃₋₆cycloalkyl, C₆₋₁₀aryl-C₁₋₆alkylene-O—; preferably Y^{1a} is a moiety of formula (A), or is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is a moiety of formula (A), or is selected from the group comprising —OH, —NH₂, —O—C₁₋₁₂alkyl, and —NHC₁₋₁₂alkyl; preferably Y^{1a} is a moiety of formula (A), or is selected from the group comprising —OH, —NH₂, —O—C₁₋₁₀alkyl, and —NHC₁₋₁₀alkyl; preferably Y^{1a} is a moiety of formula (A).

In some embodiments Y^{1a} is —OR^{a}, or —NHR^{a}; preferably Y^{1a} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1a} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1a} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1a} is selected from the group comprising —OH, —NH₂, —O—C₁₋₁₂alkyl, and —NHC₁₋₁₂alkyl; preferably Y^{1a} is selected from the group comprising —OH, —NH₂, —O—C₁₋₁₂alkyl, and —NHC₁₋₁₀alkyl; preferably Y^{1a} is selected from the group comprising —OH, —NH₂, —O—C₁₋₁₂alkyl, and —NHC₁₋₆alkyl; preferably Y^{1a} is —O—C₁₋₁₂alkyl or OH; preferably Y^{1a} is —O—C₁₋₁₀alkyl or OH; preferably Y^{1a} is —O—C₁₋₆alkyl or OH.

In some embodiments, Y^{1a} is a moiety of formula (A) and Ar² is phenyl or pyridyl; wherein said phenyl or pyridyl can be unsubstituted or substituted with one, two, three or four R^{zb}; preferably Ar² is phenyl which can be unsubstituted or substituted with one, two, three or four R^{zb}.

In some embodiments, each R^{zb} is independently selected from the group comprising hydrogen, halo, C₁₋₆alkyl, haloC₁₋₆alkyl, and —OR^{a}; preferably each R^{zb} is independently selected from the group comprising hydrogen, halo, C₁₋₆alkyl, haloC₁₋₆alkyl, and haloC₁₋₆alkyloxy; preferably each R^{zb} is independently selected from the group comprising hydrogen, halo, C₁₋₄alkyl, haloC₁₋₄alkyl, and haloC₁₋₄alkyloxy; preferably each R^{zb} is independently selected from the group comprising hydrogen, halo, and C₁₋₄alkyl; preferably each R^{zb} is independently selected hydrogen, halo, or C₁₋₆alkyl; preferably each R^{zb} is hydrogen.

In some embodiments, X^{1b} is selected from —NR^{b}—, —O—, or —S—; preferably X^{1b} is selected from —NR^{b}—, or —O—; preferably X^{1b} is —NR^{b}—, preferably —NH—;

In some embodiments, Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, C₆₋₁₀aryl-C₂₋₆alkenylene, C₆₋₁₀aryl-C₂₋₆alkynylene, 5-10 membered heteroaryl-C₁₋₆alkylene, 5-10 membered heteroaryl-C₂₋₆alkenylene, 5-10 membered heteroaryl-C₂₋₆alkynylene, 3-10 membered heterocyclyl-C₁₋₆alkylene, 3-10 membered heterocyclyl-C₂₋₆alkenylene, 3-10 membered heterocyclyl-C₂₋₆alkynylene, C₃₋₆cycloalkyl-C₁₋₆alkylene, C₃₋₆cycloalkyl-C₂₋₆alkenylene, and C₃₋₆cycloalkyl-C₂₋₆alkynylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₂alkyl, haloC₁₋₆alkyl, C₁₋₁₂alkylcarbonyl, C₂₋₁₀alkenylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₁₋₁₀alkylcarbonyl, C₂₋₈alkenylcarbonyl, C₂₋₁₀alkenyl, C₂₋₁₀alkynyl, C₆₋₁₀aryl, 3-6 membered heterocyclyl, 5-6 membered heteroaryl, C₃₋₆cycloalkyl, C₆₋₁₀aryl-C₁₋₆alkylene, 5-6 membered heteroaryl-C₁₋₆alkylene, 3-6 membered heterocyclyl-C₁₋₆alkylene, and C₃₋₆cycloalkyl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₁₀alkyl, haloC₁₋₆alkyl, C₆₋₁₀alkylcarbonyl, C₂₋₆alkenylcarbonyl, C₆₋₁₀aryl, 3-6 membered heterocyclyl, 3-6 membered heterocyclyl-C₁₋₆alkylene, 5-6 membered heteroaryl, C₃₋₆cycloalkyl, C₆₋₁₀aryl-C₁₋₆alkylene, and 5-6 membered heteroaryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, 3-6 membered heterocyclyl containing at least one N or one O, 3-6 membered heterocyclyl-C₁₋₆alkylene containing at least one N or one O, 5-6 membered heteroaryl containing at least one N, C₃₋₆cycloalkyl, and C₆₋₁₀aryl-C₁₋₆alkylene; preferably Z^{1b} is selected from the group comprising hydrogen, —CO—R^{a}, —CO—R^{e}—CO—O—R^{b}, —CO—R^{f}-R^{g}, —R^{f}-R^{g}, halo, C₁₋₆alkyl, haloC₁₋₆alkyl, C₁₋₆alkylcarbonyl, C₂₋₆alkenylcarbonyl, C₆₋₁₀aryl, C₃₋₆cycloalkyl, 3-6 membered heterocyclyl-C₁₋₆alkylene containing at least one O, and C₆₋₁₀aryl-C₁₋₆alkylene; preferably Z^{1b} is selected from the group comprising hydrogen, —CO—R^{a}, —CO—C₂₋₆alkenylene-CO—O—R^{b}, —CO—R^{f}-maleimidyl, halo, C₁₋₆alkyl, haloC₁₋₆alkyl, 3-5 membered heterocyclyl-C₁₋₆alkylene containing at least one O, C₁₋₆alkylcarbonyl, and C₂₋₆alkenylcarbonyl; preferably Z^{1b} is selected from the group comprising hydrogen, —CO—R^{a}, —CO—C₂₋₆alkenylene-CO—O—R^{b}, —CO—R^{f}-maleimidyl, C₁₋₆alkyl, 3-4 membered heterocyclyl-C₁₋₆alkylene containing one O, C₁₋₈alkylcarbonyl, and C₂₋₄alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₄alkylene, C₁₋₆alkyl, C₁₋₆alkylcarbonyl, and C₂₋₄alkenylcarbonyl; preferably Z^{1b} is selected from the group comprising hydrogen, —CO—C₂₋₄alkenylene-CO—O—C₁₋₄alkyl, —CO—C₁₋₆alkylene-maleimidyl, oxiranyl-C₁₋₂alkylene-, and C₁₋₆alkylcarbonyl, and C₂₋₄alkenylcarbonyl.

In some embodiments, X^{2b} is —(CR^{c}R^{d})ₘ—; preferably X^{2b} is —(CR^{c}H)ₘ—; preferably X^{2b} is —(CH₂)ₘ—; preferably X^{2b} is —(CH₂)ₘ—; wherein m is an integer selected from 1, 2 or 3; preferably 1 or 2; preferably m is 1; preferably X^{2b} is —CH₂—.

In some embodiments, Y^{1b} is selected from OR^{a}, —NR^{b}R^{a}, or R^{a}; preferably Y^{1b} is —OR^{a}, or —NHR^{a}; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₂₋₁₂alkenyl, —O—C₂₋₁₂alkynyl, —O—C₆₋₁₀aryl, 3-10 membered —O-heterocyclyl, 5-10 membered —O-heteroaryl, —O—C₃₋₆cycloalkyl, C₆₋₁₀aryl-C₁₋₆alkylene-O—, 5-10 membered heteroaryl-C₁₋₆alkylene-O—, 3-10 membered heterocyclyl-C₁₋₆alkylene-O—, and C₃₋₆cycloalkyl-C₁₋₆alkylene-O—; preferably Y^{1b} is selected from the group comprising —OH, —NH₂, —O—C₁₋₁₂alkyl, —NHC₁₋₁₂alkyl, —O—CO—C₁₋₁₂alkyl, —NH—CO—C₁₋₁₂alkyl, —O—C₆₋₁₀aryl, —O—C₃₋₆cycloalkyl, C₆₋₁₀aryl-C₁₋₆alkylene-O—; preferably Y^{1b} is selected from the group comprising —OH, —NH₂, —O—C₁₋₁₂alkyl, —NHC₁₋₁₂alkyl, —O—C₆₋₁₀aryl, —O—C₃₋₆cycloalkyl; preferably Y^{1b} is selected from the group comprising —OH, —NH₂, —O—C₁₋₁₂alkyl, and —NHC₁₋₁₂alkyl; preferably Y^{1b} is selected from the group comprising —OH, —NH₂, —O—C₁₋₁₀alkyl, and —NHC₁₋₁₀alkyl; preferably Y^{1b} is —O—C₁₋₁₀alkyl, or —NH—C₁₋₁₀alkyl; preferably Y^{1b} is —O—C₁₋₈alkyl, or —NH—C₁₋₈alkyl, preferably —O—C₁₋₈alkyl.

In some embodiments, the invention provides compound of formula (IIA1), (IIA2), (IIA3), (IIA4), (IIA5), (IIA6), (IIA7), (IIA8), (IIA9), (IIA10) or (IIA11):

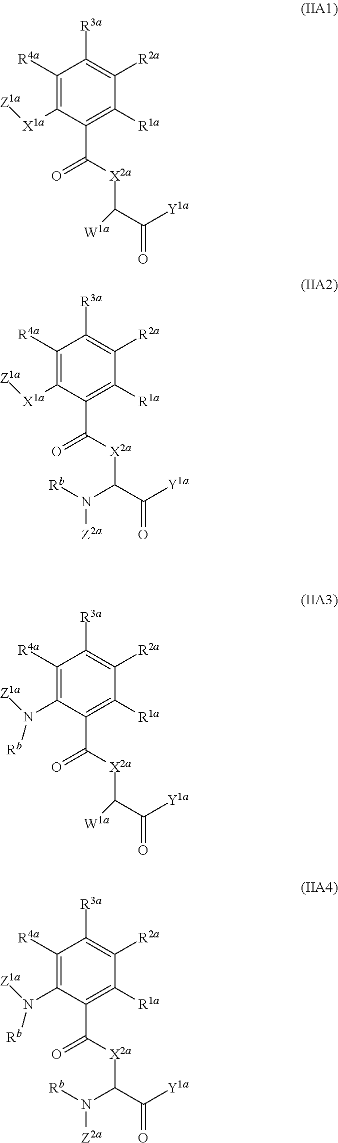

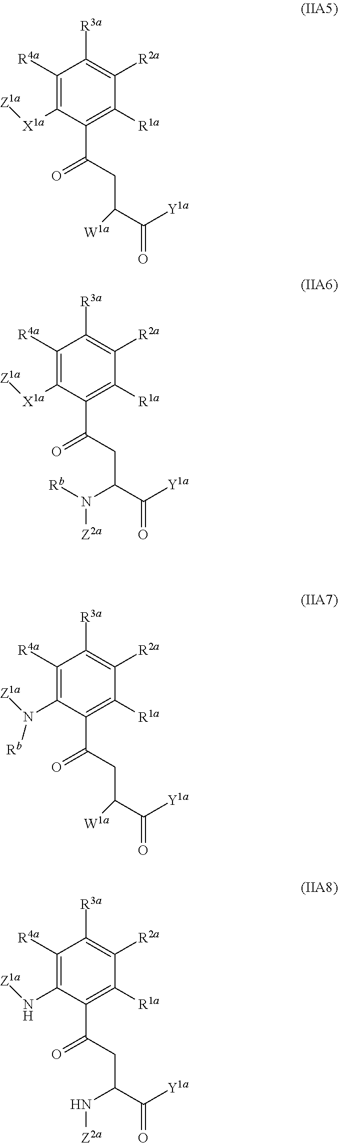

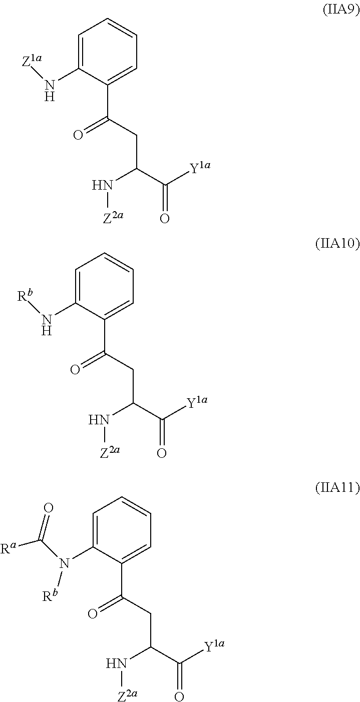

wherein R^{1a}, R^{2a}, R^{3a}, and R^{4a}, Z^{1a}, Z^{2a}, X^{1a}, X^{2a}, W^{1a}, Y^{1a}, R^{a}, R^{b}, have the same meaning as that defined herein.

The present invention also encompasses processes for preparing compounds of formula (I) as defined herein (including all embodiments thereof as described herein).

The compounds can be prepared while using a series of chemical reactions well known to those skilled in the art, altogether making up the process for preparing said compounds and exemplified further. The processes described further are only meant as examples and by no means are meant to limit the scope of the present invention.

The starting materials and reagents used in preparing these compounds are either available from commercial suppliers or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989).

The schemes are merely illustrative of some processes by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., in a subclass from about 0° C. to about 125° C. and most preferably at about room (or ambient) temperature, e.g., about 20-25° C.

In some embodiments, some compounds of the invention may be prepared according to the general procedure outlined in Scheme 1 below.

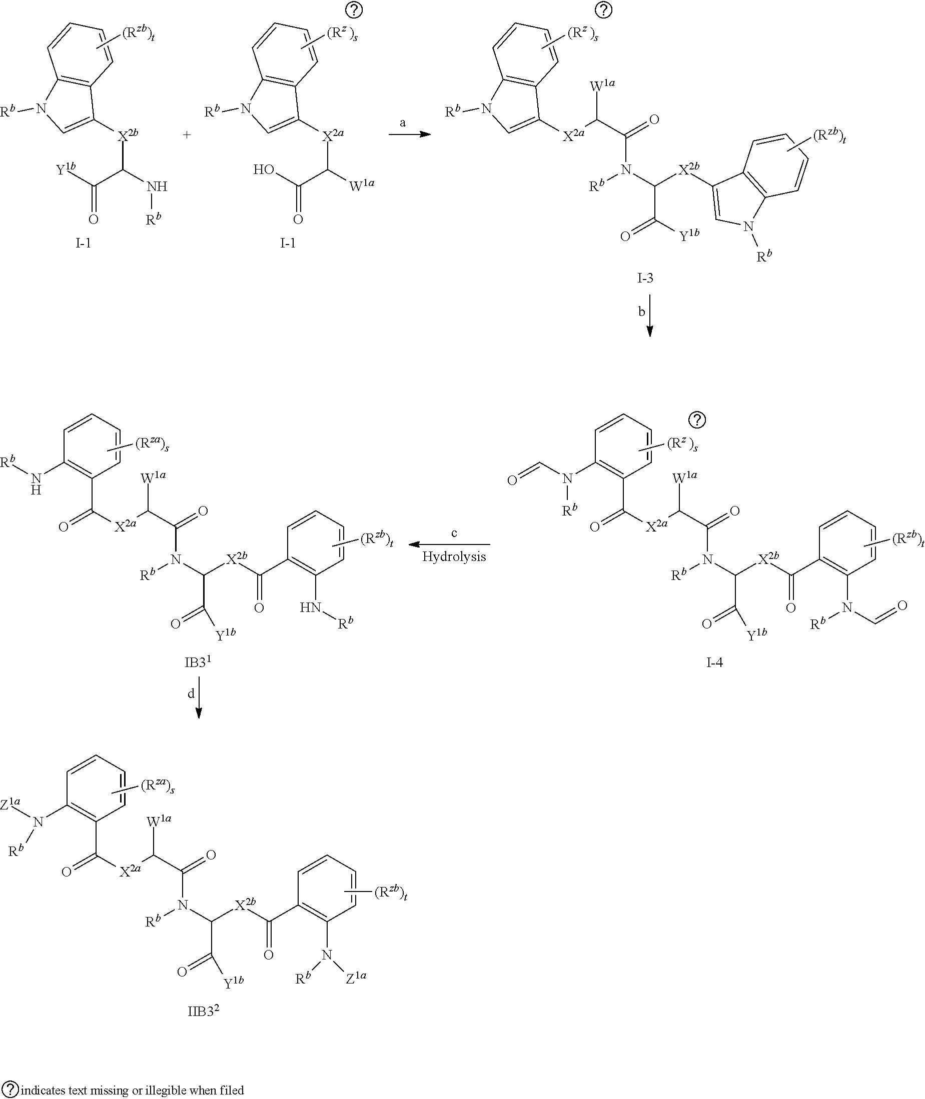

Scheme 1: Z^{1a}, R^{b}, X^{2a}, X^{2b}, W^{1a}, Y^{1b}, R^{za}, R^{zb} are as described for the compounds of the present invention. Each s and t is an integer selected from 1, 2, 3 or 4.

As shown in Scheme 1, compound of formula (IIB3¹) and (IIB3²) can be prepared using a process comprising the steps of:
a) coupling two tryptophan derivatives (I-1 and I-2) using at least one suitable coupling agent to obtain intermediate (I-3),
b) oxidizing the indoles of intermediate (I-3) using at least one suitable oxidizing agent to obtain intermediate (I-4), followed by the hydrolyzing intermediate (I-4) to obtain compound of formula (IIB3¹). In some embodiments, compound of formula (I-4) can be formed transiently as hydrolysis to their respective anilines derivatives (IIB3²) can occur simultaneously.

Compound of formula (IIB3²) can then be prepared by coupling the amines of the anilines of compound (IIB3¹) with compound of formula Z^{1a}-LG, wherein Z^{1a} is as defined herein and LG is a leaving group. Preferably the amines of the anilines of compound (IIB31) can be acylated to yield compound of formula (IIB2) wherein Z^{1a} is R^{a}—CO—. The acylating reagent can be advantageously an anhydride of formula (R^{a}CO)₂O, wherein R^{a} is as defined herein. A carboxylic acid chloride (R^{a}COCl) can also be used.

Step a) can be preferably performed by way of a carbodiimide-mediated coupling reaction. Any carbodiimide commonly used as a coupling reagent in peptide chemistry can be used. Such carbodiimides are well-known and include dicyclohexyl carbodiimide, di-isopropyl carbodiimide, 1-(3-dimethylaminopropyl)-3-ethyl carbodiimide hydrochloride, N-cyclohexyl-N′-(2′-morpho-linoethyl) carbodiimide-metho-p-toluene sulfonate, N-benzyl-N′-3′-dimethylaminopropyl-carbodiimide hydrochloride, 1-ethyl-3-(3-dimethylaminopropyl) carbodiimide methiodide, N-ethyl carbodiimide hydrochloride, and the like. The preferred carbodiimides are dicyclohexyl carbodiimide (DCC) and diisopropyl carbodiimide (DIC).

Step b) can be performed using at least one oxidizing agent selected from ozone, sodium periodate, peroxides, potassium permanganate, CrO₃, and the like; preferably ozone. Step b) can also be performed under Fenton reaction conditions, for example using peroxides in the presence of transition metal ions including chromium, manganese, and iron. For example, the oxidation can be done using a hydrogen peroxide and iron (II) system. Step b) can also be performed using photocatalytic oxidation to effect the production of singlet oxygen optionally in the presence of catalysts such as titanium dioxide under UV light.

In some embodiments, some compounds of the invention can be prepared, similarly to what is described in scheme 1, starting from compounds of formula II-1 and II-2:

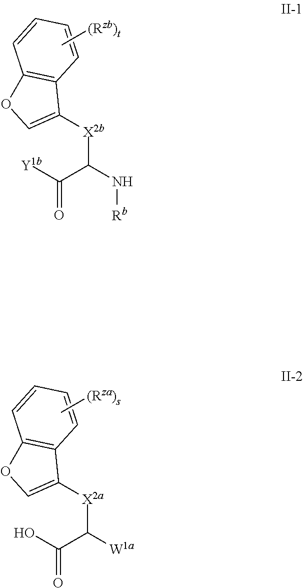

The process can comprise coupling two benzofuran derivatives (II-1 and II-2) using at least one suitable coupling agent, as described herein above for scheme 1, and oxidizing the benzofuran rings of the formed intermediate using at least one suitable oxidant. The oxidation of the benzofuran rings can be done for example with m-chloroperoxybenzoic acid (m-CPBA) or dimethyldioxirane as oxidants, leading to reactive epoxides that can be further oxidized, affording ring-opening keto esters.

In some embodiments, some compounds of the invention can be prepared, similarly to what is described in scheme 1, starting from compounds of formula III-1 and III-2:

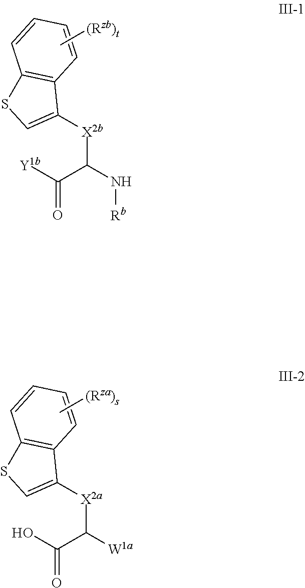

The process can comprise coupling two benzothiophene derivatives (III-1 and III-2) using at least one suitable coupling agent, as described herein above for scheme 1, and oxidizing the benzothiophene rings of the formed intermediate using at least one suitable oxidant. The oxidizing step can be done using photo-oxidation mechanism of benzothiophene using for example anthroquinone as a photosensitizer leading to aryl sulfinic or sulfonic acid in place of an aryl amine as in Scheme 1.

In some embodiments, some compounds of the invention may be prepared according to the general procedure outlined in Scheme 2 below.

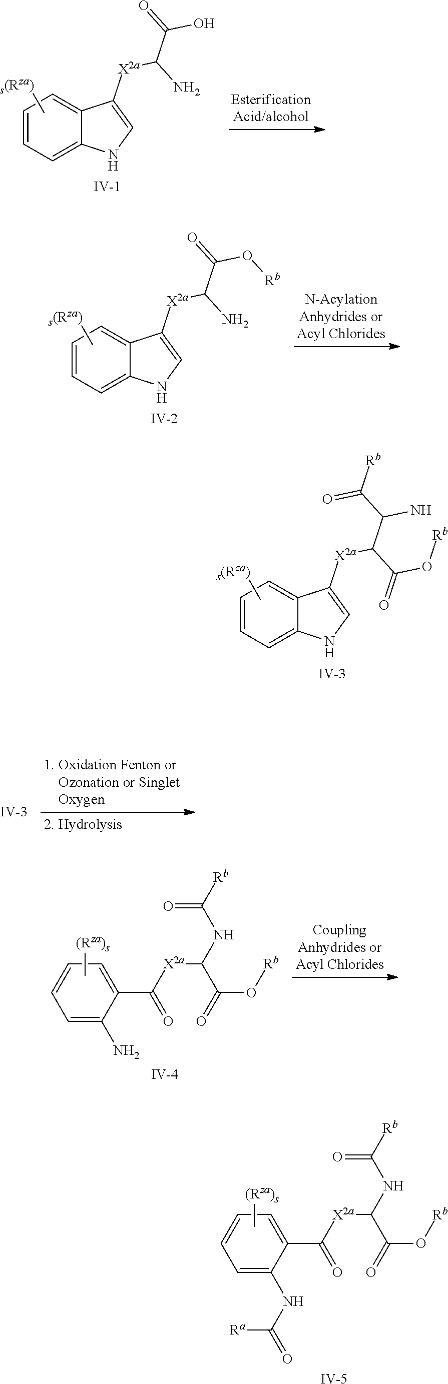

Scheme 2: R^{b}, X^{2a}, R^{za}, are as described for the compounds of the present invention. Each s is an integer selected from 1, 2, 3 or 4.

As shown in Scheme 2, compound of formula (IV-5) can be prepared using a process comprising the steps of: esterification of compound IV-1 with a suitable esterification agent such as an alcohol or and acid thereby forming intermediate (IV-2), next, intermediate (IV-2) can be N-acylated with suitable anhydrides or acyl chlorides, thereby forming intermediate (IV-3). Intermediate (IV-3) can then be oxidized using at least one suitable oxidizing agent followed by hydrolysis thereby forming compound (IV-4). The oxidation can be performed using at least one oxidizing agent selected from ozone, sodium periodate, peroxides, potassium permanganate, CrO₃, and the like; preferably ozone. The oxidation can also be performed under Fenton reaction conditions, for example using peroxides in the presence of transition metal ions including chromium, manganese, and iron. For example, the oxidation can be done using hydrogen peroxide and iron (II) system. The oxidation can also be performed using photocatalytic oxidation to effect the production of singlet oxygen optionally in the presence of catalysts such as titanium dioxide under UV light. Compound IV-5 can be obtained by coupling compound (IV-4) with suitable anhydrides or acyl chloride reagent. The reagent can be advantageously an anhydride of formula (R^{a}CO)₂O, or a carboxylic acid chloride (R^{a}COCl), wherein R^{a} is as defined herein.

In some embodiments, some compounds of the invention may be prepared according to the general procedure outlined in Scheme 3 below.

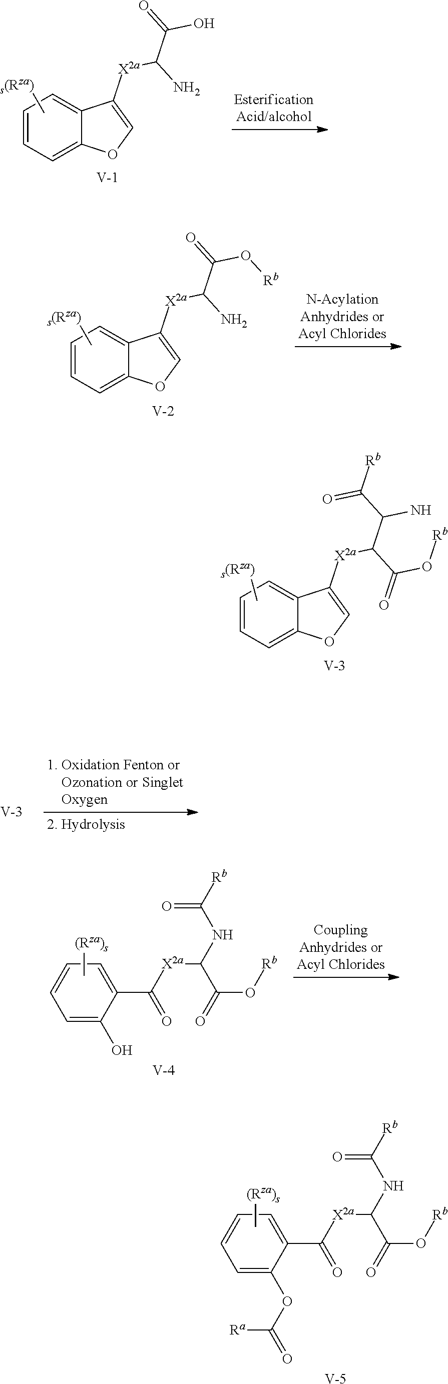

Scheme 3: R^{b}, X^{2a}, R^{za}, are as described for the compounds of the present invention. Each s is an integer selected from 1, 2, 3 or 4.

As shown in Scheme 3, compound of formula (V-5) can be prepared using a process comprising the steps of: esterification of compound V-1 with a suitable esterification agent such as an alcohol or and acid thereby forming intermediate (V-2). Next, intermediate (V-2) can be N-acylated with suitable anhydrides or acyl chlorides, thereby forming intermediate (V-3). Intermediate (V-3) can then be oxidized using at least one suitable oxidizing agent followed by hydrolysis thereby forming compound (V-4). The oxidation can be performed, as described before for Scheme 1, using at least one oxidizing agent selected from ozone, sodium periodate, peroxides, potassium permanganate, CrO₃, and the like; preferably ozone. The oxidation can also be performed under Fenton reaction conditions, for example using peroxides in the presence of transition metal ions including chromium, manganese, and iron. For example, the oxidation can be done using hydrogen peroxide and iron (II) system. The oxidation can also be performed using photocatalytic oxidation to effect the production of singlet oxygen optionally in the presence of catalysts such as titanium dioxide under UV light. Compound V-5 can be obtained by coupling compound (V-4) with suitable anhydrides or acyl chloride reagent. The reagent can be advantageously an anhydride of formula (R^{a}CO)₂O, or a carboxylic acid chloride (R^{a}COCl), wherein R^{a} is as defined herein.

In some embodiments, some compounds of the invention may be prepared according to the general procedure outlined in Scheme 4 below.

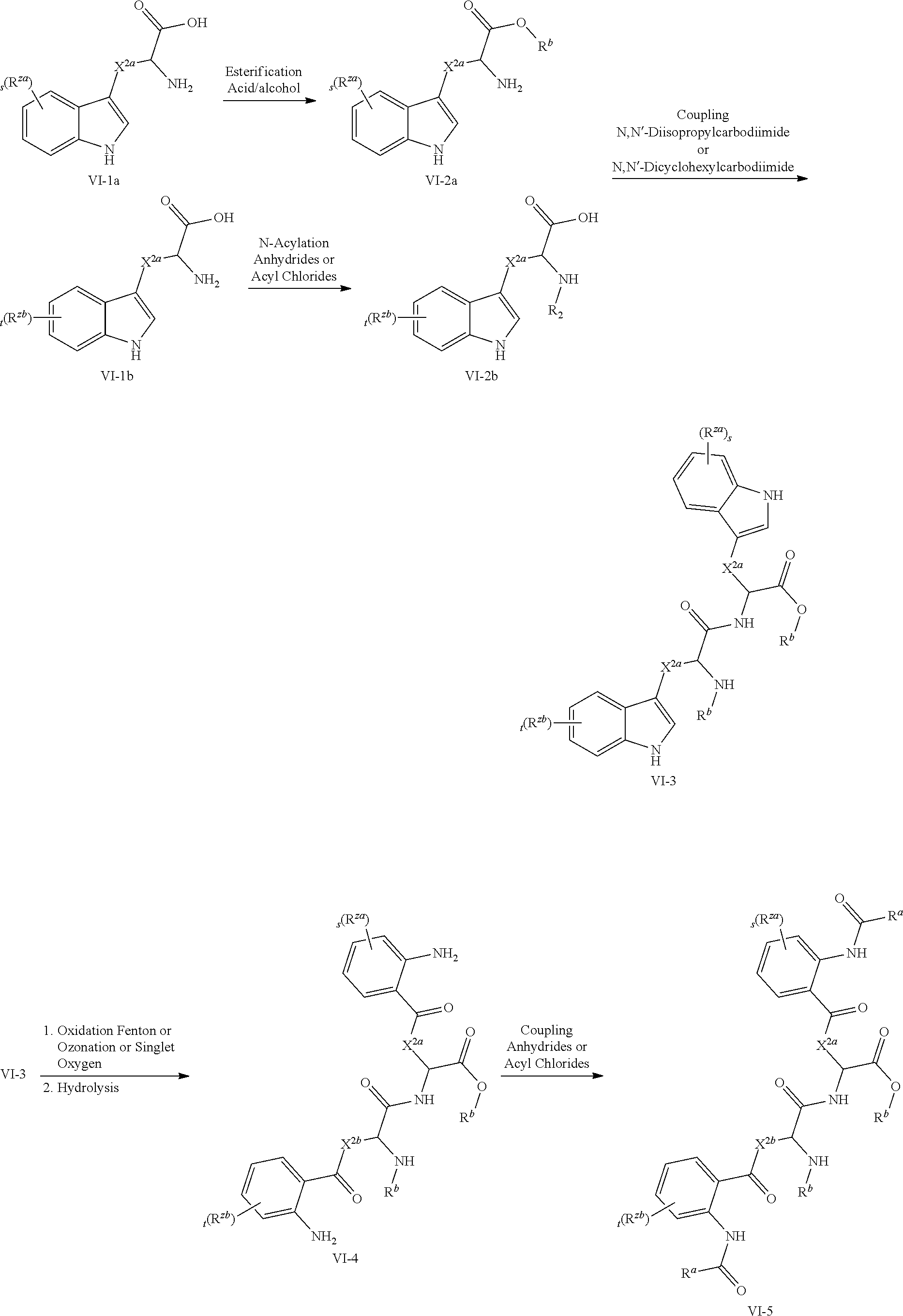

Scheme 4: Z^{1a}, R^{b}, X^{2a}, X^{2b}, R^{za}, R^{zb} are as described for the compounds of the present invention. Each s and t is an integer selected from 1, 2, 3 or 4.

As shown in Scheme 4, compound of formula (VI-5) can be prepared using a process comprising the steps of: preparing intermediate (VI-2a) by esterification of compound (VI-1a) with a suitable esterification agent such as an alcohol or and acid. Preparing intermediate (VI-2b) by N-acylation of compound (VI-1 b) with a suitable anhydride or acid chloride.

Then, coupling the two intermediates (VI-2a and VI-2b) using at least one suitable coupling agent to obtain intermediate (VI-3). Suitable coupling agents are the same as described for Scheme 1.

Next, intermediate (VI-3) can be oxidized using at least one suitable oxidizing agent followed by hydrolysis thereby forming compound (VI-4). The oxidation can be performed, as described before for Scheme 1, using at least one oxidizing agent selected from ozone, sodium periodate, peroxides, potassium permanganate, CrO₃, and the like; preferably ozone. The oxidation can also be performed under Fenton reaction conditions, for example using peroxides in the presence of transition metal ions including chromium, manganese, and iron. For example, the oxidation can be done using hydrogen peroxide and iron (II) system. The oxidation can also be performed using photocatalytic oxidation to effect the production of singlet oxygen optionally in the presence of catalysts such as titanium dioxide under UV light. Compound VI-5 can be obtained by coupling compound (VI-4) with suitable anhydrides or acyl chloride reagent. The reagent can be advantageously an anhydride of formula (R^{a}CO)₂O, or a carboxylic acid chloride (R^{a}COCl), wherein R^{a} is as defined herein.

A compound of formula (I) as defined herein (including all embodiments thereof as described herein) can be prepared as an acid addition salt, for example a cosmetically or pharmaceutically acceptable acid addition salt, by reacting the free base form of the compound with an inorganic or organic acid. Alternatively, a base addition salt, such as a cosmetically or pharmaceutically acceptable base addition salt, of a compound of formula (I) as defined herein (including all embodiments thereof as described herein) can be prepared by reacting the free acid form of the compound with an inorganic or organic base. Inorganic and organic acids and bases suitable for the preparation of the pharmaceutically acceptable salts of compounds of formula (I) are set forth in the definitions section of this application.

The free acid or free base forms of the compounds of formula (I) can be prepared from the corresponding base addition salt or acid addition salt form. For example, a compound of formula (I) in an acid addition salt form can be converted to the corresponding free base by treating with a suitable base (e.g., ammonium hydroxide solution, sodium hydroxide, etc.). A compound of formula (I) in a base addition salt form can be converted to the corresponding free acid by treating with a suitable acid (e.g., hydrochloric acid, etc.).

The N-oxides of compounds of formula (I) can be prepared by methods known to those of ordinary skill in the art. For example, N-oxides can be prepared by treating an unoxidized form of the compound of formula (I) with an oxidizing agent (e.g., trifluoroperacetic acid, permaleic acid, perbenzoic acid, peracetic acid, meta-chloroperoxybenzoic acid, etc.) in a suitable inert organic solvent (e.g., a halogenated hydrocarbon such as methylene chloride) at approximately 0° C. Alternatively, the N-oxides of the compounds of formula (I) can be prepared from the N-oxide of an appropriate starting material. Compounds of formula (I) in unoxidized form can also be prepared from N-oxides of compounds of formula (I) by treating the N-oxides with a reducing agent (e.g., sulfur, sulfur dioxide, triphenyl phosphine, lithium borohydride, sodium borohydride, phosphorus trichloride, tribromide, etc.) in a suitable inert organic solvent (e.g., acetonitrile, ethanol, aqueous dioxane, etc.), for example at temperature ranging 0 to 80° C.

Protected derivatives of the compounds of formula (I) can be made by means known to those of ordinary skill in the art. A detailed description of the techniques applicable to the creation of protective groups and their removal can be found in T. W. Greene, Protective Groups in Organic Synthesis, John Wiley & Sons, Inc. 1981.

Compounds of formula (I) can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of formula (I), dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, Honh Wiley & Sons, Inc. (1981).

The present invention further relates to a composition comprising at least one compound of formula (I) as defined herein (including all embodiments thereof as described herein).

The present invention also relates to and encompasses the compounds as listed in Table 1.

Preferably, the present invention further relates to cosmetic or pharmaceutical composition, comprising compound of formula (I) as defined herein (including all embodiments thereof as described herein) and optionally at least one dermatologically or pharmaceutically acceptable carrier. Preferably the cosmetic composition is a sunscreen composition.

Typically, one or more compounds of formula (I) may be employed in these compositions in varying amounts that will depend on the nature of the composition and the particular end use application. In some embodiments, compound of formula (I) is present in the cosmetic or pharmaceutical composition in an amount ranging from about 0.001% to about 50.0% by weight, preferably from about 0.001% to about 40.0% by weight, preferably from about 0.001% to about 30.0% by weight, preferably from about 0.005% to about 25.0% by weight, more preferably from 0.01% to 20.0% by weight, relative to the total weight of the composition.

In the preparation of such composition, at least one compound of formula (I) may be combined with other ingredients typically employed. For example, it can be formulated with one or more UV filters. In addition, the composition may contain one or more carriers, excipients or adjuvants, which are typically included in such composition or products in order to aid in their manufacture, storage, application or performance.

In some embodiments, said composition may contain a carrier material that may be hydrophobic or hydrophilic, solid or liquid depending on the nature of the composition or product and the use for which it is intended. Hydrophobic carrier materials include silicones, that is, polyorganosiloxanes. Such materials may be cyclic or linear, examples include Dow Corning silicone fluids 344, 345, 244, 245, 246, 556, and the 200 series; Union Carbide Corporation Silicones 7207 and 7158; and General Electric silicone SF1202. Alternatively, or additionally, non-silicone hydrophobic materials may be used. Such materials include mineral oils, hydrogenated polyisobutene, polydecene, paraffins, isoparaffins of at least 10 carbon atoms, aliphatic or aromatic ester oils (e.g. isopropyl myristate, lauryl myristate, isopropyl palmitate, diisopropyl sebecate, diisopropyl adipate, or C₈ to C₁₋₈ alkyl benzoates), and polyglycol ethers, for example polyglycol butanol ethers. Hydrophilic carrier materials, for example water, may also be employed. Water can be distilled, deionized, or tap water. Water, containing a small amount of low molecular weight monohydric alcohols, e.g., ethanol, methanol, and isopropanol, or polyols, such as ethylene glycol and propylene glycol, can also be useful. Organic solvents might also be employed. They include organic solvents that are aliphatic alcohols, for example monohydric or polyhydric alcohols having 2 to 8 carbon atoms, and polyglycol ethers, such as oligoglycol ethers having 2 to 5 repeat units. Examples include dipropylene glycol, glycerol, propylene glycol, butylene glycol, ethanol, propanol, isopropanol, and industrial methylated spirits. Mixtures of carrier materials may also be used. The amount of carrier material employed is dependent on the nature of the composition and can range from 1.0-99.0% by weight, more typically 10.0-95.0% by weight, relative to the total weight of the composition.

Structurants and emulsifiers may also be employed in the composition of the invention. They may be present at levels from 0.01-50.0% by weight, more typically 0.1-10.0% by weight, relative to the total weight of the composition.

The compositions according to the invention may contain solubilizing aids if needed. A suitable solubilizing aid can be a surfactant. Suitable surfactants can be nonionic surfactants, anionic surfactants, cationic surfactants, amphoteric surfactants, zwitterionic surfactants, and mixtures thereof. Non-limiting examples of suitable surfactants can be found described further below. The compositions according to the invention may contain preservatives, pH control agents (such as buffers), chelating agents, defoaming agents, antifoaming agents, antistatic agents, colorants, antioxidants, aesthetic agents such as opacifiers, pearlizers, dyes and mixtures thereof.

The present invention relates also to the use of at least one compound of formula (I) as defined herein (including all embodiments thereof as described herein) or a cosmetic or pharmaceutical composition as defined herein (including all embodiments thereof as described herein) as UV absorbing filter and/or high-energy visible light filters.

Sunlight or ultraviolet radiation in the UV-B range has a wavelength of 290 nm to 320 nm. Ultraviolet rays at a wavelength of 320 nm to 400 nm, are known as UV-A radiation. High-energy visible (HEV) light is a high frequency light in the violet/blue band from 400 nm to 450 nm in the visible spectrum (400 nm-700 nm).

In some embodiments, compound of formula (I) as defined herein (including all embodiments thereof as described herein) having maximal absorbance around 330 nm, can provide UVB protection and decrease the visible component of absorption allowing for a filter that appears with less coloration.

In some embodiments, compound of formula (I) as defined herein (including all embodiments thereof as described herein) can be susceptible to hydrolysis catalyzed by esterases and proteases present in human tissue. This can free the aniline derivative component, alcohols, and carboxylic acids, which can then be further metabolized. Therefore, in some embodiments, compound of formula (I) as defined herein (including all embodiments thereof as described herein) are particularly suitable as non-persistent, biodegradable, and bio-safe UV and/or high-energy visible light filters.

In some embodiments, compounds of formula (I) as defined herein (including all embodiments thereof as described herein) having maximal absorbance around 360 nm can provide UVA protection while not preventing moderate amounts of UVB to pass through to skin to allow for natural vitamin D synthesis.

In some embodiments, a compound of formula (I) as defined herein (including all embodiments thereof as described herein) or a cosmetic or pharmaceutical composition as defined herein (including all embodiments thereof as described herein) can provide UVB protection.

In some embodiments, a compound of formula (I) as defined herein (including all embodiments thereof as described herein) or a cosmetic or pharmaceutical composition as defined herein (including all embodiments thereof as described herein) can provide UVA protection.

In some embodiments, a compound of formula (I) as defined herein (including all embodiments thereof as described herein) or a cosmetic or pharmaceutical composition as defined herein (including all embodiments thereof as described herein) can provide UVA and UVB (broad spectrum) protection.

In some embodiments, a compound of formula (I) as defined herein (including all embodiments thereof as described herein) or a cosmetic or pharmaceutical composition as defined herein (including all embodiments thereof as described herein) can provide high-energy visible light protection.

In some embodiments, the compound of formula (I) as defined herein (including all embodiments thereof as described herein) or the cosmetic as defined herein (including all embodiments thereof as described herein) can be used as an UV and/or high-energy visible light absorbing filter in a consumer product.

The present invention also relates to the use of at least one compound of formula (I) as defined herein (including all embodiments thereof as described herein) or at least one cosmetic or pharmaceutical composition as defined herein (including all embodiments thereof as described herein) for protecting from UV and/or high-energy visible radiations, preferably to protect a substrate against UV and/or high-energy visible radiations. The use is preferably non-therapeutic, more preferably cosmetic.

The present invention also encompasses a method of protecting a substrate from UV and/or high-energy visible radiation, comprising the step of applying a compound of formula (I) as defined herein (including all embodiments thereof as described herein) or a cosmetic or pharmaceutical composition as defined herein (including all embodiments thereof as described herein) onto said substrate. In some embodiments, the substrate is skin.

The present invention also relates to the use of a compound of formula (I) as defined herein (including all embodiments thereof as described herein) or a cosmetic or pharmaceutical composition as defined herein (including all embodiments thereof as described herein) to protect skin against UV and/or high-energy visible radiations. In connection with this embodiment, the compound of formula (I) as defined herein is used for protecting the skin from UV and/or high-energy visible radiation, preferably for protecting human skin from UV and/or high-energy visible radiation. Preferably, the use is non-therapeutic.

In another aspect, the present invention relates to at least one compound as defined herein (including all embodiments thereof as described herein) or the cosmetic or pharmaceutical composition as defined herein (including all embodiments thereof as described herein) for use to protect skin against UV and/or high-energy visible radiations.

The present invention also encompasses a method of protecting skin from UV and/or high-energy visible radiation, comprising the step of applying a compound of formula (I) as defined herein (including all embodiments thereof as described herein) or a cosmetic or pharmaceutical composition as defined herein (including all embodiments thereof as described herein) onto skin. In a preferred embodiment, the compound of formula (I) as defined herein is used for protecting human skin from UV and/or high-energy visible radiation. The method can be therapeutic or non-therapeutic in nature.

Suitable human skin includes facial skin, hands, arms, feet, legs, neck, chest, back including the lower back, and scalp. Of interest is facial skin including the forehead, perioral, chin, periorbital, nose, and/or cheeks. The compound or composition may be applied and left on the skin for a sufficient time or may be applied repeatedly a sufficient number of times.

In some embodiments, because of the molecular weight of the compound of formula (I), they are more likely to remain on the surface of the skin, and they are less likely to be absorbed into tissue and enter circulation in the body. In some embodiments, unlike many compounds approved for use as topical UV filters by the FDA, the biodegradation of the compound of formula (I) can produce molecules that are not xenobiotics (thereby not posing a health risk if consumed or absorbed), said molecules being less ecologically damaging to sensitive aquatic life threatened by accumulation of current sunscreen dyes from swimmers.

The present invention also relates to a consumer product comprising at least one compound of formula (I) as defined herein (including all embodiments thereof as described herein). The compounds can be used, inter alia, as active ingredients, in consumer products, and/or as inactive ingredients to photostabilize other ingredients in the formulations of the products.

The present invention further relates to the use of at least one compound of formula (I) as defined herein (including all embodiments thereof as described herein) in a consumer product as a light stabilizer to protect ingredients against photolytic degradation. In some embodiments, the compound of formula (I) as defined herein (including all embodiments thereof as described herein) can be used as an alternative to organic dyes and inorganic particles in consumer product.

They can be used as UV filters in UV-protective packaging, UV-protective filters on plastics or glass, or as UV-protective food preservatives.

The term “photostability” refers to the ability of a UV filter or any other molecule, which is exposed to UV and visible light, to stay stable upon irradiation. In particular, this means that the compound does not undergo a degradation process upon UV radiation.

The term “consumer product” means any article, or component part thereof, produced or distributed for sale to a consumer for use in or around a residence, a school, in recreation, or otherwise. Non-limiting examples of consumer products are body-care products such as cosmetics (body care products), foodstuffs, pharmaceuticals, textiles, household chemicals and lawn and gardening products.

Without the examples being limiting, the consumer products according to the invention may be cosmetic products including both cosmetic formulations and application supports or articles comprising such formulations, such as patches, wipes, nonwoven supports; cosmetic or dermatological products: creams, milks, lotions, balms, sticks, talcs; makeup products; hair products; pharmaceutical products and also medical and paramedical articles such as dressings, patches, prostheses; products for veterinary use, such as animal litters; animal hygiene and/or care products; household products such as laundry care and/or cleaning products (laundry detergents, softeners), washing-up products, products for cleaning and/or maintaining household appliances, products for cleaning and/or maintaining floors, tiles, wood, etc.; sanitary products; textile materials, clothing, fine leather goods such as shoes, soles and products for the maintenance thereof; products resulting from the food-processing industry; products resulting from agriculture; phytosanitary products; products resulting from the wood and paper industry; paints; inks.

The compounds, compositions, products, can be produced or used for human or non-human animal.

In some embodiments, the consumer product can be a cosmetic product. Non-limiting cosmetic products include sunscreens, moisturizers, makeup, lip protection products, shampoos, conditioners, and the like. Preferably the consumer product is a sunscreen.

In some embodiments, the product comprises from about 0.001% to about 50.0% by weight of the compound of formula (I) as defined herein (including all embodiments thereof as described herein), relative to the total weight of the product. In some embodiments, the product can comprise from about 0.001% to about 45.0% by weight, preferably from about 0.001% to about 40.0% by weight, preferably from about 0.01% to about 35.0% by weight, preferably from about 0.01% to about 30.0% by weight, preferably from about 0.1% to about 25.0% by weight of the compound, for example from about 0.1% to about 20.0% by weight, for example from about 0.1% to about 15.0% by weight, for example from about 0.1% to about 10.0% by weight, for example from about 0.1% to about 5.0% by weight, for example from about 0.1% to about 2.5% by weight, relative to the total weight of the product. In some embodiments, the product comprises from about 0.1% to about 0.5% by weight, for example from about 0.1% to about 1.0% by weight, for example from about 0.1% to about 1.5% by weight, for example from about 0.1% to about 2.0% by weight, for example from about 0.1% to about 2.5% by weight, for example from about 0.5% to about 1.0% by weight, for example from about 0.5% to about 1.5% by weight, for example from about 0.5% to about 2.0% by weight, for example from about 0.5% to about 2.5% by weight, for example from about 1.0% to about 1.5% by weight, for example from about 1.0% to about 2.0% by weight, for example from about 1.0% to about 2.5% by weight, for example from about 1.5% to about 2.0% by weight, for example from about 1.5% to about 2.5% by weight, or for example from about 2.0% to about 2.5% by weight, relative to the total weight of the product.

In some embodiments, the product provides UVA and/or UVB protection centered at from about 350 nm to about 370 nm. In some embodiments, the protection is centered at from about 300 nm to about 320 nm, for example from about 310 nm to about 330 nm, for example from about 320 nm to about 340 nm, for example from about 330 nm to about 350 nm, for example from about 340 nm to about 360 nm, for example from about 360 nm to about 380 nm, for example from about 370 nm to about 390 nm, for example from about 380 nm to about 400 nm, or for example from about 390 nm to about 410 nm.

In some embodiments, UV transmission through the product is at most 10.0% at each wavelength under 390 nm. In some embodiments, UV transmission through the packaging material is at most 20.0%, for example at most 25.0%, for example at most 30.0%, for example at most 35.0%, for example at most 40.0%, for example at most 45.0%, for example at most 50.0%, for example at most 55.0%, for example at most 60.0%, for example at most 65.0%, for example at most 70.0%, for example at most 75.0%, for example at most 80.0%, for example at most 85.0%, or for example at most 90.0% at each wavelength under 390 nm.

The present invention also provides a cosmetic or a pharmaceutic product comprising at least one compound of formula (I) as defined herein (including all embodiments thereof as described herein) or a cosmetic or pharmaceutical composition as defined herein (including all embodiments thereof as described herein).

In some embodiments, the cosmetic or pharmaceutical product comprises from about 0.001% to about 50.0% by weight of the compound of formula (I) as defined herein (including all embodiments thereof as described herein), relative to the total weight of the product. In some embodiments, the cosmetic product comprises from about 0.001% to about 40.0% by weight of the compound, for example from about 0.01% to about 30.0% by weight of the compound, for example from about 0.1% to about 25.0% by weight of the compound, for example from about 0.1% to about 20.0% by weight, for example from about 0.1% to about 15.0% by weight, for example from about 0.1% to about 10.0% by weight, for example from about 0.1% to about 5.0% by weight, for example from about 0.1% to about 2.5% by weight, relative to the total weight of the product. In some embodiments, the product comprises from about 0.1% to about 0.5% by weight, for example from about 0.1% to about 1.0% by weight, for example from about 0.1% to about 1.5% by weight, for example from about 0.1% to about 2.0% by weight, for example from about 0.1% to about 2.5% by weight, for example from about 0.5% to about 1.0% by weight, for example from about 0.5% to about 1.5% by weight, for example from about 0.5% to about 2.0% by weight, for example from about 0.5% to about 2.5% by weight, for example from about 1.0% to about 1.5% by weight, for example from about 1.0% to about 2.0% by weight, for example from about 1.0% to about 2.5% by weight, for example from about 1.5% to about 2.0% by weight, for example from about 1.5% to about 2.5% by weight, or for example from about 2.0% to about 2.5% by weight, relative to the total weight of the product.

The cosmetic or pharmaceutic product may comprise the herein defined compound in combination with a cosmetically or pharmaceutically acceptable carrier, diluent and/or excipient.

In some embodiments, the cosmetic product, can be in the form of a cream, a lotion, a light ointment, an oil, a gel, an emulsion, a suspension, a mousse, an aerosol, a powder, a pack, a sheet, a patch, a stick, or a cake. The packaging could be a patch, bottle, tube, roll-ball applicator, propellant driven aerosol device, squeeze container or lidded jar. Suitable cosmetic product includes but is not limited to a sunscreen, a moisturizer, makeup, a lip protection product, a shampoo, a conditioner, and the like.

The present invention also relates to the use of at least one compound of formula (I) as defined herein (including all embodiments thereof as described herein) or at least one cosmetic or pharmaceutical composition as defined herein (including all embodiments thereof as described herein) as an UV and/or high-energy visible light absorbing filter in a cosmetic product, preferably in a sunscreen.

The term “sunscreen composition” or “sunscreen” or “skin-care product” refers to any topical product, which reflects and/or absorbs certain parts of UV radiation. Thus, the term “sunscreen composition” is to be understood as not only including sunscreen compositions, but also any cosmetic compositions or product that provide UV protection. In some embodiments, the sunscreen is characterized by photostability and nonradiative relaxation that provides UV and/or high-energy visible light protection.

The compounds, compositions, and products can be administered by different administration forms. They can be preferably in the form of preparations for topical application to the skin.

Furthermore, in connection with the above embodiments, it is to be understood that the cosmetic product (e.g., a sunscreen composition) or pharmaceutical product may further comprise at least one additive.

In some embodiments, the product can further comprise a product vehicle. The vehicle can act as diluent or dispersant for the ingredients of the product. The vehicle may be aqueous-based, anhydrous, or an emulsion, such as water-in-oil or oil-in-water emulsion. For example, the vehicle is selected from the group comprising water, a solvent or diluent, an oil, and a combination thereof. In addition to water, organic solvents can also be used such as alkanols like ethyl and isopropyl alcohol, and propane diol. Other suitable organic solvents include ester oils like isopropyl myristate, cetyl myristate, 2-octyldodecyl myristate, avocado oil, almond oil, olive oil and neopentylglycol dicaprate. Fatty acids having from 10 to 30 carbon atoms may also be included as carriers. Examples of such fatty acids include pelargonic, lauric, myristic, palmitic, stearic, isostearic, oleic, linoleic, arachidic, behenic or erucic acid, and mixtures thereof.

In some embodiments, the product further comprises an oil component. Non-limiting examples of oil components include hydrocarbon oils, higher fatty acids, higher alcohols, synthetic esters, silicone oils, liquid fats and oils, solid fats and oils, and waxes. Non-limiting examples of the hydrocarbon oils include liquid petrolatum, ozocerite, squalane, pristane, paraffin, ceresin, squalene, petrolatum, and microcrystalline wax. Non-limiting examples of the higher fatty acids include lauric acid, myristic acid, palmitic acid, stearic acid, behenic acid, oleic acid, undecylenic acid, tall oil, isostearic acid, linolic acid, linoleic acid, eicosapentaenoic acid (EPA), and docosahexaenoic acid (DHA). Non-limiting examples of the higher alcohols include straight chain alcohols (for example, lauryl alcohol, cetyl alcohol, stearyl alcohol, behenyl alcohol, myristyl alcohol, oleyl alcohol, and cetostearyl alcohol) and branched chain ethyl alcohols (for example, mono stearyl glyceryl ether (batyl alcohol), 2-decyltetradecynol, lanolin alcohol, cholesterol, phytosterol, hexyl dodecanol, isostearyl alcohol, and octyl dodecanol). Non-limiting examples of the synthetic ester oils include octyl octanoate, nonyl nonanoate, cetyl octanoate, isopropyl myristate, octyl dodecyl myristate, isopropyl palmitate, butyl stearate, hexyl laurate, myristyl myristate, decyl oleate, dimethyl hexyl decyl octanoate, cetyl lactate, myristyl lactate, lanolin acetate, isocetyl stearate, isocetyl isostearate, cholesteryl 12-hydroxystearate, di-2-ethylene glycol ethylhexanoate, dipentaerythritol fatty acid ester, N-alkylene glycol monoisostearate, neopentyl glycol dicaprate, tripropylene glycol pivalate, diisostearyl malate, glyceryl di-2-heptylundecanoate, glyceryl diisostearate, trimethylolpropane tri-2-ethyl hexanoate, trimethylolpropane triisostearate, tetra-2-pentaerythritol ethylhexanoate, glyceryl tri-2-ethylhexanoate, glyceryl trioctanoate, glyceryl triisopalmitate, trimethylolpropane triisostearate, cetyl 2-ethyl hexanoate 2-ethylhexyl palmitate, glyceryl trimyristate, tri-2-heptyl undecanoic acid glyceride, methyl castor oil fatty acid, oleyl oleate, aceto glyceride, 2-heptylundecyl palmitate, diisobutyl adipate, 2-octyldodecyl N-lauroyl-L-glutamate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate. Non-limiting examples of the silicone oils include chain polysiloxanes (for example, dimethylpolysiloxane, methylphenyl polysiloxane, and diphenyl polysiloxane); ring polysiloxanes (for example, octamethylcyclotetrasiloxane, decamethyl cyclopentasiloxane, and dodecamethyl cyclohexasiloxane), silicone resins having a three-dimensional network structure, silicone rubbers, various modified polysiloxanes (amino-modified polysiloxane, polyether-modified polysiloxane, alkyl-modified polysiloxane, and fluorine-modified polysiloxane), and acryl silicones. Non-limiting examples of the liquid fats and oils include avocado oil, tsubaki oil, turtle fatty acid, macademia nut oil, corn oil, mink oil, olive oil, rapeseed oil, egg yolk oil, sesame oil, persic oil, wheat germ oil, sasanqua oil, castor oil, linseed oil, safflower oil, cotton seed oil, perilla oil, soybean oil, peanut oil, tea seed oil, Japanese nutmeg oil, rice bran oil, Chinese gimlet oil, Japanese gimlet oil, jojoba oil, germ oil, and triglycerides. Non-limiting examples of the solid fats and oils include cacao butter, coconut oil, horse fat, hydrogenated coconut oil, palm oil, beef tallow, mutton tallow, hydrogenated beef tallow, palm kernel oil, lard, beef bone fat, Japanese core wax nucleus oil, hydrogenated oil, neatsfoot oil, Japanese core wax, and hydrogenated castor oil. Non-limiting examples of the waxes include beeswax, candelilla wax, cotton wax, carnauba wax, bayberry wax, tree wax, whale wax, montan wax, bran wax, lanolin, kapok wax, lanolin acetate, liquid lanolin, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, and POE hydrogenated lanolin ethyl alcohol ether.

Humectants of the polyhydric alcohol type may also be employed. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol, and mixtures thereof. For best results, the humectant is preferably propylene glycol or sodium hyaluronate. Other humectants which may be used include hydroxyethyl urea.

In some embodiments, the product further comprises one or more moisturizers. Moisturizers include, but are not limited to, C₁₋₂₀alkyl esters of fatty acids, C₁₀₋₂₂ fatty acids (i.e., stearyl, palmityl, lauryl, myristyl acids), C₁₀₋₂₂ fatty alcohols (stearyl, palmityl, lauryl, myristyl, oleyl alcohols), and C₁₀₋₂₂ fatty alcohol ethers, C₁₆₋₂₂alkanoic triglycerides (e.g., sunflower seed oil), sterols such as cholesterol and soy sterol, silicones (e.g., dimethicone), petroleum jelly, and mineral oils.

Typical thickeners include cross-linked acrylates (e.g., Carbopol® 982), hydrophobically-modified acrylates (e.g., Carbopol®1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums.

In some embodiments, the product further comprises one or more emollients. Non-limiting examples of suitable emollients include those agents known for softening the skin which may be selected from hydrocarbons, fatty acids, fatty alcohols and esters. For example, petrolatum is a common hydrocarbon type of emollient conditioning agent. Other agents that may be employed include, but are not limited to, alkyl benzoate, mineral oil, polyolefins such as polydecene, and paraffins, such as isohexadecane. Suitable fatty acids and alcohols which can be used typically have from about 10 to 30 carbon atoms. Oily ester emollients may be those selected from one or more of the following: triglyceride esters, acetoglyceride esters, ethoxylated glycerides, alkenyl or alkyl esters of fatty acids having 10 to 20 carbon atoms (such as isoarachidyl neopentanoate, isononyl isonanonoate, oleyl myristate, oleyl stearate, and oleyl oleate), ether-esters (such as fatty acid esters of ethoxylated fatty alcohols), polyhydric alcohol esters (such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are suitable polyhydric alcohol esters), wax esters (such as beeswax, spermaceti, stearyl stearate and arachidyl behenate), and sterols esters (of which cholesterol fatty acid esters are examples). Additional emollients or hydrophobic agents include, but are not limited to, C₁₂₋₁₅alkyl benzoate, dioctyladipate, octyl stearate, octyidodecanol, hexyl laurate, octyldodecyl neopentanoate, cyclomethicone, dicapryl ether, dimethicone, phenyl trimethicone, isopropyl myristate, capriylic/capric triglycerides, propylene glycol dicaprylate/dicaprate and decyl oleate, cyclomethicones and other silicone derivatives. Non-limiting examples of additional emollients include cetearyl alcohol, isoamyl laurate, glyceryl stearate citrate, glyceryl caprylate, caprylic/capric triglyceride, and cetearyl isononanoate. Other emollients include silicone oils and synthetic esters. Silicone oils suitable for use include cyclic or linear polydimethylsiloxanes containing from 3 to 9, preferably from 4 to 5 silicon atoms. Non-volatile silicone oils useful as emollients include polyalkyl siloxanes, polyalkylaryl siloxanes and polyether siloxane copolymers. Silicone elastomers may also be used.

In some embodiments, the product further comprises a surfactant. The surfactant may be anionic, cationic, ampholytic, or non-ionic. Non-limiting examples of anionic surfactants include fatty acid soaps (for example, sodium laurate and sodium palmitate); higher alkyl sulfuric acid ester salts (for example, sodium lauryl sulfate and potassium lauryl sulfate); alkyl ether sulfuric acid ester salts (for example, POE-triethanolamine lauryl sulfate and sodium POE-lauryl sulfate); N-acyl sarcosinic acids (for example, sodium lauroyl sarcosinate); higher fatty acid amide sulfonic acid salts (for example, sodium N-myristoyl N-methyl taurate, sodium N-cocoyl-N-methyl taurate, and sodium jauroylmethyl taurate); phosphoric ester salts (for example, sodium POE-oleyl ether phosphate and POE stearyl ether phosphoric acid); sulfosuccinates (for example sodium di-2-ethylhexylsulfosuccinate, sodium monolauroyl monoethanol amide polyoxyethylene sulfosuccinate, and sodium lauryl polypropylene glycol sulfosuccinate); alkyl benzene sulfonates (for example, sodium linear dodecyl benzene sulfonate, triethanolamine linear dodecyl benzene sulfonate, and linear dodecyl benzene sulfonic acid); higher fatty acid ester sulfates (for example, hydrogenated coconut oil aliphatic acid glyceryl sodium sulfate); N-acyl glutamates (for example, mono sodium N-lauroylglutamate, disodium N-stearoylglutamate, and sodium N-myristoyl-L-glutamate); sulfated oils (for example, turkey red oil); POE-alkyl ether carboxylic acid; POE-alkyl aryl ether carboxylate; α-olefin sulfonate; higher fatty acid ester sulfonates; sec-alcohol sulfates; higher fatty acid alkyl amide sulfates; sodium lauroyl monoethanolamine succinates; ditriethanolamine N-palmitoylaspartate; and sodium caseinate. Non-limiting examples of cationic surfactants include quaternary ammonium salts such as cetyltrimethylammonium chloride, stearyltrimethylammonium chloride, benenyltrimethylammonium chloride, behenyldimethylhydroxyethylammonium chloride, stearyldimethylbenzylammonium chloride, and cetyltrimethylammonium methylsulfate. Other examples include amide amine compounds such as stearic diethylaminoethylamide, stearic dimethylaminoethylamide, palmitic diethylaminoethylamide, palmitic dimethylaminoethylamide, myristic diethylaminoethylamide, myristic dimethylaminoethylamide, behenic diethylaminoethylamide, behenic dimethylaminoethylamide, stearic diethylaminopropylamide, stearic dimethylaminopropylamide, palmitic diethylaminopropylamide, palmitic dimethylaminopropylamide, myristic diethylaminopropylamide, myristic dimethylaminopropylamide, behenic diethylaminopropylamide, and behenic dimethylaminopropylamide. Non-limiting examples of ampholytic surfactants include imidazoline type ampholytic surfactants (for example, 2-undecyl-1-hydroxyethyl-1-carboxymethyl-4,5-dihydro-2-imidazolium sodium salt and 1-[2-(carboxymethoxy)ethyl]-1-(carboxymethyl)-4,5-dihydro-2-norcocoalkylimidazolium hydroxide disodium salt) and betaine type surfactants (for example, 2-heptadecyl-N-carboxymethyl-N-hydroxyethyl imidazolinium betaine, lauryldimethylarninoacetic acid betaine, alkyl betaine, amide betaine, and sulfobetaine). Non-limiting examples of non-ionic surfactants include glycerol fatty acid esters, ethylene oxide derivatives of glyceryl fatty acid esters, polyglycerol fatty acid esters, propylene glycol fatty acid esters, ethylene oxide derivatives of propylene glycol fatty acid esters, polyethylene glycol fatty acid esters, polyethylene glycol alkyl ethers, polyethylene glycol alkyl phenyl ethers, polyethylene glycol castor oil derivatives, polyethylene glycol hydrogenated castor oil derivatives, polyether-modified silicone and polyglycerin-modified silicone. Further examples include polyoxyethylene-sorbitan fatty acid esters (for example, POE-sorbitan monooleate, POE-sorbitan monostearate, POE-sorbitan monooleate, and POE-sorbitan tetraoleate); POE sorbitol fatty acid esters (for example, POE sorbitol monolaurate, POE-sorbitol monooleate, POE-sorbitolpentaoleate, and POE-sorbitol monostearate); POE-glyceryl fatty acid esters (for example, POE-monooleates such as POE-glyceryl monostearate, POE-glyceryl monoisostearate, and POE glycerin glyceryl triisostearate); POE-fatty acid esters (for example, POE-distearate, POE-monodioleate, and ethylene glycol distearate); POE-alkylethers (for example, POE-lauryl ether, POE-oleyl ether, POE-stearyl ether, POE-behenyl ether, POE 2-octyl dodecyl ether, and POE-cholestanol ether); pluaronics (for example, pluaronic); POE⋅POP-alkylethers (for example, POE⋅POP-cetyl ether, POE⋅POP2-decyl tetradecyl ether, POE⋅POP-monobutyl ether, POE⋅POP-lanolin hydrate, and POE⋅POP glycerin glyceryl ether); tetra POE⋅tetra POP-ethylenediamino condensates (for example, tetronic); POE-castor oil hydrogenated castor oil derivatives (for example, POE-castor oil, POE-hydrogenated castor oil, POE-hydrogenated castor oil monoisostearate, POE-hydrogenated castor oil triisostearate, POE-hydrogenated castor oil monopyroglutamic monoisostearic diester, and POE-hydrogenated castor oil maleic acid); POE-beeswax⋅lanolin derivatives (for example, POE-sorbitol beeswax); alkanol amides (for example, palm oil fatty acid diethanol amide, laurate monoethanolamide, and fatty acid isopropanol amide); POE-propylene glycol fatty acid esters; POE-alkylamines; POE-fatty acid amides; sucrose fatty acid esters; alkyl ethoxydimethylamine oxides; and trioleyl phosphoric acid.

Various other ingredients may also be used. Non-limiting examples of additional ingredients can be coating agent, binder, extender, disintegrator, lubricant, osmotic pressure regulator, pH regulator, dispersant, preservative, stabilizer, antioxidant, colorant, additional ultraviolet absorber, activity enhancer, anti-inflammatory agent, disinfecting agent, perfume, flavor, odor improver, and the like.

Other additional ingredients such as cosmetic ingredients may also be used such as a skin tanning agent, cell activator, cleaner, keratolytic agent, and make-up components (e.g., a makeup base, foundation, face finishing powder, powder, cheek color, rouge, eye makeup, eyebrow pencil, mascara, etc.).

In some aspect, the product has a pH of from about 2.0 to about 11.0, for example from about 3.0 to about 10.5, for example from about 4.0 to about 10.0, for example from about 5.0 to about 9.0. In some embodiments, the product has a pH of from about 6.5 to about 7.5, for example from about 6.0 to about 7.0, for example from about 7.0 to about 8.0, or for example from about 7.5 to about 8.5.

The products can be produced by a routine method combining the compounds as necessary with the carrier, diluent, and/or excipient, and/or other active ingredients, cosmetic ingredients, or pharmacological ingredients.

For example, the product for topical application to the skin can be prepared using ingredients typically added to preparations for topical application, pharmaceutical products for topical application or cosmetic products for the skin such as oils or oily substances (e.g., fats or oils, waxes, higher fatty acids, essential oils, silicone oils, or the like), moisturizers (e.g., glycerol, sorbitol, gelatin, polyethylene glycol, or the like), powders (e.g., chalks, talcs, Fuller's earth, kaolin, starch, rubber, or the like), dye, emulsifier, solubilizer, cleaner, ultraviolet absorber, thickener, medicinal component, perfume, resin, antibacterial and antifungal agent, other plant extracts (e.g., crude drugs, Kampo products, herbs), alcohols, polyvalent alcohols, inorganic acids (e.g., bicarbonate, carbonate, sodium chloride, potassium chloride, sodium sulfate, or the like), organic acids (e.g., succinic acid, glutaric acid, fumaric acid, glutamic acid, malic acid, citric acid, ascorbic acid, or the like), vitamins (e.g., vitamin As, vitamin Es, vitamin Bs, vitamin C, folic acid, or the like), water-soluble polymers, anionic surfactants (e.g., alkylbenzene sulfonate, alkylsulfate, or the like), cationic surfactants (e.g., alkyl quaternary ammonium salt, alkyl dimethyl benzyl ammonium salt, or the like), nonionic surfactants (e.g., polyoxyethylene alkyl ether, polyoxyethylene alkyl phenyl ether, or the like), and amphoteric surfactants (e.g., imidazoline or carbobetaine containing an alkyl group, or the like).

The present invention also relates to the use of at least one compound of formula (I) as defined herein (including all embodiments thereof as described herein) for the protection of plants. In some embodiments, the at least one compound of formula (I) as defined herein (including all embodiments thereof as described herein) can be sprayed upon plants.

The present invention relates to the use of at least one compound of formula (I) as defined herein (including all embodiments thereof as described herein) as a UV and/or high-energy visible light absorbing filter in packaging; preferably food packaging, cosmetics packaging, or medical products packaging.

The present invention also provides a packaging material comprising at least one compound of formula (I) as defined herein (including all embodiments thereof as described herein, or at least one composition comprising at least one compound of formula (I) as defined herein. Preferably, the packaging material is used for food packaging, cosmetics, or medical products.

In some embodiments, the compound of formula (I) as defined herein (including all embodiments thereof as described herein) or the composition can be a coating applied to the surface of the packaging material. In some embodiments, the coating can be applied by spraying a solution comprising the compound or composition onto the surface of the packaging material.

In some embodiments, the compound or composition can be dispersed throughout the packaging material. In some embodiments, the compound or composition can be a dry powder that may be added to a mixture or solution, wherein said mixture or solution is used to form the packaging material.

In some embodiments, the mixture or solution can comprise one or more polymers. In some embodiments, the packaging material comprises a thermoplastic polymer, polyvinyl chloride (PVC), polyethylene, polyethylene terephthalate (PET), low density polyethylene (LDPE), high density polyethylene (HDPE), polystyrene (PS), plastic wrap, aluminum, aluminum foil, laminates and metallized films, tin-free steel, tin-coated steel, ceramics, glass, protein derived plastics (such as protein derived thermoplastics, for example from sources such as wheat, rice, corn, soy, and casein), or combinations thereof.

The term “thermoplastic polymer” encompasses any synthetic polymeric material that exhibits a modification in physical state from solid to liquid upon exposure to sufficiently high temperatures. Non-limiting examples of thermoplastic polymers include polyolefins (i.e., polypropylene, polyethylene, and the like), polyester (i.e., polyethylene terephthalate, and the like), polyamides (i.e., nylon-1,1, nylon-1,2, nylon-6 or nylon-6,6), polystyrenes, polyurethanes, polycarbonates, polyvinyl halides (i.e., polyvinyl chloride and polyvinvyl difluoride, as merely examples), and the like.

In some embodiments, the packaging material comprises at least one cross-linking agent. In some embodiments, non-limiting examples of cross-linking agent can be selected from the group comprising tetraethylorthosilicate, malonic acid, glutaric acid, adipic acid, citric acid, butanetetracarboxylic acid, maleic acid, and combinations thereof.

In some embodiments, the packaging material comprises from about 0.1% to about 25.0% by weight of the compound as defined herein, relative to the total weight of the material. In some embodiments, the packaging material comprises from about 0.1% to about 20.0% by weight, for example from about 0.1% to about 15.0% by weight, for example from about 0.1% to about 10.0% by weight, for example from about 0.1% to about 7.5% by weight, for example from about 0.1% to about 5.0% by weight, for example from about 0.1% to about 2.5% by weight, for example from about 0.5% to about 1.0% by weight, for example from about 0.5% to about 1.5% by weight, for example from about 0.5% to about 2.5% by weight, for example from about 0.5% to about 5.0% by weight, for example from about 0.5% to about 7.5% by weight, for example from about 0.5% to about 10.0% by weight, for example from about 1.0% to about 1.5% by weight, for example from about 1.0% to about 2.5% by weight, for example from about 1.0% to about 5.0% by weight, for example from about 1.0% to about 7.5% by weight, for example from about 1.0% to about 10.0% by weight, for example from about 1.5% to about 2.5% by weight, for example from about 1.5% to about 5.0% by weight, for example from about 1.5% to about 7.5% by weight, for example from about 1.5% to about 10.0% by weight, for example from about 2.5% to about 5.0% by weight, for example from about 2.5% to about 7.5% by weight, for example from about 2.5% to about 10.0% by weight, for example from about 5.0% to about 7.5% by weight, for example from about 5.0% to about 10.0%, or from about 7.5% to about 10.0% of the compound or composition, relative to the total weight of the packaging material. In some embodiments, the packaging material comprises from about 10.0% to about 25.0% by weight, for example from about 12.5% to about 25.0% by weight, for example from about 15.0% to about 25.0% by weight, for example from about 17.5% to about 25.0% by weight, for example from about 20.0% to about 25.0% by weight, for example from about 10.0% to about 20.0% by weight, for example from about 12.5% to about 20.0% by weight, for example from about 15.0% to about 20.0% by weight, for example from about 17.5% to about 20.0% by weight, for example from about 10.0% to about 17.5% by weight, for example from about 12.5% to about 17.5% by weight, for example from about 15.0% to about 17.5% by weight, for example from about 10.0% to about 15.0% by weight, for example from about 12.5% to about 15.0% by weight, or for example from about 10.0% to about 12.5% by weight of the compound or composition, relative to the total weight of the packaging material. In some embodiments, the packaging material comprises from about 1.0% to about 12.5% by weight, for example from about 1.0% to about 15.0% by weight, for example from about 1.0% to about 17.5% by weight, for example from about 1.0% to about 20.0% by weight, for example from about 1.0% to about 25.0% by weight, for example from about 5.0% to about 12.5% by weight, for example from about 5.0% to about 15.0% by weight, for example from about 5.0% to about 17.5% by weight, for example from about 5.0% to about 20.0% by weight, for example from about 5.0% to about 25.0% by weight, for example from about 7.5% to about 12.5% by weight, for example from about 7.5% to about 15.0% by weight, for example from about 7.5% to about 17.5% by weight, for example from about 7.5% to about 20.0% by weight, for example from about 7.5% to about 25% by weight of the compound or composition, relative to the total weight of the packaging material.

In some embodiments, the thickness of the packaging material is from about 0.01 mm to about 0.1 mm, for example from about 0.01 mm to about 0.5 mm, for example from about 0.01 mm to about 1 mm, for example from about 0.01 mm to about 2.5 mm, for example from about 0.01 mm to about 5 mm, for example from about 0.01 mm to about 10 mm, for example from about 0.1 mm to about 0.5 mm, for example from about 0.1 mm to about 1 mm, for example from about 0.1 mm to about 2.5 mm, for example from about 0.1 mm to about 5 mm, for example from about 0.1 mm to about 10 mm, for example from about 0.5 mm to about 1 mm, for example from about 0.5 mm to about 2.5 mm, for example from about 0.5 mm to about 5 mm, for example from about 0.5 mm to about 10 mm, for example from about 1 mm to about 2.5 mm, for example from about 1 mm to about 5 mm, for example from about 1 mm to about 10 mm, for example from about 2.5 mm to about 5 mm, for example from about 2.5 mm to about 10 mm, or for example from about 5 mm to about 10 mm.

In some embodiments, UV transmission through the packaging material is at most 10.0% at each wavelength under 390 nm. In some embodiments, UV transmission through the packaging material is at most 20.0%, for example at most 25.0%, for example at most 30.0%, for example at most 35.0%, for example at most 40.0%, for example at most 45.0%, for example at most 50.0%, for example at most 55.0%, for example at most 60.0%, for example at most 65.0%, or for example at most 70.0% at each wavelength under 390 nm.

In some embodiments, the compound can provide from about 12 to 24 months stability to a packaged product.

In some embodiments, the packaging material can be transparent or translucent. In some embodiments, the packaging material can be very low in color. In some embodiments, the packaging material does not exhibit a b* value (indicating a degree of yellowing in this instance) above 2.5 on the CieLab scale.

In some embodiments, the compound as defined herein can exhibit low migratory levels when incorporated into a packaging material comprising a polymeric material. In some embodiments, the compound as defined herein can exhibit low migratory levels due to the high hydrophilicity of the compound. In some embodiment, the compound as defined herein can exhibit low migratory levels due to the high molecular weight of the compound.

In some embodiments, the packaging material can be manufactured in the form of a bottle, a storage container, a sheet, a film, a fiber, a plaque, a hose, a tube, or a syringe. Included within this list would be polyester, polystyrene and other like clear resinous materials in sheet form which are present within windows for strength and resiliency functions. In such an instance, the low-color UV absorbers of this invention would provide or contribute to excellent UV protection for contents with target packaging articles (such as bottles, containers, and the like) or persons located indoors (such as within houses, buildings, cars, and the like, comprising windows with such additives included therein).

The compounds defined herein, can be typically added to such packaging material during injection molding (or other type of molding, such as blow molding, extrusion), including, and without limitation, by mixing the compounds with resin pellets and melting the entire coated pellets, or through a masterbatch melting step while the resin and compound are pre-mixed and incorporated together in pellet form. Such resins include, without limitation, polyolefins, polyesters, polyamides, polyurethanes, polycarbonates, and other resins, such as those disclosed within U.S. Pat. Nos. 4,640,690 4,507,407, herein incorporated by reference. The resins including the compound, can be formed through any number of various extrusion techniques, such as those disclosed in the aforementioned U.S. patents.

In some embodiment, the compounds as defined herein have antioxidant activity against reactive oxygen species, especially hydroxyl radical species, as demonstrated in the hydroxyl oxygen radical absorbance capacity (HORAC) assay (expressed as mole gallic acid equivalent/mole of sample).

These surprising antioxidant effects, in combination with the other known benefits of the compounds individually, can be employed advantageously in the herein described compositions and consumer products.

Therefore, the present invention also encompasses the use of compound of formula (I) as defined herein (including all embodiments thereof as described herein) as antioxidant.

The present invention also encompasses a method to protect a composition from oxidation or to impart antioxidant properties to a composition, comprising adding at least one compound of formula (I) as defined herein (including all embodiments thereof as described herein) to said composition.

The following examples are provided for the purpose of illustrating the present invention and by no means should be interpreted to limit the scope of the present invention.

### EXAMPLES

TABLE 1
Structures of compounds of the invention and their respective codes

| Compound | Structure |
|---|---|
| 1 | 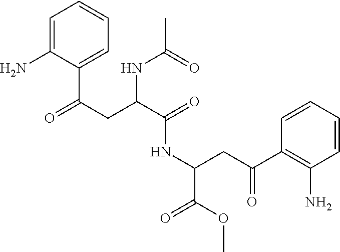 [SMILES: COC(=O)C(CC(=O)c1ccccc1N)NC(=O)C(CC(=O)c1ccccc1N)NC(C)=O] |
| 2 | 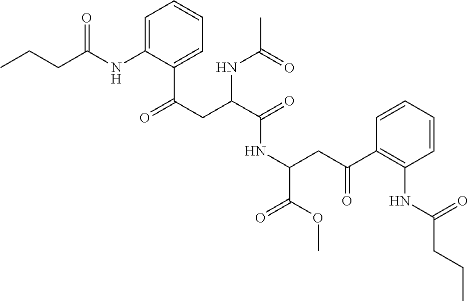 [SMILES: CCCC(=O)Nc1ccccc1C(=O)CC(NC(C)=O)C(=O)NC(CC(=O)c1ccccc1NC(=O)CCC)C(=O)OC] |
| 3 | 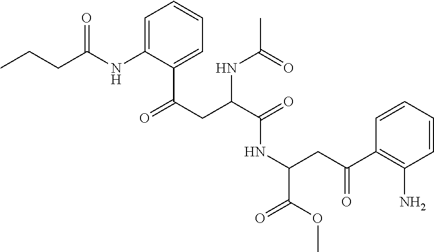 [SMILES: CCCC(=O)Nc1ccccc1C(=O)CC(NC(C)=O)C(=O)NC(CC(=O)c1ccccc1N)C(=O)OC] |
| 4 | 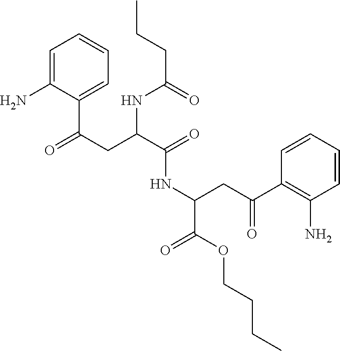 [SMILES: CCCCOC(=O)C(CC(=O)c1ccccc1N)NC(=O)C(CC(=O)c1ccccc1N)NC(=O)CCC] |
| 5 | 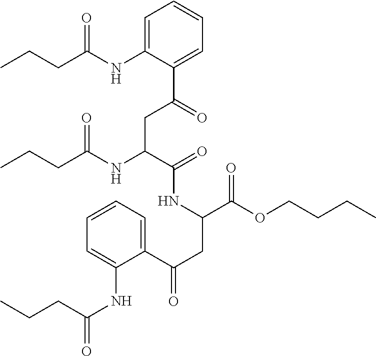 [SMILES: CCCCOC(=O)C(CC(=O)c1ccccc1NC(=O)CCC)NC(=O)C(CC(=O)c1ccccc1NC(=O)CCC)NC(=O)CCC] |
| 6 | 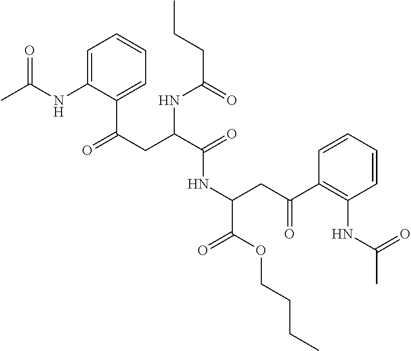 [SMILES: CCCCOC(=O)C(CC(=O)c1ccccc1NC(C)=O)NC(=O)C(CC(=O)c1ccccc1NC(C)=O)NC(=O)CCC] |
| 7 | 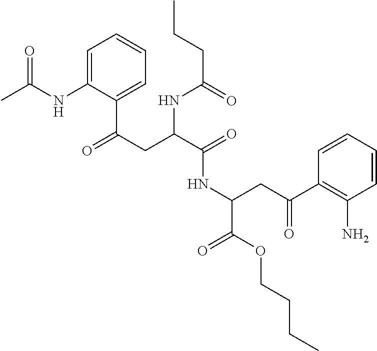 [SMILES: CCCCOC(=O)C(CC(=O)c1ccccc1N)NC(=O)C(CC(=O)c1ccccc1NC(C)=O)NC(=O)CCC] |
| 8 | 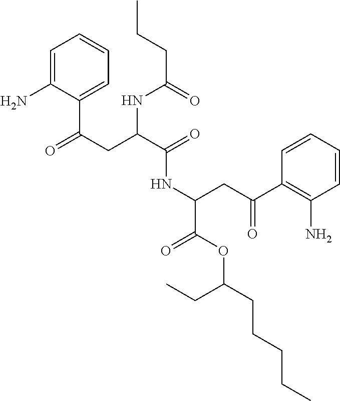 [SMILES: CCCCCC(CC)OC(=O)C(CC(=O)c1ccccc1N)NC(=O)C(CC(=O)c1ccccc1N)NC(=O)CCC] |
| 9 | 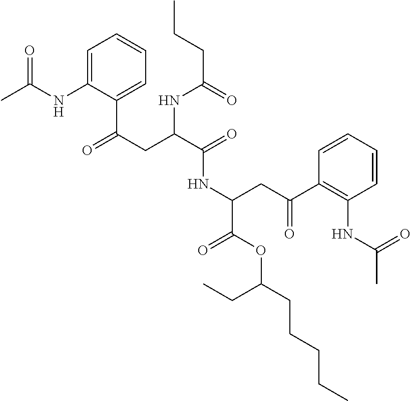 [SMILES: CCCCCC(CC)OC(=O)C(CC(=O)c1ccccc1NC(C)=O)NC(=O)C(CC(=O)c1ccccc1NC(C)=O)NC(=O)CCC] |
| 10 | 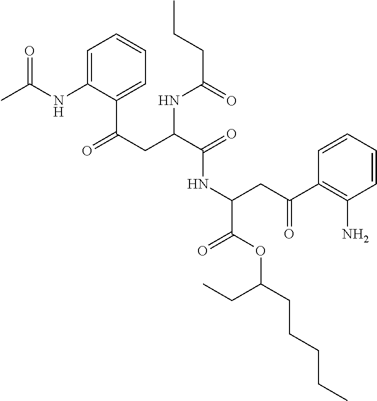 [SMILES: CCCCCC(CC)OC(=O)C(CC(=O)c1ccccc1N)NC(=O)C(CC(=O)c1ccccc1NC(C)=O)NC(=O)CCC] |
| 11 | 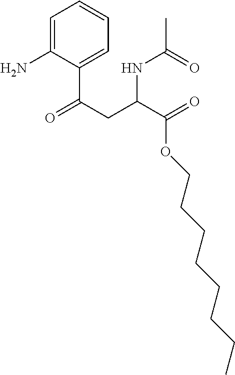 [SMILES: CCCCCCCCOC(=O)C(CC(=O)c1ccccc1N)NC(C)=O] |
| 12 | 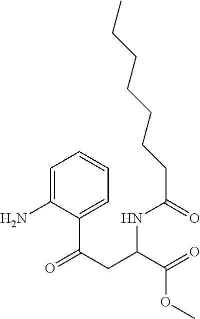 [SMILES: CCCCCCCC(=O)NC(CC(=O)c1ccccc1N)C(=O)OC] |
| 13 | 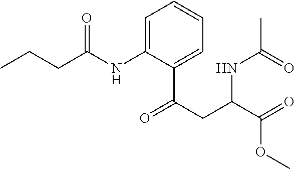 [SMILES: CCCC(=O)Nc1ccccc1C(=O)CC(NC(C)=O)C(=O)OC] |
| 14 | 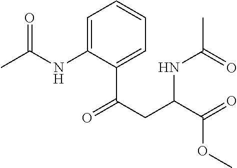 [SMILES: COC(=O)C(CC(=O)c1ccccc1NC(C)=O)NC(C)=O] |
| 15 | 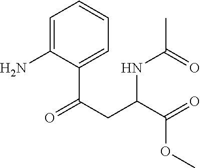 [SMILES: COC(=O)C(CC(=O)c1ccccc1N)NC(C)=O] |
| 16 | 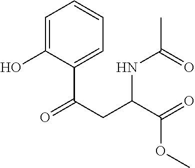 [SMILES: COC(=O)C(CC(=O)c1ccccc1O)NC(C)=O] |
| 17 | 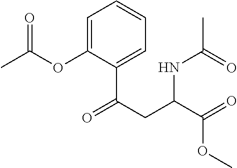 [SMILES: COC(=O)C(CC(=O)c1ccccc1OC(C)=O)NC(C)=O] |
| 18 | 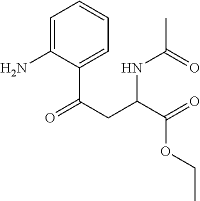 [SMILES: CCOC(=O)C(CC(=O)c1ccccc1N)NC(C)=O] |
| 19 | 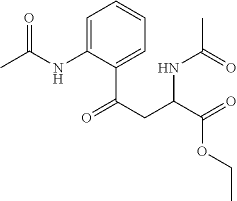 [SMILES: CCOC(=O)C(CC(=O)c1ccccc1NC(C)=O)NC(C)=O] |
| 20 | 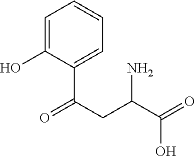 [SMILES: NC(CC(=O)c1ccccc1O)C(=O)O] |
| 21 | 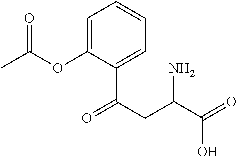 [SMILES: CC(=O)Oc1ccccc1C(=O)CC(N)C(=O)O] |
| 22 | 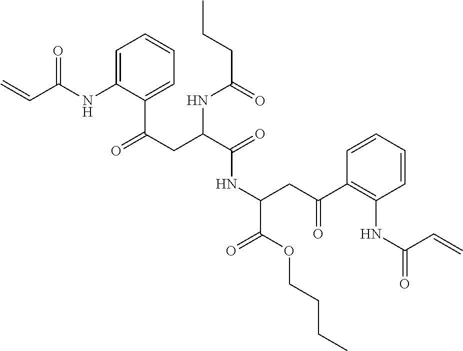 [SMILES: C=CC(=O)Nc1ccccc1C(=O)CC(NC(=O)CCC)C(=O)NC(CC(=O)c1ccccc1NC(=O)C=C)C(=O)OCCCC] |
| 23 | 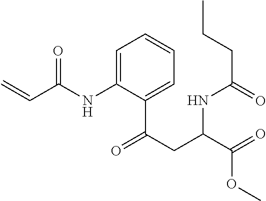 [SMILES: C=CC(=O)Nc1ccccc1C(=O)CC(NC(=O)CCC)C(=O)OC] |
| 24 | 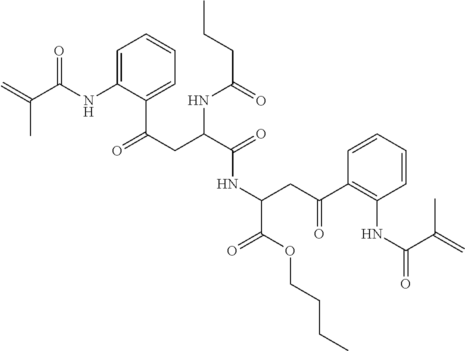 [SMILES: C=C(C)C(=O)Nc1ccccc1C(=O)CC(NC(=O)CCC)C(=O)NC(CC(=O)c1ccccc1NC(=O)C(=C)C)C(=O)OCCCC] |
| 25 | 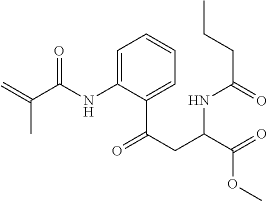 [SMILES: C=C(C)C(=O)Nc1ccccc1C(=O)CC(NC(=O)CCC)C(=O)OC] |
| 26 | 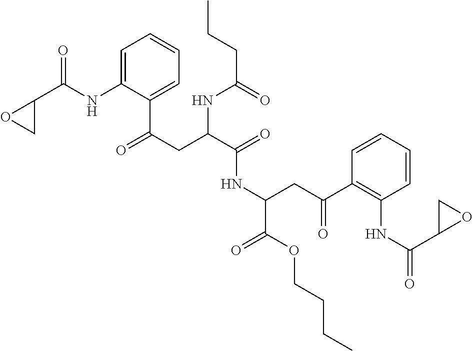 [SMILES: CCCCOC(=O)C(CC(=O)c1ccccc1NC(=O)C1CO1)NC(=O)C(CC(=O)c1ccccc1NC(=O)C1CO1)NC(=O)CCC] |
| 27 | 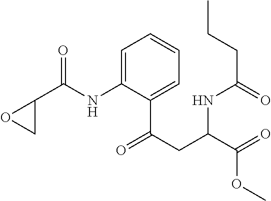 [SMILES: CCCC(=O)NC(CC(=O)c1ccccc1NC(=O)C1CO1)C(=O)OC] |
| 28 | 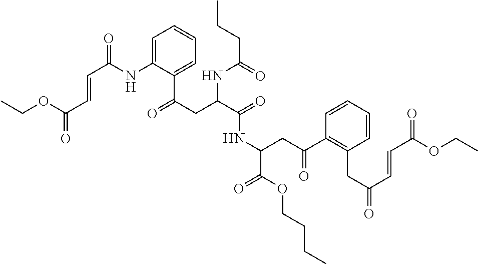 [SMILES: CCCCOC(=O)C(CC(=O)c1ccccc1CC(=O)/C=C/C(=O)OCC)NC(=O)C(CC(=O)c1ccccc1NC(=O)/C=C/C(=O)OCC)NC(=O)CCC] |
| 29 | 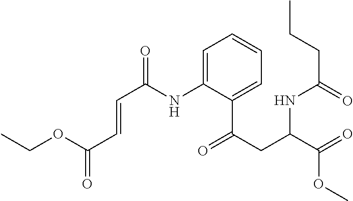 [SMILES: CCCC(=O)NC(CC(=O)c1ccccc1NC(=O)/C=C/C(=O)OCC)C(=O)OC] |
| 30 | 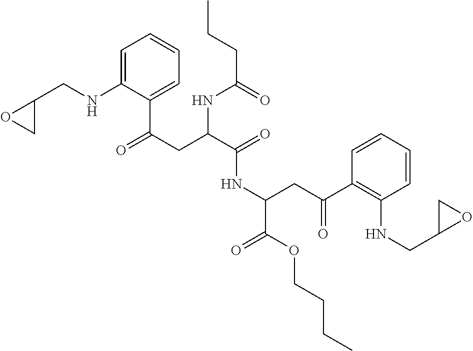 [SMILES: CCCCOC(=O)C(CC(=O)c1ccccc1NCC1CO1)NC(=O)C(CC(=O)c1ccccc1NCC1CO1)NC(=O)CCC] |
| 31 | 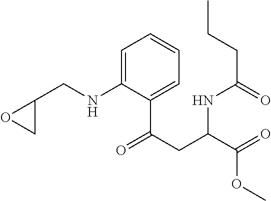 [SMILES: CCCC(=O)NC(CC(=O)c1ccccc1NCC1CO1)C(=O)OC] |
| 32 | 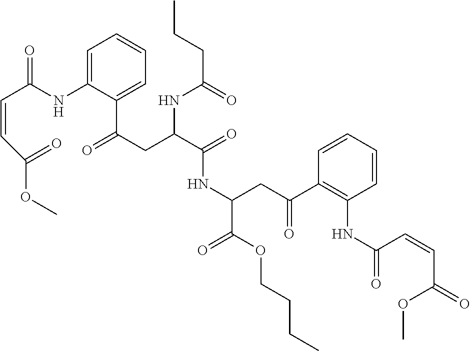 [SMILES: CCCCOC(=O)C(CC(=O)c1ccccc1NC(=O)/C=C\C(=O)OC)NC(=O)C(CC(=O)c1ccccc1NC(=O)/C=C\C(=O)OC)NC(=O)CCC] |
| 33 | 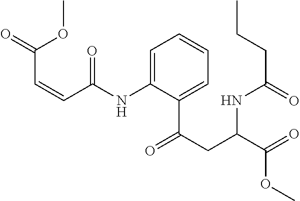 [SMILES: CCCC(=O)NC(CC(=O)c1ccccc1NC(=O)/C=C\C(=O)OC)C(=O)OC] |
| 34 | 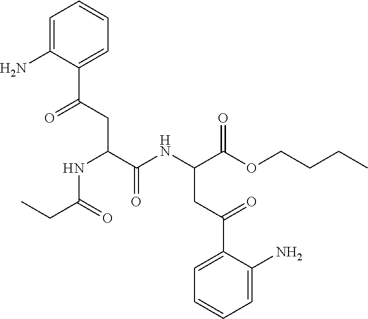 [SMILES: CCCCOC(=O)C(CC(=O)c1ccccc1N)NC(=O)C(CC(=O)c1ccccc1N)NC(=O)CC] |
| 35 | 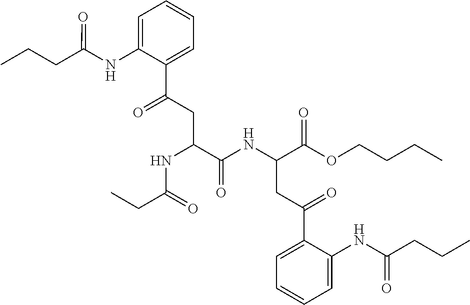 [SMILES: CCCCOC(=O)C(CC(=O)c1ccccc1NC(=O)CCC)NC(=O)C(CC(=O)c1ccccc1NC(=O)CCC)NC(=O)CC] |
| 36 | 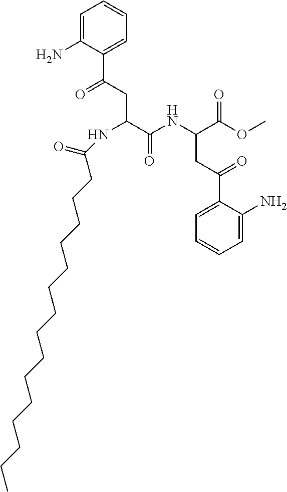 [SMILES: CCCCCCCCCCCCCCCC(=O)NC(CC(=O)c1ccccc1N)C(=O)NC(CC(=O)c1ccccc1N)C(=O)OC] |
| 37 | 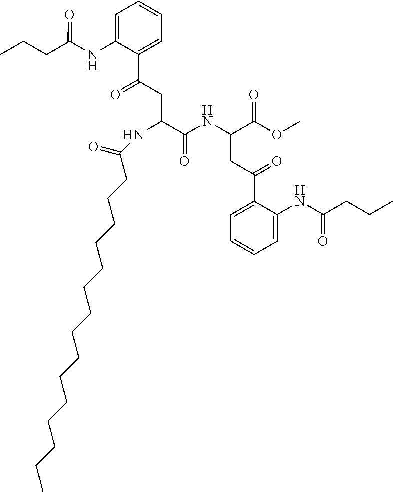 [SMILES: CCCCCCCCCCCCCCCC(=O)NC(CC(=O)c1ccccc1NC(=O)CCC)C(=O)NC(CC(=O)c1ccccc1NC(=O)CCC)C(=O)OC] |
| 38 | 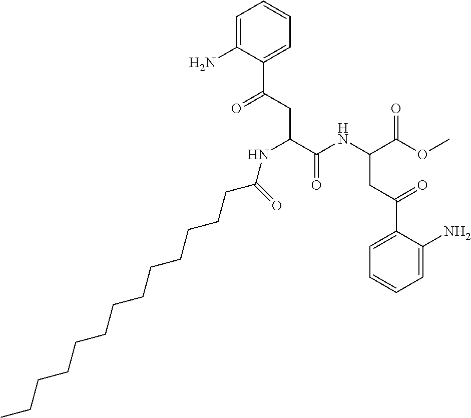 [SMILES: CCCCCCCCCCCCCC(=O)NC(CC(=O)c1ccccc1N)C(=O)NC(CC(=O)c1ccccc1N)C(=O)OC] |
| 39 | 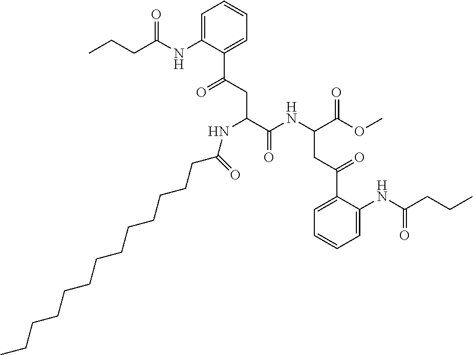 [SMILES: CCCCCCCCCCCCCC(=O)NC(CC(=O)c1ccccc1NC(=O)CCC)C(=O)NC(CC(=O)c1ccccc1NC(=O)CCC)C(=O)OC] |
| 40 | 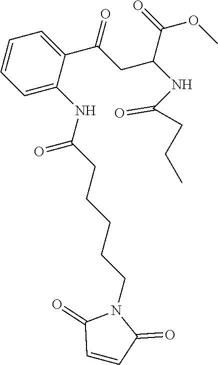 [SMILES: CCCC(=O)NC(CC(=O)c1ccccc1NC(=O)CCCCCN1C(=O)C=CC1=O)C(=O)OC] |
| 41 | 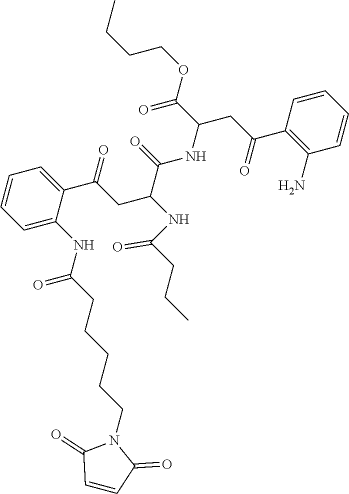 [SMILES: CCCCOC(=O)C(CC(=O)c1ccccc1N)NC(=O)C(CC(=O)c1ccccc1NC(=O)CCCCCN1C(=O)C=CC1=O)NC(=O)CCC] |
| 42 | 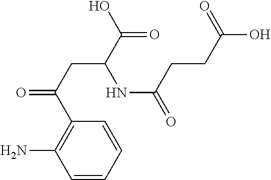 [SMILES: Nc1ccccc1C(=O)CC(NC(=O)CCC(=O)O)C(=O)O] |
| 43 | 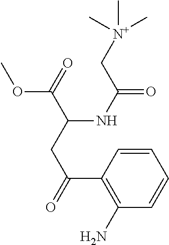 [SMILES: COC(=O)C(CC(=O)c1ccccc1N)NC(=O)C[N+](C)(C)C] |
| 44 | 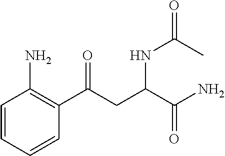 [SMILES: CC(=O)NC(CC(=O)c1ccccc1N)C(N)=O] |
| 45 | 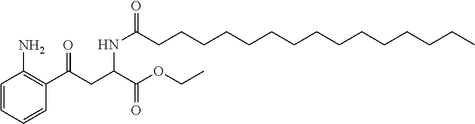 [SMILES: CCCCCCCCCCCCCCCC(=O)NC(CC(=O)c1ccccc1N)C(=O)OCC] |
| 46 | 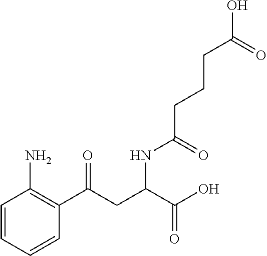 [SMILES: Nc1ccccc1C(=O)CC(NC(=O)CCCC(=O)O)C(=O)O] |
| 47 | 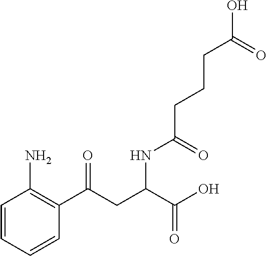 [SMILES: Nc1ccccc1C(=O)CC(NC(=O)CCCC(=O)O)C(=O)O] |
| 48 | 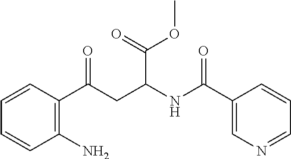 [SMILES: COC(=O)C(CC(=O)c1ccccc1N)NC(=O)c1cccnc1] |
| 49 | 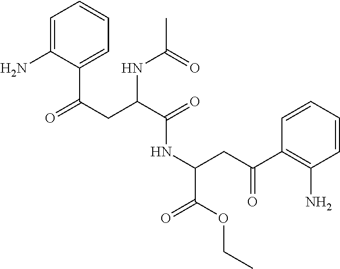 [SMILES: CCOC(=O)C(CC(=O)c1ccccc1N)NC(=O)C(CC(=O)c1ccccc1N)NC(C)=O] |
| 50 | 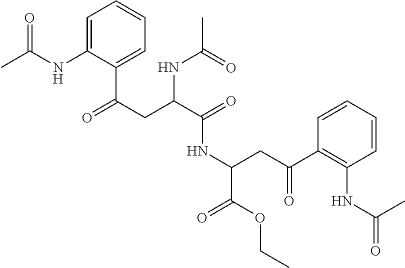 [SMILES: CCOC(=O)C(CC(=O)c1ccccc1NC(C)=O)NC(=O)C(CC(=O)c1ccccc1NC(C)=O)NC(C)=O] |

The above listed compounds can be suitably prepared as described hereinabove, for example, following the procedure described under any one of Schemes 1 to 4, or as described herein below.

All starting materials which are not explicitly described were either commercially available (the details of suppliers such as for example Sigma Aldrich, Tokyo Chemical Industry, Alsa Asesar, Fisher Chemical etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂). The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods. The yields of the compounds prepared are not optimized.

Abbreviations used in the description, particularly in the Schemes and Examples, are as follows: DCC—N,N′-dicyclohexylcarbodiimide, DIC—Diisopropylcarbodiimide, HPLC—High performance liquid chromatography.

### Methods

High pressure liquid chromatography: Samples were suspended in ethanol (Sigma Aldrich 103343081, CAS 64-17-5) and injected onto a reverse phase base deactivated silica C-18 column, 5 μM particle size, 150×4.6 mm (Hypersil 9867). This column was temperature controlled at 35° C. using a Shimadzu CTO-10A column oven. Samples were injected with a Shimadzu SIL-10AD VP auto injector. The program was run using a Shimadzu LC1-20AD pump system, and detection was monitored with ultraviolet-visible spectroscopy from 200 to 700 nm using a Shimadzu SPD-M20A diode array detector.

The flow rate was set to 1.0 mL per minute. From 0 to 1 minute the solvent was deionized water with 0.1% (volume/volume) trifluoroacetic acid (Sigma Aldrich 8.08260, CAS 76-05-1), from 1 to 15 minutes a gradient was effected from 0 to 100% acetonitrile (Fisher Chemical Optima Grade A955-4, CAS 75-05-8) with 0.1% trifluoroacetic acid, the column was maintained at 100% acetonitrile and 0.1% trifluoroacetic acid for from 15 to 17 minutes, and finally the column was re-equilibrated to 100% starting aqueous solvent from 17 to 19 minutes.

### Synthesis of Compounds 1 and 2

Compounds 1 and 2 were prepared as shown in Scheme A and described below:

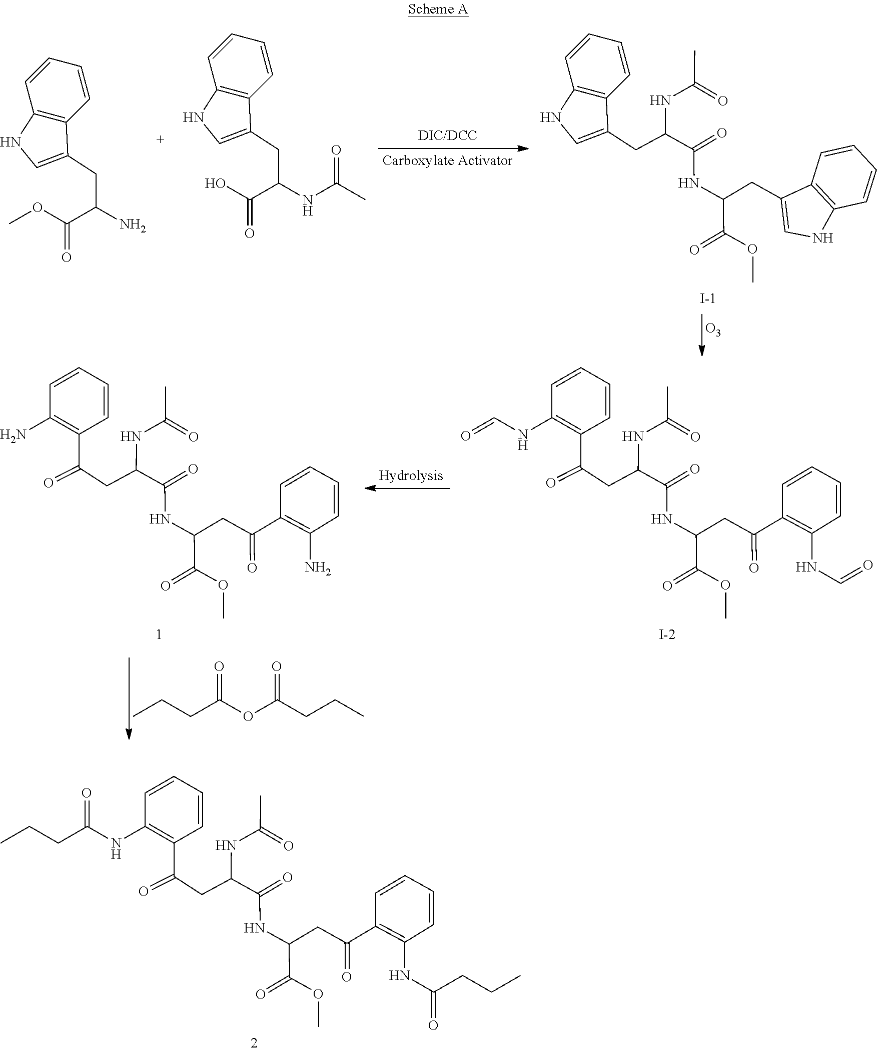

Methyl acetyltryptophyltryptophanate (I-1)

N-acetyl-L-tryptophan (Tokyo Chemical Industry A0121, CAS 1218-34-4) (1.0 g, 4.0 mol) was combined with tryptophan methyl ester (Tokyo Chemical Industry T1675, CAS 7524-52-9) (1.0 g, 3.9 mmol) in 10 mL N,N-dimethylformamide (Sigma Aldrich D4551, CAS 68-12-2), and to this was added N,N′-dicyclohexylcarbodiimide (Sigma Aldrich 36650, CAS 538-75-0) (0.82 g, 4 mmol) and 4-methylmorpholine (0.65 mL, 60 mmol) (Sigma Aldrich 67870, CAS 109-02-4). This was allowed to react at room temperature for 3 hours after which insoluble dicyclohexylurea side product was removed by centrifugation in 15 mL conical polypropylene tubes (Genesee Scientific 28101) using an Eppendorf A-4-44 rotor (4,000×g, 10 minutes). Product was precipitated by addition of 5 mL deionized water and removed by centrifugation in 15 mL conical polypropylene tubes (Genesee Scientific 28101) using an Eppendorf A-4-44 rotor (4,000×g, 10 minutes).

Product was washed twice with water being removed as before with centrifugation. Product 1-1 was confirmed and distinguished from starting material using HPLC, eluting at 8.4 minutes with peak absorbance at 287 nm.

### Compound 1: methyl 2-(2-acetamido-4-(2-aminophenyl)-4-oxobutanamido)-4-(2-aminophenyl)-4-oxobutanoate

Methyl acetyltryptophyltryptophanate (I-1) (50 mg, 0.11 mmol) was suspended in a water:ethanol (33%:66%) solution (pH 5) and oxidized under a stream of ozone (A2Z Ozone Inc., MP3000) for 1 hour (600 mg ozone from ambient air source). The formamide product, methyl 2-(2-acetamido-4-(2-formamidophenyl)-4-oxobutanamido)-4-(2-formamidophenyl)-4-oxobutanoate (I-2), was formed transiently as hydrolysis occurred simultaneously and was nearly complete after 24 hours incubation at room temperature. Solvent was removed under reduced pressure (50 tor, 24 hours). Compound 1 was confirmed and distinguished from starting material and intermediates using HPLC, eluting at 7.6 minutes with peak absorbances at 226 nm, 255 nm (shoulder), and 364 nm.

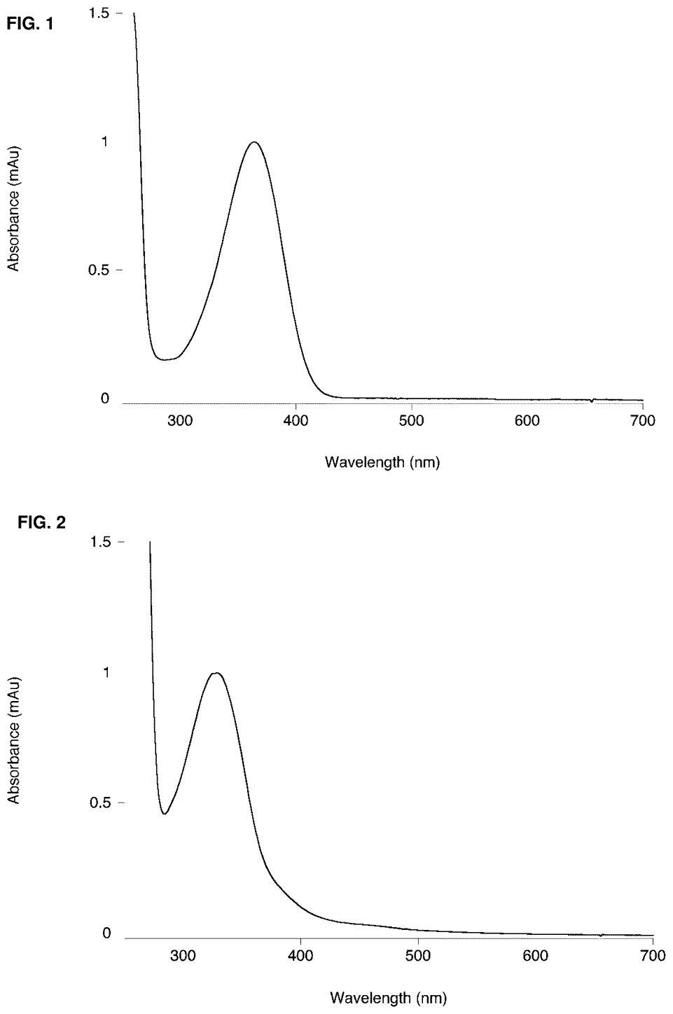

FIG. 1 shows the absorbance spectrum of compound 1. The compound has a strong absorbance in the UVA region. The absorbance is up to 410 nm, suitable for high-energy visible lights protection.

### Compound 2: Methyl 2-(2-acetamido-4-(2-butyramidophenyl)-4-oxobutanamido)-4-(2-butyramidophenyl)-4-oxobutanoate

Methyl 2-(2-acetamido-4-(2-aminophenyl)-4-oxobutanamido)-4-(2-aminophenyl)-4-oxobutanoate (compound 1) (10 mg, 0.022 mmol) was suspended directly into butyric anhydride (Alsa Asesar A12377.AE, CAS 106-31-0) and incubated at room temperature in a sealed glass container for 24 hours. Compound 2 was confirmed and distinguished from starting material using HPLC, eluting at 10.5 minutes with peak absorbances at 229 nm, 260 nm, and 329 nm.

FIG. 2 shows the absorbance spectrum of compound 2. It can be seen that acylation of the amines of the anilines of compound 1 shifted the absorbance spectrum, producing a compound which can provides protection in both the UVA and UVB region.

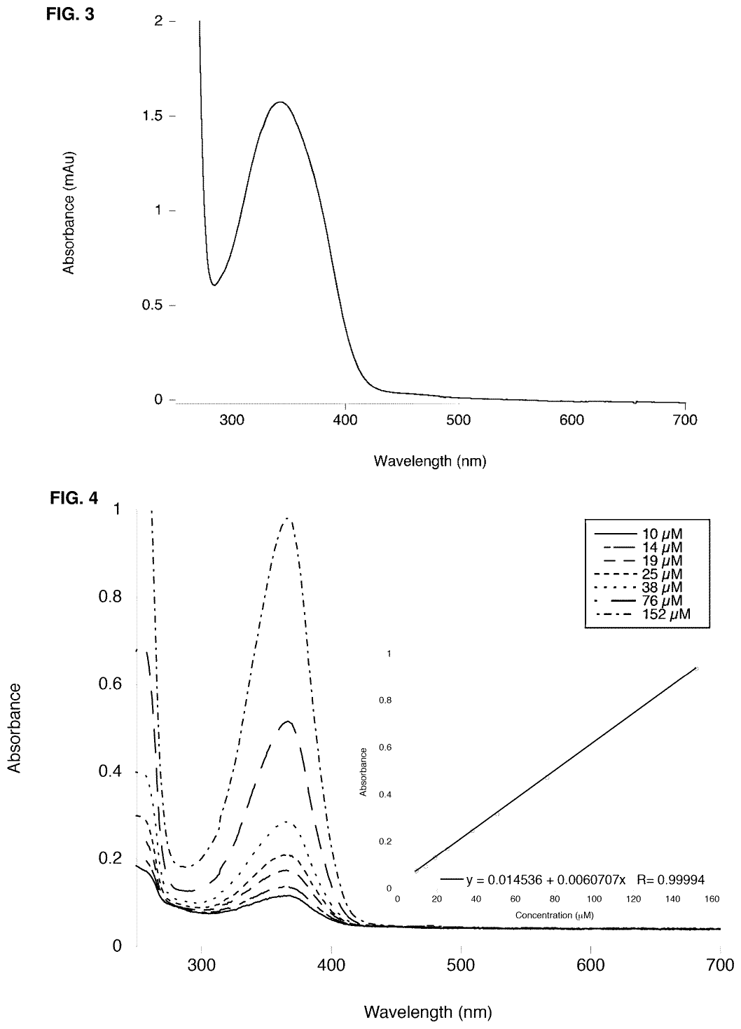

The absorbance spectrum of compounds 1 and 2 were combined to give broad spectrum UVB and UVA protection shown in FIG. 3.

### Synthesis of Compound 4

Compound 4 was prepared as shown in Scheme B and described below:

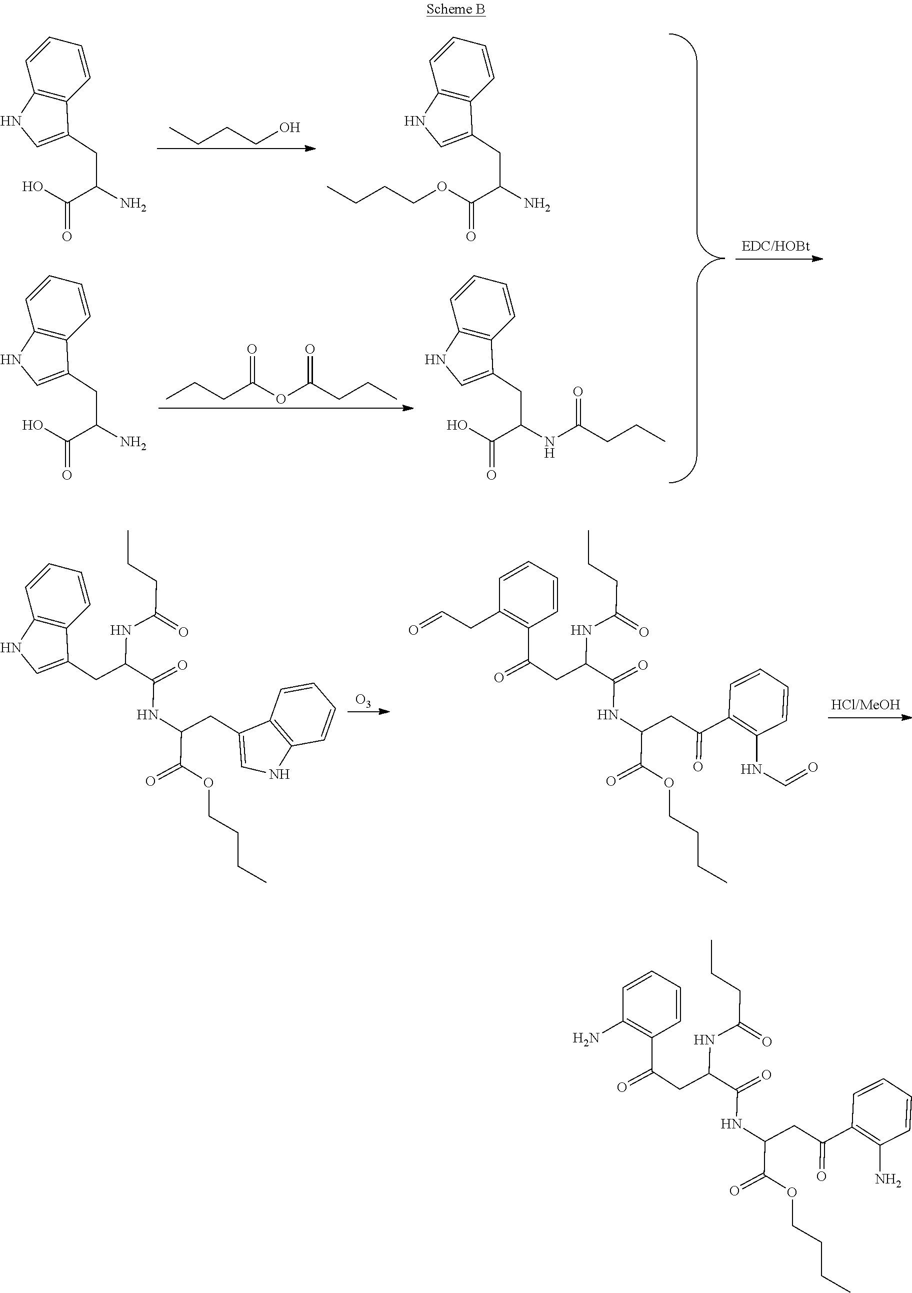

### Preparation of Butyl Tryptophanate

L-Tryptophane (CAS 72-22-3; 20 g; 98 mmol) was dissolved in n-butanol (CAS 138373-86-1; 300 ml) and thionyl chloride (CAS 7719-09-7; 20 ml; 290 mmol) was added dropwise over 10 minutes. The reaction mixture was stirred at 90° C. overnight. The white solid, precipitated after cooling to room temperature, was filtered off and washed with ice cold butanol. The product butyl tryptophanate was dried in vacuum oven, yielding 24 g of butyl tryptophanate (91%).

ESI/APCI(+): 261.20 (M+H)

### Preparation of Butyryltryptophan

L-Tryptophane (CAS 72-22-3; 20 g; 98 mmol) was dissolved in ACN (CAS 75-05-8; 200 ml). Triethylamine (CAS 121-44-8; 40 ml; 290 mmol) and butyric anhydride (CAS 106-32-0; 18.6; 117.5 mmol) was slowly added. The reaction mixture was stirred at 60° C. for 3 hours and concentrated under reduced pressure. The crude material was reparted between ethyl acetate (CAS 141-78-6) and water; the phases were separated and the aqueous were extracted with ethyl acetate once. The organic layers were combined, dried, and concentrated under reduced pressure and crude product was purified by flash chromatography on silica gel (eluent 20 to 100% ethyl acetate in heptane CAS 142-85-5;) to afford butyryltryptophan as yellow oil (25 g, 92%).

ESI/APCI(+): 275.30 (M+H)

### Preparation of butyl butyryltryptophyltryptophanate

A mixture of butyl tryptophanate (18 g; 61 mmol), butyryltryptophan (22 g; 80 mmol), 1-Ethyl-3-(3′-dimethylaminopropyl)carbodiimide hydrochloride (CAS 25952-53-8; 26 g; 132 mmol), Hydroxybenzotriazole (CAS 123333-53-9; 20 g; 132 mmol), and Triethylamine (CAS 121-44-8; 50 ml; 360 mmol) in dimethylformamide (CAS 68-12-2; 200 mL) was stirred at room temperature for 4 days. The reaction crude was quenched with water (300 mL) and extracted with dichloromethane (CAS 75-09-2; 200 mL*2). The organic layers were combined and washed with HCl (CAS 7647-01-0; 0.5N), NaHCO₃ (CAS 144-55-8; sat.) and brine; then dried and concentrated under reduced pressure. The crude product was purified by flash chromatography on silica gel (eluent 20 to 100% ethyl acetate in toluene CAS 108-88-3) to afford butyl butyryltryptophyltryptophanate as off-white solid (21 g, 65%).

ESI/APCI(+): 517.25 (M+H)

¹H-NMR (CDCl₃): δ 8.08-7.67 (m, 3H), 7.43-7.27 (m, 2H), 7.27-6.97 (m, 5H), 6.82-6.44 (m, 2H), 6.13 (dt, J=20.6, 7.4 Hz, 2H), 4.76 (qt, J=6.2, 4.3 Hz, 2H), 4.01 (dtt, J=17.6, 7.2, 3.8 Hz, 2H), 3.46-2.89 (m, 4H), 2.18-1.82 (m, 2H), 1.60-1.15 (m, 7H), 0.95-0.76 (m, 6H).

### Preparation of butyl 2-(2-butyramido-4-(2-formamidophenyl)-4-oxobutanamido)-4-(2-formamidophenyl)-4-oxobutanoate

A mixture of butyl butyryltryptophyltryptophanate (11 g; 21 mmol) in dichloromethane (CAS 75-09-2; 450 mL) was stirred at room temperature O₃ gas from ozone generator (PCI ozone corp., GI-L) was bubbled into solution for 90 minutes. The reaction was monitored by HPLC and quenched with water (300 mL) and nitrogen gas flow. The phases were separated, and the organic layer was dried and concentrated under reduced pressure. The crude product was directly used in next step without further purification.

ESI/APCI(+): 581.35 (M+H)

### Compound 4: butyl 4-(2-aminophenyl)-2-(4-(2-aminophenyl)-2-butyramido-4-oxobutanamido)-4-oxobutanoate

The crude product butyl 2-(2-butyramido-4-(2-formamidophenyl)-4-oxobutanamido)-4-(2-formamidophenyl)-4-oxobutanoate (10 g) was dissolved MeOH (CAS 67-56-1; 100 ml) and HCl (1N Aqu.; 100 mL) was added. The reaction mixture was stirred at room temperature for 1 hour. HPLC showed the reaction was completed. The reaction mixture was quenched with NaHCO₃ (CAS 144-55-8; sat.) and extracted with dichloromethane (CAS 75-09-2; 200 mL*2). The organic layers were combined, dried, and concentrated under reduced pressure. The crude product was purified by flash chromatography on silica gel (eluent 20 to 100% ethyl acetate in toluene) to afford butyl 4-(2-aminophenyl)-2-(4-(2-aminophenyl)-2-butyramido-4-oxobutanamido)-4-oxobutanoate as brown oil (4 g, 36% over two steps).

ESI/APCI(+): 525.18 (M+H)

### Synthesis of Compound 5

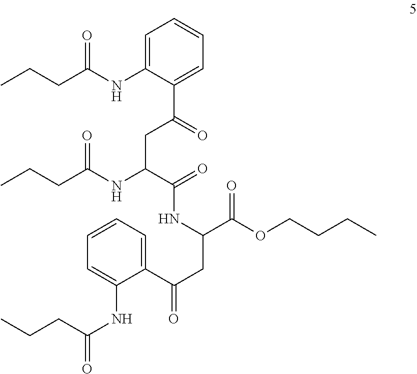 [SMILES: CCCCOC(=O)C(CC(=O)c1ccccc1NC(=O)CCC)NC(=O)C(CC(=O)c1ccccc1NC(=O)CCC)NC(=O)CCC]

Compound 5 was prepared using a protocol similar to what was described for the synthesis of compound 2, except that compound 4 was used as starting material.

Butyl 4-(2-aminophenyl)-2-(4-(2-aminophenyl)-2-butyramido-4-oxobutanamido)-4-oxobutanoate (compound 4) was suspended directly into butyric anhydride (Alsa Asesar A12377.AE, CAS 106-31-0) and incubated at room temperature in a sealed glass container for 24 hours. Compound 5 was confirmed and distinguished from starting material using HPLC.

### Determination of Maximal UV Absorbance, Extinction Coefficients, and Critical Wavelengths

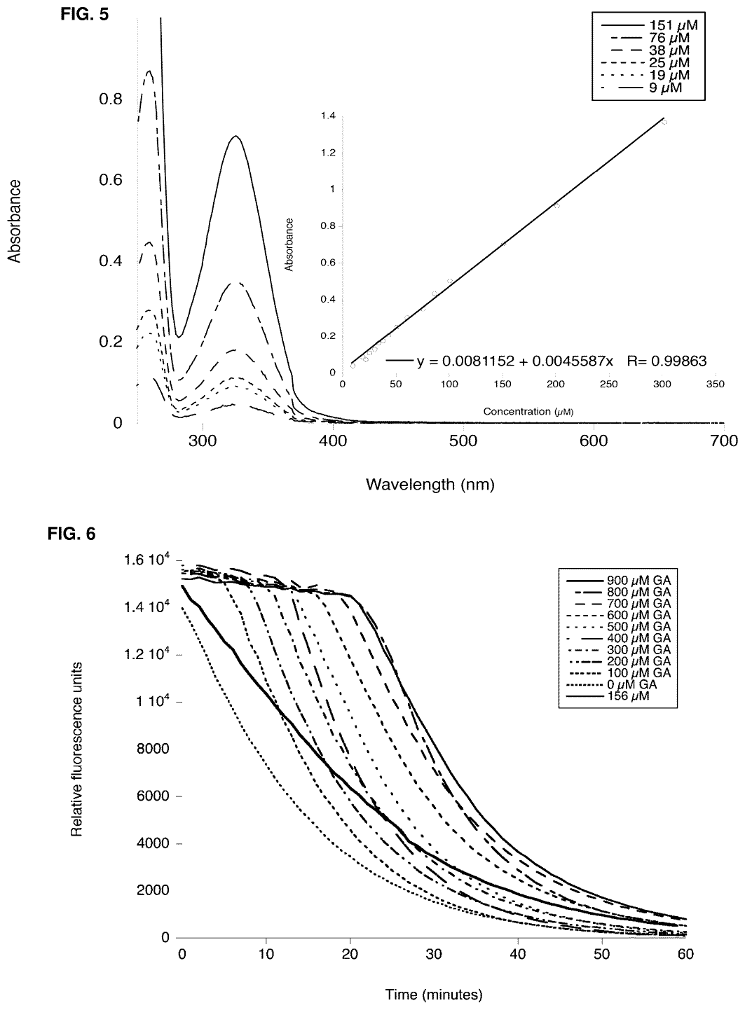

Extinction coefficients were determined by measuring the dry mass of each compounds and diluting it serially in ethanol (CAS 64-17-12). A series of concentrations were tested with UV visible spectroscopy. The results for compound 4 are shown in FIG. 4. The results for compound 5 are shown in FIG. 5. This showed an increase in absorbance that was fit to a line, the slope of which was the extinction coefficient.

In vitro critical wavelengths were determined by integrating the total absorbance from 290 to 400 nm and finding the region under that curve from higher energy to lower energy that is equal to 90% of the total absorbance, similar to that described by Diffey B. L., et al. J. Am. Acad. Dermatol. 2000, 43, 1024-1035.

The peak absorbance for compound 4 (butyl 4-(2-aminophenyl)-2-(4-(2-aminophenyl)-2-butyramido-4-oxobutanamido)-4-oxobutanoate) was around 368 nm. The extinction coefficient was 6.1×10³ M⁻¹ cm⁻¹. The critical wavelength was 385 nm.

The peak absorbance for compound 5 (butyl 2-(2-butyramido-4-(2-butyramidophenyl)-4-oxobutanamido)-4-(2-butyramidophenyl)-4-oxobutanoate) was around 325 nm. The extinction coefficient was 4.6×10³ M⁻¹ cm⁻¹. The critical wavelength was 355 nm.

### Antioxidant Activity

An OxiSelect Hydroxyl Radical Antioxidant Capacity (HORAC) Activity Assay (Cell Biolabs Inc. catalog #STA-346-T) was used to determine the antioxidant potential of compound 4 (butyl 4-(2-aminophenyl)-2-(4-(2-aminophenyl)-2-butyramido-4-oxobutanamido)-4-oxobutanoate) following the manufacturer's protocol. Gallic Acid (GA) was used as antioxidant Calibrator: The HORAC assay was used to evaluate the application of the compounds of the invention as antioxidant.

A small amount of compound 4 was resuspended in assay diluent and the concentration determined using the extinction coefficient previously determined at 368 nm (6070 M⁻¹ cm⁻¹). The concentration of the stock solution was 624 μM. Three concentrations of the compound were tested in the assay: 468 μM, 312 μM, and 156 μM. The assay was run in a clear bottomed 96-well plate using a Molecular Devices SpectraMax M2 with an excitation wavelength of 480 nm and an emission wavelength of 530 nm. The emission wavelength cutoff was set to 515 nm. The photomultiplier sensitivity set to medium. The plate was read once a minute for 60 minutes.

FIG. 6 illustrates the antioxidant capacity of Compound 4. The antioxidant potential was found to be 1.8 gallic acid equivalents per mole of compound 4 showing its potential as an effective antioxidant.

### Synthesis of Compound 6

Compound 6 (butyl 4-(2-acetamidophenyl)-2-(4-(2-acetamidophenyl)-2-butyramido-4-oxobutanamido)-4-oxobutanoate) was prepared as shown in scheme K and as described below.

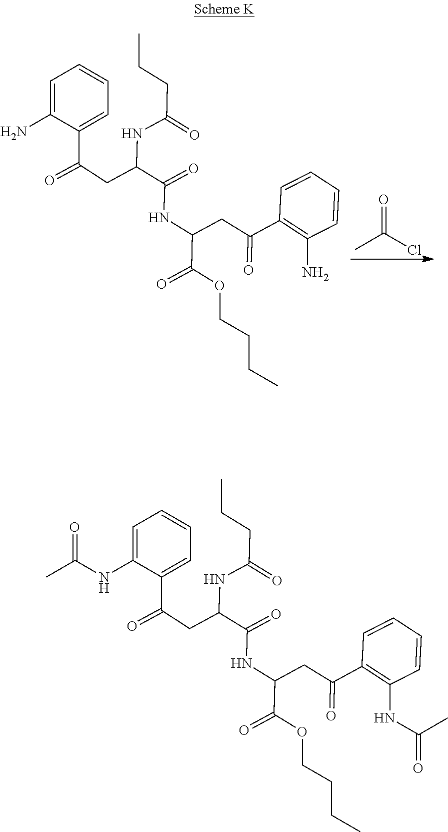

Butyl 4-(2-aminophenyl)-2-(4-(2-aminophenyl)-2-butyramido-4-oxobutanamido)-4-oxobutanoate (Compound 6; 23 mg; 0.049 mmol) was dissolved in ethyl acetate (CAS 141-78-6; 50 ml). Triethylamine (CAS 121-44-8; 0.037 ml; 0.263 mmol) and acetyl chloride (CAS 75-36-5; 0.013 ml; 0.176 mmol) were added dropwise. The reaction proceeded at reflux for 4 hours, and the product (butyl 4-(2-acetamidophenyl)-2-(4-(2-acetamidophenyl)-2-butyramido-4-oxobutanamido)-4-oxobutanoate) was distinguished from starting material by HPLC, eluting at 11.6 minutes.

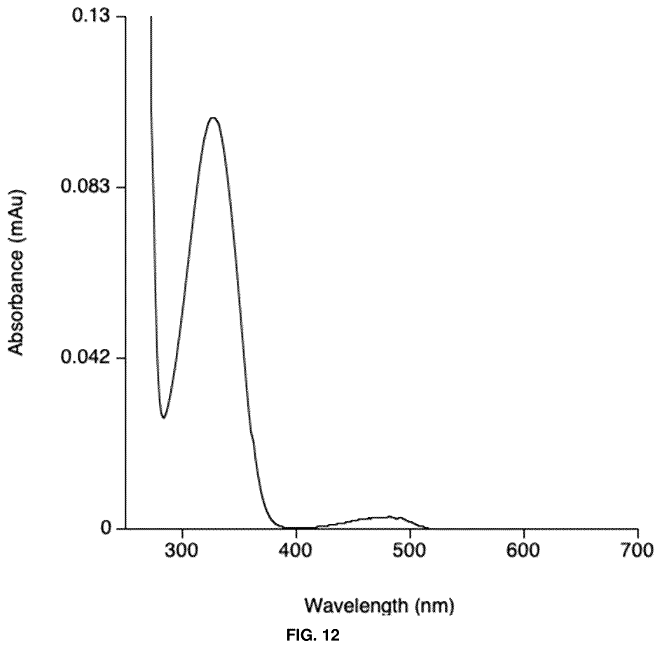

The absorbance spectrum of compound 6 is shown in FIG. 12.

### Synthesis of Compound 25

Compound 25 can be prepared as shown in Scheme C:

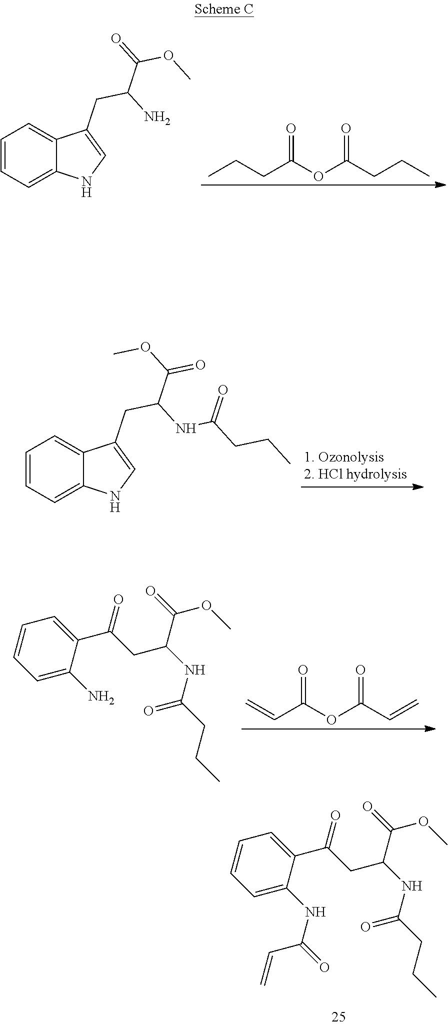

L-Tryptophan methyl ester (CAS 7524-52-9) can be N-acylated with butyric anhydride (CAS 106-32-0) thereby forming methyl butyryltryptophanate, which can then be oxidized by ozonolysis followed by HCl hydrolysis thereby forming methyl 4-(2-aminophenyl)-2-butyramido-4-oxobutanoate. Compound 25 (methyl 4-(2-acrylamidophenyl)-2-butyramido-4-oxobutanoate) can be obtained by reacting methyl 4-(2-aminophenyl)-2-butyramido-4-oxobutanoate with acrylic anhydride.

### Synthesis of Compound 33

Compound 33 can be prepared as shown in Scheme D:

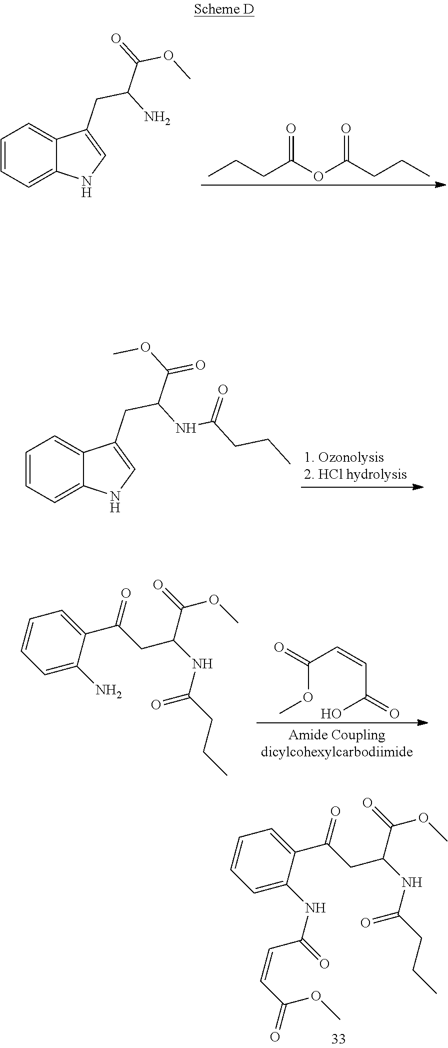

L-Tryptophan methyl ester (CAS 7524-52-9) can be N-acylated with butyric anhydride (CAS 106-32-0) thereby forming methyl butyryltryptophanate, which can then be oxidized by ozonolysis followed by HCl hydrolysis thereby forming methyl 4-(2-aminophenyl)-2-butyramido-4-oxobutanoate. Compound 33 (methyl (Z)-4-((2-(3-butyramido-4-methoxy-4-oxobutanoyl)phenyl)amino)-4-oxobut-2-enoate) can be obtained by reacting methyl 4-(2-aminophenyl)-2-butyramido-4-oxobutanoate with (Z)-4-methoxy-4-oxobut-2-enoic acid, in the presence of dicylcohexylcarbodiimide.

### Synthesis of Compound 40

Compound 40 can be prepared as shown in Scheme E:

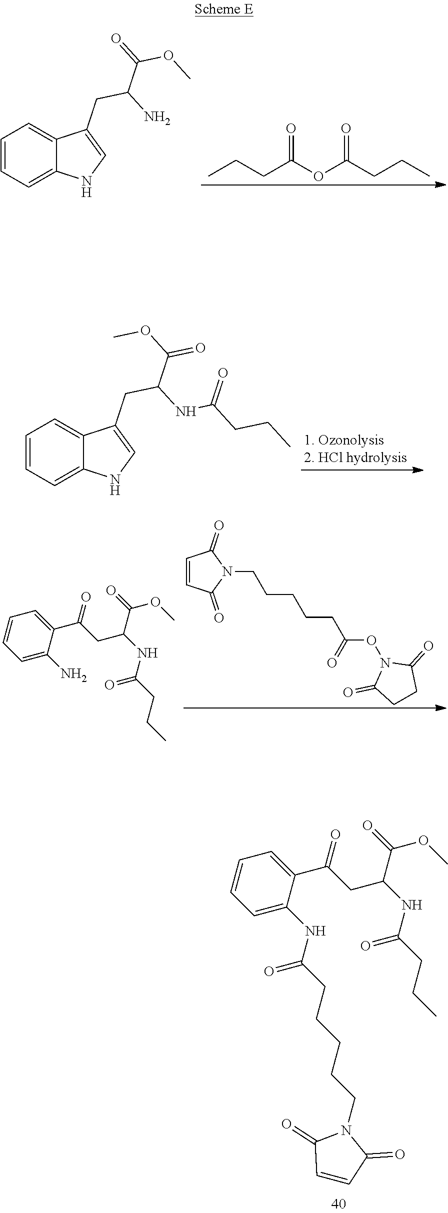

L-Tryptophan methyl ester (CAS 7524-52-9) can be N-acylated with butyric anhydride (CAS 106-32-0) thereby forming methyl butyryltryptophanate, which can then be oxidized by ozonolysis followed by HCl hydrolysis thereby forming methyl 4-(2-aminophenyl)-2-butyramido-4-oxobutanoate. Compound 40 (methyl 2-butyramido-4-(2-(6-(2,5-dioxo-2,5-dihydro-1H-pyrrol-1-yl)hexanamido)phenyl)-4-oxobutanoate) can be obtained by reacting methyl 4-(2-aminophenyl)-2-butyramido-4-oxobutanoate with a carboxylic acid ester of N-hydroxysuccinimide such as 2,5-dioxopyrrolidin-1-yl 6-(2,5-dioxo-2,5-dihydro-1H-pyrrol-1-yl)hexanoate.

### Synthesis of Compound 42

Compound 42 was prepared as shown in Scheme F and as described below.

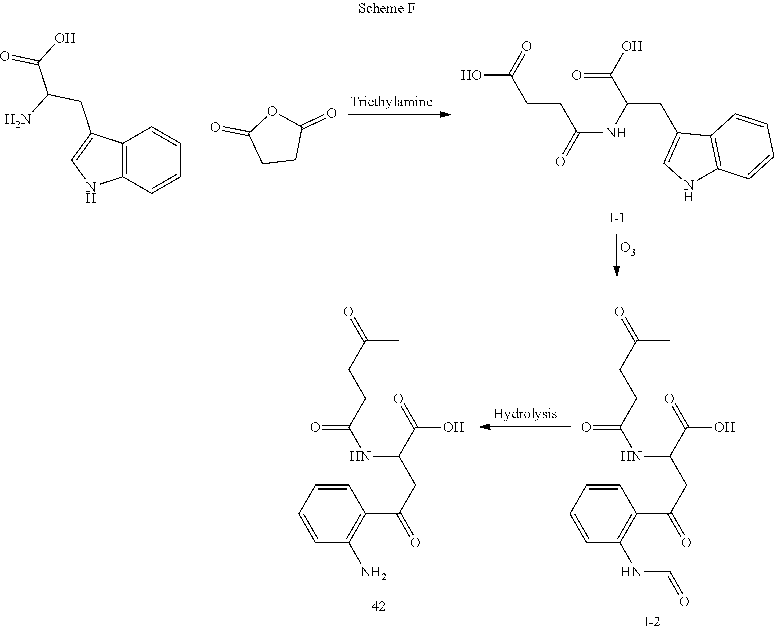

### 4-((1-carboxy-2-(1H-indol-3-yl)ethyl)amino)-4-oxobutanoic acid (I-1)

L-tryptophan (Thermo Scientific A10230.36, CAS 73-22-3) (1.0 g, 4.9 mmol) was combined with succinic anhydride (Thermo Scientific A12245.36, CAS 108-30-5) (0.59 g, 5.9 mmol) in 10 mL acetonitrile (Fisher A21-1, CAS 75-05-8), and to this was added triethylamine (1.9 mL, 14.7 mmol). The mixture was allowed to react at room temperature for 12 hours, after which solvent was removed under reduced pressure. Intermediate I-1 was confirmed and distinguished from starting material using HPLC, eluting at 6.5 minutes with a peak absorbance at 279 nm.

### 2-(3-carboxypropanamido)-4-(2-formamidophenyl)-4-oxobutanoic acid (I-2)

4-((1-carboxy-2-(1H-indol-3-yl)ethyl)amino)-4-oxobutanoic acid (I-1) (1 g, 3.3 mmol) was suspended in 50 mL methanol (Fisher A452-4, CAS 67-56-1) and oxidized under a stream of ozone (A2Z Ozone Inc., MP3000) for 20 minutes (12.5 mmol ozone per hour from ambient air source). The formamide product, 2-(3-carboxypropanamido)-4-(2-formamidophenyl)-4-oxobutanoic acid (I-2), was formed and confirmed via HPLC, eluting at 5.9 minutes with peak absorbance at 319 nm.

### 4-(2-aminophenyl)-2-(3-carboxypropanamido)-4-oxobutanoic acid (42)

Hydrochloric acid (Sigma 258148-2.5L, CAS 7647-01-0) was added to the 2-(3-carboxypropanamido)-4-(2-formamidophenyl)-4-oxobutanoic acid (I-2) in methanol to give a final concentration of 1 M. The mixture was allowed to react for 3 hours at room temperature with formation of 4-(2-aminophenyl)-2-(3-carboxypropanamido)-4-oxobutanoic acid (42) monitored via HPLC, eluting at 5.7 minutes with a peak absorbance at 362 nm. The hydrochloric acid was neutralized by addition of 6 M sodium hydroxide (Fisher BP359-500, CAS 1310-73-2, CAS 497-19-8) in water.

The solvent was removed under reduced pressure. The resulting oil was purified by flash chromatography on silica gel (eluent 25% to 100% methanol (Fisher A452-4, CAS 67-56-1) in ethyl acetate (Fisher E145-4, CAS 141-78-6)). The correct compound was confirmed to be pure via HPLC and the solvent was removed under reduced pressure.

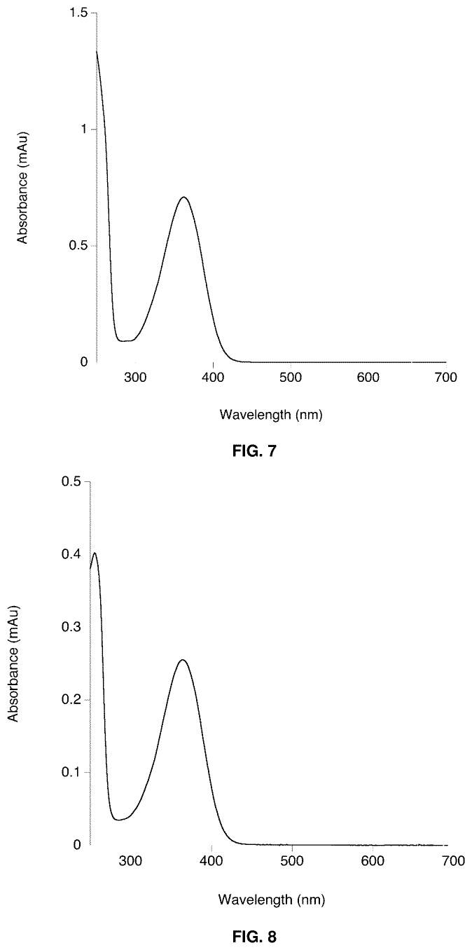

FIG. 7 shows the absorbance spectrum of compound 42

### Synthesis of Compound 43

Compound 43 was prepared as shown in Scheme G and as described below:

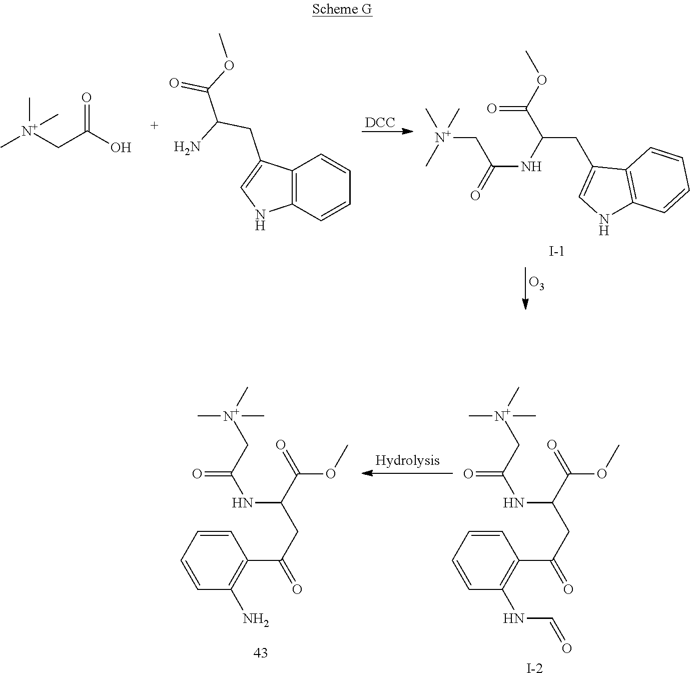

### 2-((3-(1H-indol-3-yl)-1-methoxy-1-oxopropan-2-yl)amino)-N,N,N-trimethyl-2-oxoethan-1-aminium (I-1)

Betaine (Sigma W422312, CAS 107-43-7) (1.2 g, 0.01 mols) was combined with L-tryptophan methyl ester hydrochloride (TCI T1657, CAS 7524-52-9) (2.3 g, 0.009 mols) and suspended in 50 mL of acetonitrile (Fisher A21-1, CAS 75-05-8) To this was added 4-methyl morpholine (Sigma 67870, CAS 109-02-4) (2.4 mL, 0.02 mols) and DCC (Sigma 36650, CAS 538-75-0) (2.2 g, 0.01 mols). The mixture was allowed to react at room temperature, with the reaction being monitored by HPLC. Intermediate I-1, was confirmed and distinguished from the starting material using HPLC, eluting at 7.7 minutes with a peak absorbance at 279 nm. Excess DCC was quenched by the addition of water, and precipitated material was removed by filtration. Solvent was removed under reduced pressure.

### 2-((4-(2-formamidophenyl)-1-methoxy-1,4-dioxobutan-2-yl)amino)-N,N,N-trimethyl-2-oxoethan-1-aminium (I-2)

2-((3-(1H-indol-3-yl)-1-methoxy-1-oxopropan-2-yl)amino)-N,N,N-trimethyl-2-oxoethan-1-aminium (I-1) (1 g, 3.1 mmols) was suspended in 50 mL methanol (Fisher A452-4, CAS 67-56-1) and oxidized under a stream of ozone (A2Z Ozone Inc., MP3000) for 20 minutes (12.5 mmol ozone per hour from ambient air source). The formamide product, 2-((4-(2-formamidophenyl)-1-methoxy-1,4-dioxobutan-2-yl)amino)-N,N,N-trimethyl-2-oxoethan-1-aminium (I-2), was formed and confirmed via HPLC, eluting at 7.29 minutes with peak absorbances at 321 nm.

### 2-((4-(2-aminophenyl)-1-methoxy-1,4-dioxobutan-2-yl)amino)-N,N,N-trimethyl-2-oxoethan-1-aminium (43)

Hydrochloric acid (Sigma 258148-2.5L, CAS 7647-01-0) was added to the 2-((4-(2-formamidophenyl)-1-methoxy-1,4-dioxobutan-2-yl)amino)-N,N,N-trimethyl-2-oxoethan-1-aminium (I-2) in methanol to give a final concentration of 1 M. The mixture was allowed to react for 3 hours at room temperature with formation of 2-((4-(2-aminophenyl)-1-methoxy-1,4-dioxobutan-2-yl)amino)-N,N,N-trimethyl-2-oxoethan-1-aminium (43) monitored via HPLC, eluting at 7.23 minutes with a peak absorbance at 365 nm. The hydrochloric acid was neutralized by addition of 6 M sodium hydroxide (Fisher BP359-500, CAS 1310-73-2, CAS 497-19-8) in water.

Solvent was removed under reduced pressure.

FIG. 8 shows the absorbance spectrum of compound 43.

### Synthesis of Compound 44

Compound 44 (2-acetamido-4-(2-aminophenyl)-4-oxobutanamide) was prepared as shown in Scheme H and as described below.

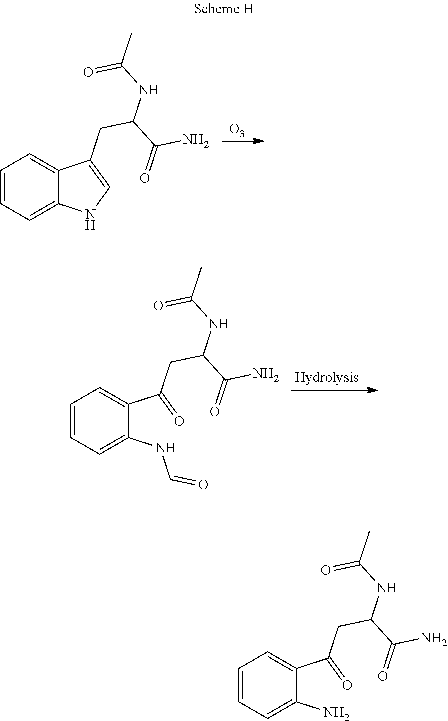

N-acetyl-L-tryptophanamide (CAS 2382-79-8; 20 mg; 0.0816 mmol) was dissolved in methanol (CAS 67-56-1; 3 ml) and oxidized under a stream of ozone (A2Z Ozone Inc., MP3000) for 10 minutes (12.5 mmol ozone per hour from ambient air source). A mixture of the formamide product, 2-acetamido-4-(2-formamidophenyl)-4-oxobutanamide, and the hydrolyzed final product, 2-acetamido-4-(2-aminophenyl)-4-oxobutanamide, were formed. Incubation for two days at room temperature resulted in complete conversion of the formamide product to 2-acetamido-4-(2-aminophenyl)-4-oxobutanamide. The formamide product and hydrolyzed product were confirmed and distinguished from starting material using HPLC, with the formamide product eluting at 6.7 minutes and the hydrolyzed product at 6.4 minutes.

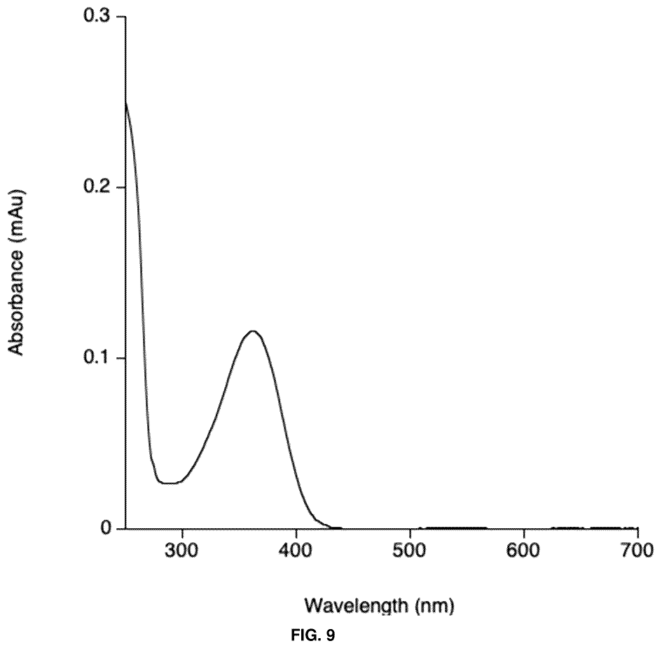

The absorbance spectrum of compound 44 is shown in FIG. 9.

### Synthesis of Compound 45

Compound 45 (ethyl 4-(2-aminophenyl)-4-oxo-2-palmitamidobutanoate) was prepared as shown in Scheme I and as described below.

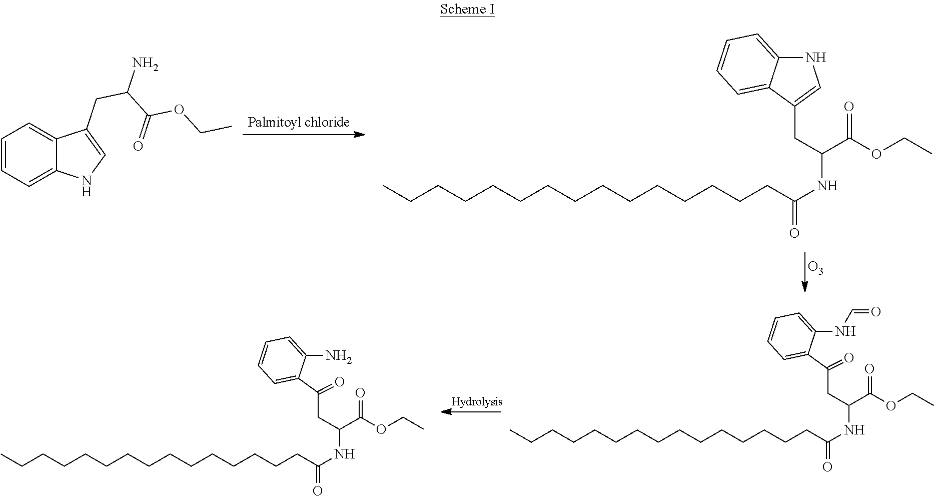

L-tryptophan ethyl ester hydrochloride (CAS 2899-28-7; 100 mg; 0.372 mmol) was dissolved in ethyl acetate (CAS 141-78-6; 5 ml), triethylamine (CAS 121-44-8; 0.155 ml; 1.12 mmol), and palmitoyl chloride (CAS 112-67-4; 0.134 ml; 0.447 mmol) were added dropwise. This was stirred overnight at room temperature. The crude material was reparted between ethyl acetate and water; the phases were separated and the aqueous phase was extracted with ethyl acetate once more. The organic layers were combined, dried, and concentrated under reduced pressure.

Ethyl palmitoyltryptophanate was dissolved in methanol (CAS 67-56-1; 20 ml) and oxidized under a stream of ozone (A2Z Ozone Inc., MP3000) for 10 minutes (12.5 mmol ozone per hour from ambient air source). This resulted in formation of the formamide product (ethyl 4-(2-formamidophenyl)-4-oxo-2-palmitamidobutanoate), to which hydrochloric acid (CAS 7647-01-0; 0.8 ml; 10 mmol) was added. Incubation at room temperature for 1 hour resulted in complete hydrolysis of the formamide product to form ethyl 4-(2-aminophenyl)-4-oxo-2-palmitamidobutanoate. This product was confirmed and distinguished from starting material using HPLC, eluting at 15.3 minutes. Hydrochloric acid was neutralized by addition of sodium hydroxide (CAS 1310-73-2), and the solvent evaporated under reduced pressure. The crude material was reparted between ethyl acetate and water; the phases were separated and the aqueous phase was extracted with ethyl acetate once more. The organic layers were combined, dried, and concentrated under reduced pressure.

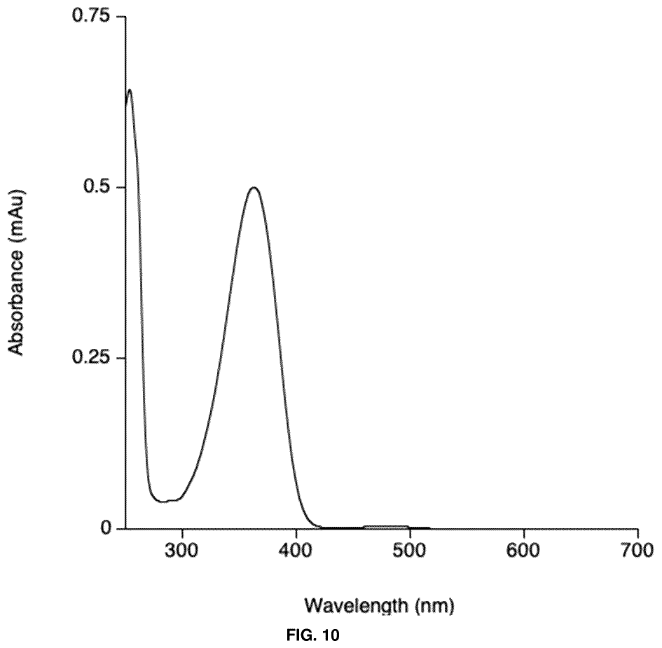

The absorbance spectrum of compound 45 is shown in FIG. 10.

### Synthesis of Compound 46

Compound 46 (5-((3-(2-aminophenyl)-1-carboxy-3-oxopropyl)amino)-5-oxopentanoic acid) was prepared as shown in scheme J and as described below.

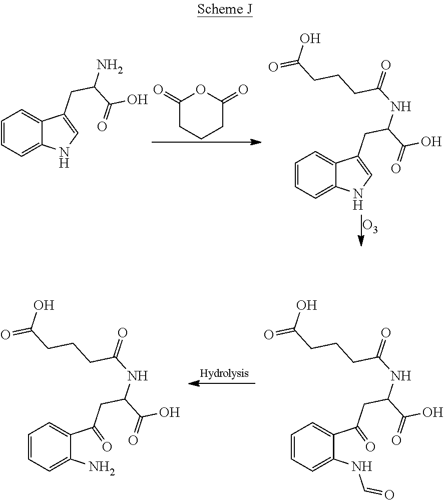

L-tryptophan (CAS 73-22-3; 1.0 g; 4.9 mmol) was combined with glutaric anhydride (CAS 108-55-4; 0.67 g; 5.9 mmol) in acetonitrile (CAS 75-05-8; 10 ml), and to this was added triethylamine (1.9 mL, 14.7 mmol). The mixture was allowed to react at room temperature for 12 hours, after which solvent was removed under reduced pressure.

5-((1-carboxy-2-(1H-indol-3-yl)ethyl)amino)-5-oxopentanoic was suspended in methanol (CAS 67-56-1, 50 ml) and oxidized under a stream of ozone (A2Z Ozone Inc., MP3000) for 20 minutes (12.5 mmol ozone per hour from ambient air source). The formamide product, 5-((1-carboxy-3-(2-formamidophenyl)-3-oxopropyl)amino)-5-oxopentanoic acid, was formed.

Hydrochloric acid (Sigma 258148-2.5L, CAS 7647-01-0) was added to the formamide product in methanol, to give a final concentration of 1 M. The mixture was allowed to react overnight, hydrolyzing the formamide product to the desired final product, 5-((3-2(aminophenyl)-1-carboxy-3-oxopropyl)amino)-5-oxopentanoic acid. This product was confirmed and distinguished from starting material using HPLC, eluting at 7.3 minutes.

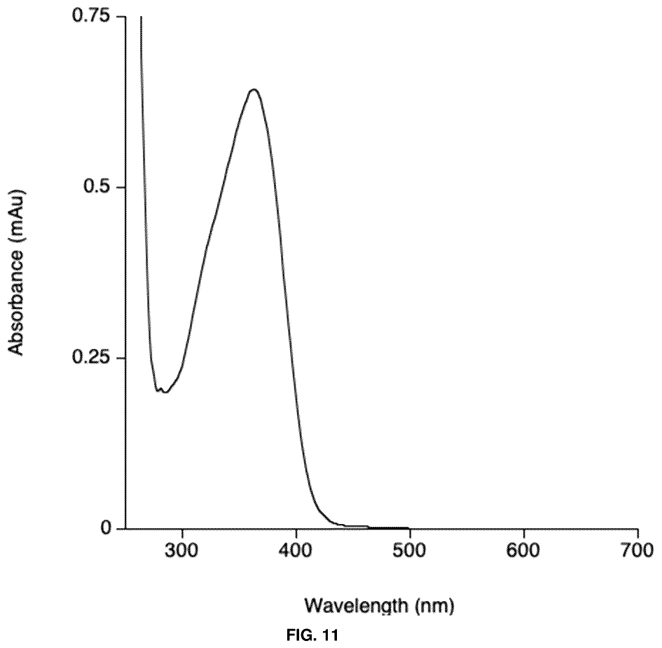

The absorbance spectrum of compound 46 is shown in FIG. 11.

### Synthesis of Compound 47

Compound 47 (butyl 4-(2-benzamidophenyl)-2-(4-(2-benzamidophenyl)-2-butyramido-4-oxobutanamido)-4-oxobutanoate) was prepared as shown in scheme L.

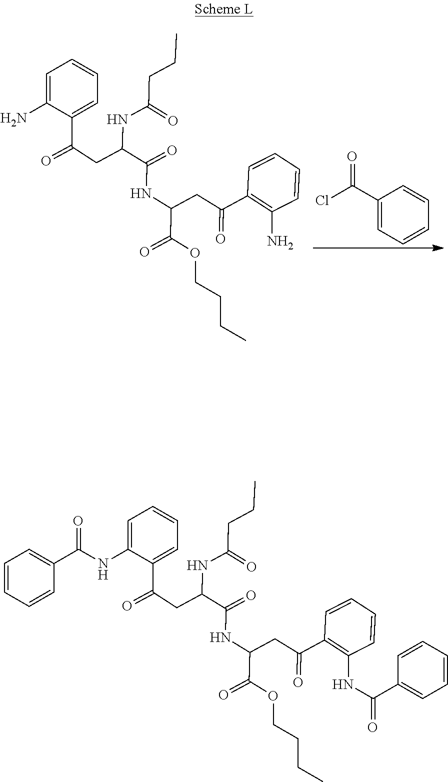

Butyl 4-(2-aminophenyl)-2-(4-(2-aminophenyl)-2-butyramido-4-oxobutanamido)-4-oxobutanoate (Compound 6; 23 mg; 0.049 mmol) was dissolved in ethyl acetate (CAS 141-78-6; 50 ml). Triethylamine (CAS 121-44-8; 0.037 ml; 0.263 mmol) and benzoyl chloride (CAS 98-88-4; 0.017 ml; 0.190 mmol) were added dropwise. The reaction proceeded at reflux for 4 hours, and the product (butyl 4-(2-benzamidophenyl)-2-(4-(2-benzamidophenyl)-2-butyramido-4-oxobutanamido)-4-oxobutanoate) was distinguished from starting material by HPLC, eluting at 14.4 minutes.

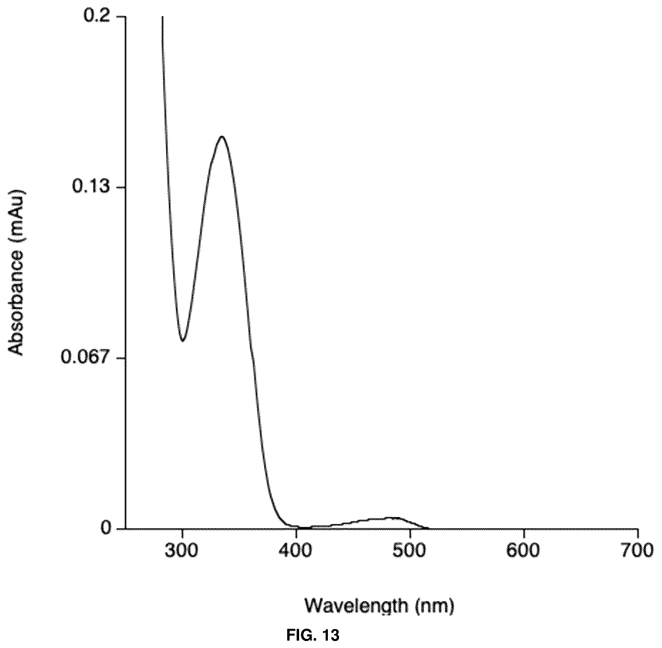

The absorbance spectrum of compound 47 is shown in FIG. 13.

### Synthesis of Compound 48

Compound 48 (methyl 4-(2-aminophenyl)-2-(nicotinamido)-4-oxobutanoate) was prepared as shown in scheme L and as described below.

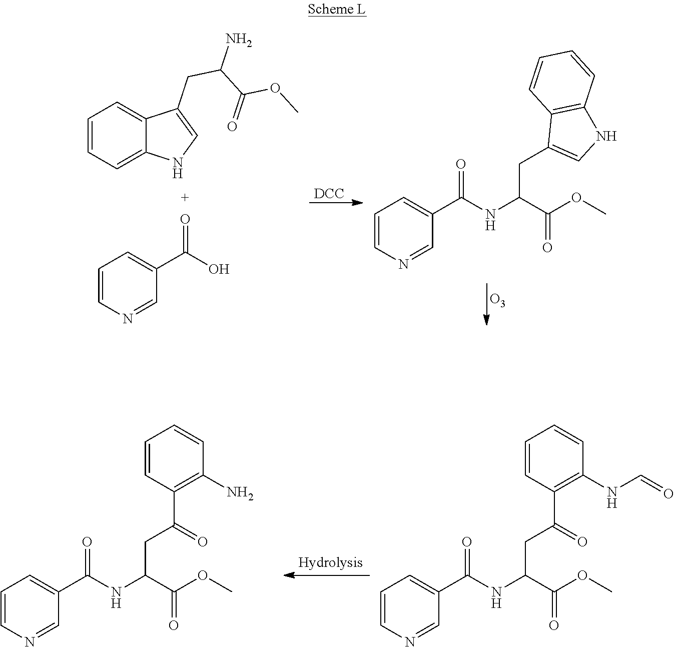

L-tryptophan methyl ester hydrochloride (CAS 7524-52-9; 1.17 g; 4.59 mmol) was combined with nicotinic acid (CAS 59-67-6; 0.68 g; 5.51 mmol) and suspended in acetonitrile (CAS 75-05-8; 50 ml). To this was added 4-methylmorpholine (CAS 109-02-4; 1 ml; 8.17 mmol) and DCC (CAS 538-75-0; 1.36 g; 6.6 mmol). The mixture was stirred at room temperature overnight, and the reaction was confirmed to be complete, forming methyl nicotinoyltryptophanate, by HPLC. Water was added to quench excess DCC, precipitate was removed by filtration, and the solvent was removed by evaporation under reduced pressure.

The intermediate, methyl nicotinoyltryptophanate, was resuspended in methanol (CAS 67-56-1, 50 ml) and oxidized under a stream of ozone (A2Z Ozone Inc., MP3000) for 30 minutes (12.5 mmol ozone per hour from ambient air source). A mixture of the formamide product (methyl 4-(2-formamidophenyl)-2-(nicotinamido)-4-oxobutanoate) and the hydrolyzed product (methyl 4-(2-aminophenyl)-2-(nicotinamido)-4-oxobutanoate) were formed. Incubation for three days at room temperature resulted in complete hydrolysis of the formamide product to methyl 4-(2-aminophenyl)-2-(nicotinamido)-4-oxobutanoate. This product was confirmed and distinguished from starting material using HPLC, eluting at 10.5 minutes.

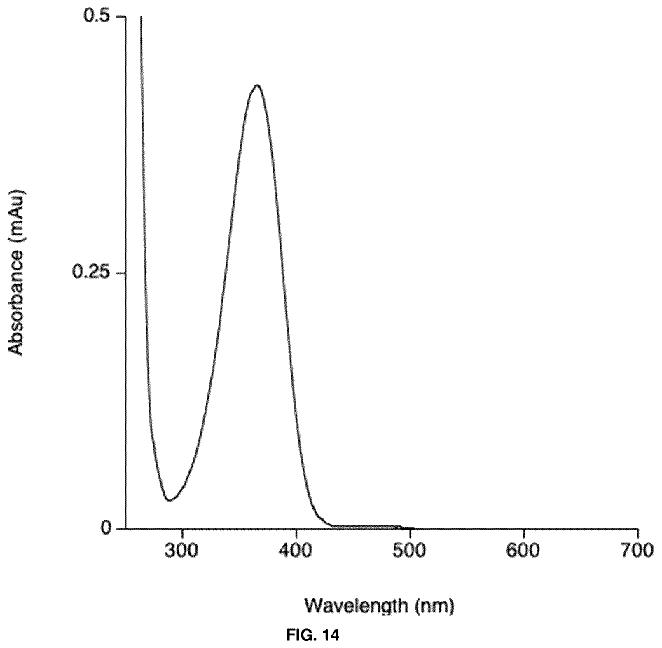

The absorbance spectrum of compound 48 is shown in FIG. 14.

### Synthesis of Compound 49

Compound 49 (ethyl 2-(2-acetomido-4-(2-aminophenyl)-4-oxobutanamido)-4-(2-aminophenyl)-4-oxobutanoate) was prepared as shown in scheme M and as described below.

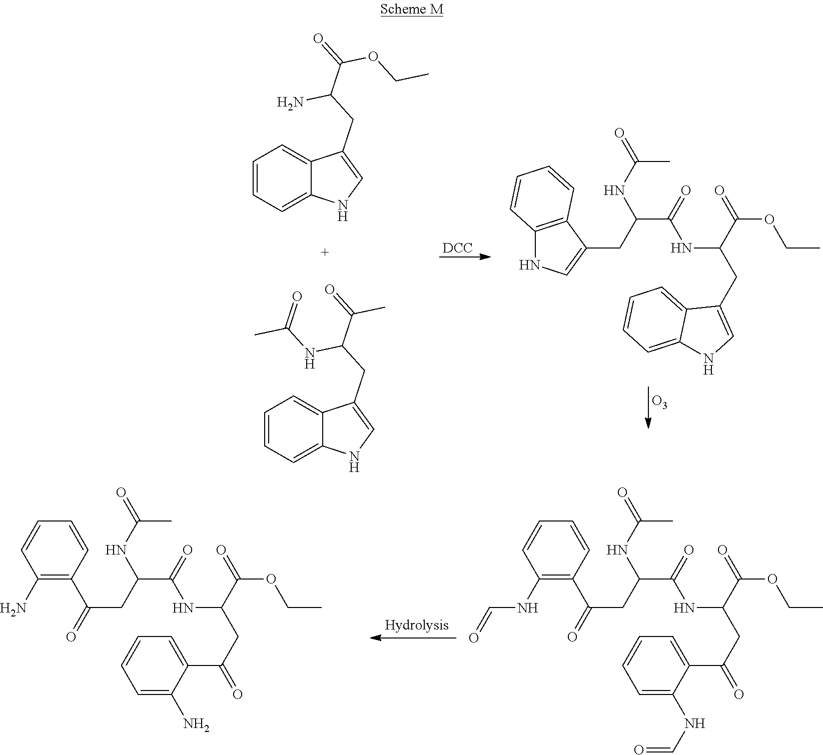

L-tryptophan ethyl ester hydrochloride (CAS 2899-28-7; 5 g; 18 mmol) was combined with N-acetyl-L-tryptophan (CAS 218-34-4; 4.4 g; 18 mmol) in acetonitrile (CAS 75-05-8; 200 ml). To this was added DCC (CAS 538-75-0; 4.4 g; 22 mmol) and 4-methylmorpholine (CAS 109-02-4; 0.65 mL; 60 mmol). This was allowed to react at room temperature overnight, at which point a small amount of water was added to quench any unreacted DCC. Insoluble dicyclohexylurea was removed by filtration and the solvent was removed by evaporation under reduced pressure.

The intermediate, ethyl acetyltryptophyltryptophanate, was resuspended in methanol (CAS 67-56-1, 50 ml) and oxidized under a stream of ozone (A2Z Ozone Inc., MP3000) for 120 minutes (12.5 mmol ozone per hour from ambient air source). A mixture of the formamide product, ethyl 2-(2-acetomido-4-(2-formamidophenyl)-4-oxobutanamido)-4-(2-formamidophenyl)-4-oxobutanoate, and the hydrolyzed product, ethyl 2-(2-acetomido-4-(2-aminophenyl)-4-oxobutanamido)-4-(2-aminophenyl)-4-oxobutanoate, were formed. Incubation at room temperature for five days resulted in hydrolysis of the formamide groups resulting in complete conversion to ethyl 2-(2-acetomido-4-(2-aminophenyl)-4-oxobutanamido)-4-(2-aminophenyl)-4-oxobutanoate. This product was confirmed and distinguished from starting material using HPLC eluting at 10.2 minutes.

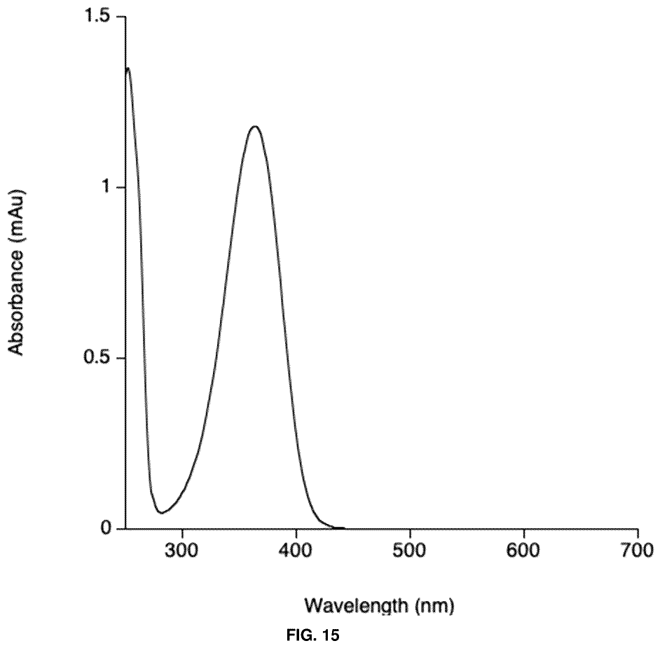

The absorbance spectrum of compound 49 is shown in FIG. 15.

### Synthesis of Compound 50

Compound 50 (ethyl 2-(2-acetomido-4-(2-acetamidophenyl)-4-oxobutanamido)-4-(2-acetamidophenyl)-4-oxobutanoate) was prepared as shown in scheme N and as described below.

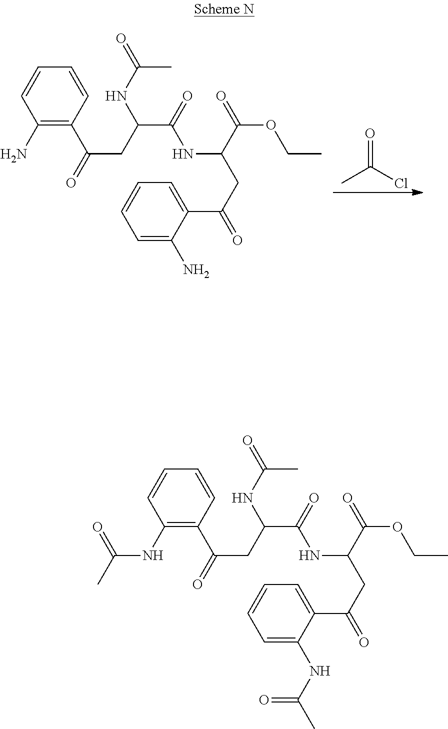

Ethyl 2-(2-acetomido-4-(2-aminophenyl)-4-oxobutanamido)-4-(2-aminophenyl)-4-oxobutanoate (Compound 12; 50 mg; 0.1 mmol) was dissolved in ethyl acetate (CAS 141-78-6; 50 ml). Triethylamine (CAS 121-44-8; 0.084 ml; 0.6 mmol) and acetyl chloride (CAS 75-36-5; 0.017 ml; 0.24 mmol) were added dropwise. The reaction proceeded at reflux for 4 hours, and the product (ethyl 2-(2-acetomido-4-(2-acetamidophenyl)-4-oxobutanamido)-4-(2-acetamidophenyl)-4-oxobutanoate) was distinguished from starting material by HPLC, eluting at 9.6 minutes.

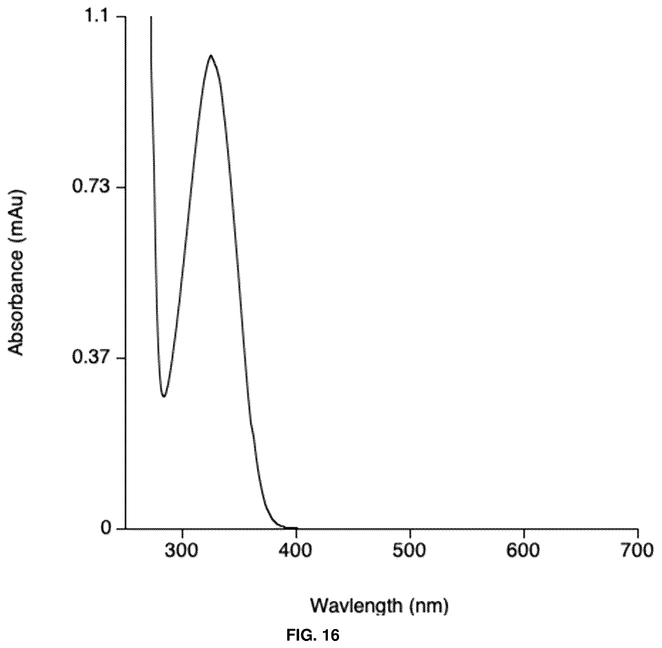

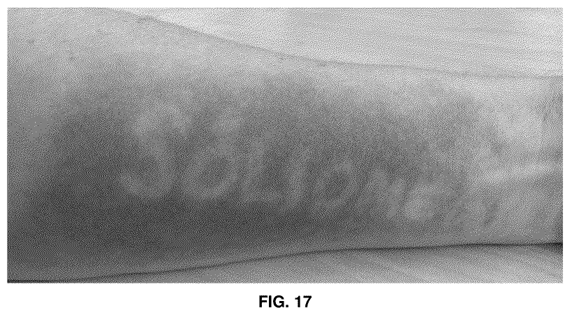

The absorbance spectrum of compound 50 is shown in FIG. 16.

### Antioxidant Activity

Compounds 42, 43, 44, and 46, were tested for their antioxidant capacity as described as previously described using an OxiSelect Hydroxyl Radical Antioxidant Capacity (HORAC) Activity Assay (Cell Biolabs Inc. catalog #sta-346-T). All of these compounds were found to have an antioxidant potential equal, or greater than, gallic acid.

### Hair Protection

Hair protection against UV was evaluated using a standard set of high-pressure differential scanning calorimetry (DSC). The experiments were conducted on samples of hair with and without UV exposure and with and without application of compounds according to the invention. This technique was used to detect any deep structural changes in the hair keratin that occurs as a result of damage, such as that caused by UV which may affect the keratin structure, such as changes to the cysteine bond arrangement or the content of strongly bound water (Milczarek, P., M. Zielinski, and M. L. Garcia. “The mechanism and stability of thermal transitions in hair keratin.” Colloid and Polymer Science 270.11 (1992): 1106-1115; Wortmann, Franz-J., Christian Springob, and Gerhard Sendelbach. “Investigations of cosmetically treated human hair by differential scanning calorimetry in water.” Journal of cosmetic science 53.4 (2002): 219-228).

### Procedure:

1 Bleached Hair tresses (3 g, 8 inches long, 1 inch wide naturally white hair tresses, 9% bleached) were soaked for 48 h while stirring in 4 liters of de-ionized water. Water was changed twice a day. Hair tresses were pretested using DSC to ensure that damage level across the tresses is similar. Selected tresses were disassembled and spread into a thin layer that was fixed using sticky, flexible tape from both sides of the top and the bottom.
2 Tresses were wet with water for 30 seconds after which 0.1 g of 15% sodium laurel sulfate (CAS 151-21-3)/g of hair was applied and massaged into the hair for 30 seconds. The hair was then rinsed for 30 seconds, and cold-blow dried using low velocity setting.
3 A solution of containing 15% (w/w) of compound 43 was then applied to the hair (0.2 ml/g of hair, 30 s massage and then it was cold-blow dried using low velocity setting. Control hair was left untreated with compound 43.
4 The hair samples were then placed into a Q-sun UV chamber (0.67 W/m2 to mimic 4.5× exaggerated conditions) for 2 hours of exposure. They were then washed and treated as specified in step 2 and 3, and placed back into the Q-sun UV chamber for another 2 hours of UV exposure. This was repeated until a total of 6 h of UV exposure were reached.
5 Before subjecting the samples of hair to DSC testing, the samples were washed with shampoo and dried.

JMP analytical software version 17.0 was used to fit the results. The denaturation temperature (Td) marked the height of the denaturation process of the fiber's protein. The denaturation temperature was used to assess the hair matrix cross-linking density. The higher the denaturation temperature, the denser and more compact was the matrix (Wortmann, Franz-J., Christian Springob, and Gerhard Sendelbach. “Investigations of cosmetically treated human hair by differential scanning calorimetry in water.” Journal of cosmetic science 53.4 (2002): 219-228).

The results (Table 2) showed that 6 hours of UV treatment inflicted damage to hair cross-linking density: The denaturation temperature of hair to which no UV was applied was statistically higher than those of other hair groups. The denaturation temperature of the hair treated with compound 43 and treated with UV was higher than that of hair subjected to UV that was untreated with compound 43. It was therefore shown that applying compound 43 to hair protects hair against UV damage.

TABLE 2

| | | Mean Denaturation | |
|---|---|---|---|
| | No. of | Temperature | Standard |
| Hair Sample | replicates | (Td) (° C.) | deviation |
| Control (no UV) | 6 | 154.0 | 0.4 |
| UV + Compound 43 | 6 | 145.8 | 0.4 |
| UV without | 6 | 141.0 | 0.3 |
| compound 43 | | | |

### Skin Protection

To test the protective capability of this class of compounds against damage to skin by UV, a 10% (w/w) solution of compounds was applied to skin at 2 mg/cm² and allowed to dry for 15 minutes. UV light was introduced using a Sperti Sun Lamp (model Miami P107). The application area on the skin was held facing the lamp at a distance of 20 cm for 10 min. Erythema was significantly reduced in areas where the compounds had been applied relative to unprotected skin.

FIG. 17 shows the results of such a test applying compound 42 to the interior forearm skin in the shape of the name “SÓLIOME.” The image was photographed 4 days after UV application showing significantly reduced erythema where the UV filter was applied and afforded protection against UV light compared to the surrounding skin to which no compound 42 was applied.

1. A compound of formula (II) 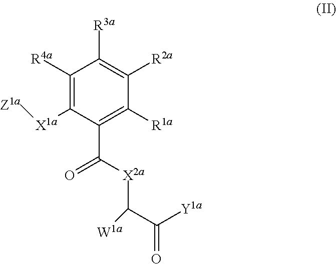
or a tautomer, a stereoisomer, a hydrate, a solvate, a salt, or a polymorph thereof, wherein R^{1a}, R^{2a}, R^{3a}, and R^{4a} are each independently R^{za};
each R^{za} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —OR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —SR^{a}, —S(O)R^{a}, —NR^{a}R^{b}, and —NR^{b}C(O)R^{a};
each R^{a} is independently selected from the group comprising hydrogen, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, and —R^{f1}-R^{g1}; wherein each of said alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene, can be unsubstituted or substituted with one or more R^{za1}; wherein each R^{za1} is independently selected from the group comprising —COO—R^{h}, 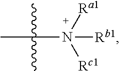
NR^{a1}R^{b}, —R^{f1}-R^{g1}, halo, alkyl, haloalkyl, alkylcarbonyl, alkenyl, and alkynyl; wherein R^{h} is selected from the group comprising hydrogen, alkyl, alkenyl, and alkynyl; R^{a1} is selected from the group comprising hydrogen, alkyl, alkenyl, and alkynyl; R^{b1} is selected from the group comprising hydrogen, alkyl, alkenyl, and alkynyl; R^{c1} is selected from the group comprising hydrogen, alkyl, alkenyl, and alkynyl;
R^{f1} is alkylene, wherein said alkylene can be interrupted by one or more O or S;
R^{g1} is hydrogen, or alkyl;
each R^{b} is independently selected from the group comprising hydrogen, halo, alkyl, haloalkyl, alkylcarbonyl, aryl, heterocyclyl, heteroaryl, cycloalkyl, aryl-alkylene, heteroaryl-alkylene, heterocyclyl-alkylene, and cycloalkyl-alkylene;
X^{1a} is —NR^{b}—;
Z^{1a} is selected from the group comprising hydrogen, —CO—R^{a}, —CO—R^{e}—CO—O—R^{b}, —CO—R^{f}—R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2a} is —(CR^{c}R^{d})ₙ—; wherein n is an integer selected from 1, 2 or 3;
each R^{c} and R^{d} is independently selected from the group comprising hydrogen, halo, alkyl, haloalkyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
R^{e} is selected from the group comprising alkylene, alkenylene, alkynylene, arylene, heterocyclylene, heteroarylene, and cycloalkylene;
R^{f} is alkylene, wherein said alkylene can be interrupted by one or more O or S;
R^{g} is selected from hydrogen, alkyl, N-maleimidyl, N-maleimidyloxy, or N-maleimidyloxycarbonyl;
W^{1a} is selected from —NR^{b}Z^{2a}, —R^{a}, or —OR^{a};
Z^{2a} is selected from the group comprising hydrogen, —CO—R^{a}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
Y^{1a} is selected from —OR^{a}, —R^{a}, or —NR^{a}R^{b}, or Y^{1a} is a moiety of formula (A); 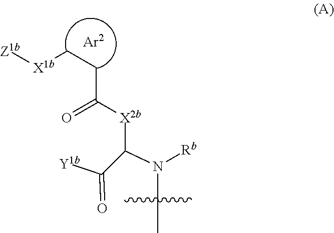
wherein
Ar² is aryl or heteroaryl; wherein each of said aryl or heteroaryl can be unsubstituted or substituted with one or more R^{zb};
each R^{zb} is independently selected from the group comprising hydrogen, halo, alkyl, haloalkyl, —OR^{a}, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, cycloalkyl-alkynylene, —SR^{a}, non-acylated or acylated glycosyl, non-acylated or acylated —O-glycosyl, non-acylated or acylated —NR^{b}-glycosyl, non-acylated or acylated —C-glycosyl, non-acylated or acylated —S-glycosyl, —C(O)—R^{a}, —S(O)R^{a}, —NR^{a}R^{b}, and —NR^{b}C(O)R^{a};
X^{1b} is selected from —NR^{b}—, —O—, —S—, SO₂—O—, or —SO—O—;
Z^{1b} is selected from the group comprising hydrogen, —CO—R^{a}, —CO—R^{e}—CO—O—R^{b}, —CO—R^{f}-R^{g}, —R^{f}-R^{g}, halo, alkyl, haloalkyl, alkylcarbonyl, alkenyl, alkynyl, aryl, heterocyclyl, heteroaryl, cycloalkyl, aryl-alkylene, aryl-alkenylene, aryl-alkynylene, heteroaryl-alkylene, heteroaryl-alkenylene, heteroaryl-alkynylene, heterocyclyl-alkylene, heterocyclyl-alkenylene, heterocyclyl-alkynylene, cycloalkyl-alkylene, cycloalkyl-alkenylene, and cycloalkyl-alkynylene;
X^{2b} is —(CR^{c}R^{d})ₘ—; wherein m is an integer selected from 1, 2 or 3; and
Y^{1b} is selected from —OR^{a}, —NR^{b}R^{a}, or —R^{a};
with the proviso that said compound is not:
2-amino-4-(2-aminophenyl)-4-oxobutanoic acid;
(R)-2-amino-4-(2-aminophenyl)-4-oxobutanoic acid;
(S)-2-amino-4-(2-aminophenyl)-4-oxobutanoic acid;
4-(2-acetamidophenyl)-2-amino-4-oxobutanoic acid;
2-acetamido-4-(2-aminophenyl)-4-oxobutanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
2-amino-4-oxo-4-(2-(((3R,4R,5S,6R)-3,4,5-trihydroxy-6-(hydroxymethyl)tetrahydro-2H-pyran-2-yl)amino)phenyl)butanoic acid;
(2S)-2-amino-4-oxo-4-(2-(((3R,4R,5S,6R)-3,4,5-trihydroxy-6-(hydroxymethyl)tetrahydro-2H-pyran-2-yl)amino)phenyl)butanoic acid;
(S)-2-amino-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
methyl 2-amino-4-(2-aminophenyl)-4-oxobutanoate;
4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
2-acetamido-4-(2-amino-3-(((3R,4S,5S,6R)-3,4,5-trihydroxy-6-(hydroxymethyl)tetrahydro-2H-pyran-2-yl)oxy)phenyl)-4-oxobutanoic acid;
(S)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(R)-2-acetamido-4-(2-amino-3-(((2S,3R,4S,5S,6R)-3,4,5-trihydroxy-6-(hydroxymethyl)tetrahydro-2H-pyran-2-yl)oxy)phenyl)-4-oxobutanoic acid;
(2R,3R,4S,5R)-2-(acetoxymethyl)-6-(2-amino-3-(4-methoxy-4-oxobutanoyl)phenoxy)tetrahydro-2H-pyran-3,4,5-triyl triacetate;
2-amino-4-(2-amino-5-hydroxyphenyl)-4-oxobutanoic acid;
(S)-2-amino-4-(2-amino-5-hydroxyphenyl)-4-oxobutanoic acid;
(R)-2-amino-4-(2-amino-5-hydroxyphenyl)-4-oxobutanoic acid;
5-(2-aminophenyl)-5-oxopentanoic acid;
4-(2-aminophenyl)-4-oxobutanoic acid;
methyl 6-(2-aminophenyl)-6-oxohexanoate;
methyl 4-(2-aminophenyl)-2-((tert-butoxycarbonyl)amino)-4-oxobutanoate;
butyl 2-amino-4-(2-aminophenyl)-4-oxobutanoate;
ethyl (S)-2-amino-4-(2-aminophenyl)-4-oxobutanoate;
(S)-2-amino-4-(2-aminophenyl)-4-oxobutanoic acid diacetate;
(R)-2-amino-4-(2-formamidophenyl)-4-oxobutanoic acid;
(S)-2-amino-4-(2-formamidophenyl)-4-oxobutanoic acid;
2-amino-4-(2-formamidophenyl)-4-oxobutanoic acid;
methyl 2-acetamido-4-(2-acetamidophenyl)-4-oxobutanoate;
4-(2-amino-3-(((2R,3S,4R,5R,6S)-3,4,5-trihydroxy-6-((((2S,3S,4R,5R,6S)-3,4,5-trihydroxy-6-(hydroxymethyl)tetrahydro-2H-pyran-2-yl)oxy)methyl)tetrahydro-2H-pyran-2-yl)oxy)phenyl)-4-oxobutanoic acid;
4-(2-amino-3-hydroxyphenyl)-4-oxobutanoic acid O-beta-D-diglucoside); 4-(2-amino-3-(((2R,3S,4R,5R,6S)-3,4-5-trihydroxy-6-(hydroxymethyl)tetrahydro-2H-pyran-2-yl)oxy)phenyl)-4-oxobutanoic acid;
4-(2-amino-3-hydroxyphenyl)-4-oxobutanoic acid O-beta-d-glucoside;
2-amino-4-(2-formamido-5-hydroxyphenyl)-4-oxobutanoic acid;
2-amino-4-(2-amino-3-chlorophenyl)-4-oxobutanoic acid;
(R)-2-amino-4-(2-amino-3-chlorophenyl)-4-oxobutanoic acid;
(S)-2-amino-4-(2-amino-3-chlorophenyl)-4-oxobutanoic acid;
2-amino-4-(2-amino-4-chlorophenyl)-4-oxobutanoic acid;
(R)-2-amino-4-(2-amino-4-chlorophenyl)-4-oxobutanoic acid;
(S)-2-amino-4-(2-amino-4-chlorophenyl)-4-oxobutanoic acid;
2-amino-4-(2-amino-5-fluorophenyl)-4-oxobutanoic acid;
(R)-2-amino-4-(2-amino-5-fluorophenyl)-4-oxobutanoic acid;
(S)-2-amino-4-(2-amino-5-fluorophenyl)-4-oxobutanoic acid;
2-amino-4-(2-amino-5-bromophenyl)-4-oxobutanoic acid;
(R)-2-amino-4-(2-amino-5-bromophenyl)-4-oxobutanoic acid;
(S)-2-amino-4-(2-amino-5-bromophenyl)-4-oxobutanoic acid;
2-amino-4-(2-amino-5-methylphenyl)-4-oxobutanoic acid;
(R)-2-amino-4-(2-amino-5-methylphenyl)-4-oxobutanoic acid;
(S)-2-amino-4-(2-amino-5-methylphenyl)-4-oxobutanoic acid;
2-amino-4-(2-amino-5-ethylphenyl)-4-oxobutanoic acid;
(R)-2-amino-4-(2-amino-5-ethylphenyl)-4-oxobutanoic acid;
(S)-2-amino-4-(2-amino-5-ethylphenyl)-4-oxobutanoic acid;
2-amino-4-(2-amino-5-propylphenyl)-4-oxobutanoic acid;
(R)-2-amino-4-(2-amino-5-propylphenyl)-4-oxobutanoic acid;
(S)-2-amino-4-(2-amino-5-propylphenyl)-4-oxobutanoic acid;
2-amino-4-(2-amino-5-isopropylphenyl)-4-oxobutanoic acid;
(R)-2-amino-4-(2-amino-5-isopropylphenyl)-4-oxobutanoic acid;
(S)-2-amino-4-(2-amino-5-isopropylphenyl)-4-oxobutanoic acid;
2-amino-4-(2-amino-5-cyclohexylphenyl)-4-oxobutanoic acid;
(R)-2-amino-4-(2-amino-5-cyclohexylphenyl)-4-oxobutanoic acid;
(S)-2-amino-4-(2-amino-5-cyclohexylphenyl)-4-oxobutanoic acid;
2-amino-4-(2-(methylamino)phenyl)-4-oxobutanoic acid;
(S)-2-amino-4-(2-(methylamino)phenyl)-4-oxobutanoic acid;
(R)-2-amino-4-(2-(methylamino)phenyl)-4-oxobutanoic acid;
2-amino-4-(2-(dimethylamino)phenyl)-4-oxobutanoic acid;
(S)-2-amino-4-(2-(dimethylamino)phenyl)-4-oxobutanoic acid;
(R)-2-amino-4-(2-(dimethylamino)phenyl)-4-oxobutanoic acid;
2-amino-4-(2-amino-5-chlorophenyl)-4-oxobutanoic acid;
(R)-2-amino-4-(2-amino-5-chlorophenyl)-4-oxobutanoic acid;
(S)-2-amino-4-(2-amino-5-chlorophenyl)-4-oxobutanoic acid;
2-amino-4-(2-amino-6-chlorophenyl)-4-oxobutanoic acid;
(R)-2-amino-4-(2-amino-6-chlorophenyl)-4-oxobutanoic acid;
(S)-2-amino-4-(2-amino-6-chlorophenyl)-4-oxobutanoic acid;
2-acetamido-4-(2-amino-5-chlorophenyl)-4-oxobutanoic acid;
(R)-2-acetamido-4-(2-amino-5-chlorophenyl)-4-oxobutanoic acid;
(S)-2-acetamido-4-(2-amino-5-chlorophenyl)-4-oxobutanoic acid;
2-amino-4-(2-amino-3-hydroxyphenyl)-4-oxobutanoic acid;
(S-)2-amino-4-(2-amino-3-hydroxyphenyl)-4-oxobutanoic acid;
(R-)2-amino-4-(2-amino-3-hydroxyphenyl)-4-oxobutanoic acid;
methyl (S-)2-amino-4-(2-aminophenyl)-4-oxobutanoate;
2-amino-4-(2-aminophenyl)-4-oxobutanamide;
(R)-2-amino-4-(2-aminophenyl)-4-oxobutanamide;
(S)-2-amino-4-(2-aminophenyl)-4-oxobutanamide;
(S)-2-acetamido-4-(2-acetamidophenyl)-4-oxobutanoic acid;
(S)-2-formamido-4-(2-formamidophenyl)-4-oxobutanoic acid;
4-(2-aminophenyl)-4-oxo-2-phenylbutanamide.

2. The compound according to claim 1, wherein
Y^{a} is a moiety of formula (A), or is selected from —OR^{a}, —R^{a}, or —NR^{a}R^{b};
Ar² is C₆₋₁₀aryl or a 5-6 membered heteroaryl, preferably containing at least one N; wherein each of said aryl or heteroaryl can be unsubstituted or substituted with one or more R^{zb};
wherein R^{za}, R^{zb}, R^{a}, R^{b}, have the same meaning as that defined in claim 1.

3. The compound according to claim 1,
wherein Y^{1a} is a moiety of formula (B), or is selected from OR^{a}, R^{a}, NR^{a}R^{b}, 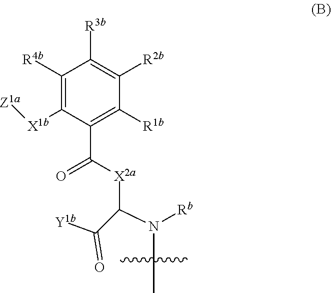
wherein Ria, R^{2a}, R^{3a}, and R^{4a} are each independently R^{za}; and R^{1b}, R^{2b}, R^{3b}, and R^{4b} are each independently R^{zb}, and
wherein Z^{1a}, X^{1a}, X^{2a}, W^{1a}, Z^{1b}, X^{1b}, X^{2b}, Y^{1b}, R^{b}, R^{za}, and R^{zb}, have the same meaning as that defined in claim 1.

4. The compound according to claim 1, wherein
each R^{a} is independently selected from the group comprising hydrogen, halo, C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, and —R^{f1}-R^{g1}; wherein each of said C₁₋₂₀alkyl, haloC₁₋₆alkyl, C₁₋₁₂alkylcarbonyl, C₂₋₂₀alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene, can be unsubstituted or substituted with one or more R^{za1}; wherein each R^{za1} is independently selected from the group comprising —COO—R^{h}, 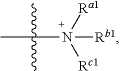
—NR^{a1}R^{b1}, —R^{f1}-R^{g1}, halo, C₁₋₆alkyl, haloC₁₋₆alkyl, C₁₋₆alkylcarbonyl, C₂₋₆alkenyl, and C₂₋₆alkynyl; R^{h} is selected from the group comprising hydrogen, C₁₋₆alkyl, C₂₋₆alkenyl, and C₂₋₆alkynyl; R^{a1} is selected from the group comprising hydrogen, C₁₋₆alkyl, C₂₋₆alkenyl, and C₂₋₆alkynyl; R^{b1} is selected from the group comprising hydrogen, C₁₋₆alkyl, C₂₋₆alkenyl, and C₂₋₆alkynyl; R^{c1} is selected from the group comprising hydrogen, C₁₋₆alkyl, C₂₋₆alkenyl, and C₂₋₆alkynyl;
R^{f1} is C₁₋₂₀alkylene, wherein said C₁₋₂₀alkylene can be interrupted by one or more O or S;
R^{g1} is hydrogen, or C₁₋₂₀alkyl;
each R^{b} is independently selected from the group comprising hydrogen, halo, C₁₋₆alkyl, haloC₁₋₆alkyl, C₁₋₆alkylcarbonyl, C₆₋₁₀aryl, 3-10 membered heterocyclyl, 5-10 membered heteroaryl, and C₃₋₆cycloalkyl;
X^{1a} is —NR^{b}—;
Z^{1a} is selected from the group comprising hydrogen, —CO—R^{a}, —CO—R^{e}—CO—O—R^{b}, —CO—R^{f1}-R^{g1}, —R^{f}-R^{g}, halo, C₁₋₁₂alkyl, haloC₁₋₆alkyl, C₁₋₁₂alkylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene;
R^{e} is selected from the group comprising C₁₋₁₂alkylene, C₂₋₁₂alkenylene, C₂₋₁₂alkynylene, C₆₋₁₀arylene, 5-6 membered heterocyclylene, 5-6 membered heteroarylene, and C₃₋₆cycloalkylene;
R^{f} is C₁₋₂₀alkylene, wherein said alkylene can be interrupted by one or more 0 or S;
R^{g} is selected from hydrogen, alkyl, N-maleimidyl, N-maleimidyloxy, or N-maleimidyloxycarbonyl;
X^{2a} is —(CR^{e}H)ₙ—; wherein n is an integer selected from 1, 2 or 3;
each R^{c} is independently selected from the group comprising hydrogen, halo, C₁₋₆alkyl, haloC₁₋₆alkyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene;
Wia is selected from —NR^{b}Z^{2a}, or —R^{a};
Z^{2a} is selected from the group comprising hydrogen, —CO—R^{a}, halo, C₁₋₁₂alkyl, haloC₁₋₆alkyl, C₁₋₁₂alkylcarbonyl, C₆₋₁₀aryl, 3-10 membered heterocyclyl, 5-10 membered heteroaryl, C₃₋₆cycloalkyl, C₆₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene;
Y^{a} is a moiety of formula (A) or (B) or is —OR^{a}, or —NHR^{a};
X^{1b} is selected from —NR^{b}—, or —O—; preferably X^{1b} is —NR^{b}—;
Z^{1b} is selected from the group comprising hydrogen, —CO—R^{a}, —CO—R^{e}—CO—O—R^{b}, —CO—R^{f1}-R^{g1}, —R^{f}-R^{g}, halo, C₁₋₁₂alkyl, haloC₁₋₆alkyl, C₁₋₁₂alkylcarbonyl, C₂₋₁₂alkenyl, C₂₋₁₂alkynyl, C₆₋₁₀aryl, 3-10 membered heterocyclyl, 5-10 membered heteroaryl, C₃₋₆cycloalkyl, C₁₋₁₀aryl-C₁₋₆alkylene, 5-10 membered heteroaryl-C₁₋₆alkylene, 3-10 membered heterocyclyl-C₁₋₆alkylene, and C₃₋₆cycloalkyl-C₁₋₆alkylene;
X^{2b} is —(CR^{c}H)ₘ—; wherein m is an integer selected from 1, 2 or 3; and
Y^{1b} is —OR^{a}, or —NHR^{a}.

5. The compound according to claim 1, having structural formula (IIA12), (IIA13), (IIA14), (IIA15), (IIA16), (IIA17), (IIA18), (IIB1), (IIB2), (IIB3), (IIB4), (IIB5), (IIB6), (IIB7), or (IIB8), 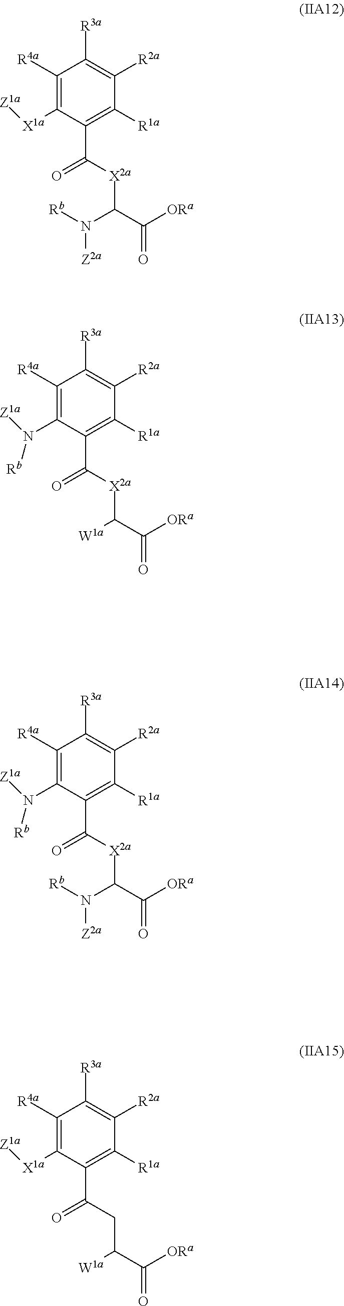 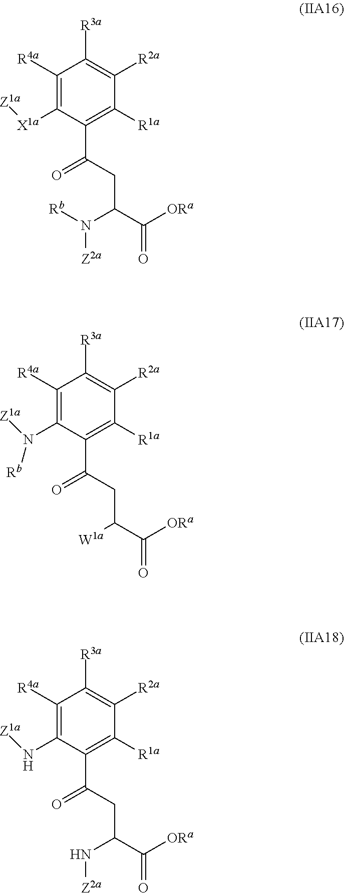 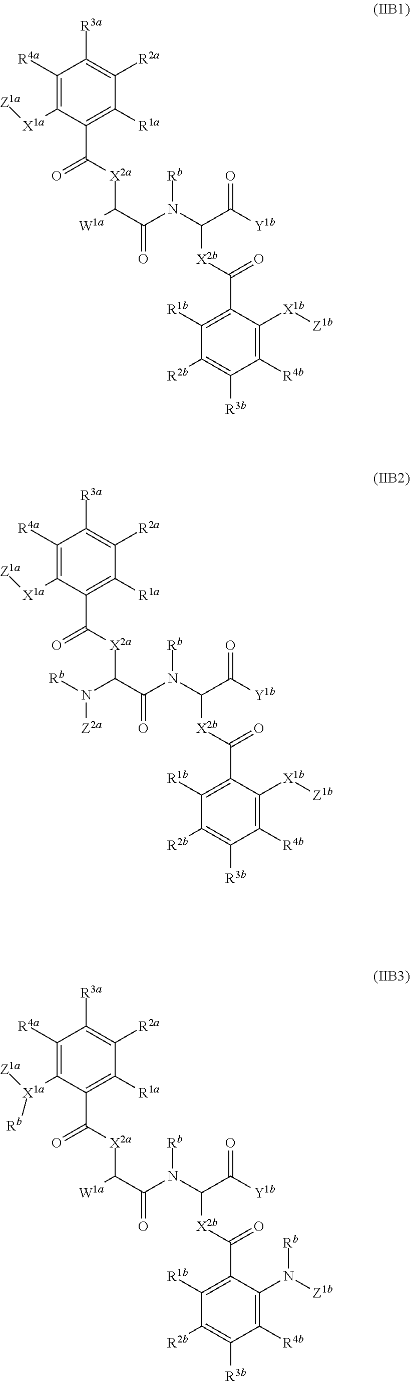 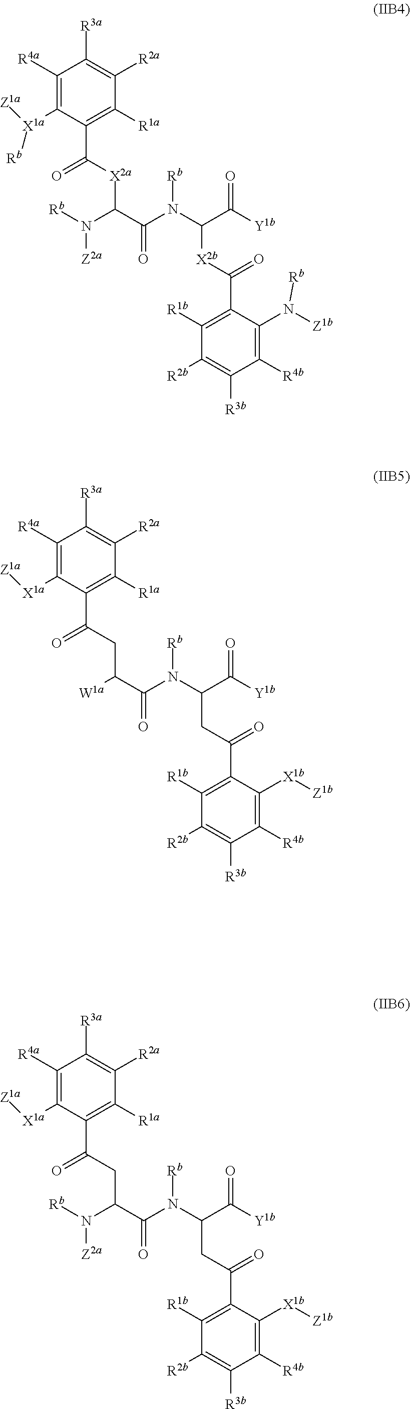 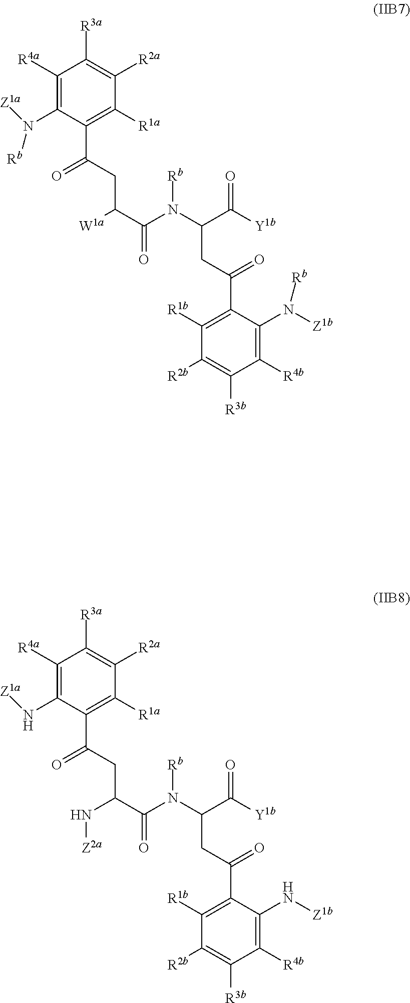
wherein Z^{1a}, X^{1a}, X^{2a}, W^{1a}, Z^{1b}, X^{1b}, X^{2b}, Y^{1a}, Y^{1b}, R^{a}, R^{b}, R^{1a}, R^{2a}, R^{3a}, R^{4a}, R^{1b}, R^{2b}, R^{3b}, R^{4b} have the same meaning as that defined in any one of claims 1-4.

6. The compound according to claim 1, wherein the compound is selected from the group comprising.

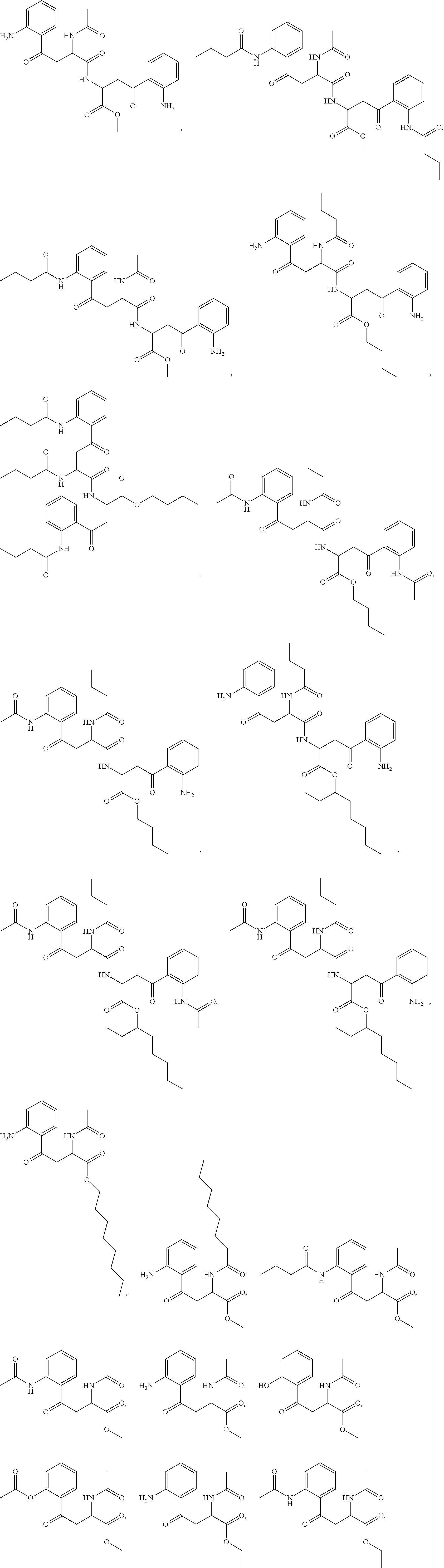

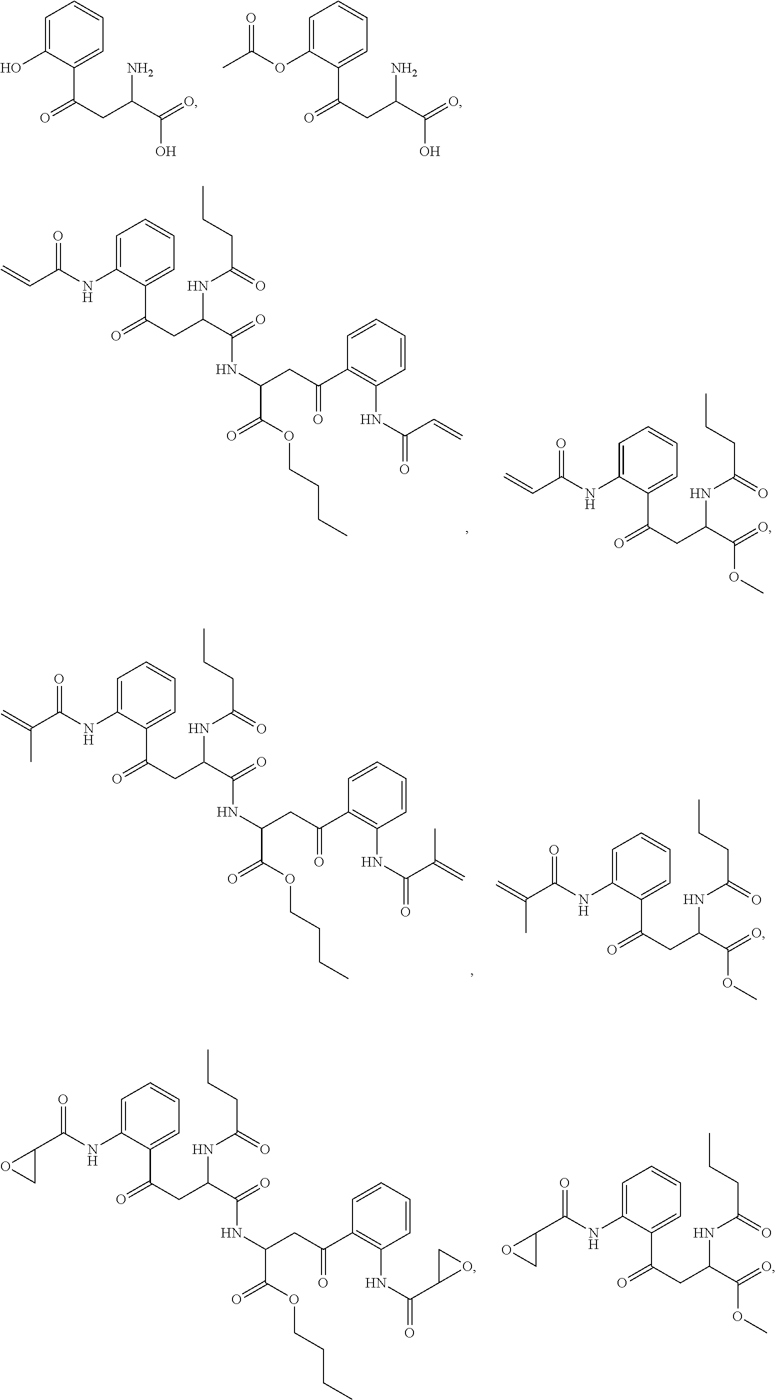

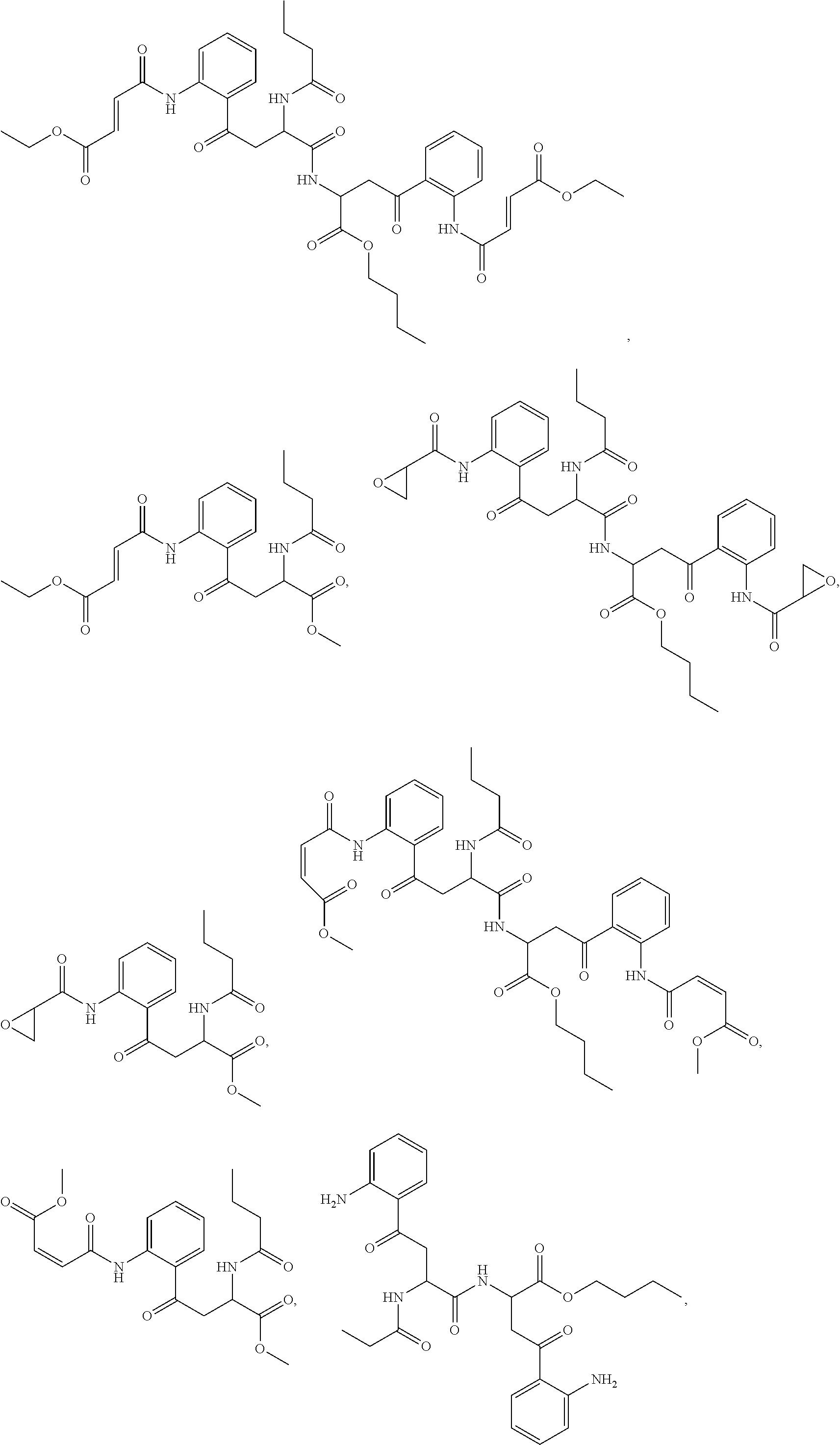

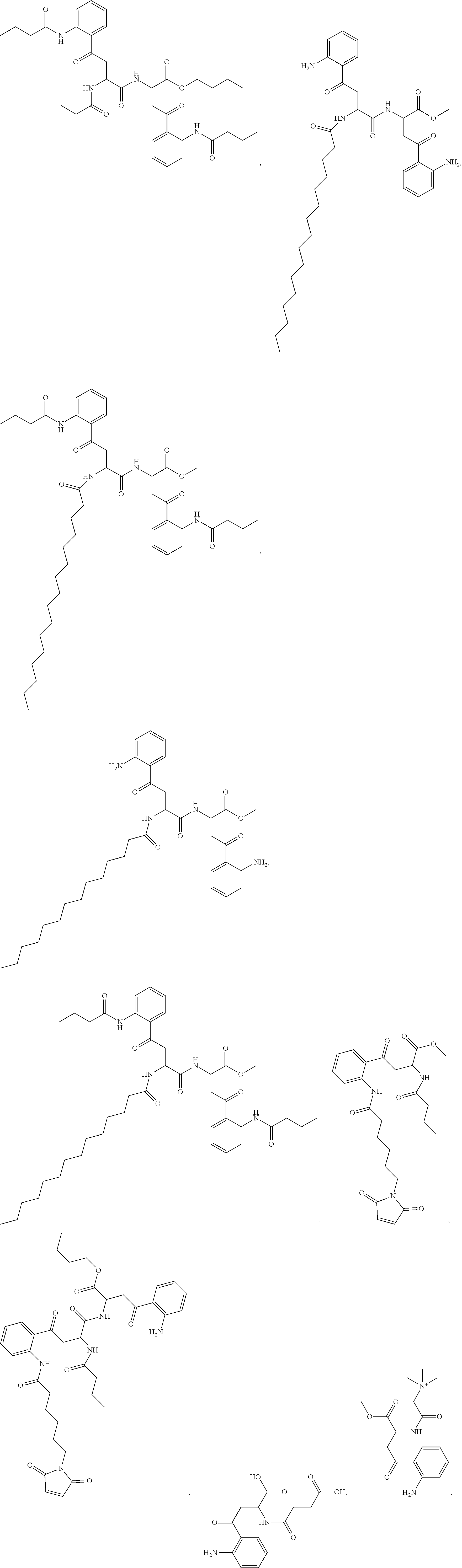

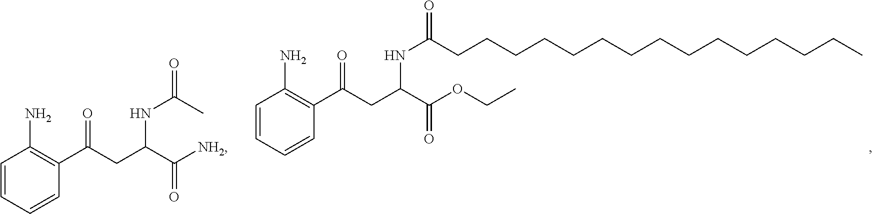 [SMILES: CC(=O)NC(CC(=O)c1ccccc1N)C(N)=O ; CCCCCCCCCCCCCCCC(=O)NC(CC(=O)c1ccccc1N)C(=O)OCC]

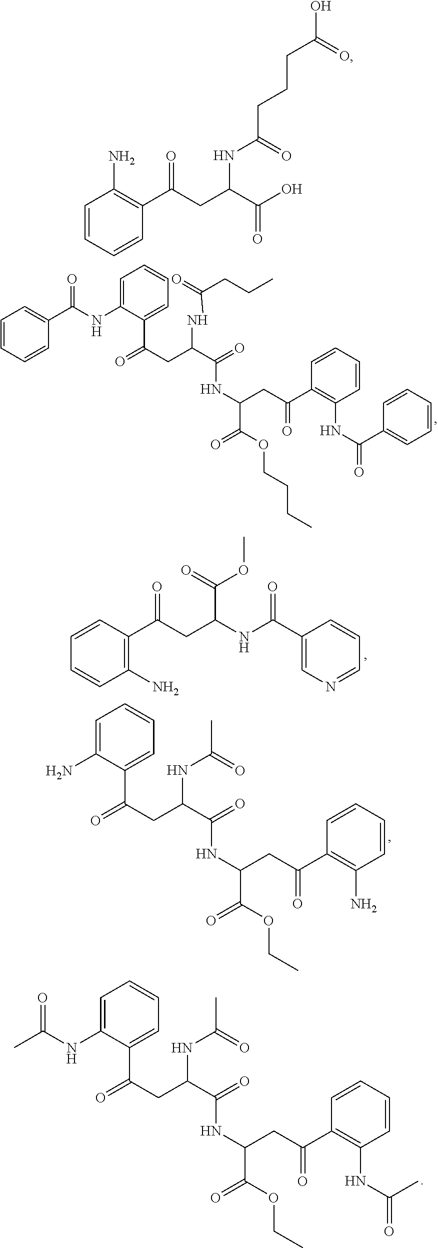

7. The compound according to claim 1, wherein the compound is comprised in a cosmetic or pharmaceutical composition or a consumer product.

8. (canceled)

9. (canceled)

10. (canceled)

11. A method for protecting an article or subject from UV and high-energy visible radiations, the method comprising applying the compound according to claim 1 to the article or subject or including the compound according to claim 1 in the formulation of the article.

12. The method according to claim 11, wherein the compound according to claim 1 acts as a light stabilizer to protect the article against photolytic degradation.

13. The method according to claim 11, wherein the compound according to claim 1 acts an UV and/or high-energy visible light absorbing filter in a consumer product.

14. The method according to claim 13, wherein the consumer product is a cosmetic product.

15. (canceled)

16. The compound according to claim 7, wherein said cosmetic is selected from the group comprising a sunscreen, a moisturizer, makeup, lip protection, a shampoo, and a conditioner.

17. (canceled)

18. (canceled)

19. The composition according to claim 1, wherein the composition is comprised in a packaging material.

20. The method of according to claim 14, wherein the cosmetic product is selected from the group comprising a sunscreen, a moisturizer, makeup, lip protection, a shampoo, and a conditioner t.

21. (canceled)

22. A method to protect a composition from oxidation or to impart antioxidant properties to a composition, the method comprising adding at least one compound according to claim 1 to said composition.
